(19) United States
(12) Patent Application Publication
(10) Pub. No.: US 2012/0276047 A1
(43) Pub. Date: Nov. 1, 2012

(54) FUSED TRICYCLIC COMPOUNDS AND DERIVATIVES THEREOF USEFUL FOR THE TREATMENT OF VIRAL DISEASES

(76) Inventors: Stuart B. Rosenblum, West Orange, NJ (US); Kevin X. Chen, Edison, NJ (US); Joseph A. Kozlowski, Princeton, NJ (US); F. George Njoroge, Warren, NJ (US)

(21) Appl. No.: 13/512,073

(22) PCT Filed: Nov. 23, 2010
(86) PCT No.: PCT/US2010/057708

§ 371 Date: May 25, 2012

Related U.S. Application Data
(60) Provisional application No. 61/264,422, filed on Nov. 25, 2009.

(51) Int. Cl. A61K 31/437; C07D 471/14; C07D 491/048; C07D 495/04; A61K 31/4188; C07D 513/04; A61K 31/429; C07D 498/04; A61K 31/424; A61K 31/4184; A61P 31/12; A61P 37/02; A61K 31/7088; A61K 39/00; A61K 39/395; A61K 38/21; C07D 471/04

(52) U.S. Cl. 424/85.4; 546/82; 514/293; 548/302.1; 514/394; 548/151; 514/366; 548/218; 514/375; 514/44 A; 424/184.1; 424/130.1; 424/141.1

(57) ABSTRACT

The present invention relates to novel fused tricyclic compounds, compositions comprising at least one fused tricyclic compound, and methods of using fused tricyclic compounds for treating or preventing a viral infection or a virus-related disorder in a patient.

### FIELD OF THE INVENTION

The present invention relates to novel Fused Tricyclic Compounds, compositions comprising at least one Fused Tricyclic Compound, and methods of using Fused Tricyclic Compounds for treating or preventing a viral infection or a virus-related disorder in a patient.

### BACKGROUND OF THE INVENTION

Hepatitis C virus (HCV) is a major human pathogen, infecting an estimated 170 million persons worldwide. A substantial fraction of these HCV-infected individuals develop serious progressive liver disease, including cirrhosis and hepatocellular carcinoma, which are often fatal. HCV is a (+)-sense single-stranded enveloped RNA virus that has been implicated as the major causative agent in non-A, non-B hepatitis (NANBH), particularly in blood-associated NANBH (BB-NANBH) (see, International Publication No. WO 89/04669 and European Patent Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

The RNA genome of HCV contains a 5′-nontranslated region (5′ NTR) of 341 nucleotides, a large open reading frame (ORF) encoding a single polypeptide of 3,010 to 3,040 amino acids, and a 3′-nontranslated region (3′-NTR) of variable length of about 230 nucleotides. HCV is similar in amino acid sequence and genome organization to flaviviruses and pestiviruses, and therefore HCV has been classified as a third genus of the family Flaviviridae.

The 5′ NTR, one of the most conserved regions of the viral genome, contains an internal ribosome entry site (IRES) which plays a pivotal role in the initiation of translation of the viral polyprotein. A single long open reading frame encodes a polyprotein, which is co- or post-translationally processed into structural (core, E1, E2 and p7) and nonstructural (NS2, NS3, NS4A, NS4B, NS5A, and NS5B) viral proteins by either cellular or viral proteinases. The 3′ NTR consists of three distinct regions: a variable region of about 38 nucleotides following the stop codon of the polyprotein, a polyuridine tract of variable length with interspersed substitutions of cytidines, and 98 nucleotides (nt) at the very 3′ end which are highly conserved among various HCV isolates. By analogy to other plus-strand RNA viruses, the 3′-NTR is thought to play an important role in viral RNA synthesis. The order of the genes within the genome is: NH₂—C-E1-E2-p7-NS2-NS3-NS4A-NS4B-NS5A-NS5B—COOH.

More recently, attention has been focused toward the identification of inhibitors of HCV NS5A. HCV NS5A is a 447 amino acid phosphoprotein which lacks a defined enzymatic function. It runs as 56 kd and 58 kd bands on gels depending on phosphorylation state (Tanji, et al. J. Virol. 69:3980-3986 (1995)). HCV NS5A resides in replication complex and may be responsible for the switch from replication of RNA to production of infectious virus (Huang, Y, et al., Virology 364:1-9 (2007)).

Multicyclic HCV NS5A inhibitors have been reported. U.S. Patent Publication No. US200810311075 A1 discloses NS5A inhibitors of formula A:

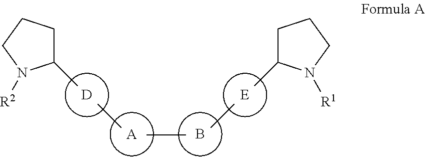

wherein A and B are each phenyl and D and E are each five-membered heteroaromatic rings.

U.S. Patent Publication No. US2008/0044379 A1 discloses NS5A inhibitors of formula B:

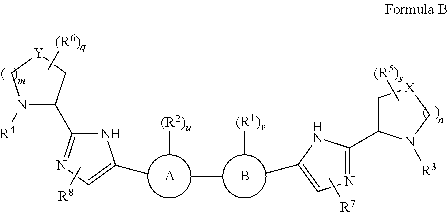

wherein A and B are selected from phenyl and a six-membered heteroaromatic ring.

U.S. Patent Publication No. US2008/0050336 discloses NS5A inhibitors of formula C:

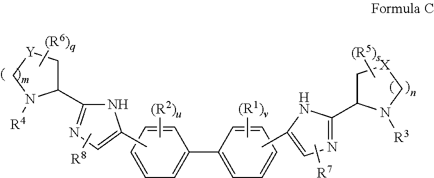

U.S. Patent Publication No. US2008/0044380 A1 discloses NS5A inhibitors of formula D:

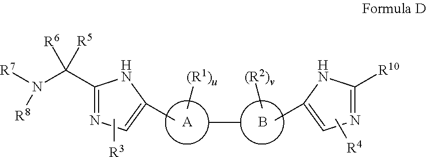

wherein A and B are independently selected from phenyl and a six-membered heteroaromatic ring.

U.S. Patent Publication No. US2009/0202483 discloses hepatitis C virus inhibitors of formula E:

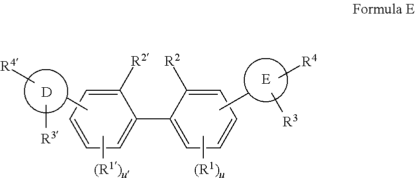

wherein D and E are independently five-membered heteroaromatic rings; R³, R^{3′}, R⁴ and R^{4′} can each be heterocycloalkyl or substituted amino groups; and wherein R² and R^{2′}, together with the carbon atoms to which they are attached, can join to form a five- to eight-membered unsaturated ring.

U.S. Patent Publication No. US2009/020478 discloses hepatitis C virus inhibitors of formula F:

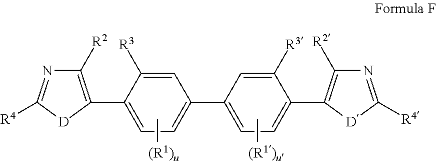

wherein D and D′ can each be NH, O or S; R² and R³, together with the carbon atoms to which they are attached, join to form a five- to eight-membered aromatic or non aromatic carbocyclic or heterocyclic ring; R^{2′} and R^{3′}, together with the carbon atoms to which they are attached, join to form a five- to eight-membered aromatic or non aromatic carbocyclic or heterocyclic ring; and R⁴ and R^{4′} are each independently selected from a nitrogen-containing non-aromatic heterocycle or a substituted aminomethyl group.

Other NS5A inhibitors and their use for reducing viral load in HCV infected humans have been described in U.S. Patent Publication No. US2006/0276511.

Despite the intensive effort directed at the treatment and prevention of HCV and related viral infections, there exists a need in the art for non-peptide, small-molecule compounds having desirable or improved physicochemical properties that are useful for inhibiting, viruses and treating viral infections and virus-related disorders. Specifically, there exists a need for compounds and methods useful for modulating the activity of serine proteases, including NS3, as well as modulating HCV NS2, NS4A, NS4B, NS5A, NS5B and IRES.

Due to the rapidly mutating rate of HCV and thus the emergence of resistant strains, there exists a need for small molecule compounds which act by different mechanisms that can be combined to treat viral infections and virus-related disorders. This invention addresses that need.

### SUMMARY OF THE INVENTION

In one aspect, the present invention provides Compounds of Formula (I):

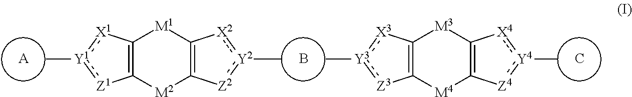

and pharmaceutically acceptable salts, solvates, prodrugs, esters and stereoisomers thereof, wherein each dotted line represents an optional and additional bond, such that only one optional and additional bond can be attached to each of Y¹, Y², Y³ and Y⁴, and wherein:

A is -alkylene-N(R¹¹)(R¹³) or heterocycloalkyl, wherein said heterocycloalkyl group can be optionally and independently substituted with from one to three R⁴ groups, and wherein said heterocycloalkyl group can be optionally fused to a cycloalkyl group or a benzene group;

B is a bond, C₁-C₃ alkylene, —C(R⁵)≡C(R⁵)—, phenylene, monocyclic cycloalkylene, monocyclic cycloalkenylene, monocyclic heterocycloalkylene, monocycle heterocycloalkenylene or monocyclic heteroarylene, wherein said phenylene group or said monocyclic cycloalkylene group can be optionally and independently substituted with one or more R¹⁴ groups, and wherein said phenylene group or said monocyclic cycloalkylene group can be optionally and independently substituted on one or more ring nitrogen atoms with R⁶ and on one or more ring carbon atoms with R¹⁴;

C is -alkylene-N(R¹¹)(R¹³) or heterocycloalkyl, wherein said heterocycloalkyl group can be optionally and independently substituted with from one to three R⁴ groups, and wherein said heterocycloalkyl group can be optionally fused to a cycloalkyl group or a benzene group;

M¹ is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—;

M² is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R₇)₂]ₘ—N(R⁶)S(O)₂N(R⁶)—[C(R⁷)₂]ₘ—, such that at least one of M¹ and M² is other than a bond, and such that the central ring of formula (I) that contains M¹ and M² has from 5 to 10 total ring atoms, and wherein two vicinal R⁷ groups of M¹ or M² together with the carbon atoms to which they are attached, can optionally join to form a 3- to 7-membered cycloalkyl group, a 3- to 7-membered heterocycloalkyl group, or a 5- to 6-membered heteroaryl group;

M³ is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷₂]ₘ—;

M⁴ is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—, such that least one of M³ and M⁴ is other than a bond, and such that the central ring of formula (I) that contains M³ and M⁴ has from 5 to 10 total ring atoms, and wherein two vicinal R⁷ groups of M³ or M⁴ together with the carbon atoms to which they are attached, can optionally join to form a 3- to 7-membered cycloalkyl group, a 3- to 7-membered heterocycloalkyl group, or a 5- to 6-membered heteroaryl group;

X¹ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X¹ is not present, and X¹ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to X¹ is present, such that X¹ and Z¹ cannot each be a bond, and such that when X¹ is —C(R⁵)— or —N—, then Z¹ is other than a bond;

X² is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X² is not present, and X² is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to X² is present, such that X² and Z² cannot each be a bond, and such that when X² is —C(R⁵)— or —N—, then Z² is other than a bond;

X³ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X³ is not present, and X³ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to X³ is present, such that X³ and Z³ cannot each be a bond, and such that when X³ is —C(R⁵)— or —N—, then Z³ is other than a bond;

X⁴ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X⁴ is not present, and X⁴ is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to X⁴ is present, such that X⁴ and Z⁴ cannot each be a bond, and such that when X⁴ is —C(R⁵)— or —N—, then Z⁴ is other than a bond;

Y¹ is —C— when an optional and additional bond to Y¹ is present, and Y¹ is —CH— when an optional and additional bond to Y¹ is absent;

Y² is —C— when an optional and additional bond to Y² is present, and Y² is —CH— when an optional and additional bond to Y² is absent;

Y³ is —C— when an optional and additional bond to Y³ is present, and Y³ is —CH— when an optional and additional bond to Y³ is absent;

Y⁴ is —C— when an optional and additional bond to Y⁴ is present, and Y⁴ is —CH— when an optional and additional bond to Y⁴ is absent;

Z¹ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z¹ is not present, and Z¹ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to Z¹ is present, such that such that the ring in formula (I) containing X¹, Y¹ and Z¹ has 5 or 6 total ring atoms;

Z² is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z² is not present, and Z² is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to Z² is present, such that the ring in formula (I) containing X², Y² and Z² has 5 or 6 total ring atoms;

Z³ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z³ is not present, and Z³ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to Z³ is present, such that such that the ring in formula (I) containing X³, Y³ and Z³ has 5 or 6 total ring atoms;

Z⁴ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z⁴ is not present, and Z⁴ is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to Z⁴ is present, such that the ring in formula (I) containing X⁴, Y⁴ and Z⁴ has 5 or 6 total ring atoms;

each occurrence of R¹ is independently alkyl, haloalkyl, aryl, cycloalkyl, heterocycloalkyl or heteroaryl, wherein said aryl group, said cycloalkyl group, said heterocycloalkyl group or said heteroaryl group can be optionally and independently substituted with R²;

each occurrence of R² is independently alkyl, halo, haloalkyl, aryl, heterocycloalkyl, heteroaryl, —CN, —OR³, —N(R³)₂, —C(O)R¹², —C(O)OR³, —C(O)N(R³)₂, —NHC(O)R¹², —NHC(O)NHR³, —NHC(O)OR³, —OC(O)R¹², —SR³ or —S(O)₂R¹²;

each occurrence of R³ is independently H, alkyl, haloalkyl, aryl, cycloalkyl, heterocycloalkyl or heteroaryl;

each occurrence of R⁴ is independently halo, —C(O)—[C(R⁵)₂]_{q}N(R⁶)₂, —C(O)—[CH(R⁵)]_{q}N(R⁶)C(O)—R¹, —C(O)—[CH(R⁵)]_{q}N(R⁶)C(O)O—R¹, —C(O)—[CH(R⁵)]_{q}C(O)O—R¹, —C(O)[CH(R⁵)]_{q}N(R⁶)SO₂—R¹ or -alkylene-N(R⁶)—[CH(R⁵)]_{q}—N(R⁶)—C(O)O—R¹;

each occurrence of R⁵ is independently H; —C₁-C₆ alkyl, 3 to 7-membered cycloalkyl, aryl or heteroaryl;

each occurrence of R⁶ is independently H, —C₁-C₆ alkyl, 3 to 7-membered cycloalkyl, 4 to 7-membered heterocycloalkyl, aryl, or heteroaryl, wherein said 3 to 7-membered cycloalkyl group, said 4 to 7-membered heterocycloalkyl group, said aryl group or said heteroaryl group can be optionally and independently substituted with up to two R⁸ groups, and wherein two R⁶ groups that are attached to a common nitrogen atom, together with the nitrogen atom to which they are attached, can optionally join to form a 4 to 7-membered heterocycloalkyl group;

each occurrence of R⁷ is independently H, —C₁-C₆ alkyl, 3 to 7-membered cycloalkyl, 3 to 7-membered heterocycloalkyl, aryl or heteroaryl, wherein said 3 to 7-membered cycloalkyl group, said 3 to 7-membered heterocycloalkyl group, said aryl group or said heteroaryl group can be optionally and independently substituted with up to 3 substituents, which can the same or different, and are selected from C₁-C₆ alkyl, halo, —C₁-C₆ haloalkyl, —C₁-C₆ hydroxyalkyl, —OH, —C(O)NH—(C₁-C₆ alkyl), —C(O)N(C₁-C₆ alkyl)₂, —O—(C₁-C₆ alkyl), —NH₂, —NH(C₁-C₆ alkyl), —N(C₁-C₆ alkyl)₂ and —NHC(O)—(C₁-C₆alkyl), and wherein two geminal R⁷ groups, together with the common carbon atom to which they are attached, can optionally join to form —C(O)—, —C(S)—, —C(═NR⁹)—, —C(═NOR⁹)—, a 3 to 7-membered cycloalkyl group or a 3 to 7-membered heterocycloalkyl group, such that no two adjacent —C(R⁷)₂— groups can join to form a —C(O)—C(O)—, —C(S)—C(S)—, —C(O)—C(S)— or —C(S)—C(O)— group;

each occurrence of R⁸ is independently H or —C₁-C₆ alkyl;

each occurrence of R⁹ is independently H, —C₁-C₆ alkyl, 3 to 6-membered cycloalkyl or 4 to 6-membered heterocycloalkyl;

R¹¹ is 3 to 7-membered cycloalkyl, 3 to 7-membered heterocycloalkyl, aryl or heteroaryl, wherein said 3 to 7-membered cycloalkyl group, said 3 to 7-membered heterocycloalkyl group, said aryl group or said heteroaryl group can be optionally and independently substituted with up to three R² groups;

each occurrence of R¹² is independently alkyl, haloalkyl, aryl, cycloalkyl, heterocycloalkyl or heteroaryl;

each occurrence of R¹³ is independently —C(O)—[C(R⁷)₂]_{q}N(R⁶)₂, —C(O)—[C(R⁷)₂]_{q}N(R⁶)C(O)—R¹, —C(O)—[C(R⁷)₂]_{q}N(R⁶)C(O)O—R¹, —C(O)—[C(R⁷)₂]_{q}C(O)O—R¹, —C(O)[C(R⁷)₂]_{q}N(R⁶)SO₂—R¹ or -alkylene-N(R⁶)—[C(R⁷)₂]_{q}-N(R⁶)—C(O)O—R¹;

each occurrence of R¹⁴ is H, —C₁-C₆ alkyl, 3 to 7-membered cycloalkyl, 3 to 7-membered heterocycloalkyl, aryl, heteroaryl, halo, haloalkyl, —CN, —OR³, —N(R³)₂, —C(O)R¹², —C(O)OR³, —C(O)N(R³)₂, —NHC(O)R¹², —NHC(O)NHR³, —NHC(O)OR³, —OC(O)R¹², —SR³ or —S(O)₂R¹²;

each occurrence of m is independently an integer ranging from 0 to 2; and

each occurrence of q is independently an integer ranging from 1 to 4,

such that at least one of the rings containing: (i) X², Y² and Z² or (ii) X³, Y³ and Z³ is other than a benzene ring.

The Compounds of Formula (I) (also referred to herein as the “Fused Tricyclic Compounds”) and pharmaceutically acceptable salts, solvates, esters and prodrugs thereof can be useful for treating or preventing a viral infection or a virus-related disorder in a patient.

The Fused Tricyclic Compounds or pharmaceutically acceptable salts, solvates, prodrugs or esters thereof can also be useful for treating or preventing a viral infection or a virus-related disorder in a patient.

Also provided by the invention are methods for treating or preventing a viral infection or a virus-related disorder in a patient, comprising administering to the patient an effective amount of at least one Fused Tricyclic Compound.

The present invention further provides pharmaceutical compositions comprising an effective amount of at least one Fused Tricyclic Compound or a pharmaceutically acceptable salt, solvate, ester or prodrug thereof, and a pharmaceutically acceptable carrier. The compositions can be useful for treating or preventing a viral infection or a virus-related disorder in a patient

The details of the invention are set forth in the accompanying detailed description below.

Although Many methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

The present invention provides Fused Tricyclic Compounds, pharmaceutical compositions comprising at least one Fused Tricyclic Compound, and methods of using the Fused Tricyclic Compounds for treating or preventing a viral infection or a virus-related disorder in a patient.

### DEFINITIONS AND ABBREVIATIONS

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,”“—O-alkyl,” etc. . . . .

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of Fused Tricyclic Compound and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl; —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched. The term “C₁-C₆ alkyl” refers to an alkyl group having from 1 to 6 carbon atoms.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkenyl group is unsubstituted. The term “C₂-C₆ alkenyl” refers to an alkenyl group having from 2 to 6 carbon atoms.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkynyl group is unsubstituted. The term “C₂-C₆ alkynyl” refers to an alkynyl group having from 2 to 6 carbon atoms.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is unsubstituted. In another embodiment, an aryl group is phenyl.

The two “arylene,” as used herein, refers to a divalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

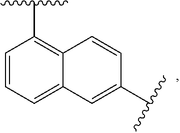

is understood to represent both:

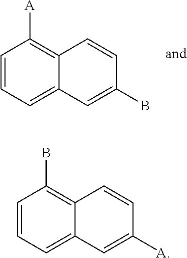

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

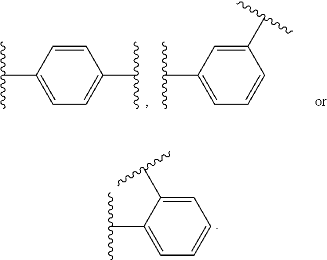

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. In one embodiment, a cycloalkyl group is monocyclic. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 7-membered cycloalkyl” refers to a cycloalkyl group having from 3 to 7 ring carbon atoms. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

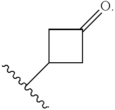

The term “cycloalkylene,” as used herein, refers to a cycloalkyl group, as defined above, wherein one of the cycloalkyl group's hydrogen atoms has been replaced with a bond. Non-limiting examples of cycloalkylene groups include cyclopropylene, cyclobutylene, cyclopentylene, cyclohexylene and cyclopentylene. In one embodiment, a cycloalkylene group has from 3 to about 8 ring carbon atoms. In one embodiment, a cycloalkylene group has from 3 to about 7 ring carbon atoms. In one embodiment, a cycloalkylene group has from 5 to about 7 ring carbon atoms. In another embodiment, a cycloalkylene group is monocyclic. In another embodiment, a cycloalkylene group is bicyclic.

The term “cycloalkenyl,” as used herein, refers to a non-aromatic mono- or multicyclic ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. In one embodiment, a cycloalkenyl group is monocyclic. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. In one embodiment, a cycloalkenyl group is unsubstituted. In another embodiment, a cycloalkenyl group is cyclopentenyl. In another embodiment, a cycloalkenyl group is cyclohexenyl. The term “4 to 7-membered cycloalkenyl” refers to a cycloalkenyl group having from 4 to 7 ring carbon atoms.

The term “cycloalkenylene,” as used herein, refers to a cycloalkenyl group, as defined above, wherein one of the cycloalkenyl group's hydrogen atoms has been replaced with a bond. Non-limiting examples of cycloalkenylene groups include cyclopropenylene, cyclobutenylene, cyclopentenylene, cyclohexenylene and cyclopentenylene. In one embodiment, a cycloalkenylene group has from 3 to about 8 ring carbon atoms. In one embodiment, a cycloalkenylene group has from 3 to about 7 ring carbon atoms. In one embodiment, a cycloalkenylene group has from 5 to about 7 ring carbon atoms. In another embodiment, a cycloalkenylene group is monocyclic. In another embodiment, a cycloalkenylene group is bicyclic.

“Halo” means —F, —Cl, —Br or —I. In one embodiment, halo refers to —F, —Cl or —Br.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein, one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term“heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is unsubstituted. In another embodiment, a heteroaryl group is a 5-membered heteroaryl. In another embodiment, a heteroaryl group is a 6-membered heteroaryl. In another embodiment, a heteroaryl group comprises a 5 or 6-membered heteroaryl group fused to a benzene ring. The term “3 to 7-membered cycloalkyl” refers to a cycloalkyl group having from 3 to 8 ring carbon atoms. The term “heteroarylene,” as used herein, refers to a divalent group derived from a heteroaryl group, as defined above, by removal of a hydrogen atom from a ring carbon or ring heteroatom of a heteroaryl group. A heteroarylene group can be derived from a monocyclic or multicyclic ring system comprising about 5 to about 14 ring atoms, wherein from 1 to 4 of the ring atoms are each independently O, N or S and the remaining ring atoms are carbon atoms. In one embodiment, a heteroarylene group has 5 to 10 ring atoms. In another embodiment, a heteroarylene group is monocyclic. In another embodiment, a heteroarylene group is monocyclic and has 5 or 6 ring atoms. In another embodiment, a heteroarylene group is bicyclic and has 9 or 10 ring atoms. A heteroarylene group can be optionally substituted by one or more “ring system substituents” which may be the same or different, and are as defined herein below. A heteroarylene group is joined via a ring carbon atom or by a nitrogen atom with an open valence, and any nitrogen atom of a heteroarylene can be optionally oxidized to the corresponding N-oxide. The term “heteroarylene” also encompasses a heteroarylene group, as defined above, which is fused to a benzene ring. Non-limiting examples of heteroarylenes include pyridylene, pyrazinylene, furanylene, thienylene, pyrimidinylene, pyridonylene (including those derived from N-substituted pyridonyls), isoxazolylene, isothiazolylene, oxazolylene, oxadiazolylene, thiazolylene, pyrazolylene, furazanylene, pyrrolylene, triazolylene, 1,2,4-thiadiazolylene, pyrazinylene, pyridazinylene, quinoxalinylene, phthalazinylene, oxindolylene, imidazo[1,2-a]pyridinylene, imidazo[2,1-b]thiazolylene, benzofurazanylene, indolylene, azaindolylene, benzimidazolylene, benzothienylene, quinolinylene, imidazolylene, benzimidazolylene, thienopyridylene, quinazolinylene, thienopyrimidylene, pyrrolopyridylene, imidazopyridylene, isoquinolinylene, benzoazaindolylene, 1,2,4-triazinylene, benzothiazolylene and the like, and all isomeric forms thereof. The term “heteroarylene” also refers to partially saturated heteroarylene moieties such as, for example, tetrahydroisoquinolylene, tetrahydroquinolylene, and the like. A heteroarylene group is divalent and either available bond on a heteroarylene ring can connect to either group flanking the heteroarylene group. For example, the group “A-heteroarylene-B,” wherein the heteroarylene group is:

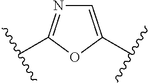

is understood to represent both:

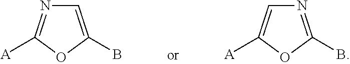

In one embodiment, a heteroarylene group is unsubstituted. In another embodiment, a heteroarylene group is a 5-membered heteroarylene. In another embodiment, a heteroarylene group is a 6-membered heteroarylene. In another embodiment, a heteroarylene group comprises a 5 or 6-membered heteroarylene group fused to a benzene ring. In another embodiment, a heteroarylene group is:

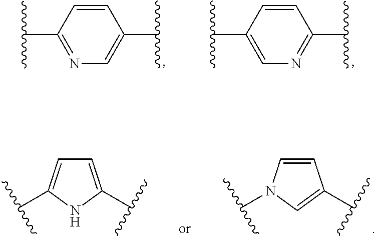

The term “heterocycloalkyl,” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. A heterocycloalkyl group can be joined via a ring carbon or ring nitrogen atom. In one embodiment, a heterocycloalkyl group has from about 3 to about 7 ring atoms. In another embodiment, a heterocycloalkyl group has 5 or 6 ring atoms. In another embodiment, a heterocycloalkyl group is monocyclic. In still another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, dioxanyl, tetrahydrofuranyl, tetrahydrothiophenyl, lactam, lactone and the like, and all isomers thereof. A ring carbon atom of a heterocycloalkyl group may be functionalized as a carbonyl group. An illustrative example of such a heterocycloalkyl group is pyrrolidonyl:

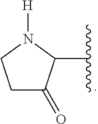

In one embodiment, a heterocycloalkyl group is unsubstituted. In another embodiment, a heterocycloalkyl group is a 5-membered heterocycloalkyl. In another embodiment, a heterocycloalkyl group is a 6-membered heterocycloalkyl. The term “3 to 7-membered cycloalkyl” refers to a heterocycloalkyl group having from 3 to 7 ring atoms.

The term “heterocycloalkylene,” as used herein, refers to a divalent group derived from a heterocycloalkyl group, as defined above, by removal of a hydrogen atom from a ring carbon or ring heteroatom of a heterocycloalkyl group. A heterocycloalkylene group can be derived from a monocyclic or multicyclic ring system comprising about 5 to about 14 ring atoms, wherein from 1 to 4 of the ring atoms are each independently O, N or S and the remaining ring atoms are carbon atoms. In one embodiment, a heterocycloalkylene group has 5 to 10 ring atoms. In another embodiment, a heterocycloalkylene group is monocyclic. In another embodiment, a heterocycloalkylene group is monocyclic and has 5 or 6 ring atoms. In another embodiment, a heterocycloalkylene group is bicyclic and has 9 or 10 ring atoms. A heterocycloalkylene group can be optionally substituted by one or more “ring system substituents” which may be the same or different, and are as defined herein below. A heterocycloalkylene group is joined via a ring carbon atom or via a ring nitrogen atom with an open valence, and any nitrogen atom of a heterocycloalkylene can be optionally oxidized to the corresponding N-oxide. The term “heterocycloalkylene” also encompasses a heterocycloalkylene group, as defined above, which is fused to a benzene ring. Non-limiting examples of heterocycloalkylenes include oxetanylene, piperidylene, pyrrolidinylene, piperazinylene, morpholinylene, thiomorpholinylene, thiazolidinylene, 1,4-dioxanylene, tetrahydrofuranylene, tetrahydrothiophenylene, and the like, and all isomeric forms thereof.

The term “heterocycloalkenyl,” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group is monocyclic. In another embodiment, a heterocycloalkenyl group has from 4 to 7 ring atoms. In another embodiment, a heterocycloalkenyl group is monocyclic and has 5 or 6 ring atoms. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrethiazolyl, 3,4-dihydro-2H-pyranyl, dihydrofuranyl, fluoro-substituted dihydrofaranyl, 7-oxabicyclo[2.2.1]heptenyl, dihydrothiophenyl, dihydrothiopyranyl, and the like: A ring carbon atom of a heterocycloalkenyl group may be functionalized as a carbonyl group. In one embodiment, a heterocycloalkenyl group is unsubstituted. In another embodiment, a heterocycloalkenyl group is a 5-membered heterocycloalkenyl. In another embodiment, a heterocycloalkenyl group is a 6-membered heterocycloalkenyl. The term “4 to 7-membered heterocycloalkenyl” refers to a heterocycloalkenyl group having from 4 to 7 ring atoms.

The term “heterocycloalkenylene,” as used herein, refers to a divalent group derived from a heterocycloalkenyl group, as defined above, by removal of a hydrogen atom from a ring carbon or ring heteroatom of a heterocycloalkenyl group. A heterocycloalkenylene group can be derived from a monocyclic or multicyclic ring system comprising about 5 to about 14 ring atoms, wherein from 1 to 4 of the ring atoms are each independently O, N or S and the remaining ring atoms are carbon atoms. In one embodiment, a heterocycloalkenylene group has 5 to 10 ring atoms. In another embodiment, a heterocycloalkenylene group is monocyclic. In another embodiment, a heterocycloalkenylene group is monocyclic and has 5 or 6 ring atoms. In another embodiment, a heterocycloalkenylene group is bicyclic and has 9 or 10 ring atoms. A heterocycloalkenylene group can be optionally substituted by one or more “ring system substituents” which may be the same or different, and are as defined herein below. A heterocycloalkenylene group is joined via a ring carbon atom or via a ring nitrogen atom with an open valence, and any nitrogen atom of a heterocycloalkenylene can be optionally oxidized to the corresponding N-oxide. The term “heterocycloalkenylene” also encompasses a heterocycloalkenylene group, as defined above, which is fused to a benzene ring. Non-limiting examples of heterocycloalkenylenes include 1,2,3,4-tetrahydropyridinylene, 1,2-dihydropyridinylene, 1,4-dihydropyridinylene, 1,2,3,6-tetrahydropyridinylene, 1,4,5,6-tetrahydropyrimidinylene, 2-pyrrolinylene, 3-pyrrolinylene, 2-imidazolinylene, 2-pyrazolinylene, dihydroimidazolylene, dihydrooxazolylene, dihydrooxadiazolylene, dihydrothiazolylene, 3,4-dihydro-2H-pyranylene, dihydrofuranylene, fluoro-substituted dihydrofuranylene, 7-oxabicyclo[2.2.1]heptenylene, dihydrothiophenylene, dihydrothiopyranylene, and the like, and all isomeric forms thereof.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -arylene-alkyl, -alkylene-heteroaryl, -alkenylene-heteroaryl, -alkynylene-heteroaryl, hydroxy, hydroxyalkyl, haloalkyl, —O-alkyl, —O-haloalkyl, -alkylene-O-alkyl, —O-aryl, aralkoxy, acyl, aroyl, halo, nitro, cyano, —SF₅, carboxy, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, Y₁Y₂N-alkyl-, Y₁Y₂NC(O)—, and Y₁Y₂NS(O)₂—, wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

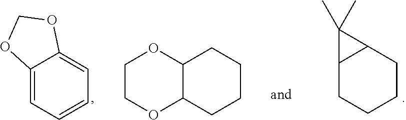

The team “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g., from a reaction mixture), or natural source or combination thereof. Thus, the term “purified”, “in purified form” or “in isolated and purified form” for a compound refers to the physical state of the compound after being obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Fused Tricyclic Compound or a pharmaceutically acceptable salt, hydrate or solvate of the compound. The transformation may occur by various mechanisms (e.g., by metabolic or chemical processes), such as, for example, through hydrolysis in blood.

For example, if a Fused Tricyclic Compound or a pharmaceutically acceptable salt; hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to g carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a Fused Tricyclic Compound contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a Fused Tricyclic Compound incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural □-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N-(C₁-C₆)alkylaminoalkyl; —C(Y⁴)Y⁵ wherein Y⁴ is H or methyl and Y⁵ is mono-N- or di-N,N-(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or —O—C₁₋₄alkyl or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Fused Tricyclic Compounds can form salts which are also within the scope of this invention. Reference to a Fused Tricyclic Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Fused Tricyclic Compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Fused Tricyclic Compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the foluiation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art; such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Fused Tricyclic Compounds may be atropisomers (e.g., substituted biaryls) and are considered as part of this invention. Enantiomers can also be directly separated using chiral chromatographic techniques.

It is also possible that the Fused Tricyclic Compounds may exist in different tautomeric forms, and all such forms are embraced within the scope of the invention. For example, all keto-enol and imine-enamine forms of the compounds are included in the invention. It should also be noted that tautomeric fowls such as, for example, the moieties:

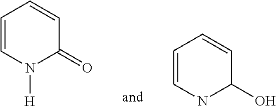

are considered equivalent in certain embodiments of this invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). If a Fused Tricyclic Compound incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹ P, ³²P, ³⁵S, ¹⁸F, and ³⁶Cl, respectively.

Certain isotopically-labelled Fused Tricyclic Compounds (e.g., those labeled with ³H and ¹⁴C) are useful in compound and/or substrate tissue distribution assays. In one embodiment, tritiated (i.e., ³H) and carbon-14 (i.e., ¹⁴C) isotopes are employed for their ease of preparation and detectability. In another embodiment, substitution with heavier isotopes such as deuterium (i.e., ²H) may afford certain therapeutic advantages resulting from greater metabolic stability (e.g., increased in vivo half-life or reduced dosage requirements). In one embodiment, a Compound of Formula (I) has one or more of its hydrogen atoms replaced with a deuterium atom. Isotopically labelled compounds of Formula (I) can generally be prepared by following procedures analogous to those disclosed in the Schemes and/or in the Examples herein below, by substituting an appropriate isotopically labelled reagent for a non-isotopically labelled reagent.

Polymorphic forms of the Fused Tricyclic Compounds, and of the salts, solvates, hydrates, esters and prodrugs of the Fused Tricyclic Compounds, are intended to be included in the present invention.

Polymorphic forms of the Fused Tricyclic Compounds, and of the salts, solvates, hydrates, esters and prodrugs of the Fused Tricyclic Compounds, are intended to be included in the present invention.

The following abbreviations are used below and have the following meanings: AcOH is acetic acid, Boe is tert-butyloxycarbonyl, Boc-Pro-OH is Boc protected praline, DBU is 1,8-diazabicyclo[5.4.0]undec-7-ene, DCC is dicyclohexylcarbodiimide, DME is dimethoxyethane, DMF is dimethylformamide, DMSO is dimethylsulfoxide, EDCI is 1-(3-dimethylaminopropyl)-3-ethylcarbodiimide, EtOAc is ethyl acetate, EtOH is ethanol, HATU is O-(7-azabenzotriazol-1-yl)-N,N,N′,N′-tetramethyluronium hexafluorophosphate, HOBt is 1-hydroxybenzotriazole, HPLC is high-performance liquid chromatography, KOAc is potassium acetate, LRMS is low resolution mass spectrometry, MeOH is methanol, MTBE is PyBrop is bromotripyrrolidinophosphonium hexafluorophosphate, TFA is trifluoroacetic acid and TLC is thin-layer chromatography.

### The Compounds of Formula (I)

The present invention provides Fused Tricycle Compounds of Formula (I):

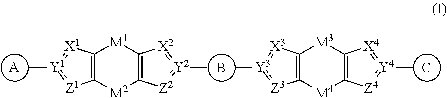

and pharmaceutically acceptable salts, solvates, prodrugs and esters thereof, wherein A, B, C, M¹, M², M³, M⁴, X¹, X², X³, X⁴, Y¹, Y², Y³, Y², Z¹, Z², Z³ and Z⁴ are defined above for the Compounds of Formula (I).

In one embodiment, A is -alkylene-N(R¹¹)(R¹³).

In another embodiment, A is heterocycloalkyl.

In another embodiment, A is 4 to 7-membered heterocycloalkyl.

In still another embodiment, A is selected from:

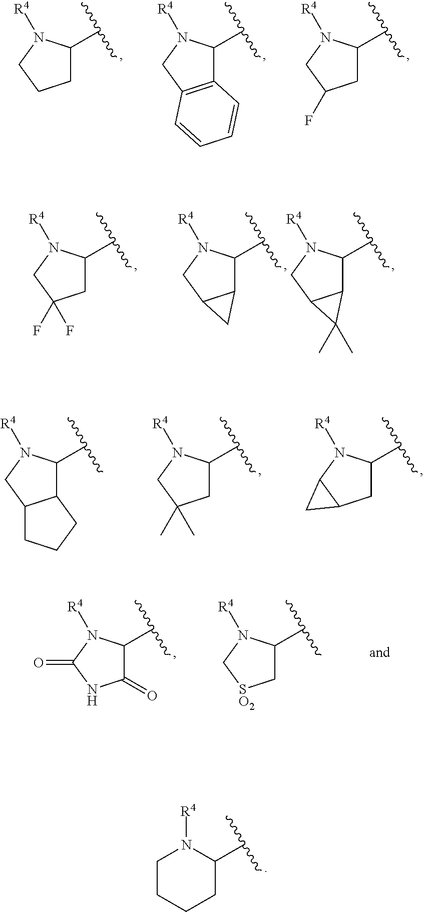

In another embodiment, A is selected from:

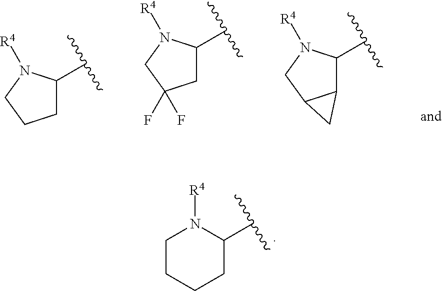

In yet another embodiment, A is selected from:

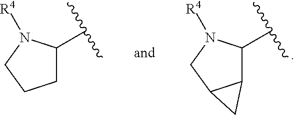

In another embodiment, A is

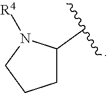

In another embodiment, A is:

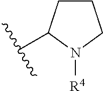

and R⁴ is —C(O)—[CH(R⁵)]_{q}N(R⁶)C(O)O—R¹.

In still another embodiment, A is:

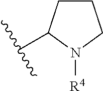

### and R⁴ is:

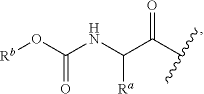

wherein R^{a} is H, alkyl, haloalkyl, cycloalkyl or aryl, and R^{b} is alkyl, haloalkyl or cycloalkyl.

In another embodiment, A is:

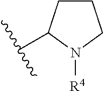

### and R⁴ is:

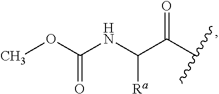

wherein R^{a} is H, methyl, ethyl, propyl, isopropyl, —CH(CH₃)(OCH₃), t-butyl, cyclopropyl, —CH₂CH₂CF₃ or phenyl.

In another embodiment, A is:

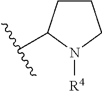

### and R⁴ is:

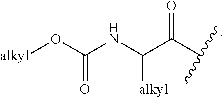

In yet another embodiment, A is:

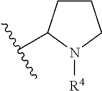

### and R⁴ is

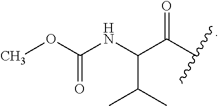

In another embodiment, A is -alkylene-N(cycloalkyl)-C(O)—CH(alkyl)—NHC(O)O-alkyl.

In another embodiment, A is -alkylene-N(cyclohexyl)-C(O)—CH(isopropyl)-NHC(O)O-methyl.

In a further embodiment, A is -alkylene-N(aryl)-C(O)—CH(alkyl)-NHC(O)O-alkyl.

In one embodiment, B is a bond.

In another embodiment, B is phenylene.

In another embodiment, B is:

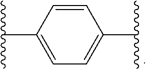

In still another embodiment, B is C₁-C₃ alkylene.

In another embodiment, B is —CH₂—.

In another embodiment, B is —C(R⁵)═C(R⁵)—.

In yet another embodiment, B is —CH═CH—.

In another embodiment, B is —C≡C—.

In a further embodiment, B is monocyclic cycloalkyl.

In another embodiment, B is cyclopentyl or cyclohexyl.

In another embodiment, B is monocyclic heterocycloalkyl.

In still another embodiment, B is monocyclic heteroarylene.

In one embodiment, C is -alkylene-N(R¹¹)(R¹³).

In another embodiment, C is heterocycloalkyl.

In another embodiment, C is 4 to 7-membered heterocycloalkyl.

In another embodiment, C is selected from:

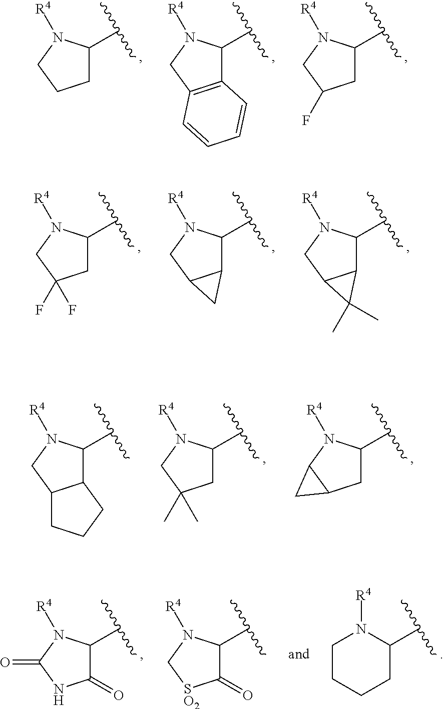

In another embodiment, C is selected from:

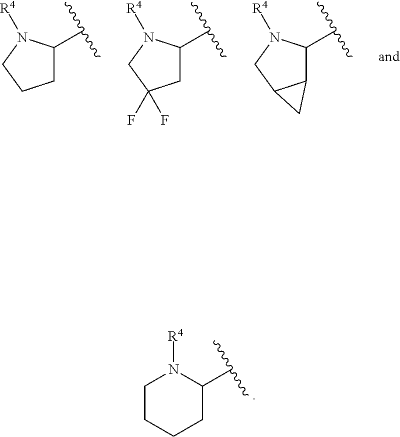

In yet another embodiment, C is selected from:

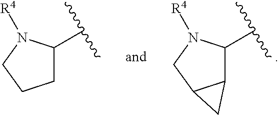

In another embodiment, C is

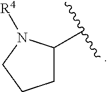

In another embodiment, C is:

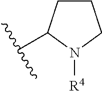

and R⁴ is —C(O)—]CH(R⁵)]_{q}N(R⁶)C(O)O—R¹.

In still another embodiment, C is:

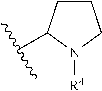

### and R⁴ is:

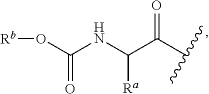

wherein R^{a} is H, alkyl, haloalkyl, cycloalkyl or aryl, and R^{b} is alkyl, haloalkyl or cycloalkyl.

In another embodiment, C is:

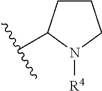

### and R⁴ is:

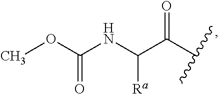

wherein R^{a} is H, methyl, ethyl, propyl,isopropyl, —CH(CH₃)(OCH₃), t-butyl, cyclopropyl, —CH₂CH₂CF₃ or phenyl.

In another embodiment, C is:

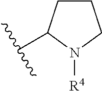

### and R⁴ is:

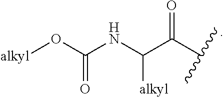

In yet another embodiment, C is:

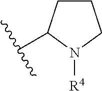

### and R⁴ is

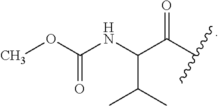

In another embodiment, C is -alkylene-N(cycloalkyl)-C(O)—CH(alkyl)-NHC(O)O-alkyl.

another embodiment, C is -alkylene-N(cyclohexyl)-C(O)—CH(isopropyl)-NHC(O)O-methyl.

In a further embodiment, C is -alkylene-N(aryl)-C(O)—CH(alkyl)-NHC(O)O-alkyl.

In one embodiment, A and C are each independently -alkylene-N(R¹¹)(R¹³).

In another embodiment, A and C are each independently a 4 to 7-membered heterocycloalkyl.

In still another embodiment, A and C are each independently selected from:

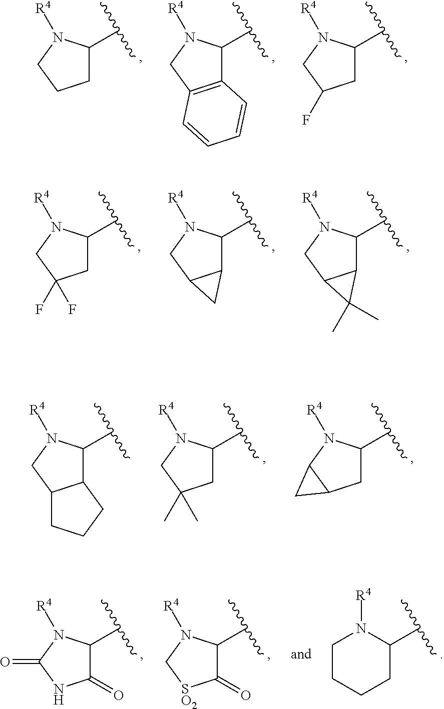

In another embodiment, A and C are each independently selected from:

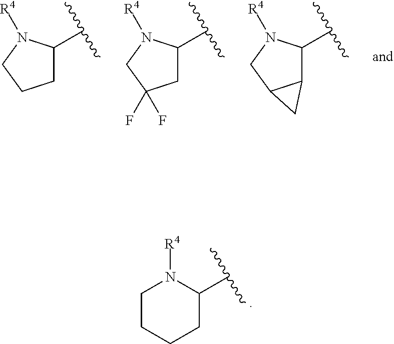

In yet another embodiment, A and C are each independently selected from:

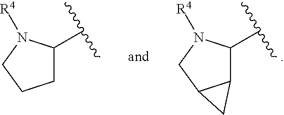

In another embodiment, A and C are each:

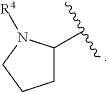

In another embodiment, A and C are each independently:

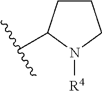

and each R⁴ is independently —C(O)—[CH(R⁵)]_{q}N(R⁶)C(O)O—R¹.

In still another embodiment, A and C are each independently

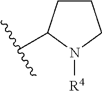

and each R⁴ is independently

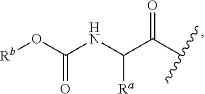

wherein R^{a} is H, alkyl, haloalkyl, cycloalkyl or aryl, and R^{b} is alkyl, haloalkyl or cycloalkyl.

In another embodiment, A and C are each independently

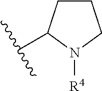

each R⁴ is independently

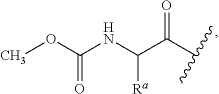

wherein R^{a} is H, methyl, ethyl, propyl, isopropyl, —CH(CH₃)(OCH₃), t-butyl, cyclopropyl, —CH₂CH₂CF₃ or phenyl.

In another embodiment, A and C are each independently

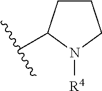

each R⁴ is independently:

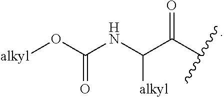

In one embodiment, A and C are each independently selected front

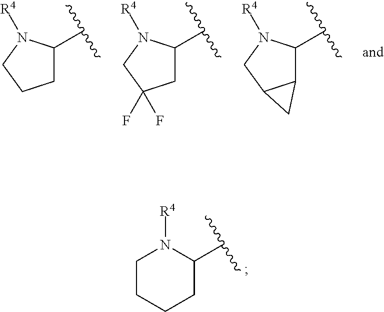

and each occurrence of R⁴ is independently selected from:

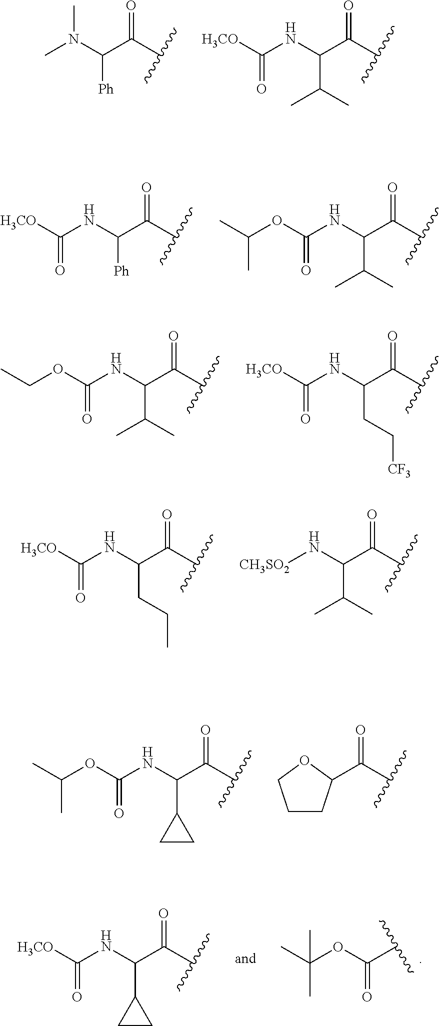

In another embodiment, A and C are each independently selected from:

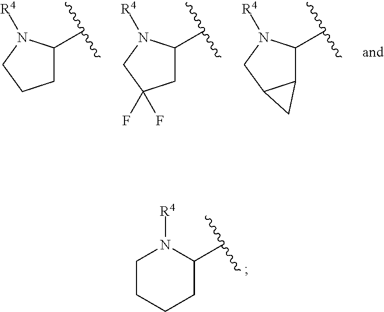

and each occurrence of R⁴ is independently selected from:

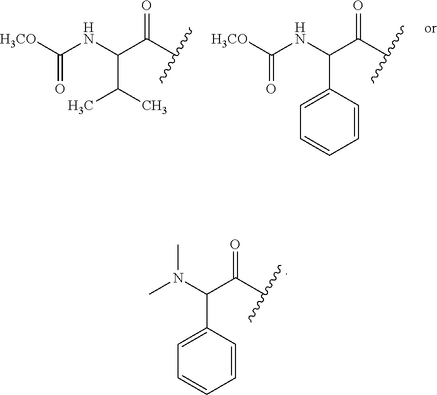

In another embodiment, A and C are each independently

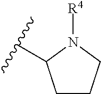

and each occurrence of R⁴ is independently selected from:

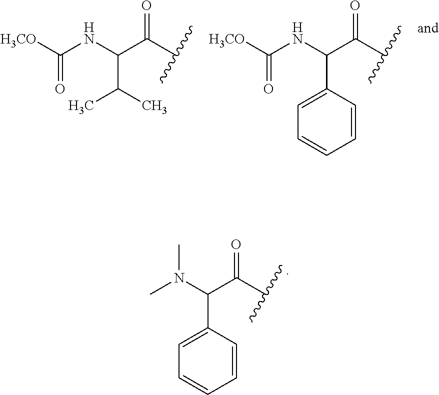

In still another embodiment, A and C are each independently:

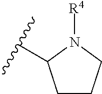

and each occurrence of R⁴ is:

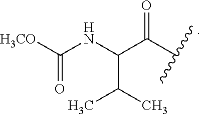

In another embodiment, A and C are each independently -alkylene-N(cycloalkyl)-C(O)—CH(alkyl)-NHC(O)O-alkyl.

In another embodiment, A and C are each independently -alkylene-N(cyclohexyl)-C(O)—CH(isopropyl)-NHC(O)O-methyl.

In a further embodiment, A and C are each independently -alkylene-N(aryl)-C(O)—CH(alkyl)-NHC(O)O-alkyl.

In one embodiment, one of A and C is -alkylene-N(R¹¹)(R¹³) and the other is a 4 to 7-membered heterocycloalkyl.

In another embodiment, one of A and C is -alkylene-N(R¹¹)(R¹³) and the other is:

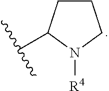

In one embodiment, at least one of M¹ and M² is —[C(R⁷)₂]_{q}—.

In another embodiment, at least one of M¹ and M² is —C(R⁷)₂C(R⁷)₂C(R⁷)₂—.

In another embodiment, at least one of M¹ and M² is —C(R⁷)₂C(R⁷)₂—.

In still another embodiment, at least one of M¹ and M² is —C(R⁷)₂—.

In another embodiment, at least one of M¹ and M² is —CH₂—.

In another embodiment, at least one of M¹ and M² is —CH₂CH₂—.

In yet another embodiment, at least one of M¹ and M² is a bond.

In another embodiment, at least one of M¹ and M² is —CH₂C(R⁷)₂CH₂—.

In a further embodiment, at least one of M¹ and M² is —C(R⁷)═C(R⁷)—.

In another embodiment, at least one of M¹ and M² is —CH═CH—.

In another embodiment, at least one of M¹ and M² is —CH═N—.

In still another embodiment, at least one of M¹ and M² is —N═CH—.

In another embodiment, at least one of M¹ and M² is —[C(R⁷)₂]ₘ—O—[C(R⁷)₂]ₘ,

In another embodiment, at least one of M¹ and M² is —C(R⁷)₂OC(R⁷)₂—.

In yet another embodiment, at least one of M¹ and M² is —CH₂OCH₂—.

In another embodiment, at least one of M¹ and M² is —[C(R⁷)₂]ₘ—N(R⁶)—[C(R⁷)₂]ₘ—.

In a further embodiment, at least one of M¹ and M² is —[C(R⁷)₂]—N(R⁶)—[C(R⁷)₂]—.

In another embodiment, at least one of M¹ and M² is —CH₂N(R⁶)CH₂—.

In another embodiment, at least one of M¹ and M² is —CH₂NHCH₂—.

In still another embodiment, at least one of M¹ and M² is —NR⁶—.

In another embodiment, at least one of M¹ and M² is [C(R⁷)₂]ₘ—S(O)₂—[C(R⁷)₂]ₘ—.

In another embodiment, at least one of M¹ and M² is [C(R⁷)₂]—S(O)₂—[C(R⁷)₂]—.

In yet another embodiment, at least one of M¹ and M² is —CH₂S(O)₂CH₂—.

In another embodiment, at least one of M¹ and M² is —CH₂CH₂S(O)₂—.

In another embodiment, at least one of M¹ and M² is —S(O)₂CH₂—.

In a further embodiment, at least one of M¹ and M² is —S(O)₂—.

In another embodiment, at least one of M¹ and M² is —S—.

In another embodiment, at least one of M¹ and M² is —[C(R⁷)₂]ₘ—OC(O)N(R⁶)—[C(R⁷)₂]ₘ—.

In yet another embodiment, at least one of M¹ and M² is —OC(O)N(R⁶)—[C(R⁷)₂]ₘ—.

In another embodiment, at least one of M¹ and M² is —OC(O)N(R⁶)CH₂—.

In another embodiment, at least one of M¹ and M² is —OC(O)N(R⁶)—.

In still another embodiment, at least one of M¹ and M² is —OC(O)NH—.

In another embodiment, at least one of M¹ and M² is —[C(R⁷)₂]ₘN(R⁶)C(O)N(R⁶)[C(R⁷)₂]ₘ—.

In another embodiment, at least one of M¹ and M² is —N(R⁶)C(O)N(R⁶)[C(R⁷)₂]ₘ—.

In a further embodiment, at least one of M¹ and M² is —N(R⁶)C(O)N(R⁶)CH₂—.

In another embodiment, at least one of M¹ and M² is —N(R⁶)C(O)N(R⁶)—.

In another embodiment, at least one of M¹ and M² is —NHC(O)NH—.

In still another embodiment, at least one of M¹ and M² is —[C(R⁷)₂]ₘ—S(O)₂N(R⁶)—[C(R⁷)₂]ₘ—.

In another embodiment, at least one of M¹ and M² is —S(O)₂N(R⁶)—[C(R⁷)₂]ₘ—.

In another embodiment, at least one of M¹ and M² is —CH₂S(O)₂N(R⁶)CH₂—.

In yet another embodiment, at least one of M¹ and M² is —S(O)₂N(R⁶)CH₂—.

In another embodiment, at least one of M¹ and M² is —CH₂S(O)₂N(R⁶)—.

In a further embodiment, at least one of M¹ and M² is —S(O)₂N(R⁶)—.

In another embodiment, at least one of M¹ and M² is —S(O)₂NH—.

In another embodiment, at least one of M¹ and M² is —[C(R⁷)₂]ₘN(R⁶)S(O)₂N(R⁶)[C(R⁷)₂]ₘ—.

In still another embodiment, at least one of M¹ and M² is —C(R⁷)₂N(R⁶)S(O)₂N(R⁶)C(R⁷)₂—.

In another embodiment, at least one of M¹ and M² is —CH₂N(R⁶)S(O)₂N(R⁶)CH₂—.

In another embodiment, at least one of M¹ and M² is —N(R⁶)S(O)₂N(R⁶)CH₂—.

In yet another embodiment, at least one of M¹ and M² is —NHS(O)₂NHCH₂—.

In another embodiment, at least one of M¹ and M² is —NHS(O)₂NH—.

In another embodiment, M¹ is a bond and M² is other than a bond.

In still another embodiment, M² is a bond and M¹ is other than a bond.

In one embodiment, M¹ and M² are each —C(R¹²)₂—.

In another embodiment, M¹ and M² are each —CH₂—.

In another embodiment, M¹ and M² are each —NH—.

In another embodiment, one of M¹ and M² is —CH₂— and the other is —NH—.

In another embodiment, one of M¹ and M² is a bond.

In another embodiment, one of M¹ and M² is a bond and the other is —CH₂—.

In another embodiment, one of M¹ and M² is a bond and the other is —NH—.

In still another embodiment, one of M¹ and M² is a bond and the other is —O—.

In one embodiment, at least one of M³ and M⁴ is —[C(R⁷)₂]_{q}—.

In another embodiment, at least one of M³ and M⁴ is —C(R⁷)₂C(R⁷)₂C(R⁷)₂—.

In another embodiment, at least one of M³ and M⁴ is —C(R⁷)₂C(R⁷)₂—.

In still another embodiment, at least one of M³ and M⁴ is —C(R⁷)₂—.

In another embodiment, at least one of M³ and M⁴ is —CH₂—.

In another embodiment, at least one of M³ and M⁴ is —CH₂CH₂—.

In yet another embodiment, at least one of M³ and M⁴ is a bond.

In another embodiment, at least one of M³ and M⁴ is —CH₂C(R⁷)₂CH₂—.

In a further embodiment, at least one of M³ and M⁴ is —C(R⁷)═C(R⁷)—.

In another embodiment, at least one of M³ and M⁴ is —CH═CH—.

In another embodiment, at least one of M³ and M⁴ is —CH═N—.

In still another embodiment, at least one of M³ and M⁴ is —N═CH—.

In another embodiment, at least one of M³ and M⁴ is —[C(R⁷)₂]ₘ—O—[C(R⁷)₂]ₘ,

In another embodiment, at least one of M³ and M⁴ is —C(R⁷)₂OC(R⁷)₂—.

In yet another embodiment, at least one of M³ and M⁴ is —CH₂OCH₂—.

In another embodiment, at least one of M³ and M⁴ is —[C(R⁷)₂]ₘ—N(R⁶)—[C(R⁷)₂]ₘ—.

In a further embodiment, at least one of M³ and M⁴ is —[C(R⁷)₂]—N(R⁶)—[C(R⁷)₂]—,

In another embodiment, at least one of M³ and M⁴ is —CH₂N(R⁶)CH₂—.

In another embodiment, at least, one of M³ and M⁴ is —CH₂NHCH₂—.

In still another embodiment, at least one of M³ and M⁴ is —NR⁶—.

In another embodiment, at least one of M³ and M⁴ is [C(R⁷)₂]ₘ—S(O)₂—[C(R⁷)₂]ₘ—.

In another embodiment, at least one of M³ and M⁴ is [C(R⁷)₂]—S(O)₂—[C(R⁷)₂]—.

In yet another embodiment, at least one of M³ and M⁴ is —CH₂S(O)₂CH₂—.

In another embodiment, at least one of M³ and M⁴ is —CH₂CH₂S(O)₂—.

In another embodiment, at least one of M³ and M⁴ is —S(O)₂CH₂—.

In a further embodiment, at least one of M³ and M⁴ is —S(O)₂—.

In another embodiment, at least one of M³ and M⁴ is —S—.

In another embodiment, at least one of M³ and M⁴ is —[C(R⁷)₂]ₘ—OC(O)N(R⁶)—[C(R⁷)₂]ₘ—.

In yet another embodiment, at least one of M³ and M⁴ is —OC(O)N(R⁶)—[C(R⁷)₂]ₘ—.

In another embodiment, at least one of M³ and M⁴ is —OC(O)N(R⁶)CH₂—.

In another embodiment, at least one of M³ and M⁴ is —OC(O)N(R⁶)—.

In still another embodiment, at least one of M³ and M⁴ is —OC(O)NH—.

In another embodiment, at least one of M³ and M⁴ is —[C(R⁷)₂]ₘN(R⁶)C(O)N(R⁶)[C(R⁷)₂]ₘ—.

In another embodiment, at least one of M³ and M⁴ is —N(R⁶)C(O)N(R⁶)[C(R⁷)₂]ₘ—.

In a further embodiment, at least one of M³ and M⁴ is —N(R⁶)C(O)N(R⁶)CH₂—.

In another embodiment, at least one of M³ and M⁴ is —N(R⁶)C(O)N(R⁶)—.

In another embodiment, at least one of M³ and M⁴ is —NHC(O)NH—.

In still another embodiment, at least one of M³ and M⁴ is —[C(R⁷)₂]ₘ—S(O)₂N(R⁶)—[C(R⁷)₂]ₘ—.

In another embodiment, at least one of M³ and M⁴ is —S(O)₂N(R⁶)—[C(R⁷)₂]ₘ—.

In another embodiment, at least one of M³ and M⁴ is —CH₂S(O)₂N(R⁶)CH₂—.

In yet another embodiment, at least one of M³ and M⁴ is —S(O)₂N(R⁶)CH₂—.

In another embodiment, at least one of M³ and M⁴ is —CH₂S(O)₂N(R⁶)—.

In a further embodiment, at least one of M³ and M⁴ is —S(O)₂N(R⁶)—.

In another embodiment, at least one of M³ and M⁴ is —S(O)₂NH—.

In another embodiment, at least one of M³ and M⁴ is —[C(R⁷)₂]ₘN(R⁶)S(O)₂N(R⁶)[C(R⁷)₂]ₘ—.

In still another embodiment, at least one of M³ and M⁴ is —C(R⁷)₂N(R⁶)S(O)₂N(R⁶)C(R)₂—.

In another embodiment, at least one of M³ and M⁴ is —CH₂N(R⁶)S(O)₂N(R⁶)CH₂—.

In another embodiment, at least one of M³ and M⁴ is —N(R⁶)S(O)₂N(R⁶)CH₂—.

In yet another embodiment, at least one of M³ and M⁴ is —NHS(O)₂NHCH₂—.

In another embodiment, M³ is —NHS(O)2NH—.

In another embodiment, M³ is a bond and M⁴ is other than a bond.

In one embodiment, M⁴ is a bond and M³ is other than a bond.

In another embodiment, M³ and M⁴ are each —C(R¹²)₂—.

In another embodiment, M³ and M⁴ are each —CH₂—.

In still another embodiment, M³ and M⁴ are each —NH—.

In another embodiment, one of M³ and M⁴ is —CH₂— and the other is —NH—.

In another embodiment, one of M³ and M⁴ is a bond and the other is —CH₂—.

In still another embodiment, one of M³ and M⁴ is a bond and the other is —NH—

In still another embodiment, one of M⁴ and M⁴ is a bond and the other is —O—.

In one embodiment, X¹ is a bond.

In another embodiment, X¹ is —C(R⁵)═C(R⁵)—.

In another embodiment, X¹ is —N═C(R⁵)—.

In still another embodiment, X¹ is —C(R⁵)═NC—.

In another embodiment, X¹ is —C(R⁵)═N—.

In another embodiment, X¹ is —O—.

In yet another embodiment X¹ is —N(R⁶)—.

In another embodiment, X¹ is —S—.

In a further embodiment, X¹ is —S(O)₂—.

In another embodiment, X¹ is —C(R⁵)(CH(R⁵))ₘ—.

In another embodiment, X¹ is —N—.

In still another embodiment, X¹ is —N—CH(R⁵)CH(R⁵)—.

In another embodiment, X¹ is —C(R⁵)NHCH(R⁵)—.

In another embodiment, X¹ is —C(R⁵)CH(R⁵)NH—.

In yet another embodiment, X¹ is —C(R⁵)O—.

In another embodiment, X¹ is —C(R⁵)N(R⁶)—.

In a further embodiment, X¹ is —N—N(R⁶)—.

In another embodiment, X¹ is —C(R⁵)S—.

In another embodiment, X¹ is —C(R⁵)S(O)₂—.

In one embodiment, X² is a bond.

In another embodiment, X² is —C(R⁵)═C(R⁵)—.

In another embodiment, X² is —N═C(R⁵)—.

In still another embodiment, X² is —C(R⁵)═NC—.

In another embodiment, X² is —C(R⁵)═N—.

In another embodiment, X² is —O—.

In yet another embodiment X² is —N(R⁶)—.

In another embodiment, X² is —S—.

In a further embodiment, X² is —S(O)₂—.

In another embodiment, X² is —(CH(R⁵))ₘC(R⁵)—.

In another embodiment, X² is —N—.

In still another embodiment, X² is —CH(R⁵)CH(R⁵)N—.

In another embodiment, X² is —CH(R⁵)NHC(R⁵)—.

In another embodiment, X² is —NHCH(R⁵)C(R⁵)—.

In yet another embodiment, X² is —O—C(R⁵)—.

In another embodiment, X² is —N(R⁶)C(R⁵)—.

In another embodiment, X² is —N(R⁶)—N—.

In a further embodiment, X² is —S—C(R⁵)—.

In another embodiment, X² is —S(O)₂C(R⁵)—.

In one embodiment, X³ is a bond.

In another embodiment, X³ is —C(R⁵)═C(R⁵)—.

In another embodiment, X³ is —N═C(R⁵)—.

In still another embodiment, X³ is —C(R⁵)═NC—.

In another embodiment, X³ is —C(R⁵)N—.

In another embodiment, X³ is —O—.

In yet another embodiment X³ is —N(R⁶)—.

In another embodiment, X³ is —S—.

In a further embodiment, X³ is —S(O)₂—.

In another embodiment, X³ is —C(R⁵)(CH(R⁵))ₘ—.

In another embodiment, X³ is —N—.

In still another embodiment, X³ is —N—CH(R⁵)CH(R⁵)—.

In another embodiment, X³ is —C(R⁵)NHCH(R⁵)—.

In another embodiment, X³ is —C(R⁵)CH(R⁵)NH—.

In yet another embodiment, X³ is —C(R⁵)O—.

In another embodiment, X³ is —C(R⁵)N(R⁶)—.

In a further embodiment, X³ is —N—N(R⁶)—.

In another embodiment, X³ is —C(R⁵)S—.

In another embodiment, X³ is —C(R⁵)S(O)₂—.

In one embodiment, X⁴ is a bond.

In another embodiment, X⁴ is —C(R⁵)═C(R⁵)—.

In another embodiment, X⁴ is —N═C(R⁵)—.

In still another embodiment, X⁴ is —C(R⁵)═NC—.

In another embodiment, X⁴ is —C(R⁵)═N—.

In another embodiment, X⁴ is —O—.

In yet another embodiment X⁴ is —N(R⁶)—.

In another embodiment, X⁴ is —S—.

In a further embodiment, X⁴ is —S(O)₂—.

In another embodiment, X⁴ is —(CH(R⁵))ₘC(R⁵)—.

In another embodiment, X⁴ is —N—.

In still another embodiment, X⁴ is —CH(R⁵)CH(R⁵)N—.

In another embodiment, X⁴ is —CH(R⁵)NHC(R⁵)—.

In another embodiment, X⁴ is —NHCH(R⁵)C(R⁵)—.

In yet another embodiment, X⁴ is —O—C(R⁵)—.

In another embodiment, X⁴ is —N(R⁶)C(R⁵)—.

In another embodiment, X⁴ is —N(R⁶)—N—.

In a further embodiment, X⁴ is —S—C(R⁵)—.

In another embodiment, X⁴ is —S(O)₂C(R⁵)—.

In one embodiment, Z¹ is a bond.

In another embodiment, Z¹ is —C(R⁵)═C(R⁵)—.

In another embodiment, Z¹ is —N═C(R⁵)—.

In still another embodiment, Z¹ is —C(R⁵)═NC—.

In another embodiment, Z¹ is —C(R⁵)N—.

In another embodiment, Z¹ is —O—.

In yet another embodiment Z¹ is —N(R⁶)—.

In another embodiment, Z¹ is —S—.

In a further embodiment, Z¹ is —S(O)₂—.

In another embodiment, Z¹ is —C(R⁵)(CH(R⁵))ₘ—.

In another embodiment, Z¹ is —N—.

In still another embodiment, Z¹ is —N—CH(R⁵)CH(R⁵)—.

In another embodiment, Z¹ is —C(R⁵)NHCH(R⁵)—.

In another embodiment, Z¹ is —C(R⁵)CH(R⁵)NH—.

In yet another embodiment, Z¹ is —C(R⁵)O—.

In another embodiment, Z¹ is —C(R⁵)N(R⁶)—.

In a further embodiment, Z¹ is —N—N(R⁶)—.

In another embodiment, Z¹ is —C(R⁵)S—.

In another embodiment, Z¹ is —C(R⁵)S(O)₂—.

In one embodiment, Z² is a bond.

In another embodiment, Z² is —C(R⁵)═C(R⁵)—.

In another embodiment, Z² is —N═C(R⁵)—.

In still another embodiment, Z² is —C(R⁵)═NC—.

In another embodiment, Z² is —C(R⁵)═N—.

In another embodiment, Z² is —O—.

In yet another embodiment Z² is —N(R⁶)—.

In another embodiment, Z² is —S—.

In a further embodiment, Z² is —S(O)₂—.

In another embodiment, Z² is —(CH(R⁵))ₘC(R⁵)—.

In another embodiment, Z² is —N—.

In still another embodiment, Z² is —CH(R⁵)CH(R⁵)N—.

In another embodiment, Z² is —CH(R⁵)NHC(R⁵)—.

In another embodiment, Z² is —NHCH(R⁵)C(R⁵)—.

In yet another embodiment, Z² is —O—C(R⁵)—.

In another embodiment, Z² is —N(R⁶)C(R⁵)—.

In another embodiment, Z² is —N(R⁶)—N—.

In a further embodiment, Z² is —S—C(R⁵)—.

In another embodiment, Z² is —S(O)₂C(R⁵)—.

In one embodiment, Z³ is a bond.

In another embodiment, Z³ is —C(R⁵)═C(R⁵)—.

In another embodiment, Z³ is —N═C(R⁵)—.

In still another embodiment, Z³ is —C(R⁵)═NC—.

In another embodiment, Z³ is —C(R⁵)═N—.

In another embodiment, Z³ is —O—.

In yet another embodiment Z³ is —N(R⁶)—.

In another embodiment, Z³ is —S—.

In a further embodiment, Z³ is —S(O)₂—.

In another embodiment, Z³ is —C(R⁵)(CH(R⁵))ₘ—.

In another embodiment, Z³ is —N—.

In still another embodiment, Z³ is —N—CH(R⁵)CH(R⁵)—.

In another embodiment, Z³ is —C(R⁵)NHCH(R⁵)—.

In another embodiment, Z³ is —C(R⁵)CH(R⁵)NH—.

In yet another embodiment, Z³ is —C(R⁵)O—.

In another embodiment, Z³ is —C(R⁵)N(R⁶)—.

In a further embodiment, Z³ is —N—N(R⁶)—.

In another embodiment, Z³ is —C(R⁵)S—.

In another embodiment, Z³ is —C(R⁵)S(O)₂—.

In one embodiment, Z⁴ is a bond.

In another embodiment, Z⁴ is —C(R⁵)═C(R⁵)—.

In another embodiment, Z⁴ is —N═C(R⁵)—.

In still another embodiment, Z⁴ is —C(R⁵)═NC—.

In another embodiment, Z⁴ is —C(R⁵)═N—.

In another embodiment, Z⁴ is —O—.

In yet another embodiment Z⁴ is —N(R⁶)—.

In another embodiment, Z⁴ is —S—.

In a further embodiment, Z⁴ is —S(O)₂—.

In another embodiment, Z⁴ is —(CH(R⁵))ₘC(R⁵)—.

In another embodiment, Z⁴ is —N—.

In still another embodiment, Z⁴ is —CH(R⁵)CH(R⁵)N—.

In another embodiment, Z⁴ is —CH(R⁵)NHC(R⁵)—.

In another embodiment, Z⁴ is —NHCH(R³)C(R⁵)—.

In yet another embodiment, Z⁴ is —O—C(R⁵)—.

In another embodiment, Z⁴ is —N(R⁶)C(R⁵)—.

In another embodiment, Z⁴ is —N(R⁶)—N—.

In a further embodiment, Z⁴ is —S—C(R⁵)—.

In another embodiment, Z⁴ is —S(O)_{z}C(R⁵)—.

In one embodiment, the group:

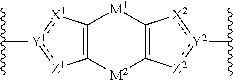

has the structure:

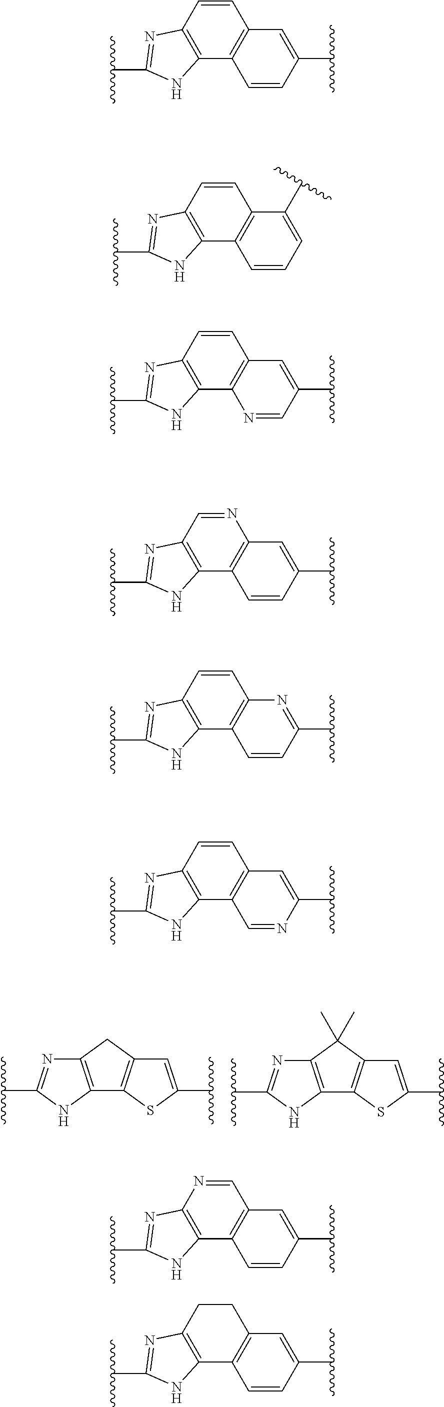

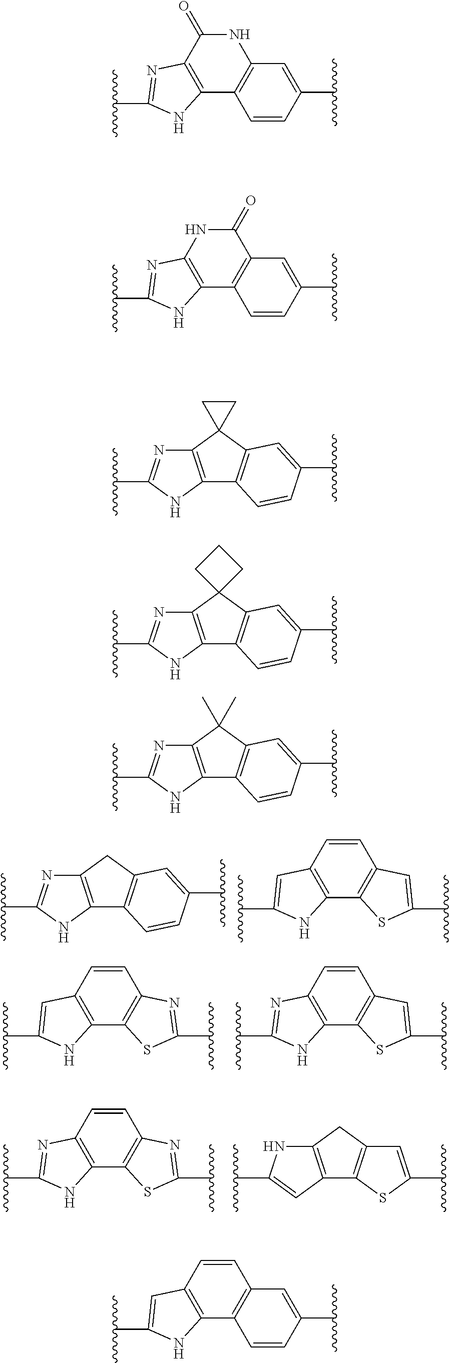

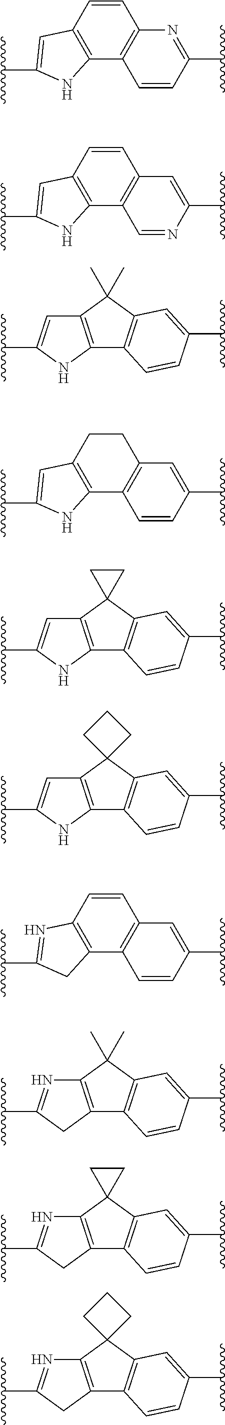

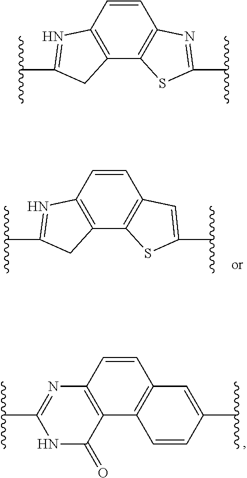

wherein either available bond on any of the above divalent groups can connect to either group flanking the above divalent groups.

In another embodiment, the group:

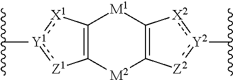

has the structure:

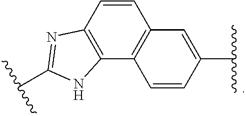

In one embodiment, the group:

A is -alkylene-N(R¹¹)(R¹³) or heterocycloalkyl, wherein said heterocycloalkyl group can be optionally and independently substituted with from one to three R⁴ groups, and wherein said heterocycloalkyl group can be optionally fused to a cycloalkyl group or a benzene group;

B is a bond, C₁-C₃ alkylene, —C(R⁵)═C(R⁵)—, phenylene, monocyclic cycloalkylene, monocyclic cycloalkenylene, monocyclic heterocycloalkylene, monocyclic heterocycloalkenylene or monocyclic heteroarylene, wherein said phenylene group or said monocyclic cycloalkylene group can be optionally and independently substituted with one or more R¹⁴ groups, and wherein said phenylene group or said monocyclic cycloalkylene group can be optionally and independently substituted on one or more ring nitrogen atoms with R⁶ and on one or more ring carbon atoms with R¹⁴;

C is -alkylene-N(R¹¹)(R¹³) or heterocycloalkyl, wherein said heterocycloalkyl group can be optionally and independently substituted with from one to three R⁴ groups; and wherein said heterocycloalkyl group can be optionally fused to a cycloalkyl group or a benzene group;

M¹ is a bond, —[C(R⁷)₂]_{q}—, —[C(R⁷)₂]ₘ—C(R)═C(R⁷)—[C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—;

M² is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—, such that at least one of M¹ and M² is other than a bond, and such that the central ring of formula (I) that contains M¹ and M² has from 5 to 10 total ring atoms, and wherein two vicinal R⁷ groups of M¹ or M² together with the carbon atoms to which they are attached, can optionally join to form a 3- to 7-membered cycloalkyl group, a 3- to 7-membered heterocycloalkyl group, or a 5- to 6-membered heteroaryl group;

M³ is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂ ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—;

M⁴ is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—, such that at least one of M³ and M⁴ is other than a bond, and such that the central ring of formula (I) that contains M³ and M⁴ has from 5 to 10 total ring atoms, and wherein two vicinal R⁷ groups of M³ or M⁴ together with the carbon atoms to which they are attached, can optionally join to form a 3- to 7-membered cycloalkyl group, a 3- to 7-membered heterocycloalkyl group, or a 5- to 6-membered heteroaryl group;

X¹ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X¹ is not present, and X¹ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to X¹ is present, such that X¹ and Z¹ cannot each be a bond, and such that when X¹ is —C(R⁵)— or —N—, then Z¹ is other than a bond;

X² is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X² is not present, and X² is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to X² is present, such that X² and Z² cannot each be a bond, and such that when X² is —C(R⁵)— or —N—, then Z² is other than a bond;

X³ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X³ is not present, and X³ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to X³ is present, such that X³ and Z³ cannot each be a bond, and such that when X³ is —C(R⁵)— or —N—, then Z³ is other than a bond;

X⁴ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X⁴ is not present, and X⁴ is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to X⁴ is present, such that X⁴ and Z⁴ cannot each be a bond, and such:that when X⁴ is —C(R⁵)— or —N—, then Z⁴ is other than a bond;

Y¹ is —C— when an optional and additional bond to Y¹ is present, and Y¹ is —CH— when an optional and additional bond to Y¹ is absent;

Y² is —C— when an optional and additional bond to Y² is present, and Y² is —CH— when an optional and additional bond to Y² is absent;

Y³ is —C— when an optional and additional bond to Y³ is present, and Y³ is —CH— when an optional and additional bond to Y³ is absent;

Y⁴ is —C— when an optional and additional bond to Y⁴ is present, and Y⁴ is —CH— when an optional and additional bond to Y⁴ is absent;

Z¹ is a bond, —C(R⁵)═C(R⁵)—, —N═C(R⁵)—, —C(R⁵)═NC—, —O—, —N(R⁶)—, —S— or —S(O)₂— when the optional and additional bond to Z¹ is not present, and Z¹ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to Z¹ is present, such that such that the ring in formula (I) containing X¹, Y¹ and Z¹ has 5 or 6 total ring atoms;

Z² is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z² is not present, and Z² is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to Z² is present, such that the ring in formula (I) containing X², Y² and Z² has 5 or 6 total ring atoms;

Z³ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z³ is not present, and Z³ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to Z³ is present, such that such that the ring in formula (I) containing X³, Y³ and Z³ has 5 or 6 total ring atoms;

Z⁴ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z⁴ is not present, and Z⁴ is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to Z⁴ is present, such that the ring in formula (I) containing X⁴, Y⁴ and Z⁴ has 5 or 6 total ring atoms;

each occurrence of R¹ is independently alkyl, haloalkyl, aryl, cycloalkyl, heterocycloalkyl or heteroaryl, wherein said aryl group, said cycloalkyl group, said heterocycloalkyl group or said heteroaryl group can be optionally and independently substituted with R²;

each occurrence of R² is independently alkyl, halo, haloalkyl, aryl, heterocycloalkyl, heteroaryl, —CN, —OR³, —N(R³)₂, —C(O)R¹², —C(O)OR³, —C(O)N(R³)₂, —NHC(O)R¹², —NHC(O)NHR³, —NHC(O)OR³, —OC(O)R¹², —SR³ or —S(O)₂R¹²;

each occurrence of R³ is independently H, alkyl, haloalkyl, aryl, cycloalkyl, heterocycloalkyl or heteroaryl;

each occurrence of R⁴ is independently halo, —C(O)—[C(R⁵)₂]_{q}N(R⁶)₂, —C(O)—[CH(R⁵)]_{q}N(R⁶)C(O)—R¹, —C(O)—[CH(R⁵)]_{q}N(R⁶)C(O)O—R¹, —C(O)—[CH(R⁵)]_{q}C(O)O—R¹, —C(O)[CH(R⁵)]_{q}N(R⁶)SO₂—R¹ or -alkylene-N(R⁶)—[CH(R⁵)]_{q}—N(R⁶)—C(O)O—R¹;

each occurrence of R⁵ is independently H, —C₁-C₆ alkyl, 3 to 7-membered cycloalkyl, aryl or heteroaryl;

each occurrence of R⁶ is independently H, —C₁-C₆ alkyl, 3 to 7-membered cycloalkyl, 4 to 7-membered heterocycloalkyl, aryl, or heteroaryl, wherein said 3 to 7-membered cycloalkyl group, said 4 to 7-membered heterocycloalkyl group, said aryl group or said heteroaryl group can be optionally and independently substituted with up to two R⁸ groups, and wherein two R⁶ groups that are attached to a common nitrogen atom, together with the nitrogen atom to which they are attached, can optionally join to form a 4 to 7-membered heterocycloalkyl group;

each occurrence of R⁷ is independently H, —C₁-C₆ alkyl, 3 to 7-membered cycloalkyl, 3 to 7-membered heterocycloalkyl, aryl or heteroaryl, wherein said 3 to 7-membered cycloalkyl group, said 3 to 7-membered heterocycloalkyl group, said aryl group or said heteroaryl group can be optionally and independently substituted with up to 3 substituents, which can be the same or different, and are selected from C₁-C₆ alkyl, halo, —C₁-C₆ haloalkyl, —C₁-C₆ hydroxyalkyl, —OH, —C(O)NH—(C₁-C₆ alkyl), —C(O)N(C₁-C₆ alkyl)₂, —O—(C₁-C₆ alkyl), —NH₂, —NH(C₁-C₆ alkyl), —N(C₁-C₆ alkyl)₂ and —NHC(O)—(C₁-C₆alkyl), and wherein two geminal R⁷ groups, together with the common carbon atom to which they are attached, can optionally join to form —C(O)—, —C(S)—, —C(═NR⁹)—, —C(═NOR⁹)—, a 3 to 7-membered cycloalkyl group or a 3 to 7-membered heterocycloalkyl group, such that no two adjacent —C(R⁷)₂— groups can join to form a —C(O)—C(O)—, —C(S)—C(S)—, —C(O)—C(S)— or —C(S)—C(O)— group;

each occurrence of R⁸ is independently H or —C₁-C₆ alkyl;

each occurrence of R⁹ is independently H, —C₁-C₆ alkyl, 3 to 6-membered cycloalkyl or 4 to 6-membered heterocycloalkyl;

R¹¹ is 3 to 7-membered cycloalkyl, 3 to 7-membered heterocycloalkyl, aryl or heteroaryl, wherein said 3 to 7-membered cycloalkyl group, said 3 to 7-membered heterocycloalkyl group, said aryl group or said heteroaryl group can be optionally and independently substituted with up to three R² groups;

each occurrence of R¹² is independently alkyl, haloalkyl, aryl, cycloalkyl, heterocycloalkyl or heteroaryl;

each occurrence of R¹³ is independently —C(O)—[C(R⁷)₂]_{q}N(R⁶)₂, —C(O)—[C(R⁷)₂]_{q}N(R⁶)C(O)—R¹, —C(O)—[C(R⁷)₂]_{q}N(R⁶)C(O)O—R¹, —C(O)—[C(R⁷)₂]_{q}C(O)O—R¹, —C(O)[C(R⁷)₂]_{q}N(R⁶)SO₂—R¹ or -alkylene-N(R⁶)—[C(R⁷)₂]_{q}—N(R⁶)—C(O)O—R¹;

each occurrence of R¹⁴ is H, —C₁-C₆ alkyl, 3 to 7-membered cycloalkyl, 3 to 7-membered heterocycloalkyl, aryl, heteroaryl, halo, haloalkyl, —CN, —OR³, —N(R³)₂, —C(O)R¹², —C(O)OR³, —C(O)N(R³)₂, —NHC(O)R¹², —NHC(O)NHR³, —NHC(O)OR³, —OC(O)R¹², —SR³ or —S(O)₂R¹²;

each occurrence of m is independently an integer ranging from 0 to 2; and

each occurrence of q is independently an integer ranging from 1 to 4,

such that at least one of the rings containing: (i) X², Y² and Z² or (ii) X³, Y³ and Z³ is other than a benzene ring.

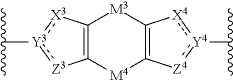

has the structure:

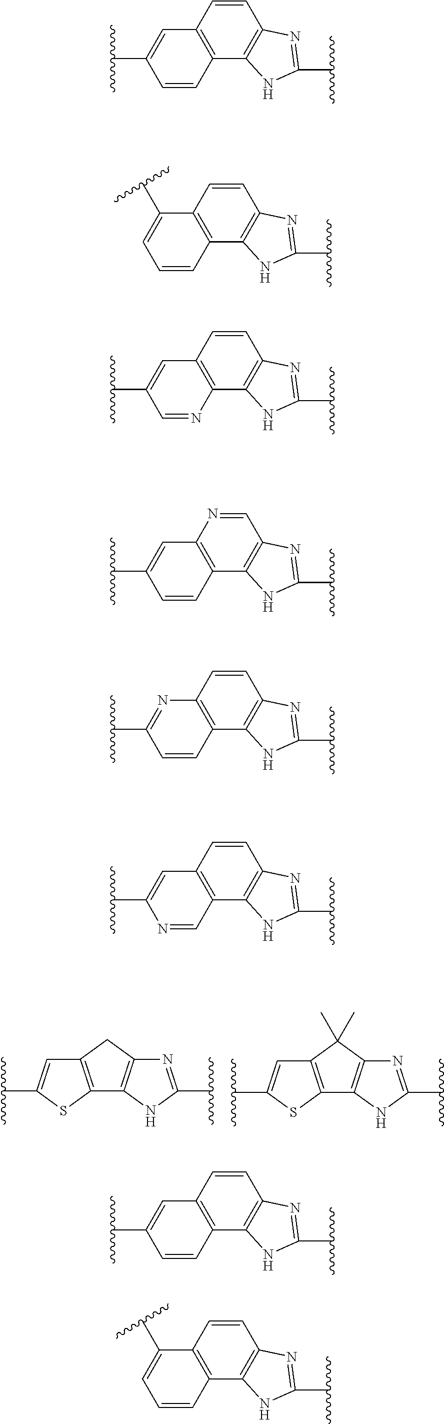

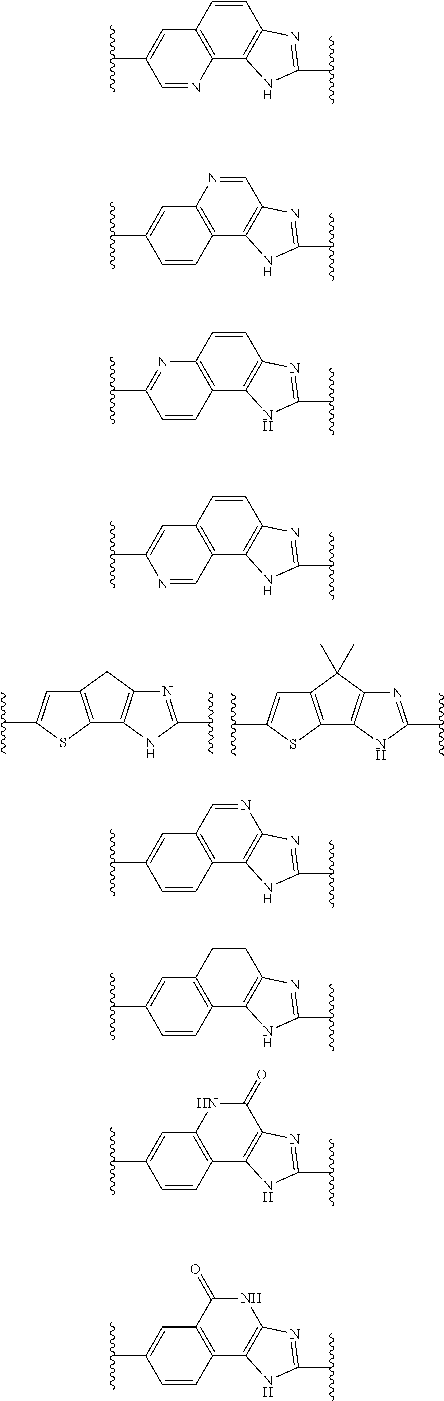

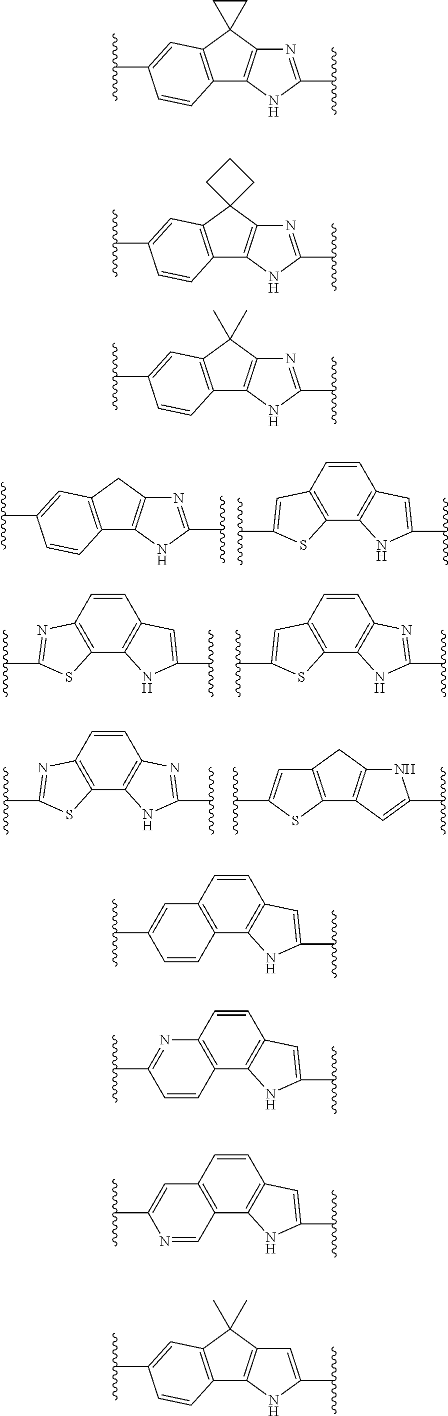

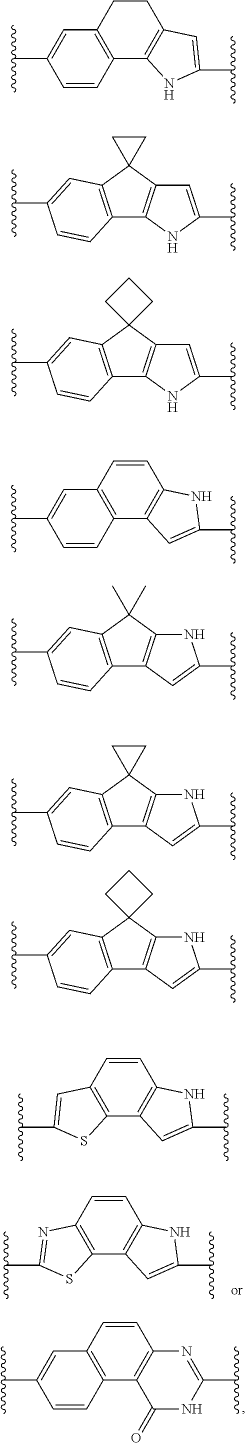

wherein either available bond on any of the above divalent groups can connect to either group flanking the above divalent groups.

In another embodiment, the group:

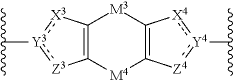

has the structure:

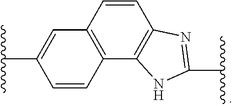

In one embodiment, B is a bond; the group:

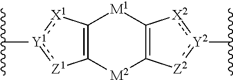

has the structure:

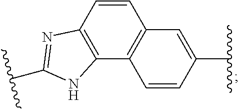

and the group:

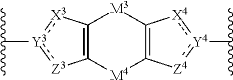

has the structure:

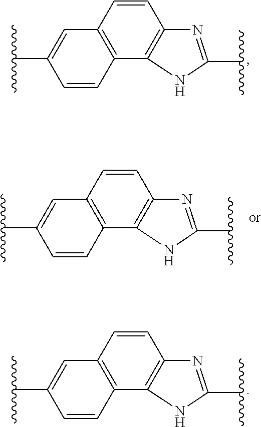

In one embodiment, R⁴ is —C₁-C₆ alkyl.

In another embodiment, R⁴ is halo.

In another embodiment, R⁴ is —C(O)—[C(R⁵₂]_{q}N(R⁶)₂.

In still another embodiment, R⁴ is —C(O)—[CH(R⁵)]_{q}N(R⁶)C(O)—R¹.

In another embodiment, R⁴ is —C(O)—[CH(R⁵)]_{q}N(R⁶)C(O)O—R¹.

In another embodiment, R⁴ is —C(O)—[CH(R⁵]_{q}C(O)O—R¹.

In yet another embodiment, R⁴ is —C(O)[CH(R⁵]_{q}N(R⁶)SO₂—R¹.

In another embodiment, R⁴ is -alkylene-N(R⁶)—[CH(R⁵]_{q}—N(R⁶)—C(O)O—R¹.

In one embodiment, each occurrence of R⁴ is independently selected from:

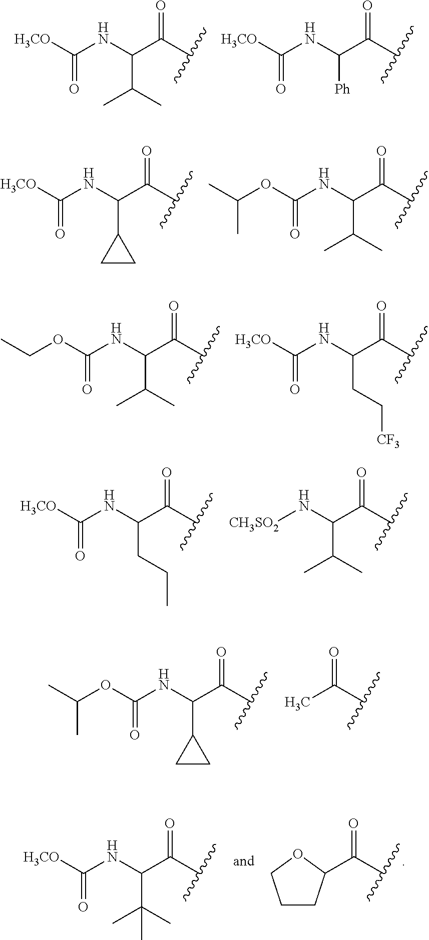

In another embodiment, each occurrence of R⁴ is independently —C(O)—[CH(R⁵)]_{q}N(R⁶)C(O)O—R¹.

In another embodiment, each occurrence of R⁴ is independently:

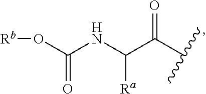

wherein R^{a} is H, alkyl, haloalkyl, cycloalkyl or aryl, and R^{b} is alkyl, haloalkyl or cycloalkyl.

In another embodiment, each occurrence of R⁴ is independently:

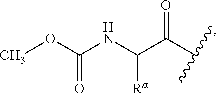

wherein R^{a} is H, methyl, ethyl, propyl, isopropyl, —CH(CH₃)(OCH₃), t-butyl, cyclopropyl, —CH₂CH₂CF₃ or phenyl.

In another embodiment, each occurrence of R⁴ is independently:

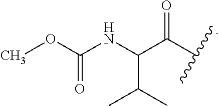

In one embodiment, A and C are each

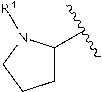

and B is a bond.

In another embodiment, A and C are each independently selected from:

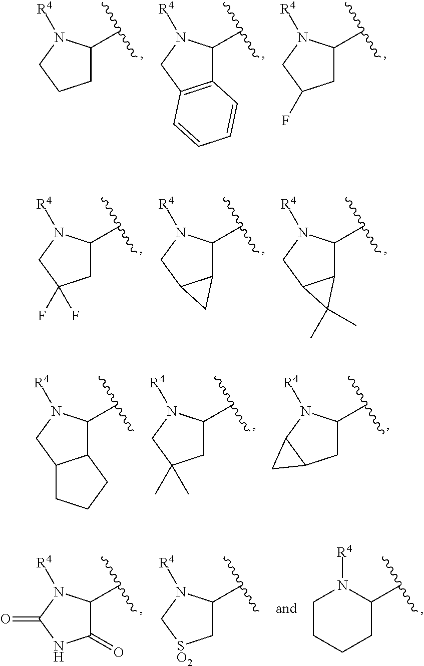

and each occurrence of R⁴ is independently selected from:

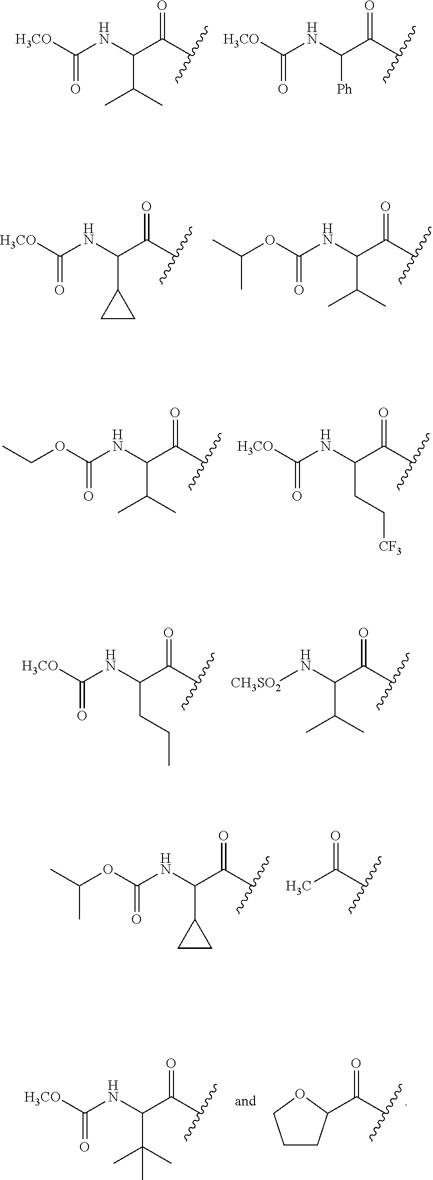

In still another embodiment, A and C are each independently selected from:

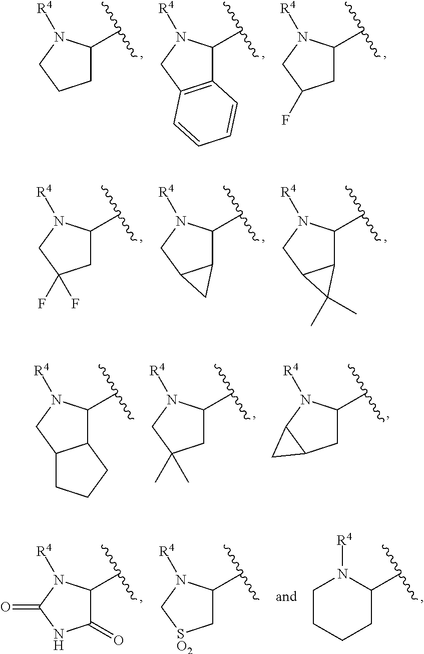

and each occurrence of R⁴ is independently:

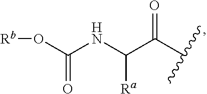

wherein R^{a} is H, alkyl, haloalkyl, cycloalkyl or aryl, and R^{b} is alkyl, haloalkyl or cycloalkyl.

In another embodiment, A and C are each independently selected from:

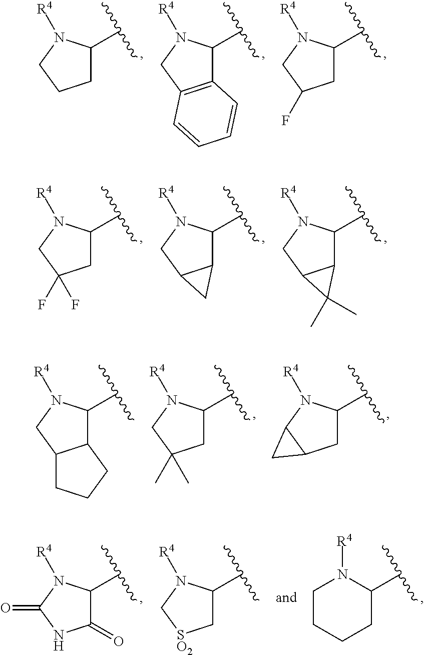

and each occurrence of R⁴ is independently:

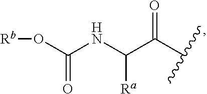

wherein R^{a} is H, methyl, ethyl, propyl, isopropyl, —CH(CH₃)(OCH₃), t-butyl, cyclopropyl, —CH₂CH₂CF₃ or phenyl, and R^{b} is methyl, ethyl, propyl, isopropyl, t-butyl, cyclopropyl, or —CH₂CH₂CF₃.

In another embodiment, A and C are each independently selected from:

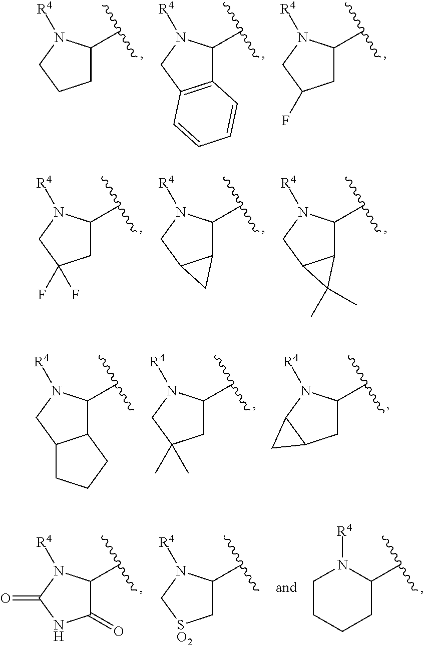

wherein each occurrence of R⁴ is:

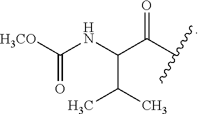

In a further embodiment, A and C are each

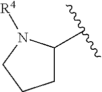

and each occurrence of R⁴ is independently selected from:

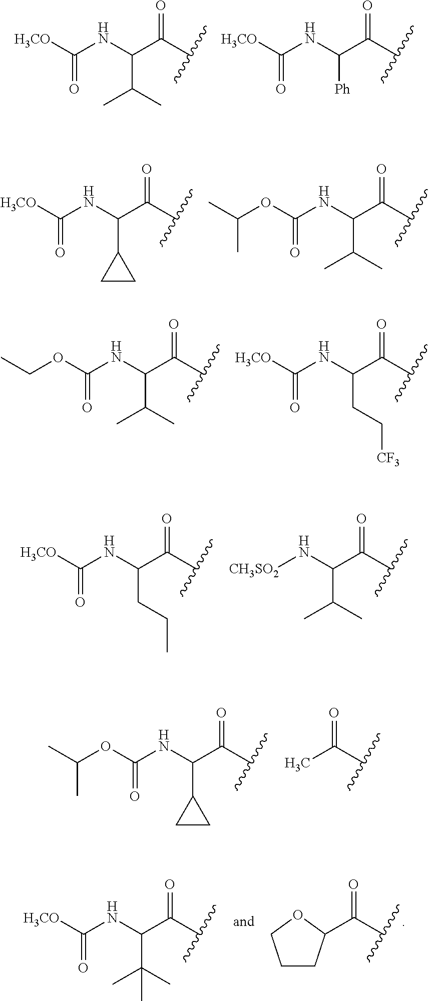

In a further embodiment, A and C are each

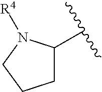

and each occurrence of R⁴ is independently selected from:

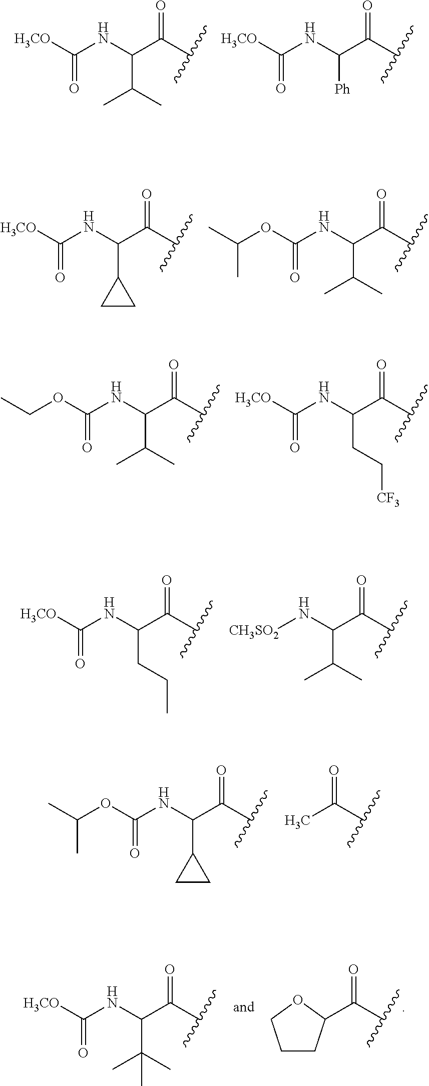

In one embodiment, A and C are each

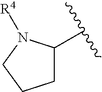

and each occurrence of R⁴ is independently:

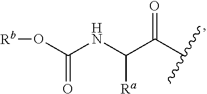

wherein R^{a} is H, alkyl, haloalkyl, cycloalkyl or aryl, and R^{b} is alkyl, haloalkyl or cycloalkyl.

In each embodiment, A and C are each

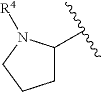

and each occurrence of R⁴ is independently:

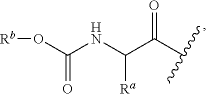

wherein R^{a}is H, methyl, ethyl, propyl, isopropyl, —CH(CH₃)(OCH₃), t-butyl, cyclopropyl, —CH₂CH₂CF₃ or phenyl, and R^{b} is methyl, ethyl, propyl, isopropyl, t-butyl, cyclopropyl, or —CH₂CH₂CF₃.

In another embodiment, A and C are each

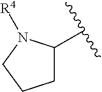

and each occurrence of R⁴ is:

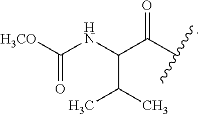

In one embodiment, the group:

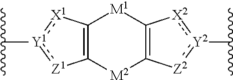

has the structure:

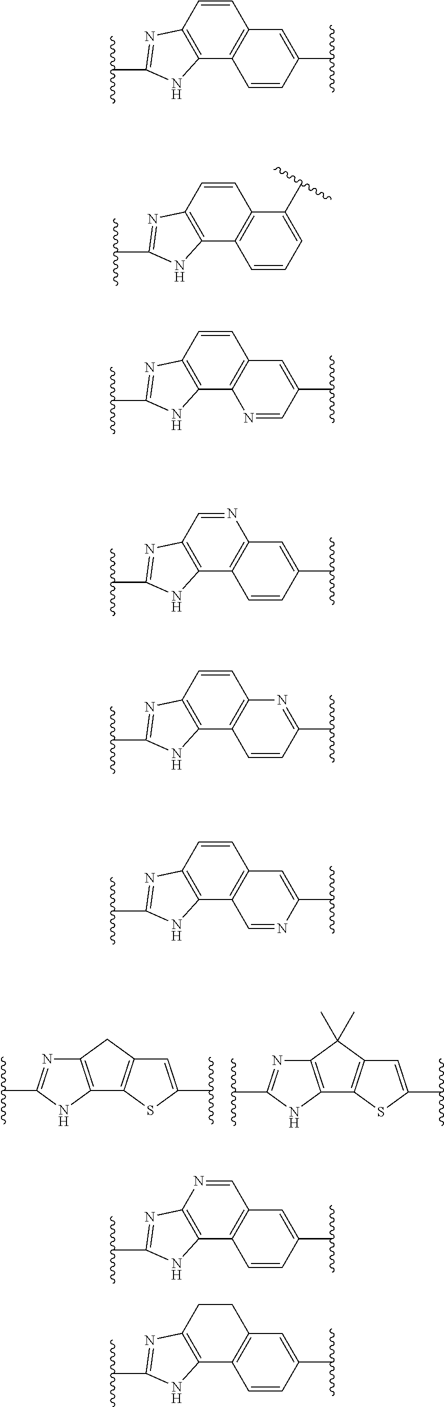

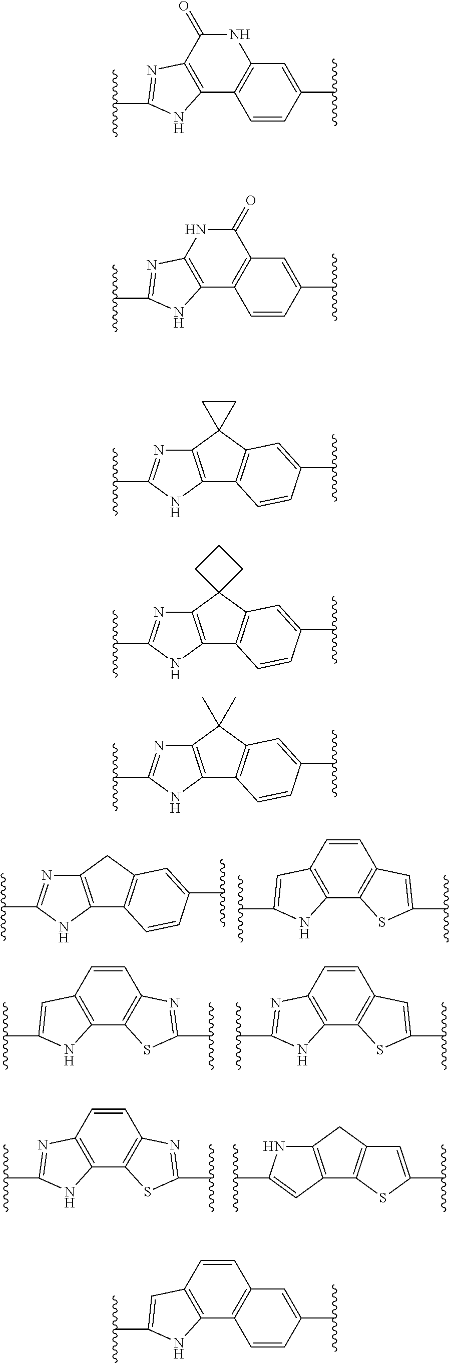

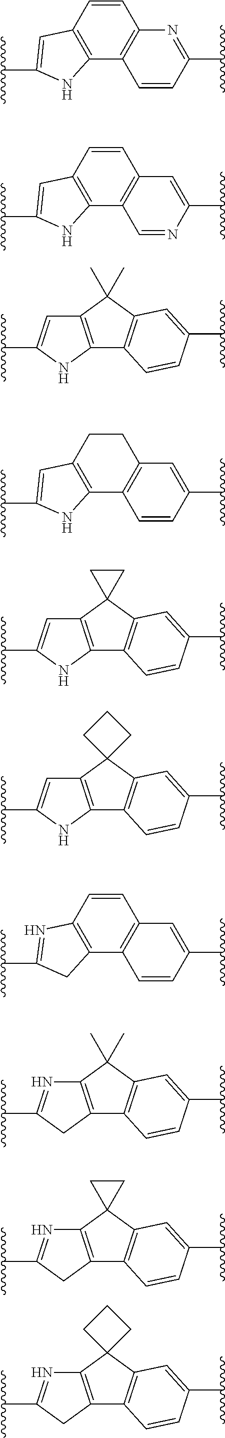

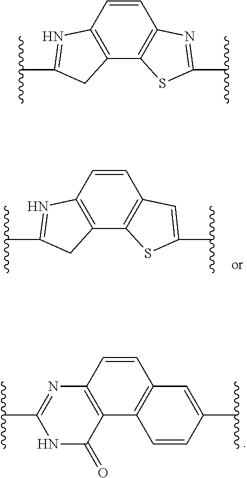

In another embodiment, the group:

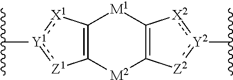

has the structure:

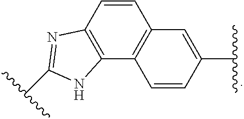

In one embodiment, the group:

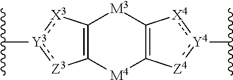

has the structure:

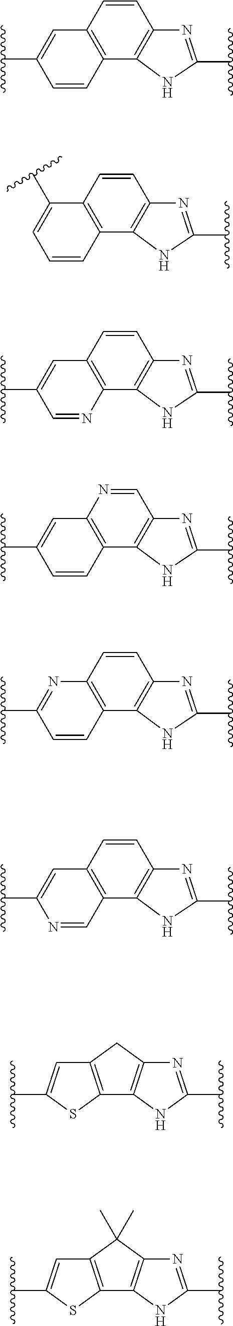

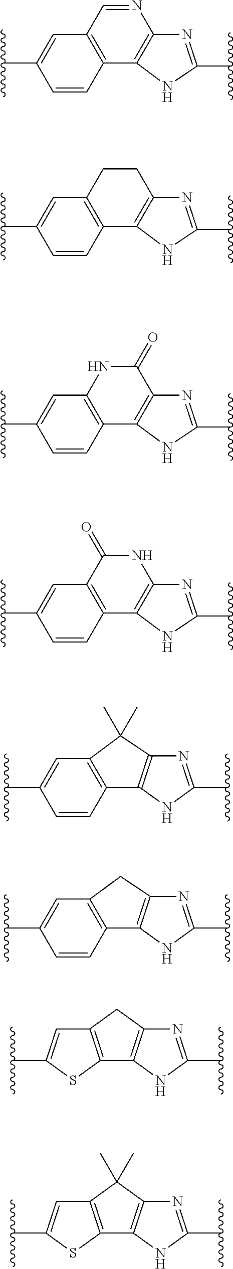

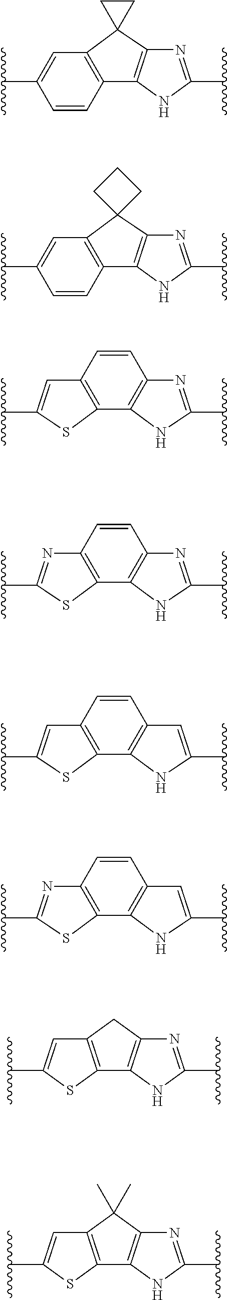

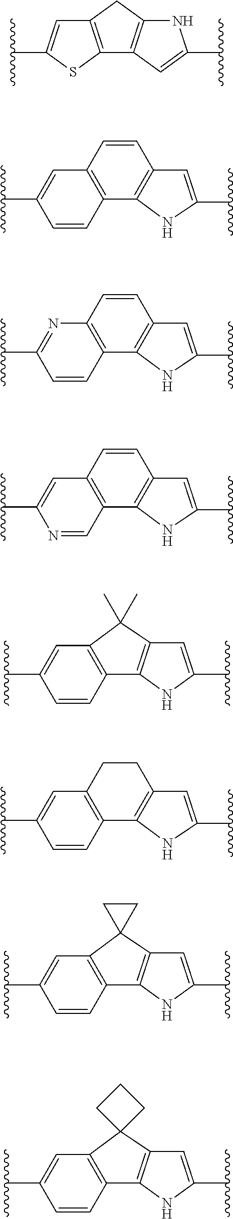

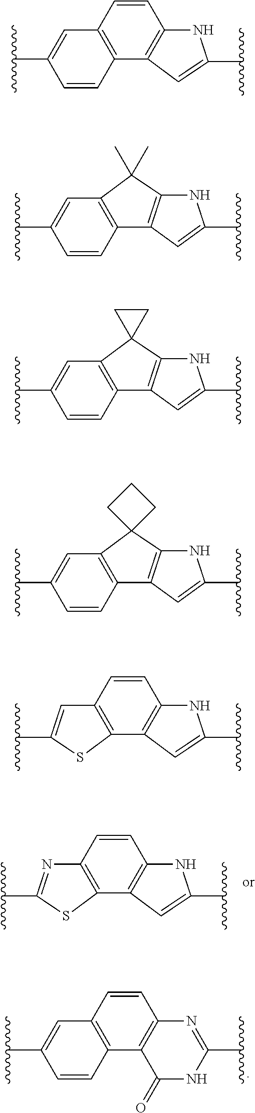

In another embodiment, the group:

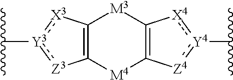

has the structure:

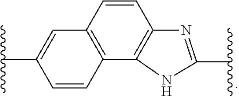

In another embodiment, the group:

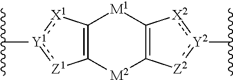

has the structure:

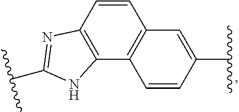

and the group:

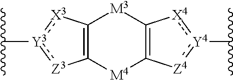

has the structure:

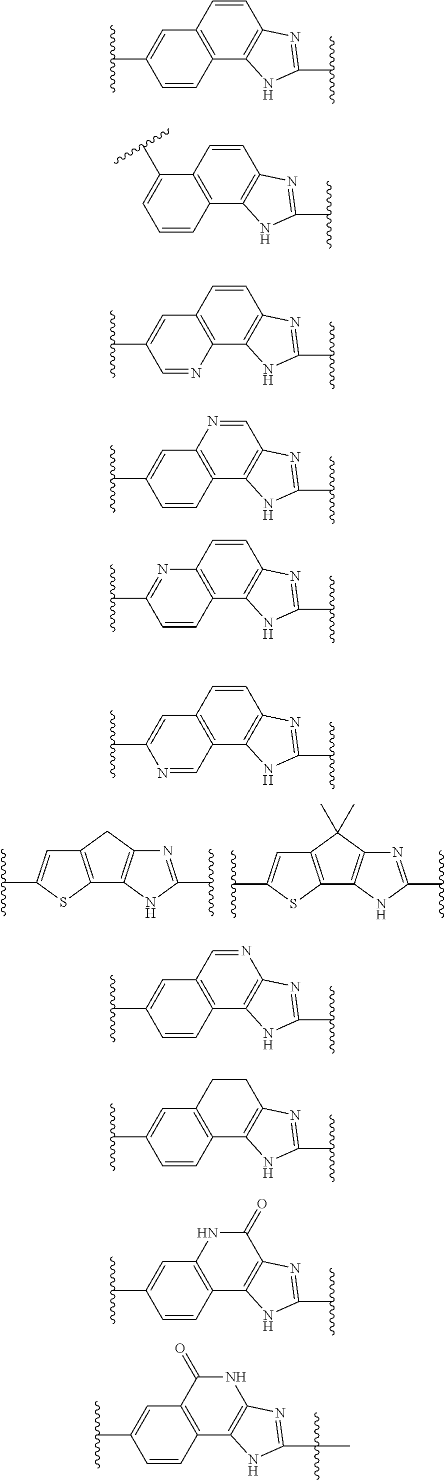

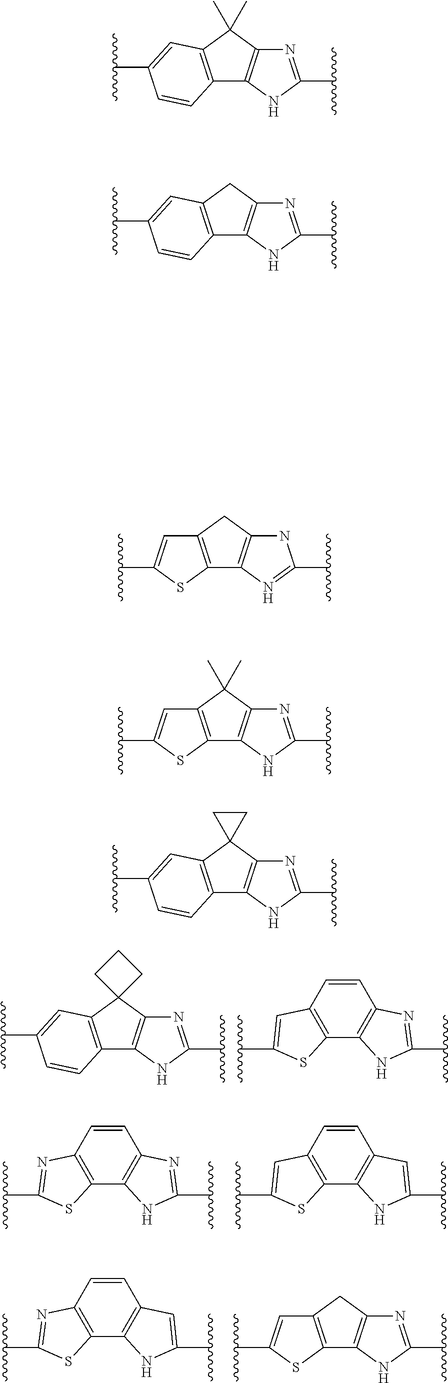

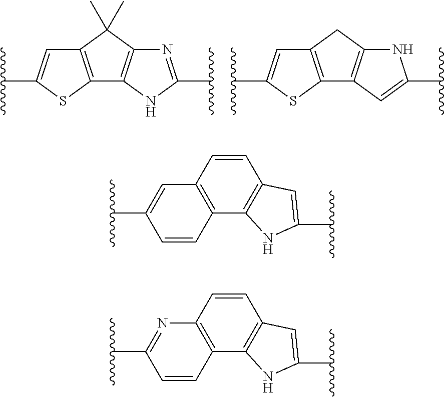

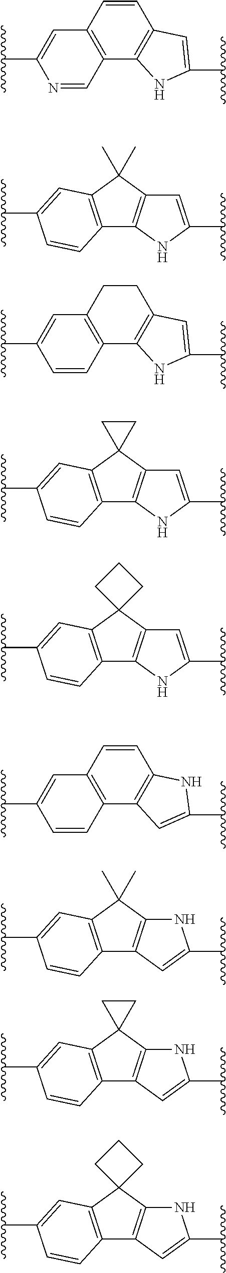

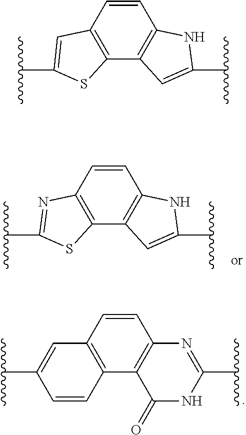

In another embodiment, the group:

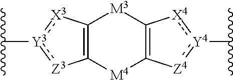

has the structure:

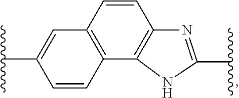

and the group:

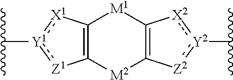

has the structure:

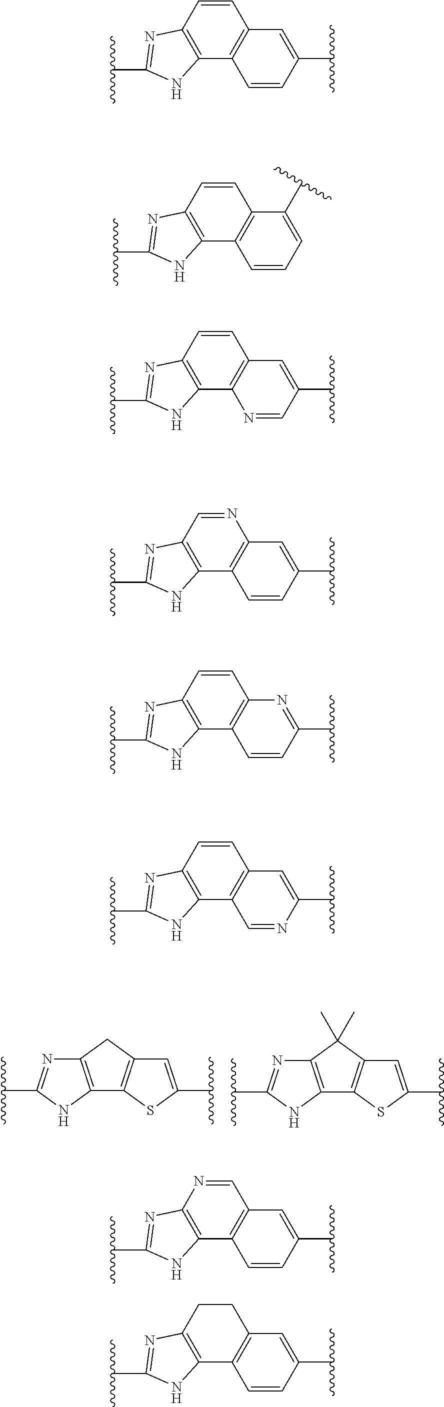

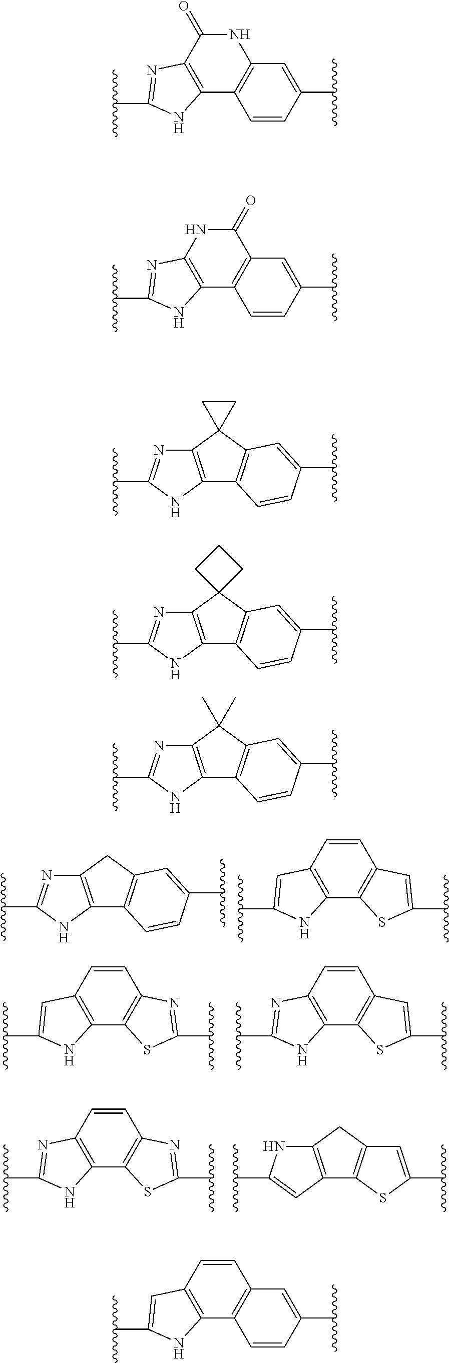

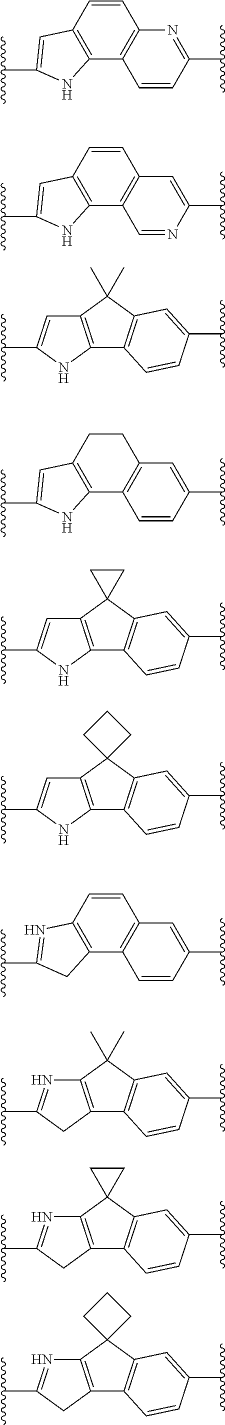

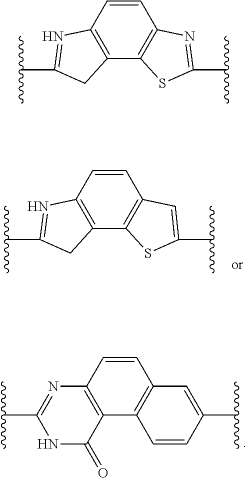

In still another embodiment, the group:

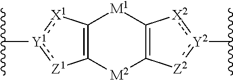

has the structure:

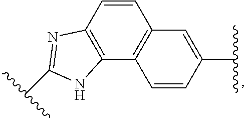

and the group:

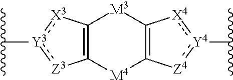

has the structure:

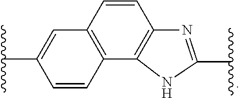

In one embodiment, A and C are each independently -alkylene-N(R¹¹)(R¹³), and the group:

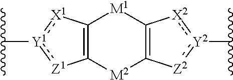

has the structure:

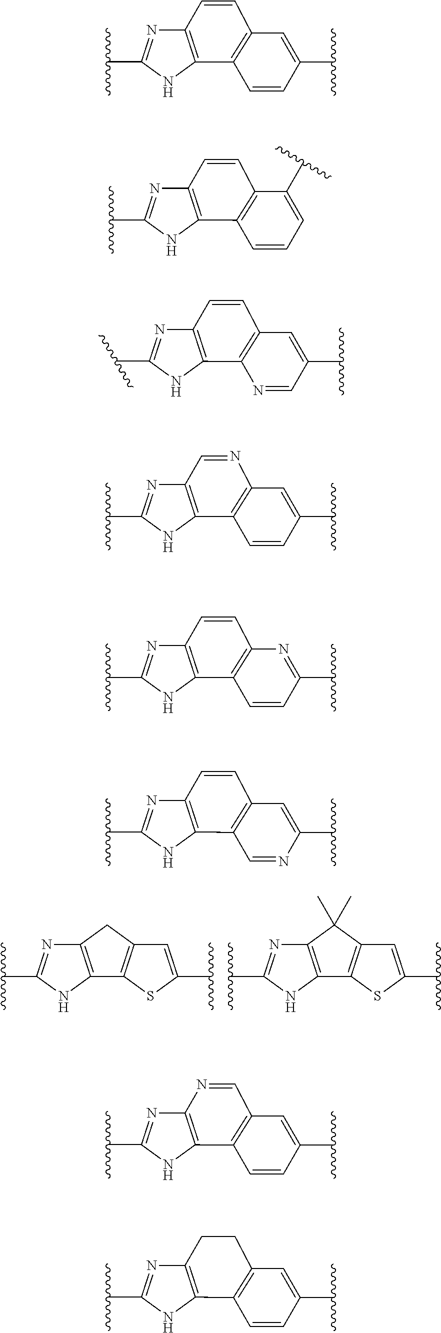

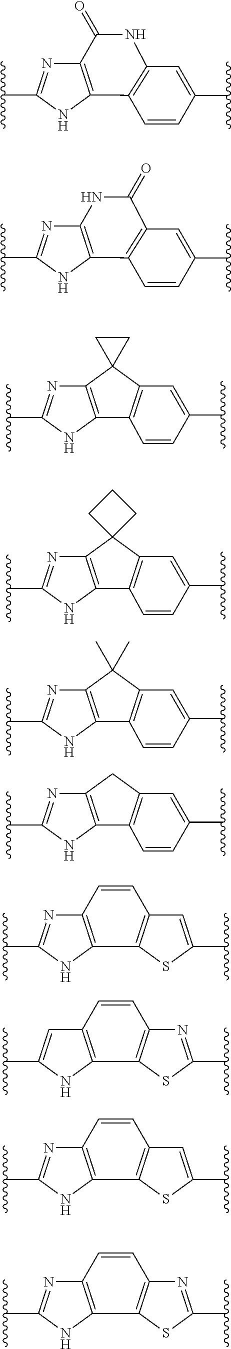

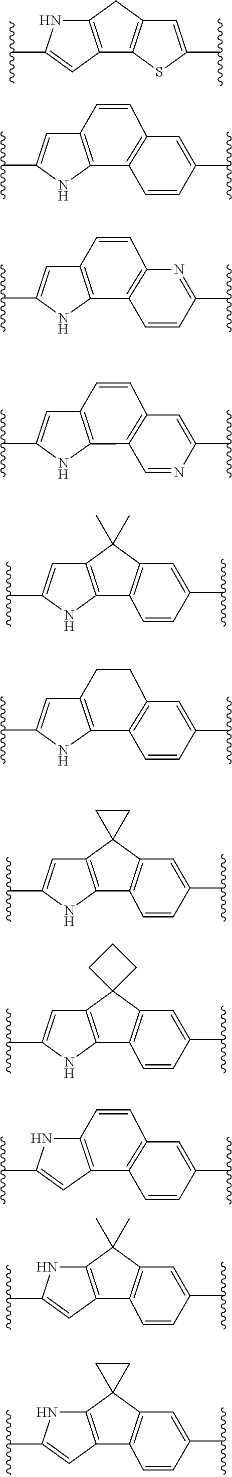

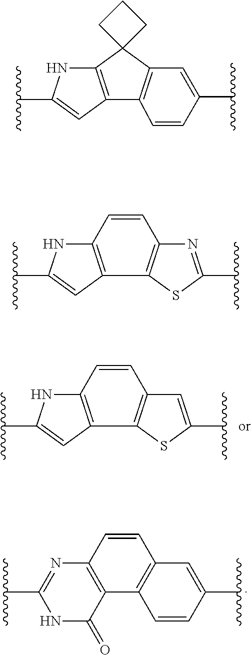

In another embodiment, A and C are each independently heterocycloalkyl, and the group:

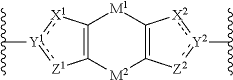

has the structure:

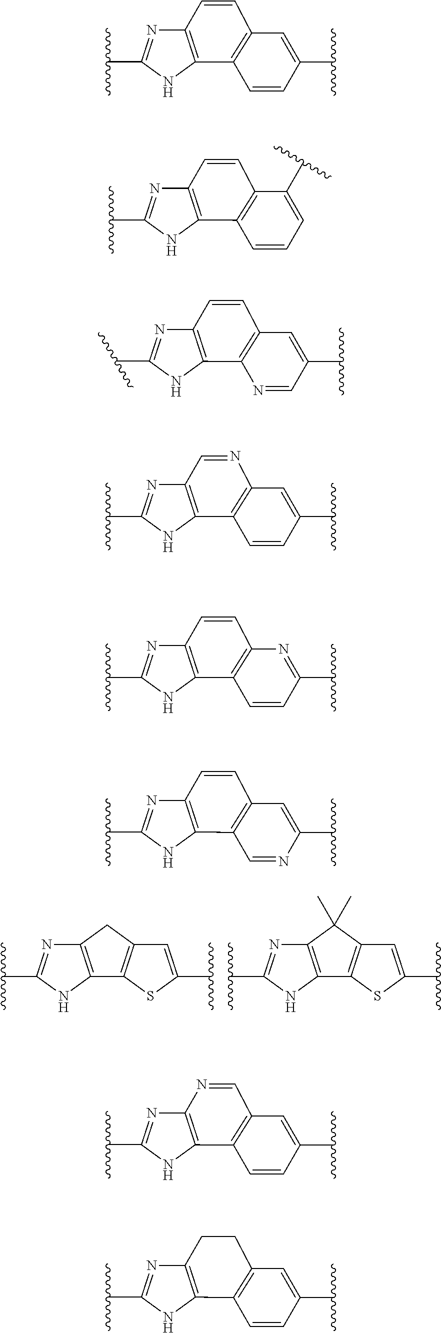

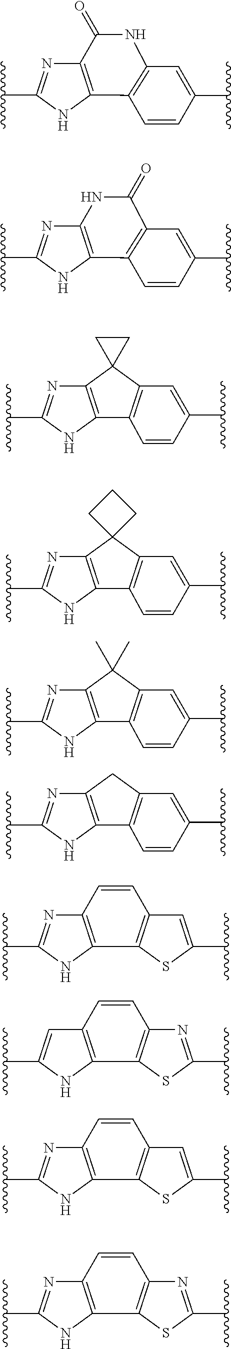

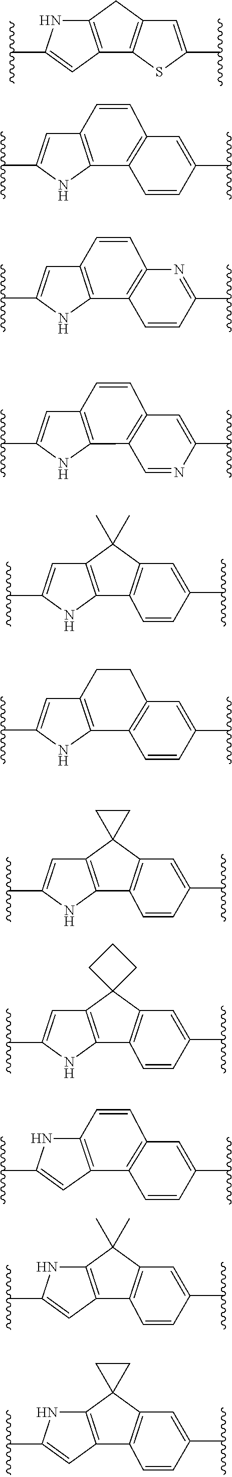

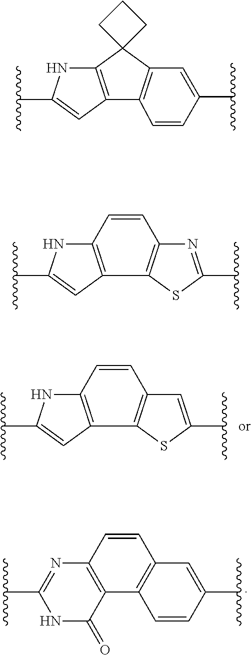

In another embodiment, A and C are each independently selected from:

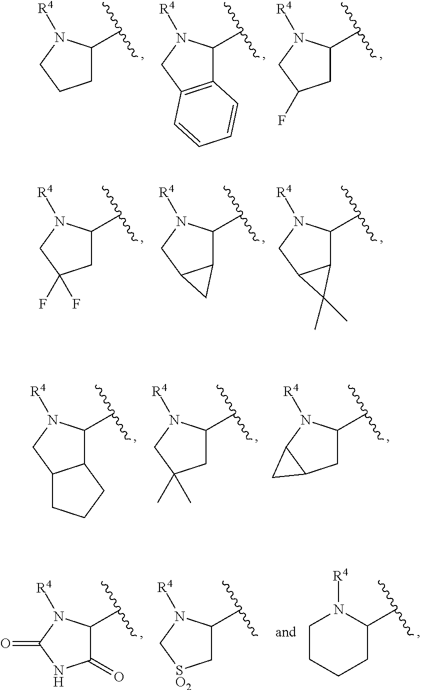

and the group:

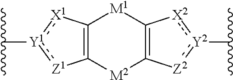

has the structure:

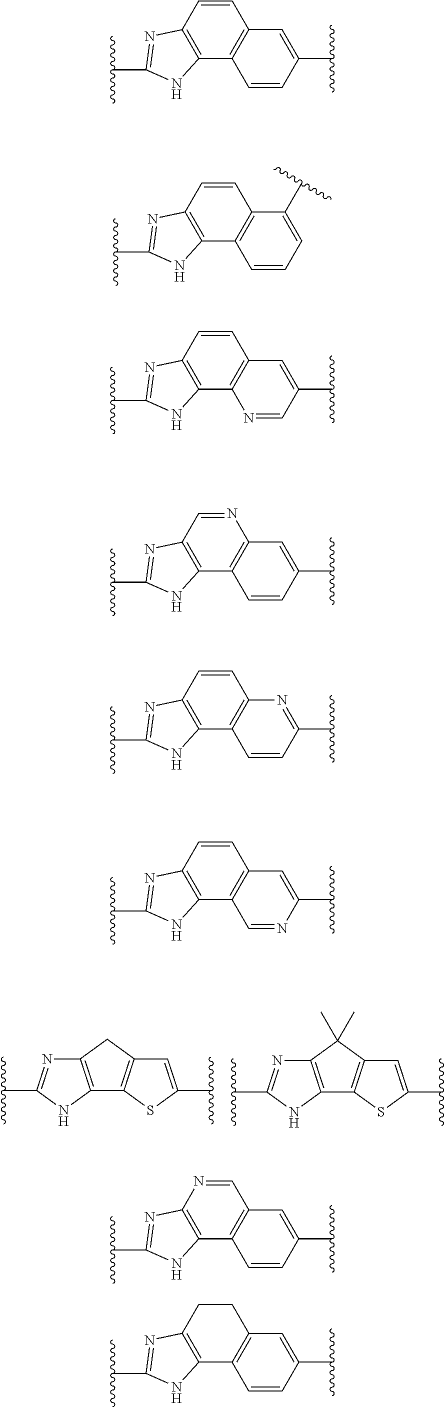

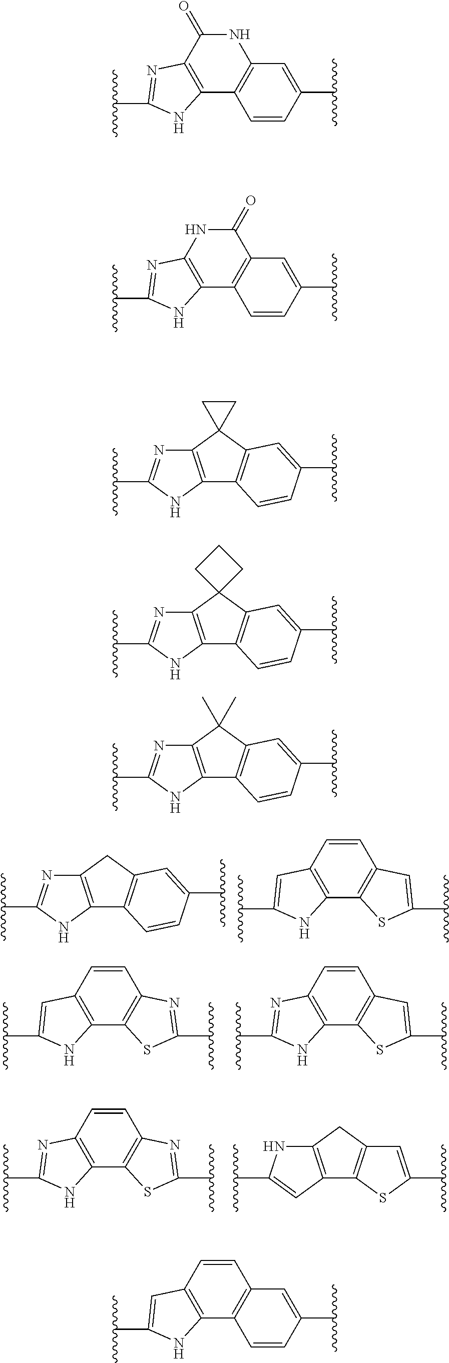

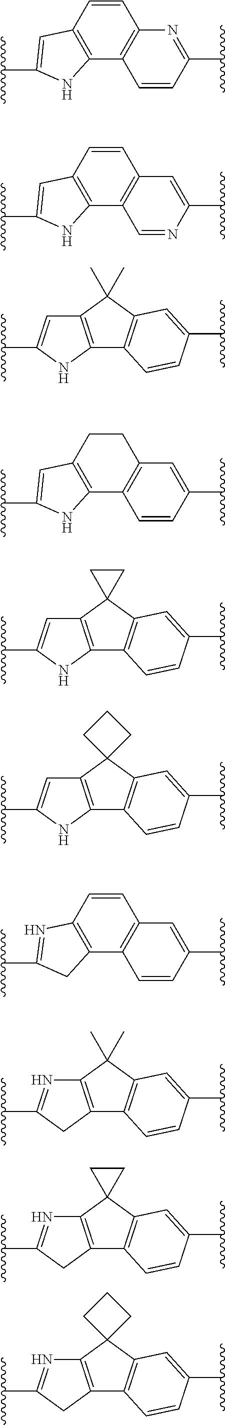

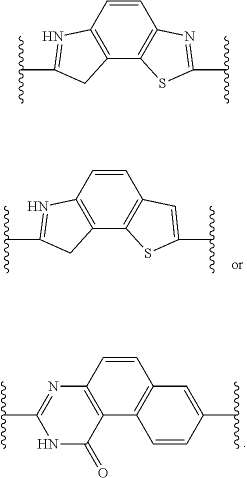

In still another embodiment, A and C are each:

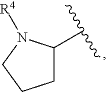

and the group:

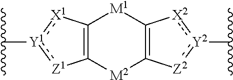

has the structure:

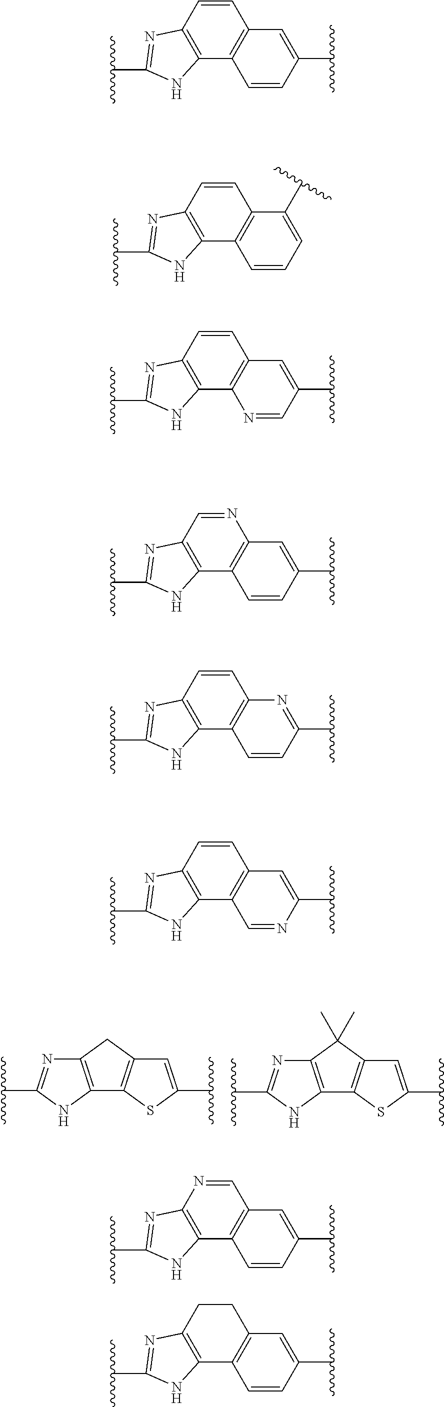

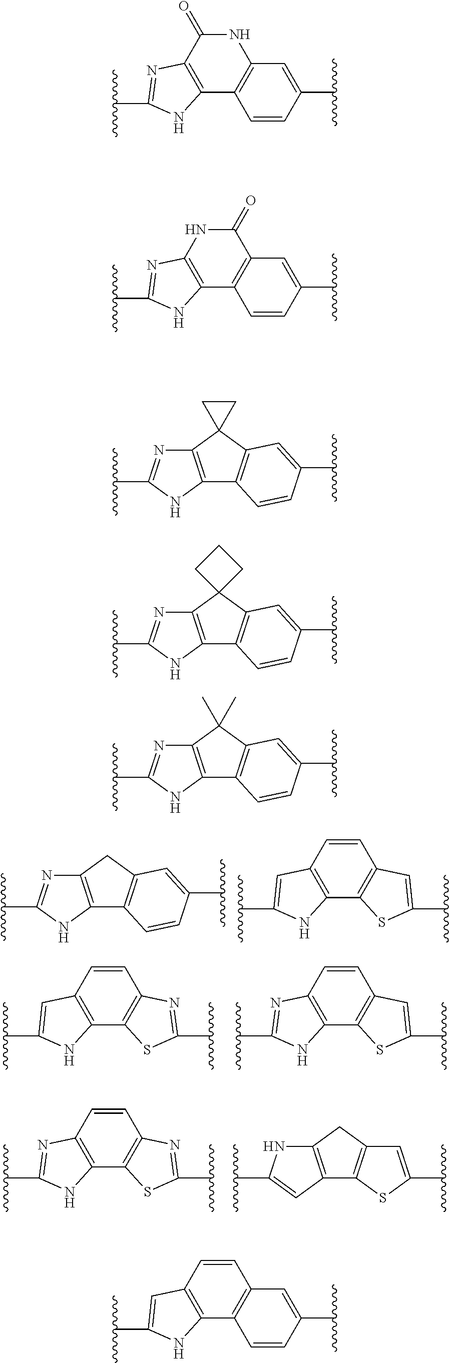

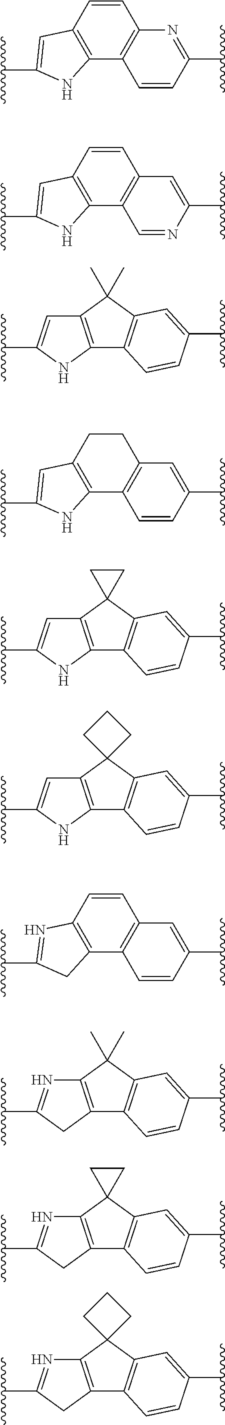

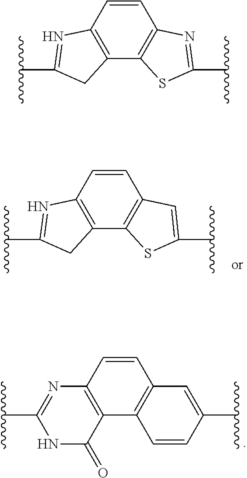

In one embodiment, A and C are each independently -alkylene-N(R¹¹)(R¹³), and the group:

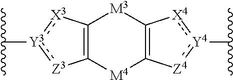

has the structure:

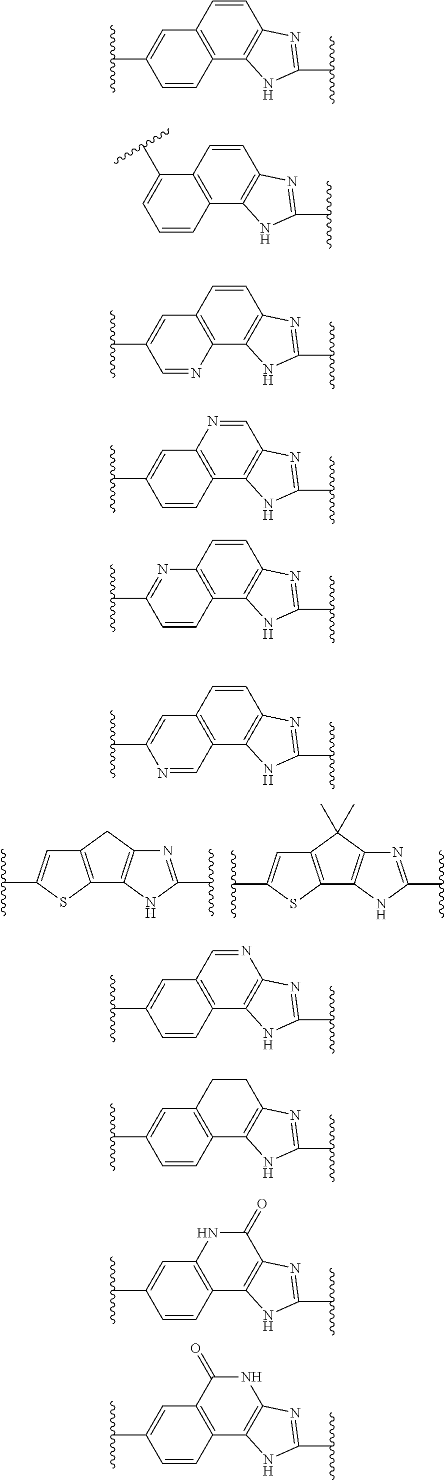

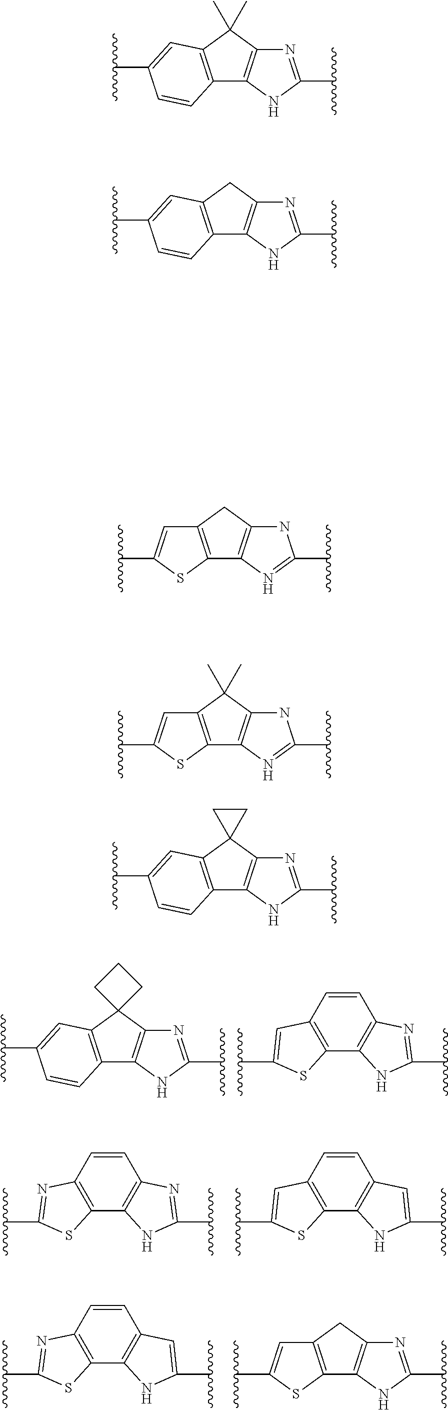

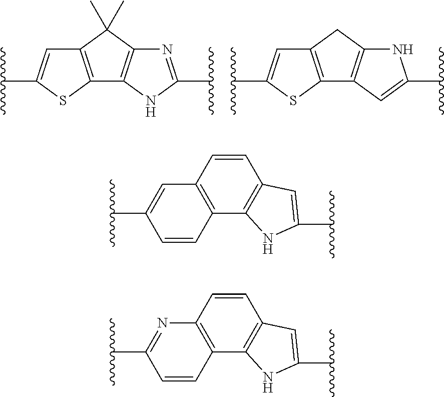

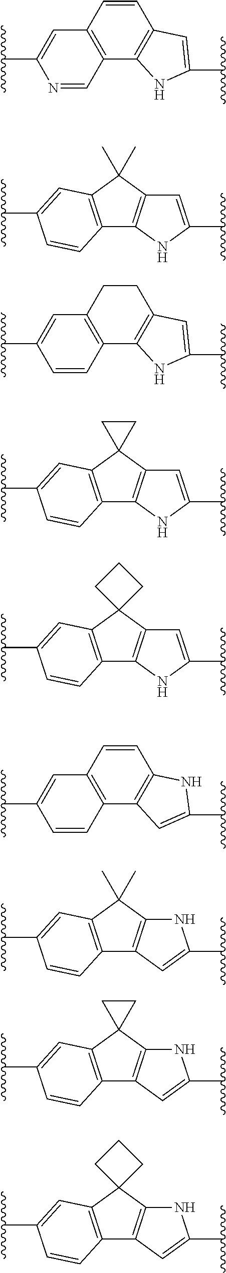

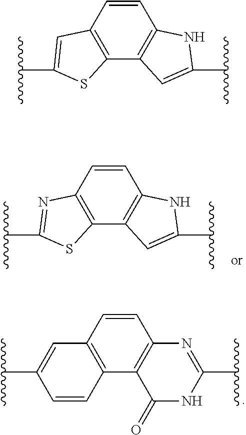

In another embodiment, A and C are each independently heterocycloalkyl, and the group:

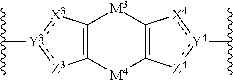

has the structure:

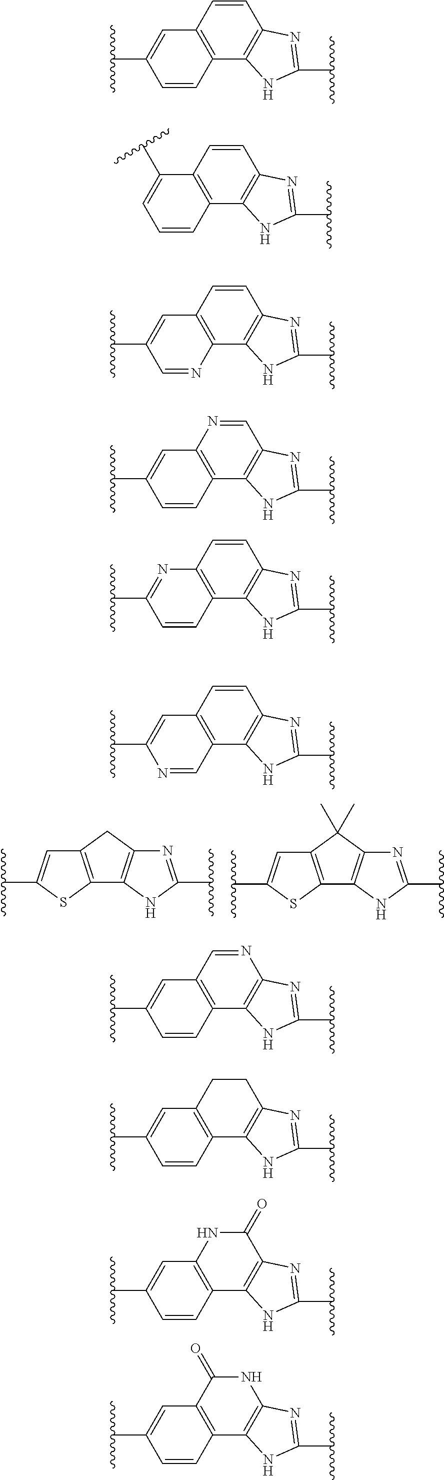

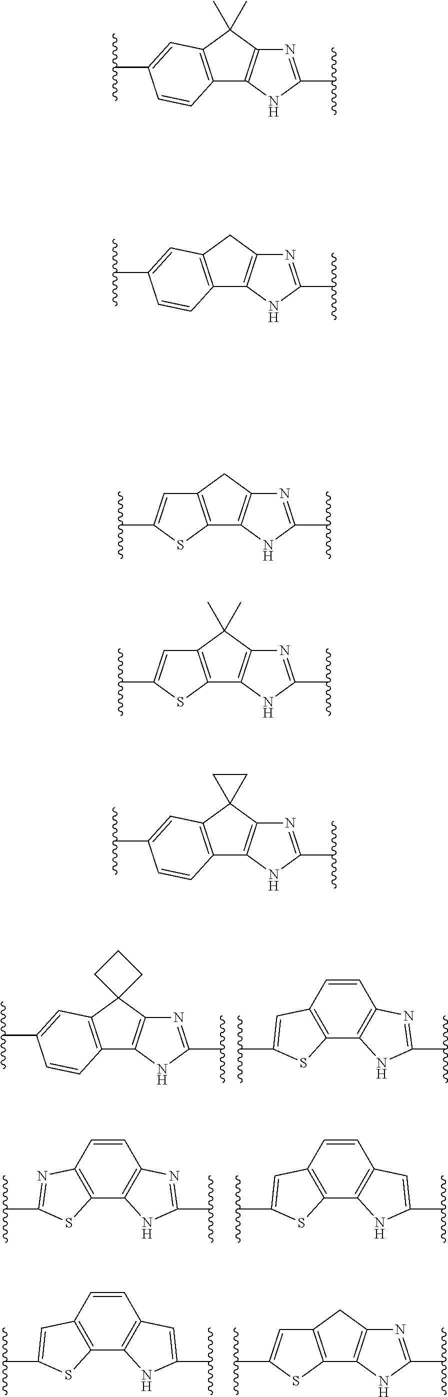

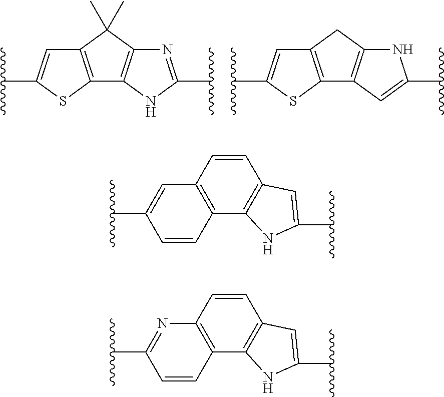

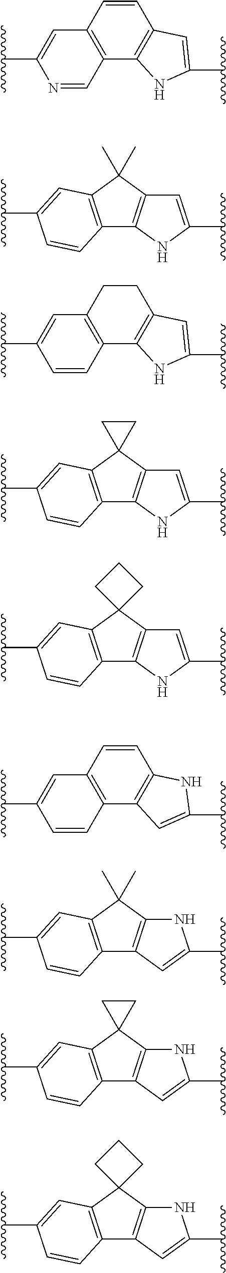

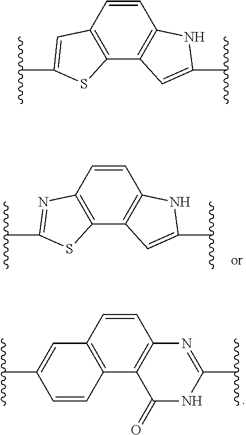

In another embodiment, A and C are each independently selected from:

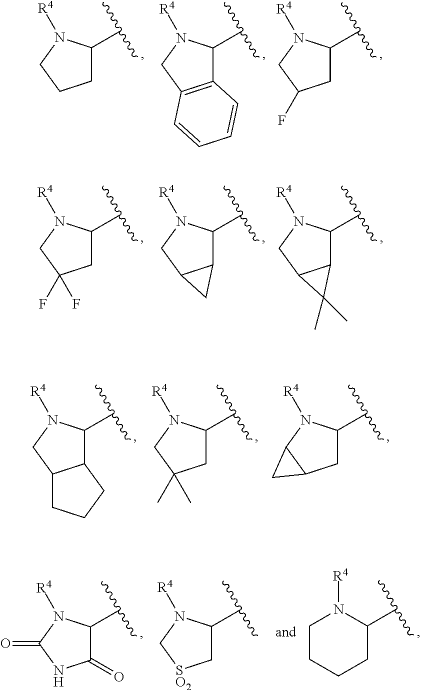

and the group:

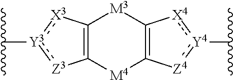

has the structure:

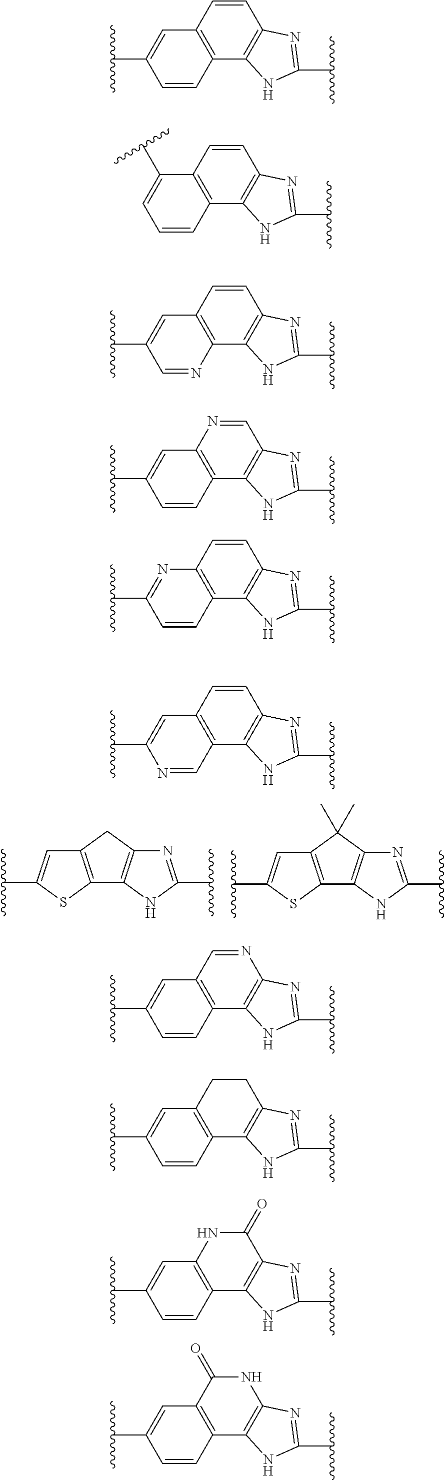

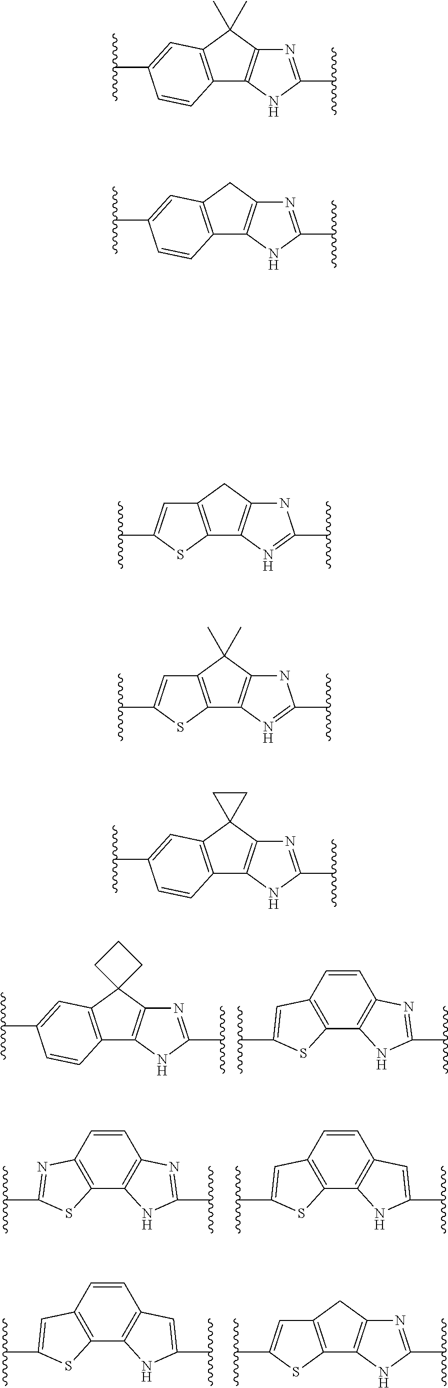

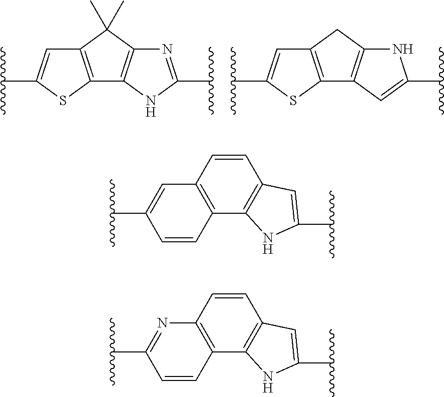

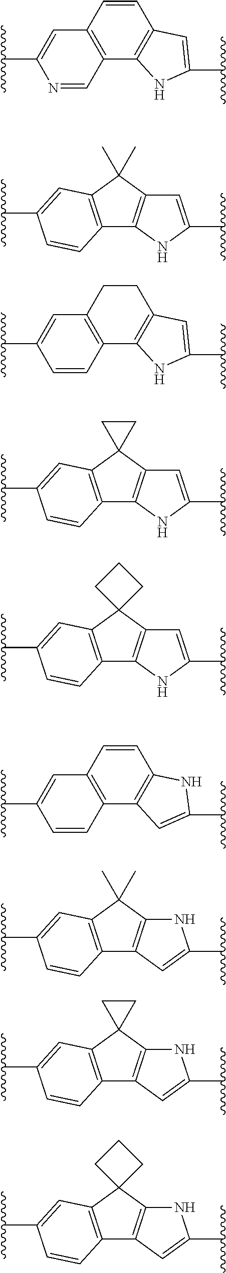

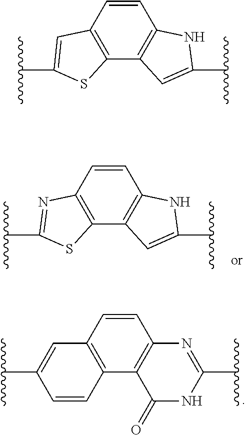

In still another embodiment, A and C are each:

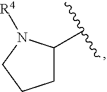

and the group:

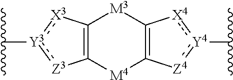

has the structure:

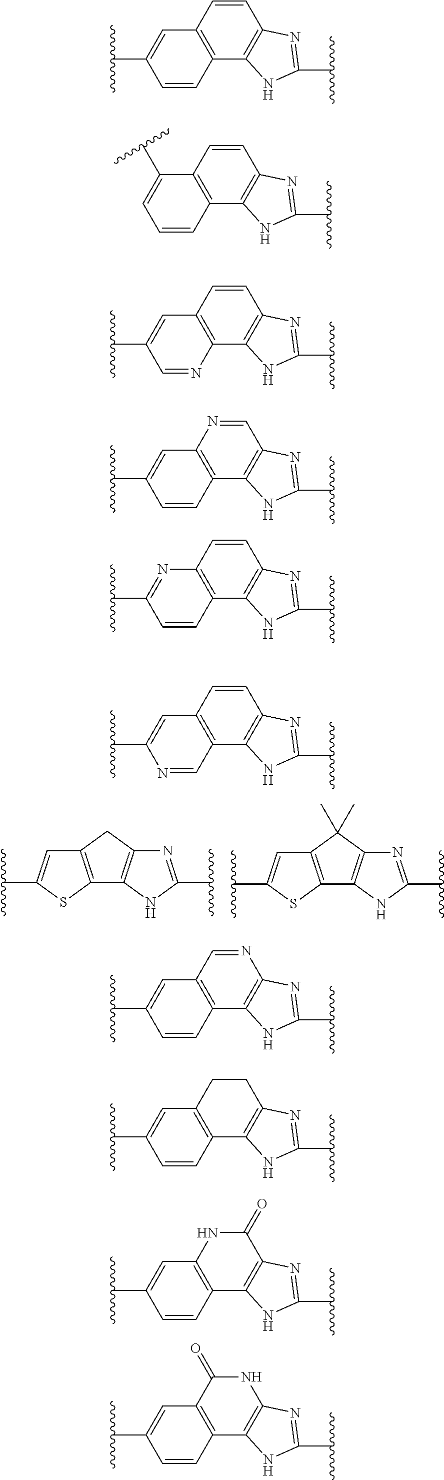

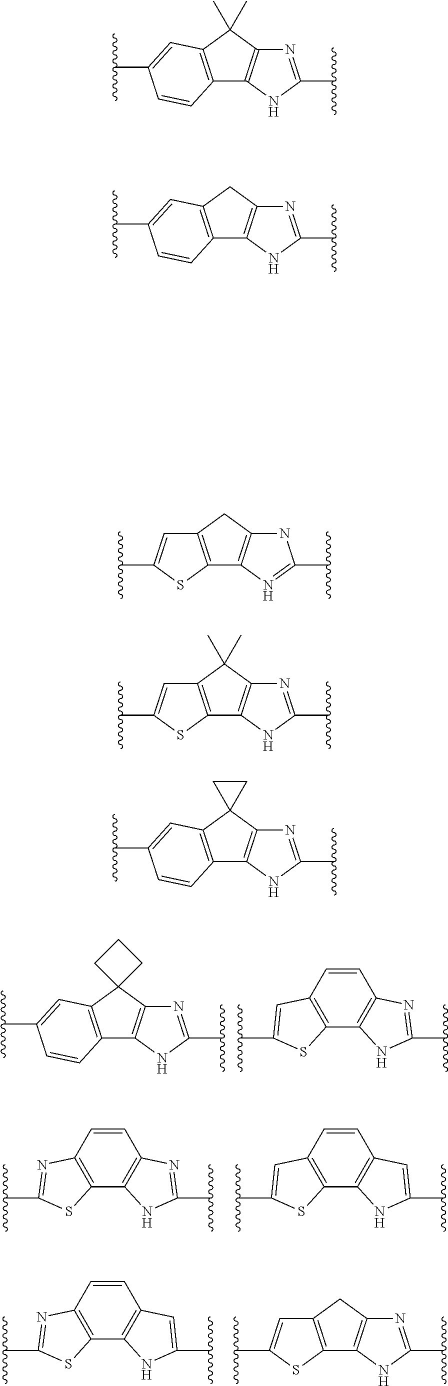

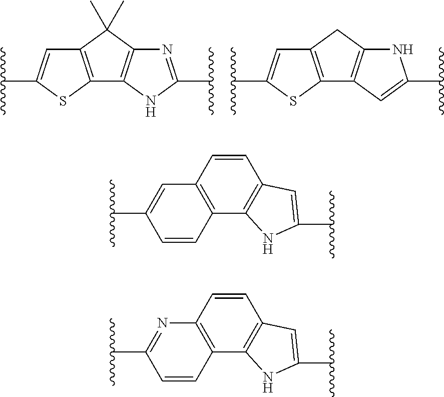

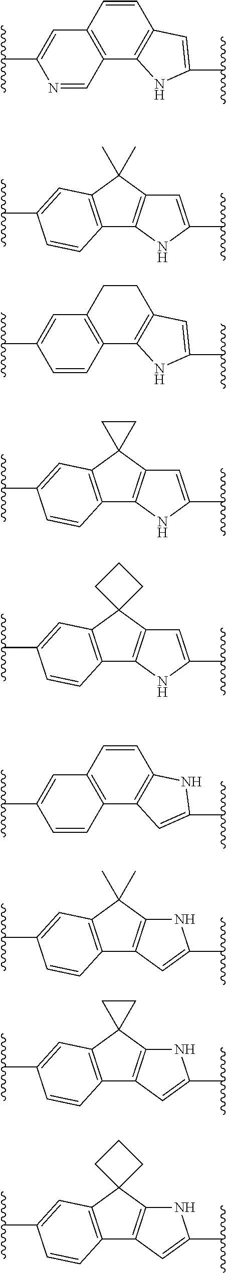

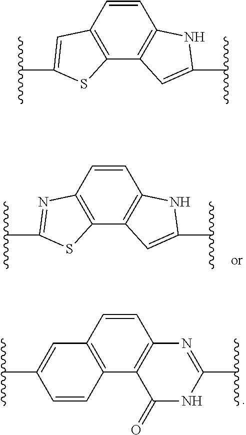

In one embodiment, A and C are each independently selected from:

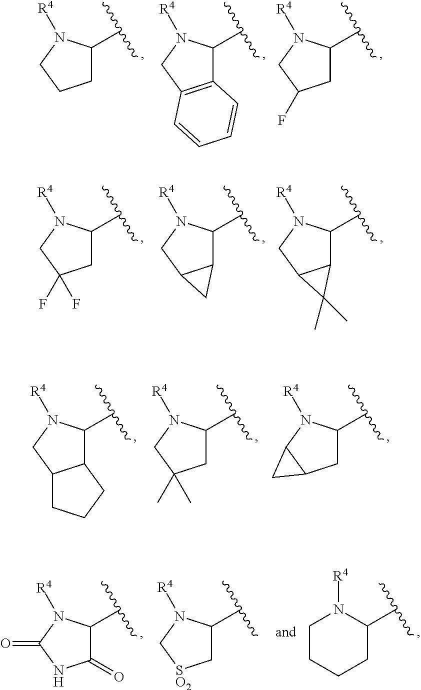

the group:

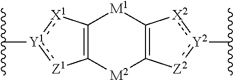

has the structure:

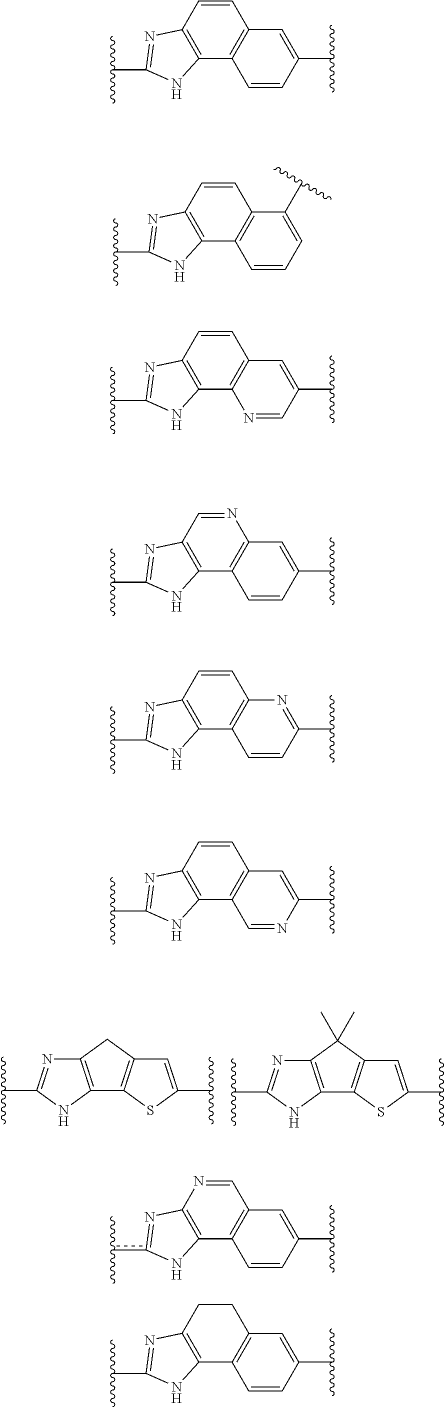

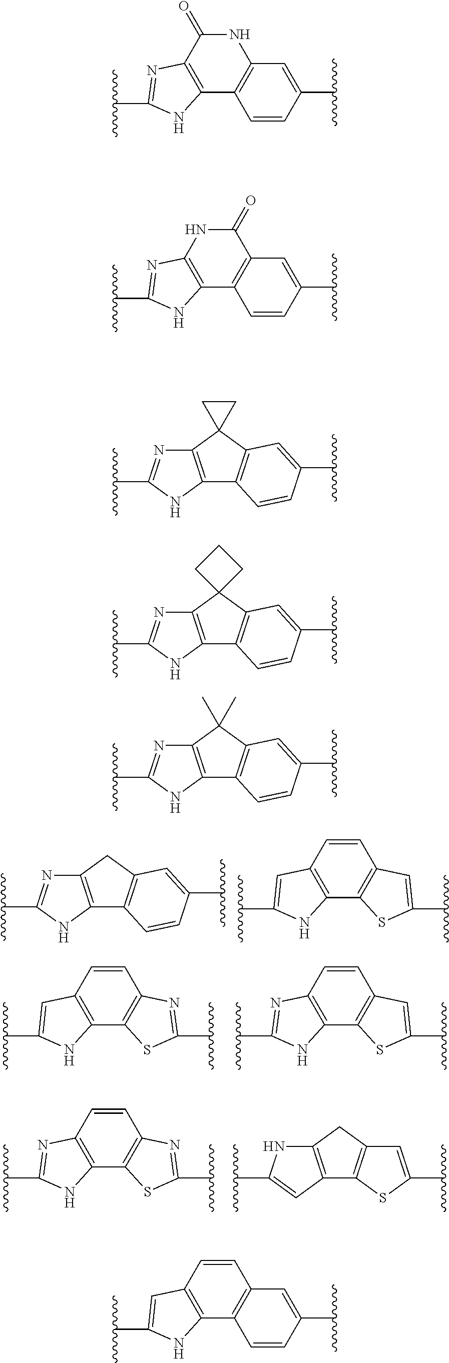

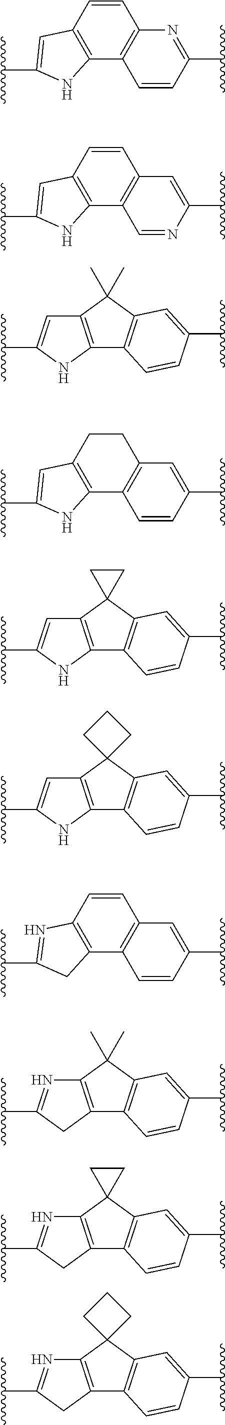

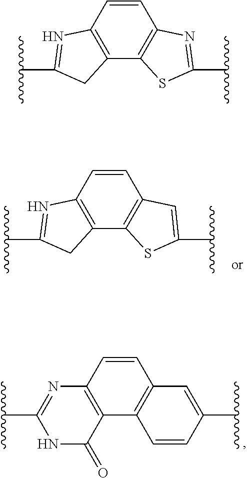

and each occurrence of R⁴ is independently selected from:

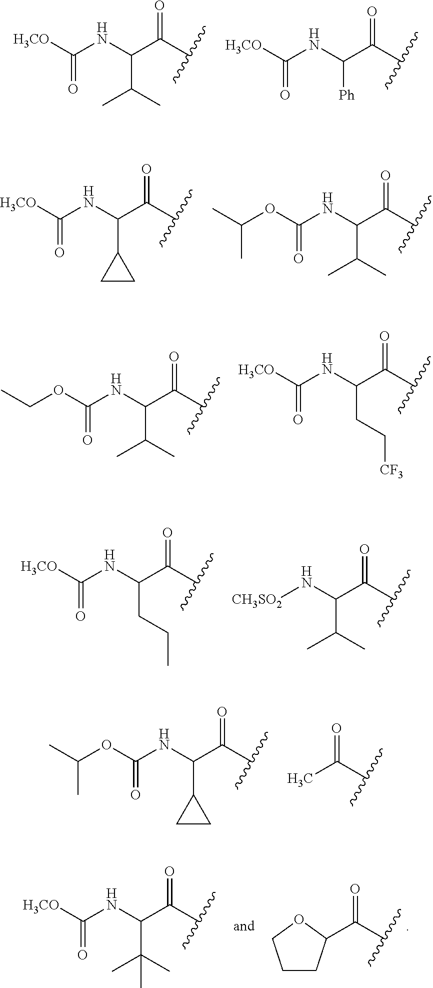

In another embodiment, A and C are each independently selected from:

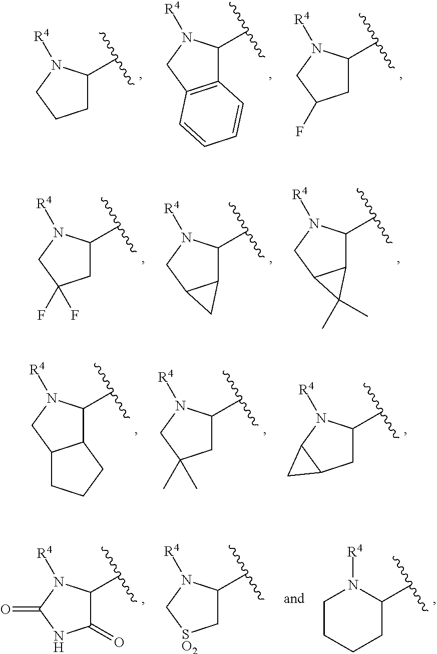

the group:

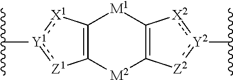

has the structure:

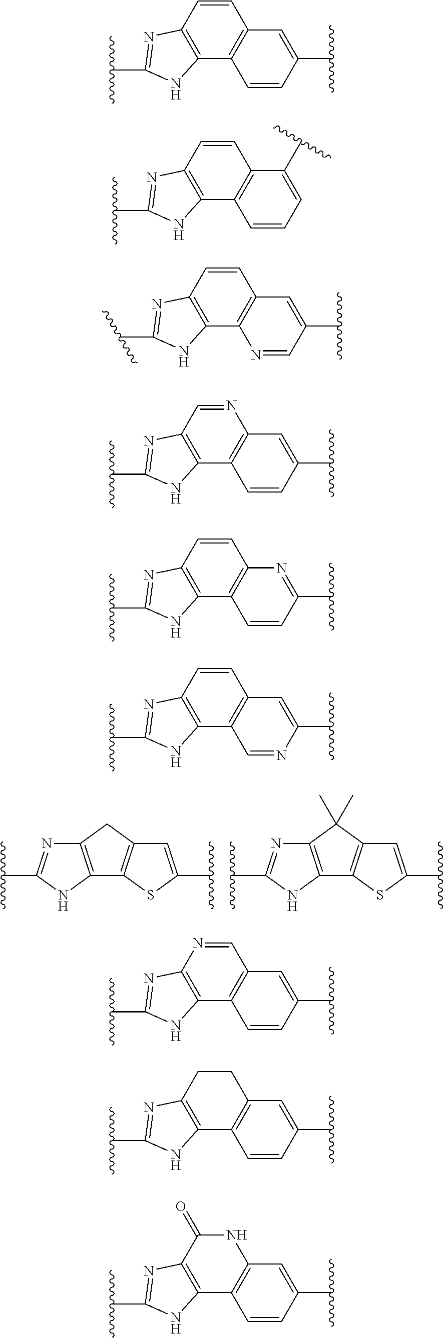

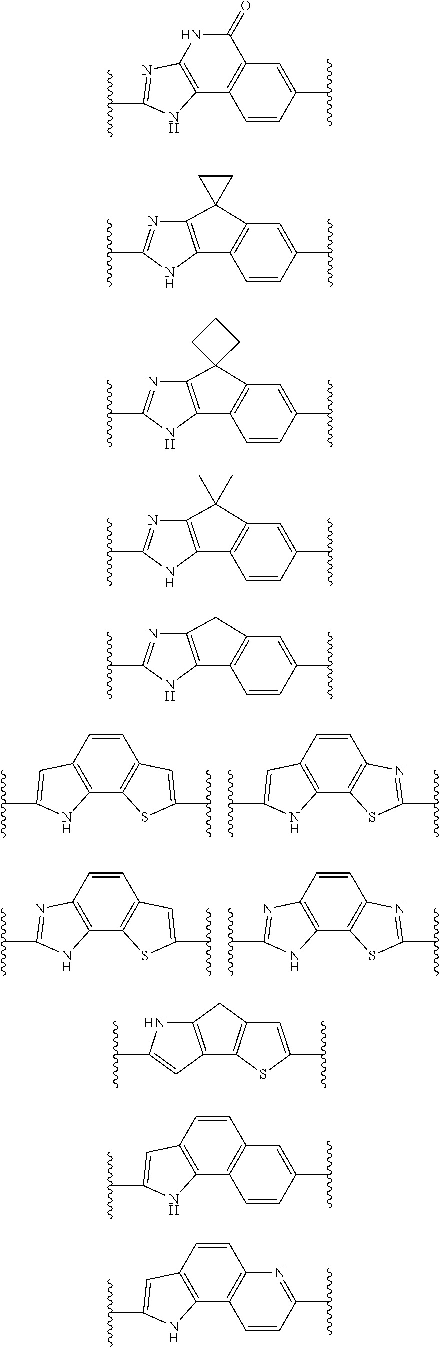

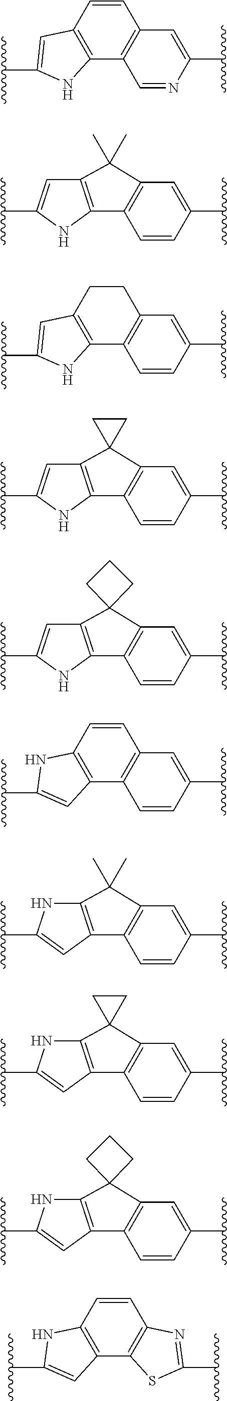

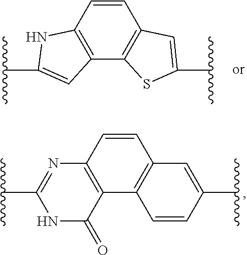

and each occurrence of R⁴ is independently:

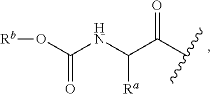

wherein R^{a} is H, alkyl, haloalkyl, cycloalkyl or aryl, and R^{b} is alkyl, haloalkyl or cycloalkyl.

In another embodiment, A and C are each independently selected from:

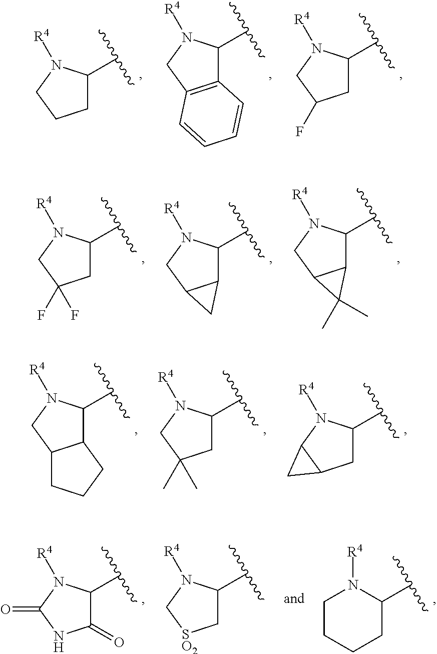

the group:

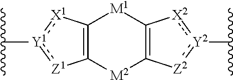

has the structure:

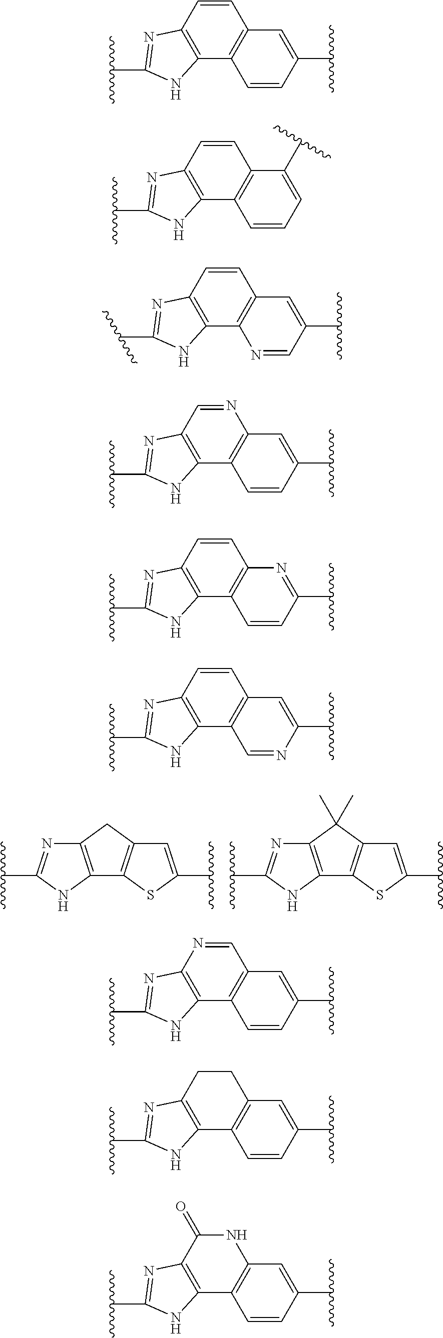

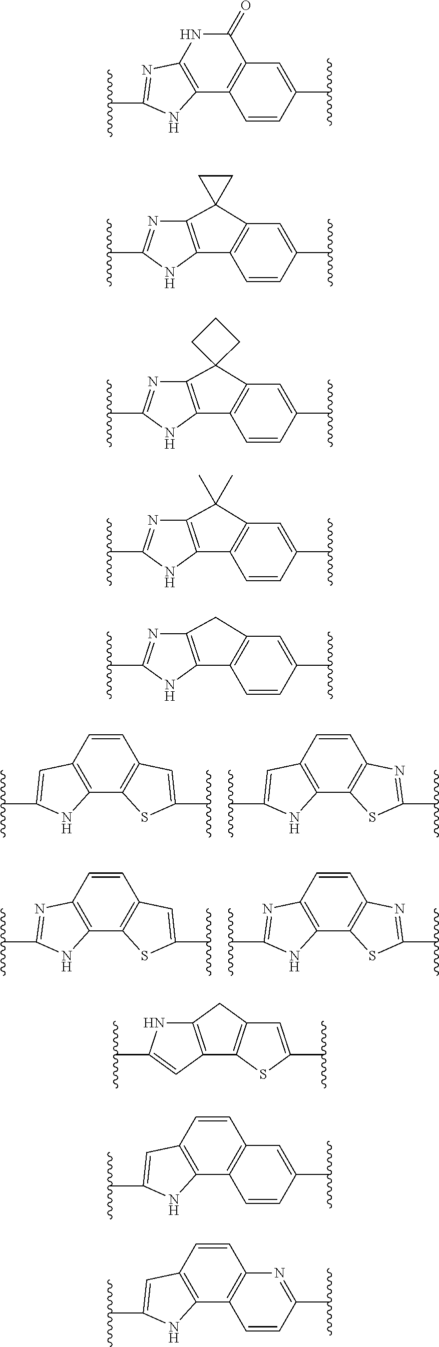

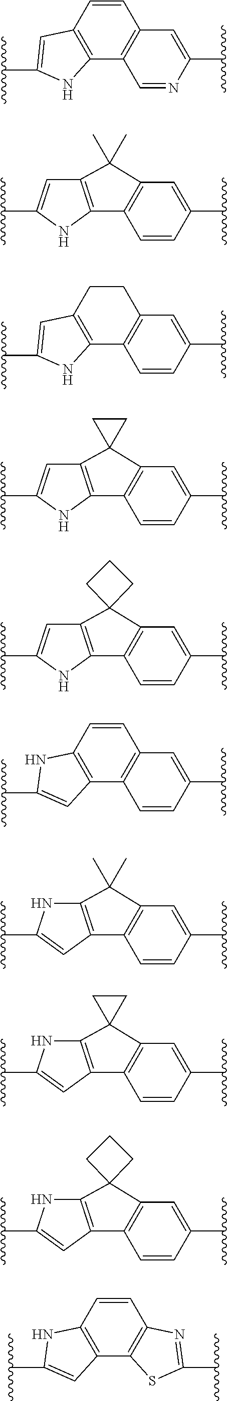

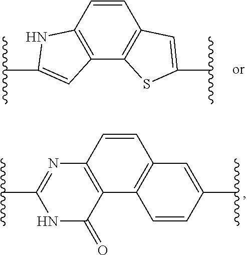

and each occurrence of R⁴ is independently:

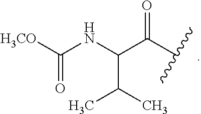

In one embodiment, A and C are each independently selected from:

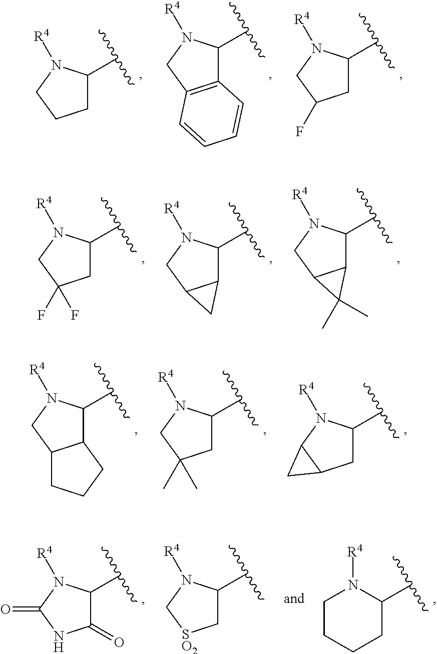

the group:

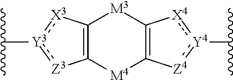

has the structure:

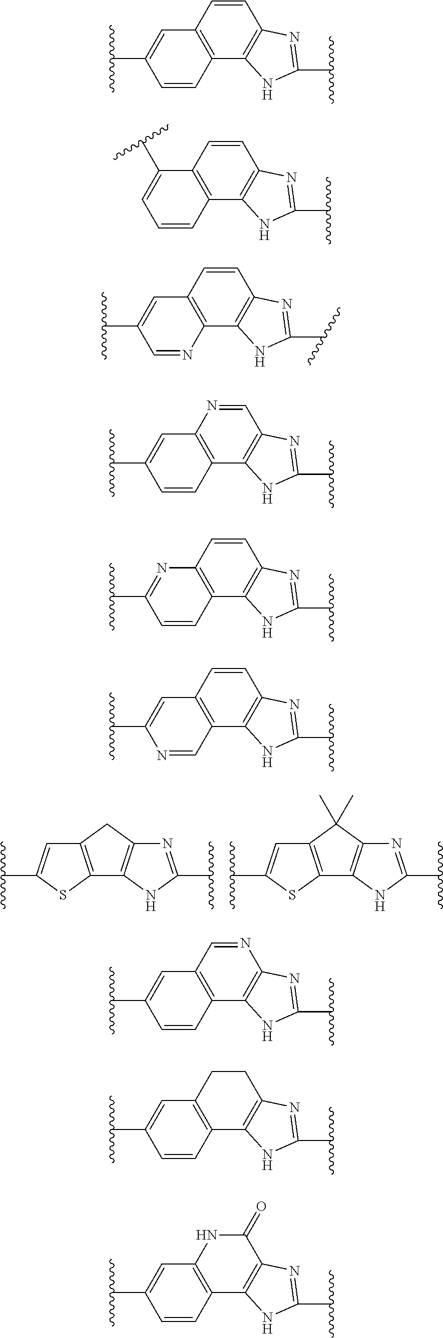

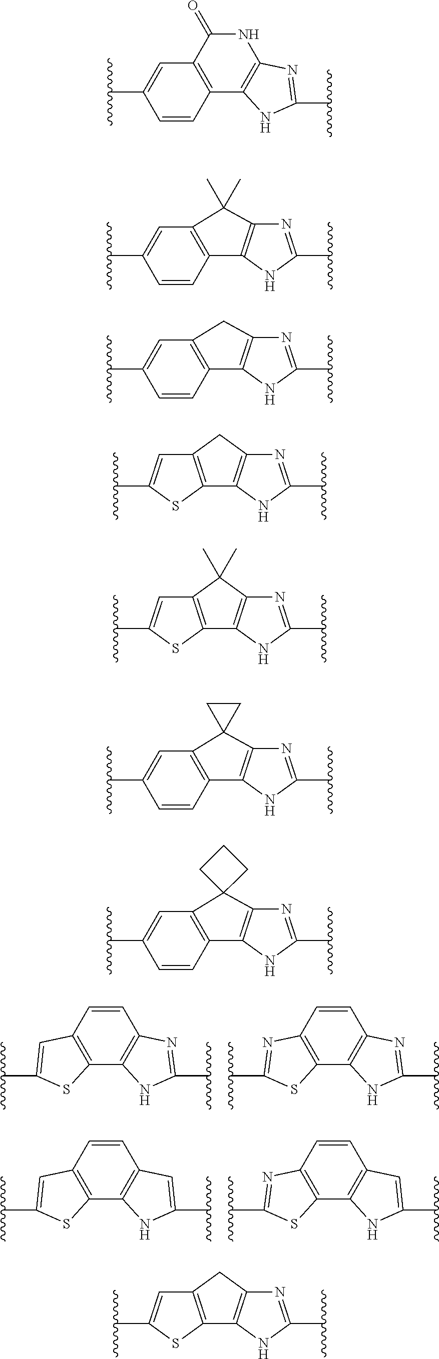

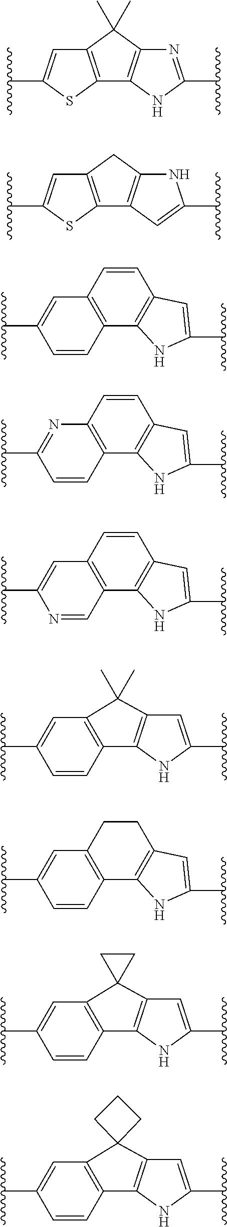

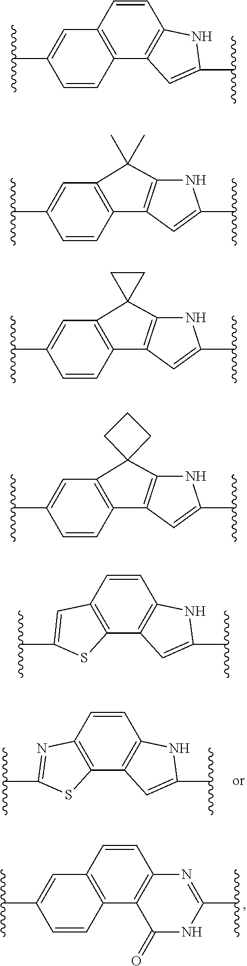

and each occurrence of R⁴ is independently selected from:

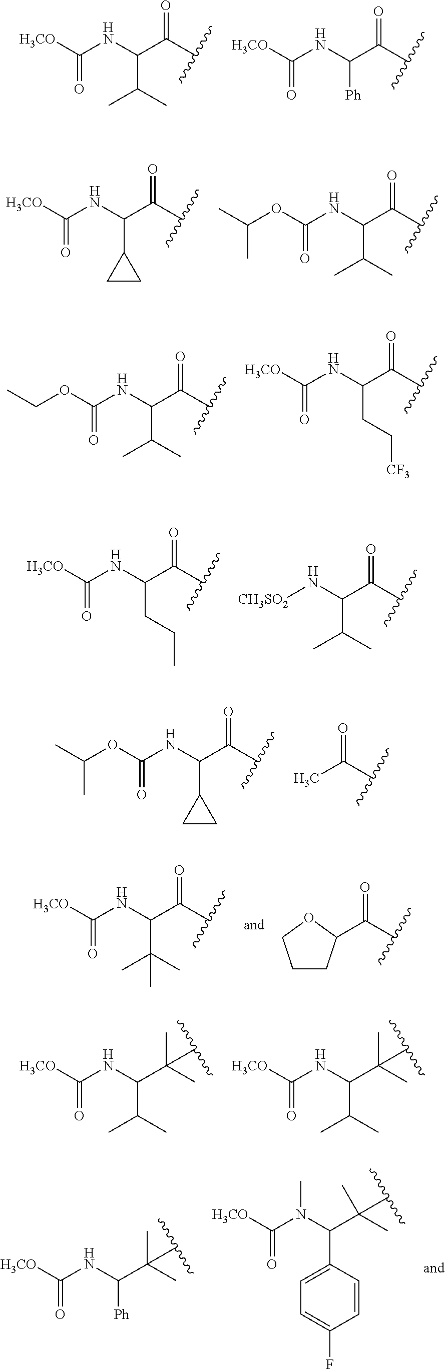

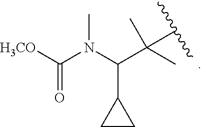

In another embodiment, A and C are each independently selected from:

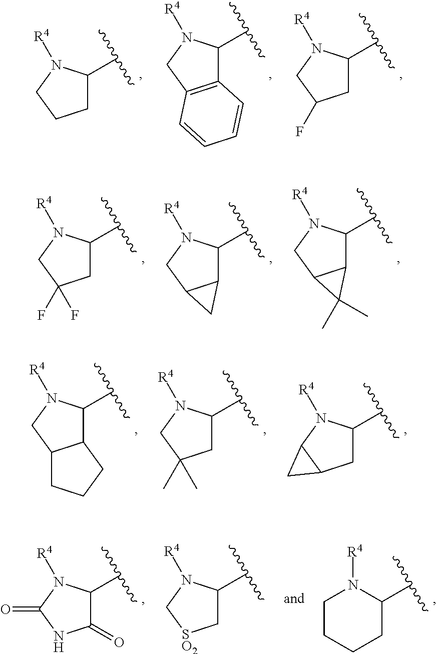

the group:

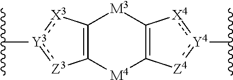

has the structure:

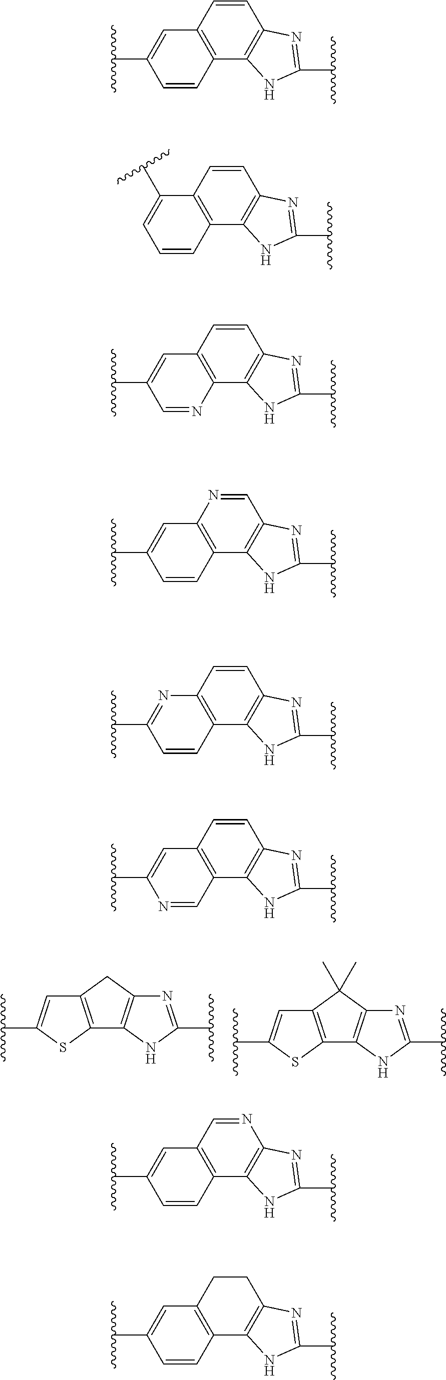

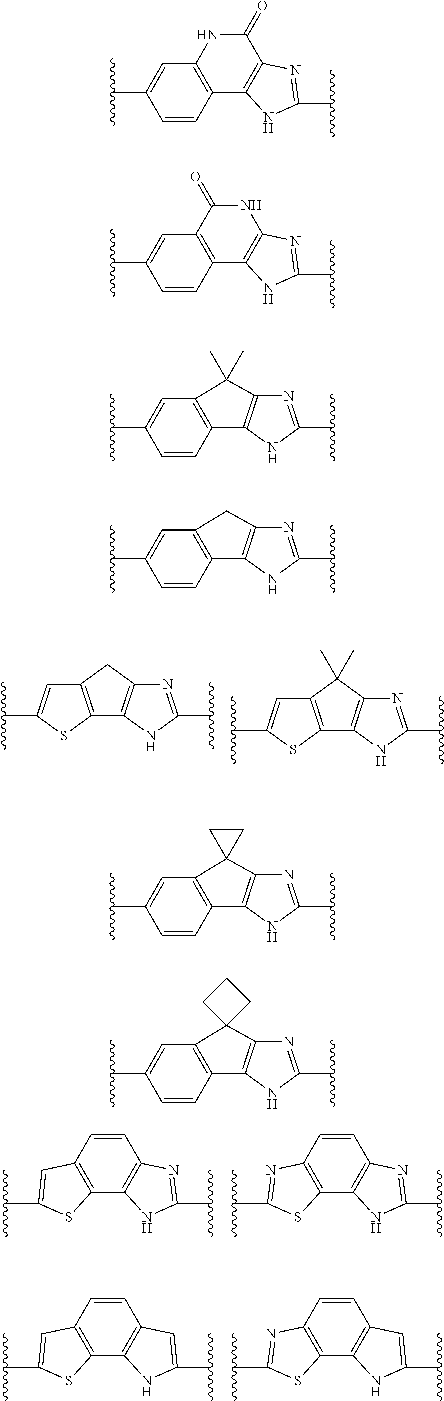

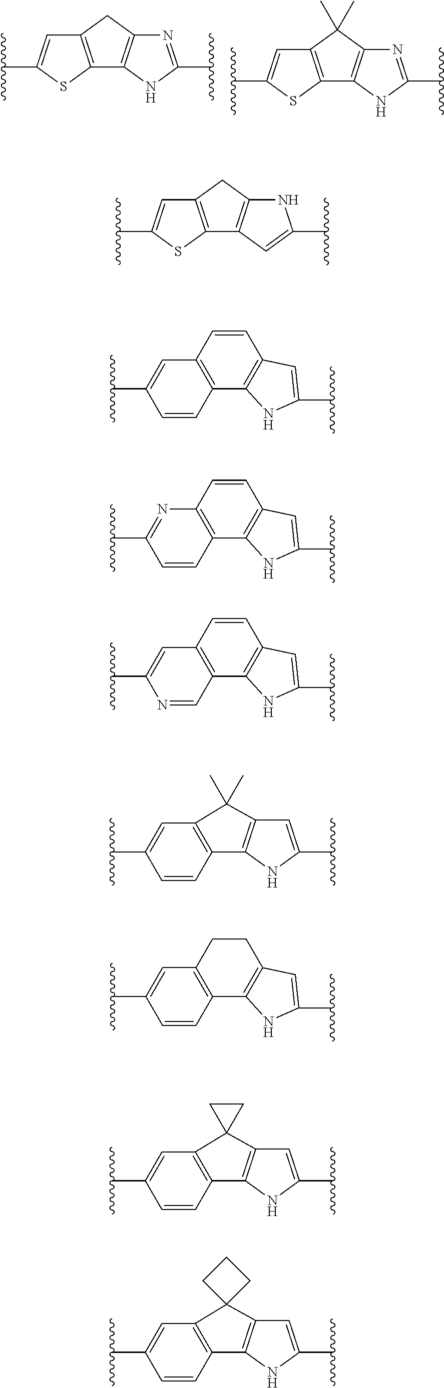

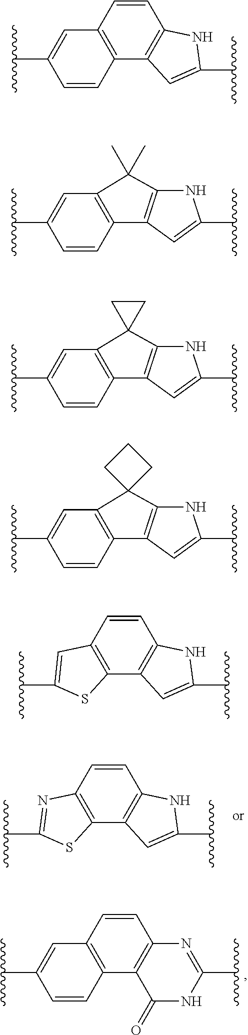

and each occurrence of R⁴ is independently:

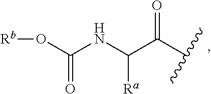

wherein R^{a} is H, alkyl, haloalkyl, cycloalkyl or aryl, and R^{b} is alkyl, haloalkyl or cycloalkyl.

In another embodiment, A and C are each independently selected from:

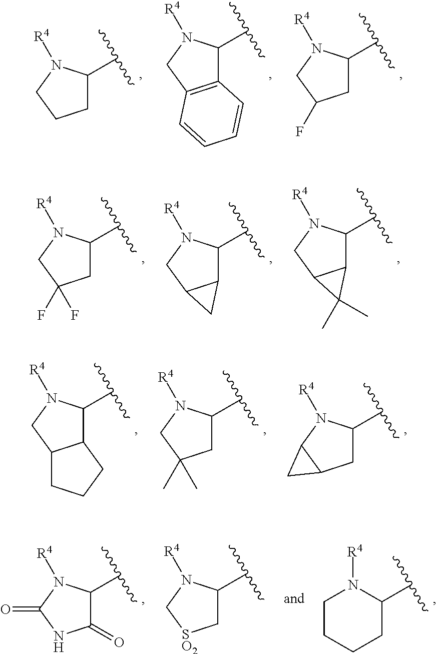

the group:

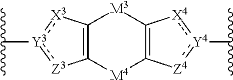

has the structure:

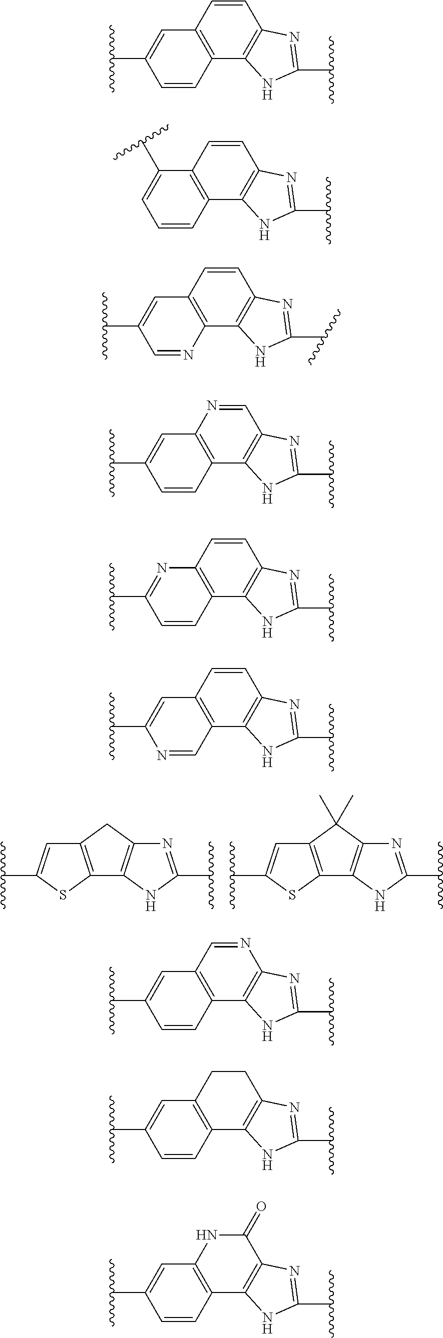

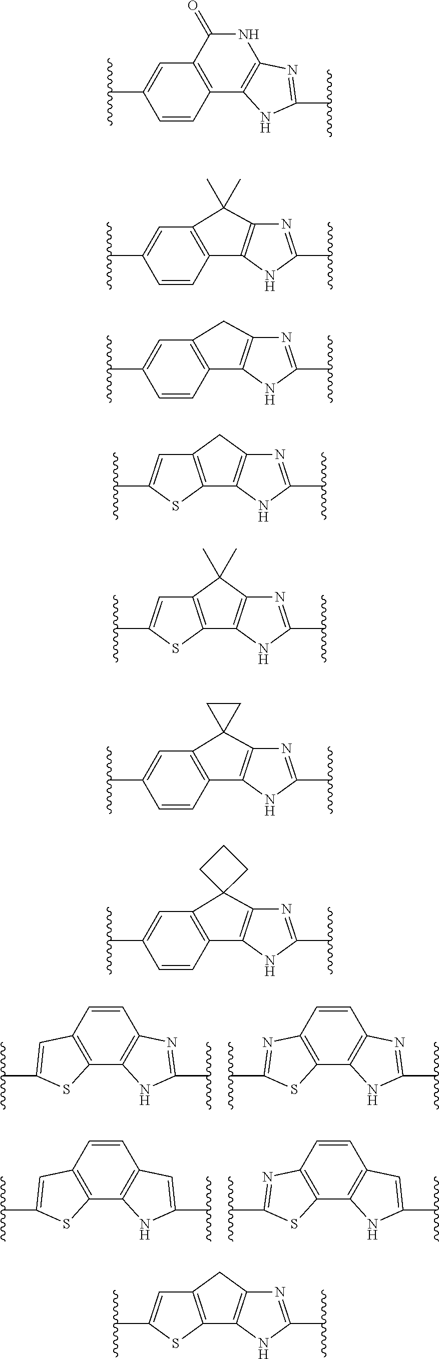

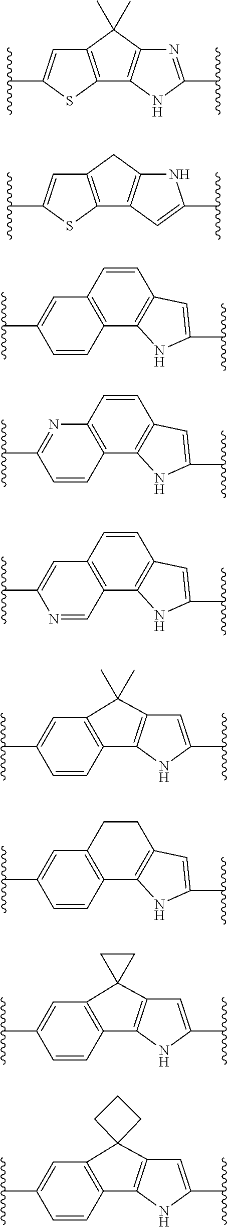

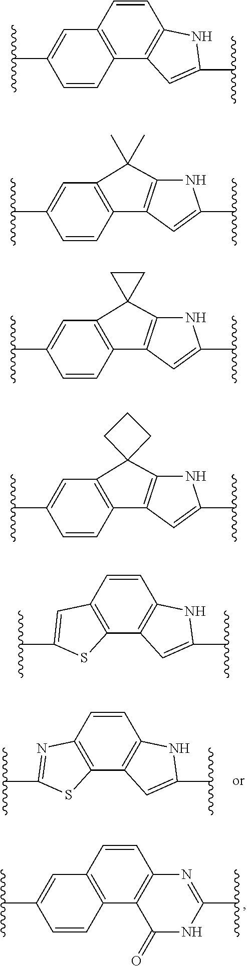

and each occurrence of R⁴ is independently:

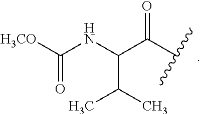

In one embodiment, A, B, C, M¹, M², M³, M⁴, X¹, X², X³, X⁴, Y¹, Y², Y³, Y², Z¹, Z², Z³ and Z⁴ in the Compounds of Formula (I) are selected independently from each other.

In another embodiment, a Compound of Formula (I) is in purified form.

In another embodiment, a Compound of Formula (I) has one or more of its hydrogen atoms replaced with a deuterium atom.

In one embodiment, the compounds of formula (I) have the formula (Ia):

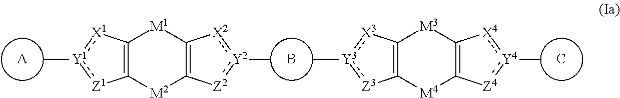

wherein A, C, X¹, X², X³, X⁴, Z¹, Z², Z³ and Z⁴ are defined above for the Compounds of Formula (1), and wherein:

B is a bond, phenylene, monocyclic cycloalkylene, monocyclic cycloalkenylene, monocyclic heterocycloalkylene, monocycle heterocycloalkenylene or monocycle heteroarylene, wherein a phenylene or monocyclic cycloalkylene can be optionally and independently substituted with one or more R¹⁴ groups, and wherein a monocycle heterocycloalkylene or monocycle heteroarylene can be optionally and independently substituted on one or more ring nitrogen atoms with R⁶ and on one or more ring carbon atoms with R¹⁴;

M¹ is a bond, —C(R⁷)₂—, —[C(R⁷)₂]₂—, —C(R⁷)═C(R⁷)—, —C(R⁷)═N—, —N═C(R⁷)—, —O—, —C(R⁷)₂—O—, —N(R⁶)—, —C(R⁷)₂—N(R⁶)—, —S—, —S(O)₂—, —C(R⁷)₂—S(O)₂—, —OC(O)N(R⁶)—, —N(R⁶)C(O)N(R⁶)—, —S(O)₂N(R⁶)— or —N(R⁶)S(O)₂N(R⁶)—;

M² is a bond, —C(R⁷)₂—, —[C(R⁷)₂]₂—, —C(R⁷)═C(R⁷)—, —C(R⁷)═N—, —N═C(R⁷)—, —O—, —C(R⁷)₂—O—, —N(R⁶)—, —C(R⁷)₂—N(R⁶)—, —S—, —S(O)₂—, —C(R⁷)₂—S(O)₂—, —OC(O)N(R⁶)—, —N(R⁶)C(O)N(R⁶)—, —S(O)₂N(R⁶)— or —N(R⁶)S(O)₂N(R⁶)—, such that at least one of M¹ and M² is other than a bond, and such that the central ring of formula (1) that contains M¹ and M² has from 5 to 10 total ring atoms, and wherein two vicinal R⁷ groups of M¹ or M² together with the carbon atoms to which they are attached, can optionally join to form a 3- to 7-membered cycloalkyl group, a 3- to 7-membered heterocycloalkyl group, or a 5- to 6-membered heteroaryl group;

M³ is a bond, —C(R⁷)₂—, —[C(R⁷)₂]₂—, —C(R⁷)═C(R⁷)—, —C(R⁷)═N—, —N═C(R⁷)—, —O—, —C(R⁷)₂—O—, —N(R⁶)—, —C(R⁷)₂—N(R⁶)—, —S—, —S(O)₂—, —C(R⁷)₂—S(O)₂—, —OC(O)N(R⁶)—, —N(R⁶)C(O)N(R⁶)—, —S(O)₂N(R⁶)— or —N(R⁶)S(O)₂N(R⁶)—;

M⁴ is a bond, —C(R⁷)₂—, —[C(R⁷)₂]₂—, —C(R⁷)═C(R⁷)—, —C(R⁷)═N—, —N═C(R⁷)—, —O—, —C(R⁷)₂—O—, —N(R⁶)—, —C(R⁷)₂—N(R⁶)—, —S—, —S(O)₂—, —C(R⁷)₂—S(O)₂—, —OC(O)N(R⁶)—, —N(R⁶)C(O)N(R⁶)—, —S(O)₂N(R⁶)— or —N(R⁶)S(O)₂N(R⁶)—, such that at least one of M³ and M⁴ is other than a bond, and such that the central ring of formula (I) that contains M³ and M⁴ has from 5 to 10 total ring atoms, and wherein two vicinal R⁷ groups of M³ or M⁴ together with the carbon atoms to which they are attached, can optionally join to form a 3- to 7-membered cycloalkyl group, a 3- to 7-membered heterocycloalkyl group, or a 5- to 6-membered heteroaryl group;

Y¹ is —C—, and an optional and additional bond to Y¹ is present;

Y² is —C—, and an optional and additional bond to Y² is present;

Y³ is —C—, and an optional and additional bond to Y³ is present; and

Y⁴ is —C—, and an optional and additional bond to Y⁴ is present.

In one embodiment,for the compounds of formula (Ia), the group:

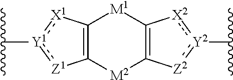

has the structure:

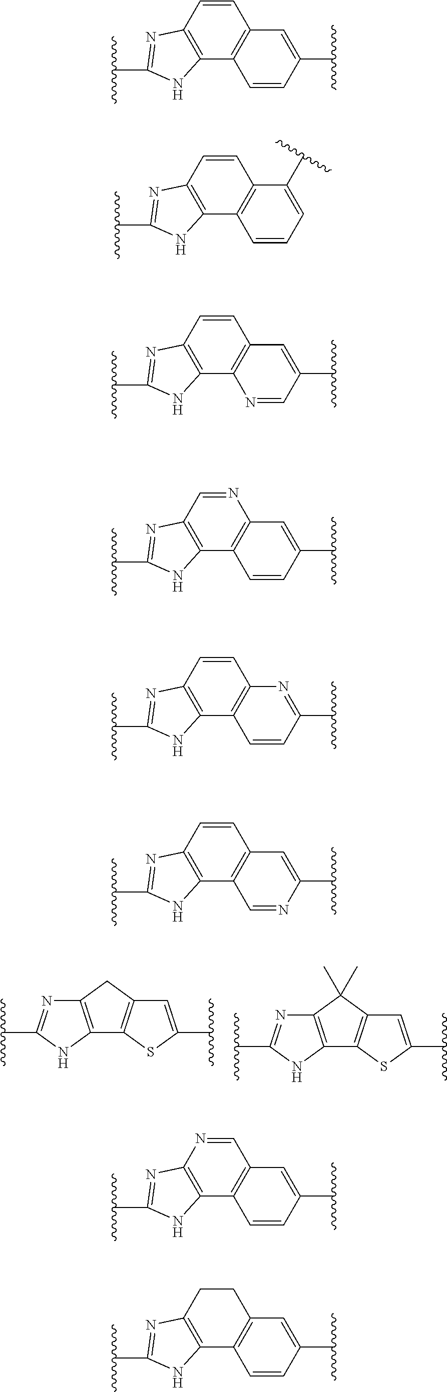

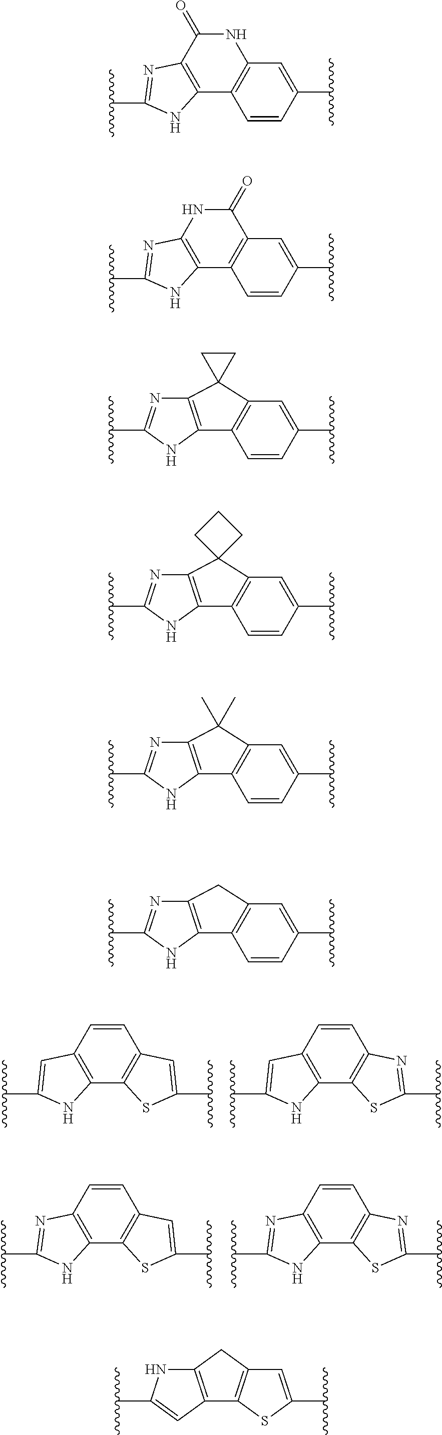

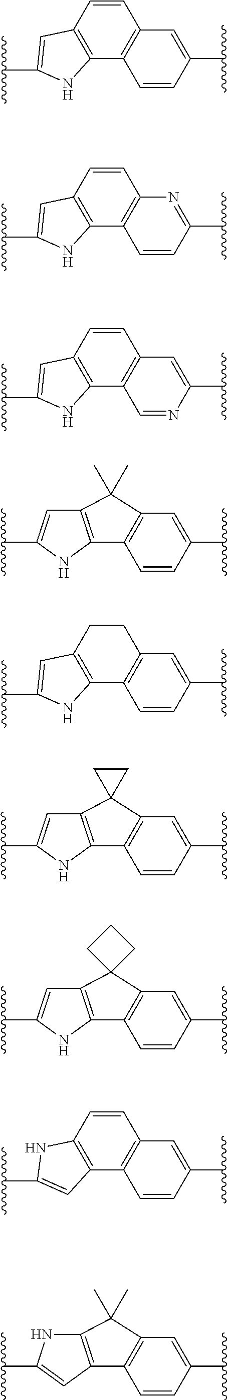

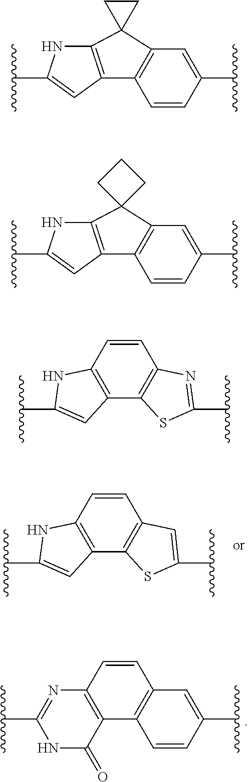

In another embodiment, for the compounds of formula (Ia), the group:

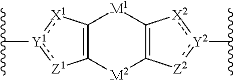

has the structure:

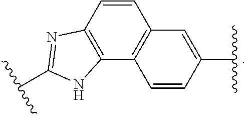

In another embodiment, for the compounds of formula (Ia), the group:

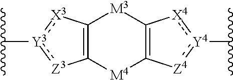

has the structure:

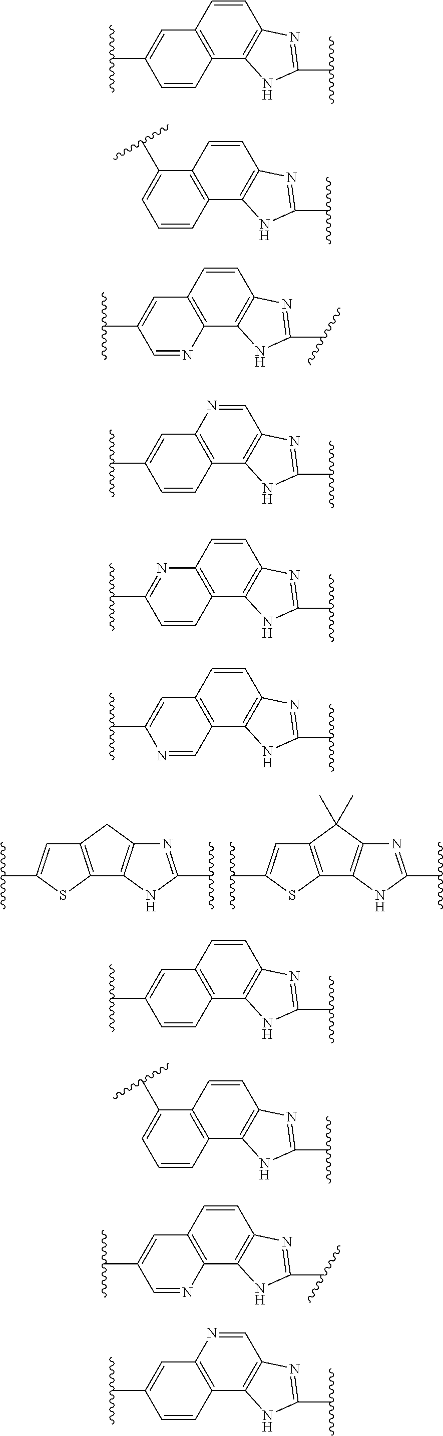

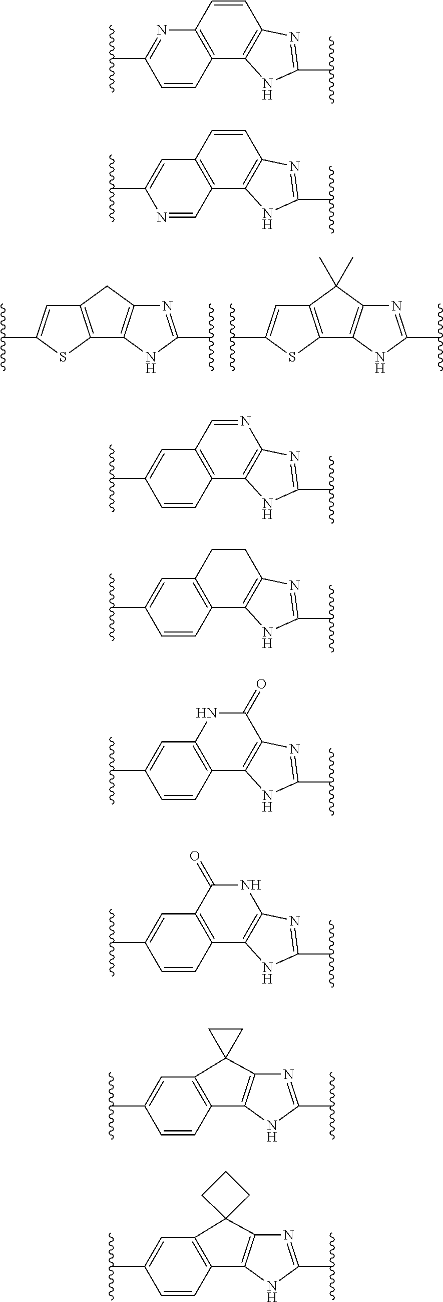

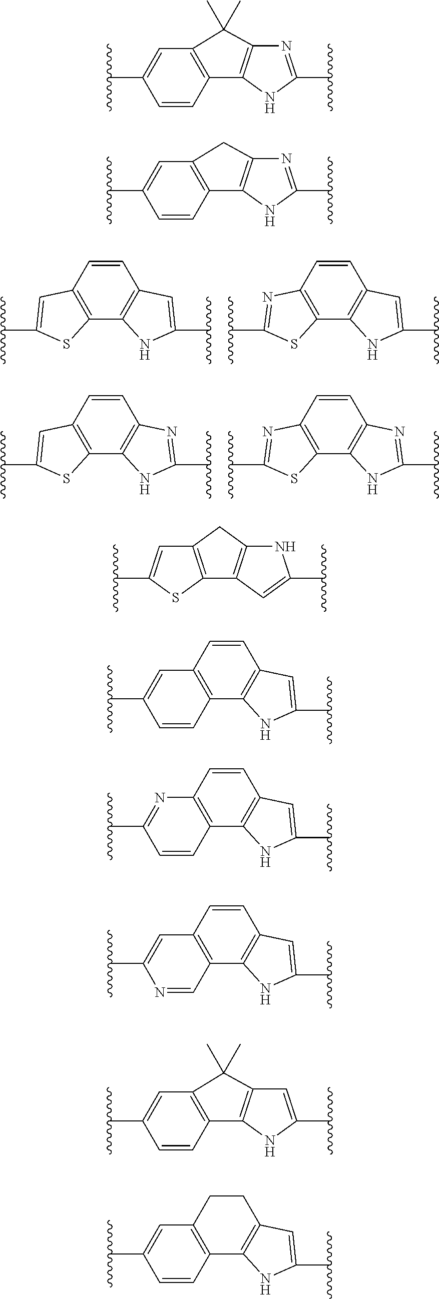

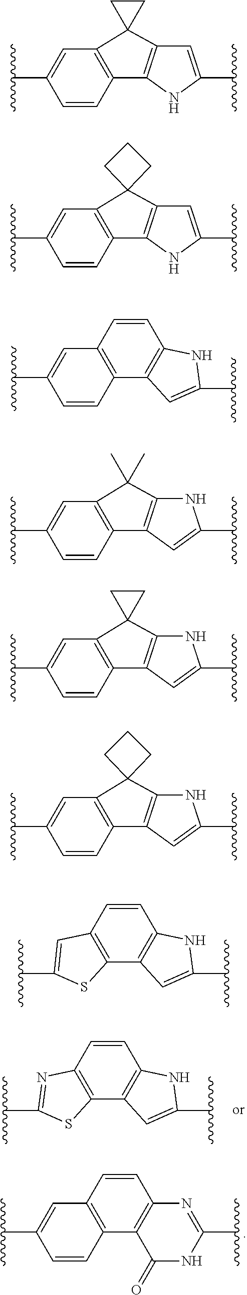

In still another embodiment, for the compounds of formula (Ia), the group:

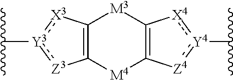

has the structure:

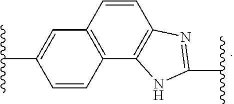

In another embodiment, for the compounds of formula (Ia), at least one of A and C is -alkylene-N(R¹¹)(R¹³).

In yet another embodiment, for the compounds of formula (Ia), A and C are each heterocycloalkyl.

In another embodiment, for the compounds of formula (Ia), A is selected from

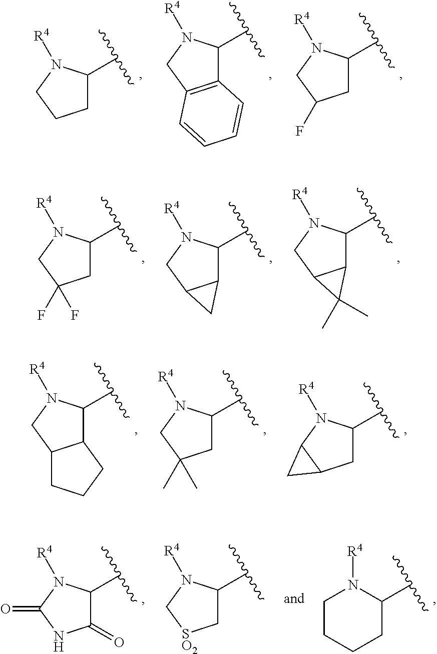

or A is —CH(R^{a})—N(R¹¹)(R¹³), wherein R^{a} is H, alkyl, haloalkyl, cycloalkyl or aryl.

In a further embodiment, for the compounds of formula (Ia), A is:

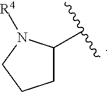

In one embodiment, for the compounds of formula (Ia), at least one of A and C is selected from

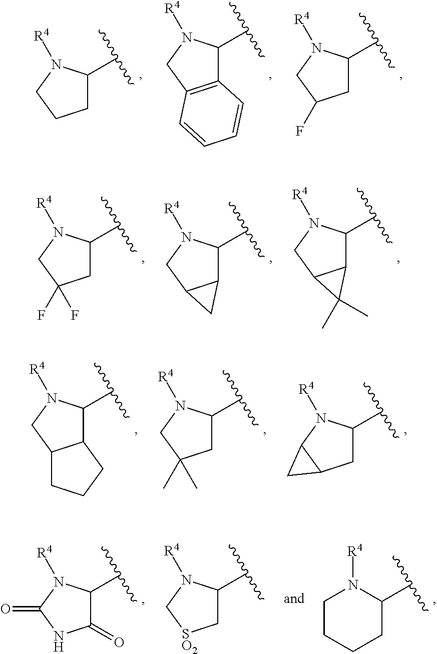

or at least one of A and C is —CH(R^{a})—N(R¹¹)(R¹³), wherein R^{a} is H, alkyl, haloalkyl, cycloalkyl or aryl.

In another embodiment, for the compounds of formula (Ia), A and C are each:

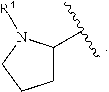

In another embodiment, for the compounds of formula (Ia), B is a bond.

In another embodiment, for the compounds of formula (Ia), each occurrence of R⁴ is independently selected from:

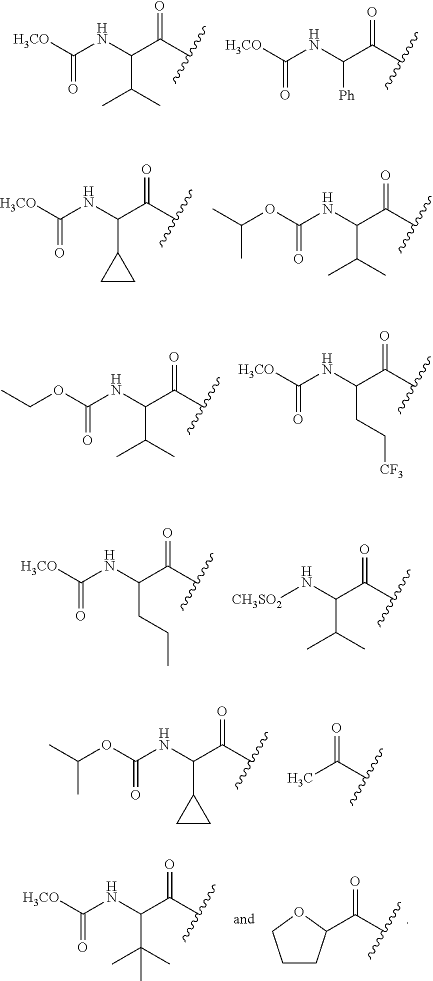

In another embodiment, for the compounds of formula (Ia), each occurrence of R⁴ is independently:

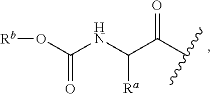

wherein R^{a} is H, alkyl, haloalkyl, cycloalkyl or aryl, and R^{b} is alkyl, haloalkyl or cycloalkyl.

In another embodiment, for the compounds of formula (Ia), each occurrence of R⁴ is independently:

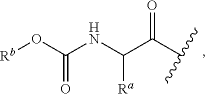

wherein R^{a} is H, methyl, ethyl, propyl, isopropyl, —CH(CH₃)(OCH₃), t-butyl, cyclopropyl, —CH₂CH₂CF₃ or phenyl, and R^{b} is methyl, ethyl, propyl, isopropyl, t-butyl, cyclopropyl, or —CH₂CH₂CF₃.

In another embodiment, for the compounds of formula (Ia), each occurrence of R⁴ is:

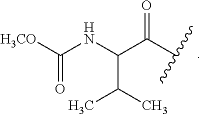

In one embodiment the compounds of formula (I) have the formula (Ib):

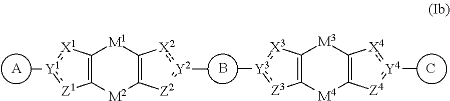

wherein the group:

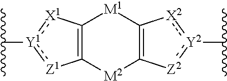

has the structure:

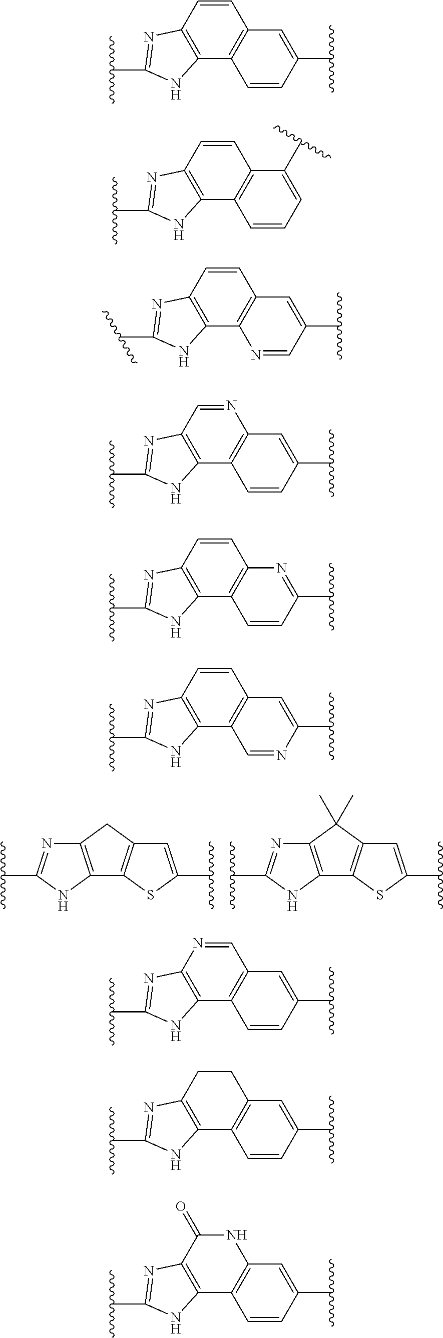

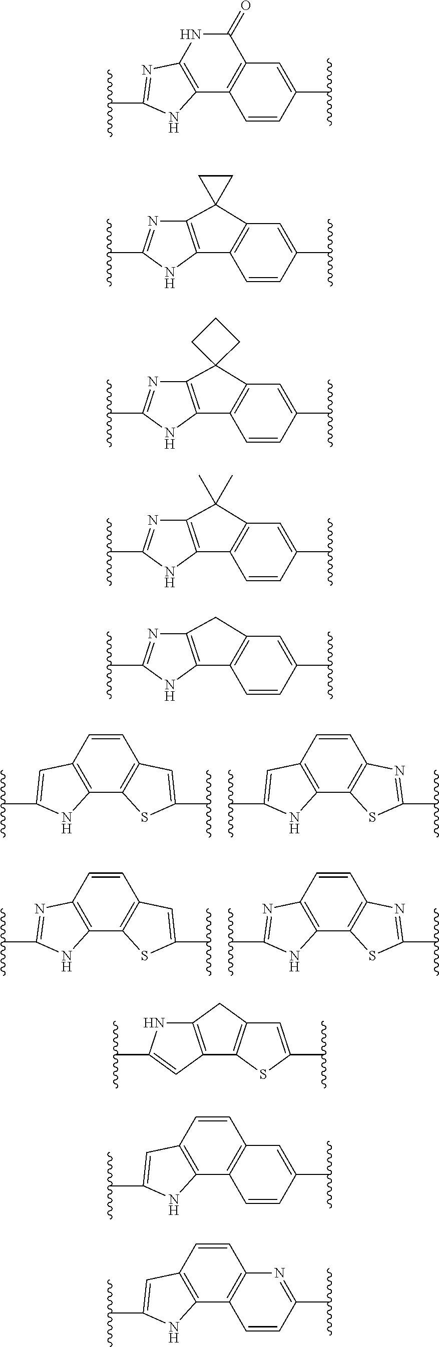

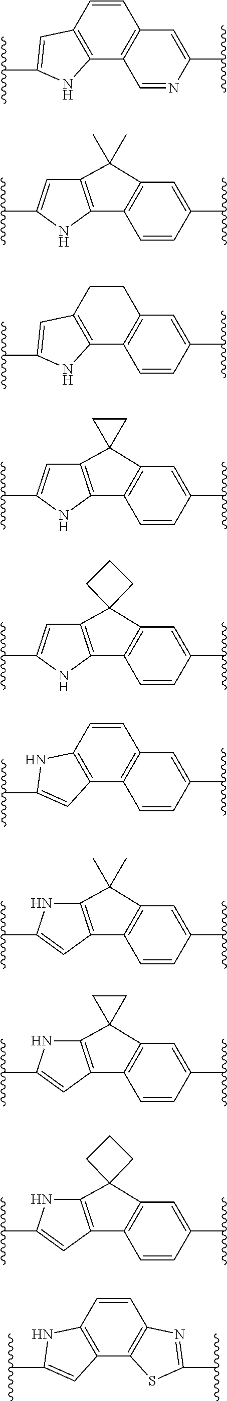

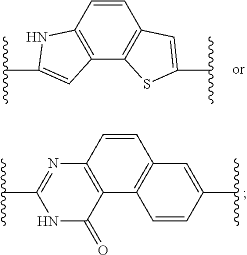

the group:

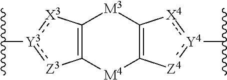

has the structure:

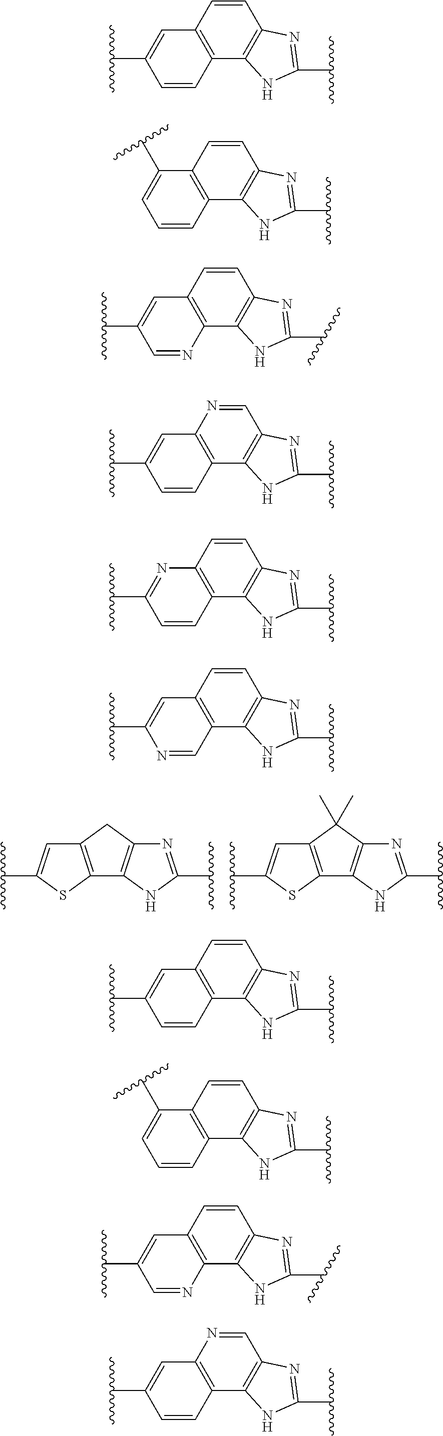

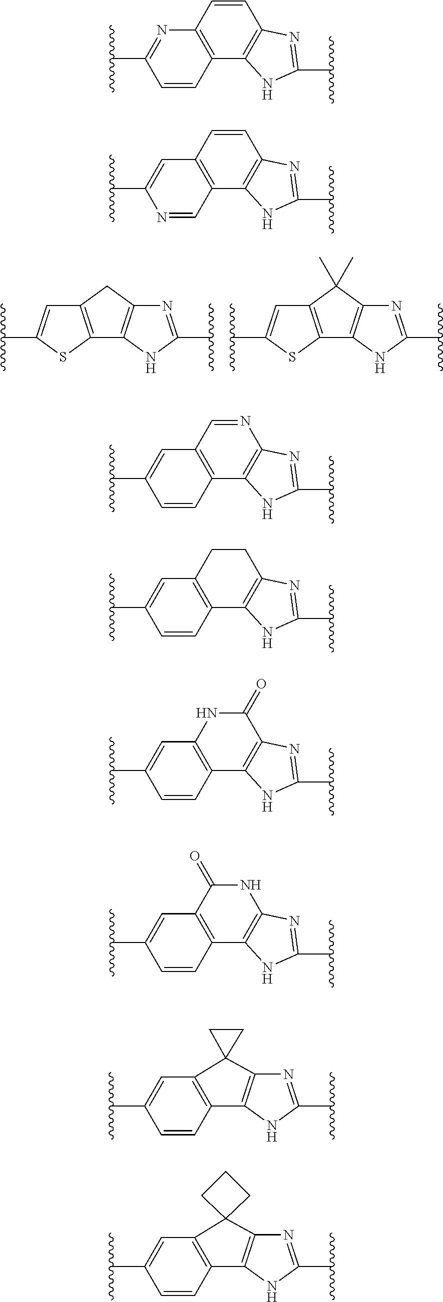

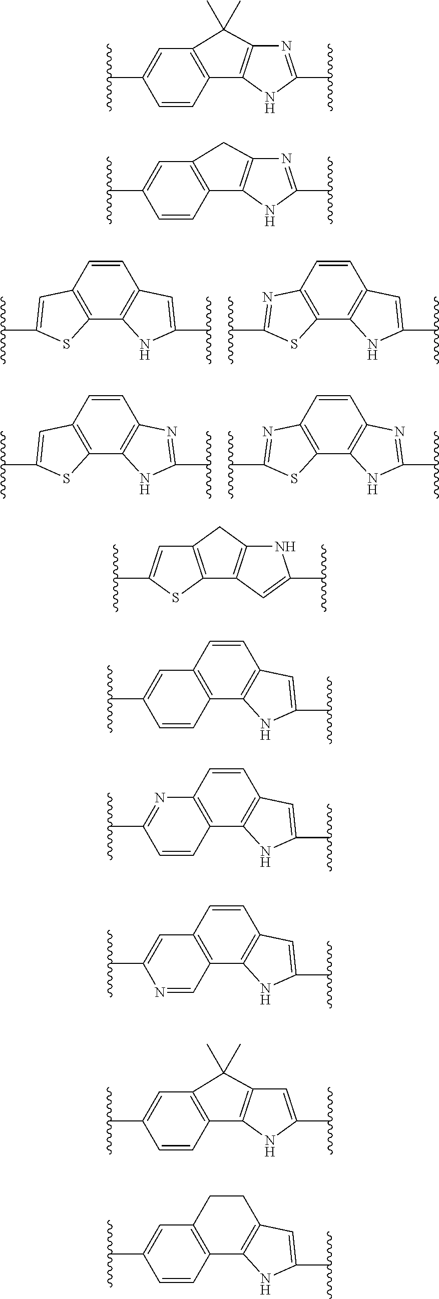

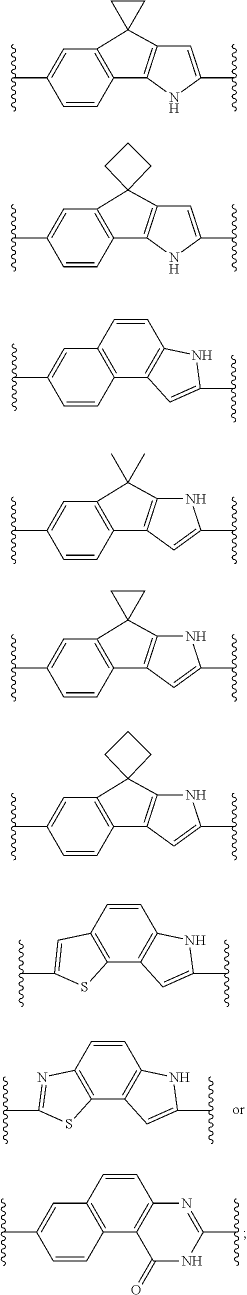

A is selected from:

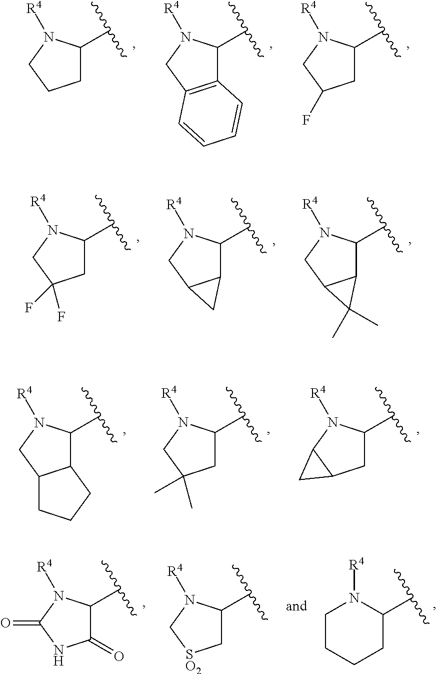

or A is —CH(R^{a})—N(R¹¹)(R¹³), wherein R^{a} is H, alkyl, haloalkyl, cycloalkyl or aryl;

B is a bond or phenylene, wherein a phenylene can be optionally and independently substituted with one or more R¹⁴ groups; and

C is selected from:

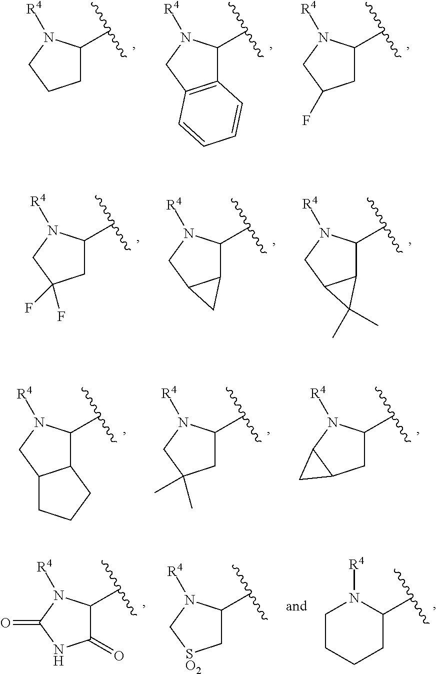

or C is —CH(R^{a})—N(R¹¹)(R¹³), wherein R^{a} is H, alkyl, haloalkyl, cycloalkyl or aryl.

In one embodiment, for the compounds of formula (Ib), the group:

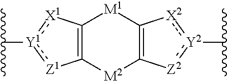

has the structure:

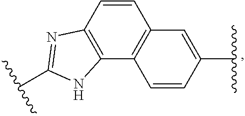

or the group

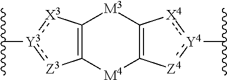

has the structure:

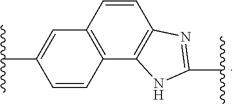

In another embodiment, for the compounds of formula (Ib), at least one of A and C is selected from:

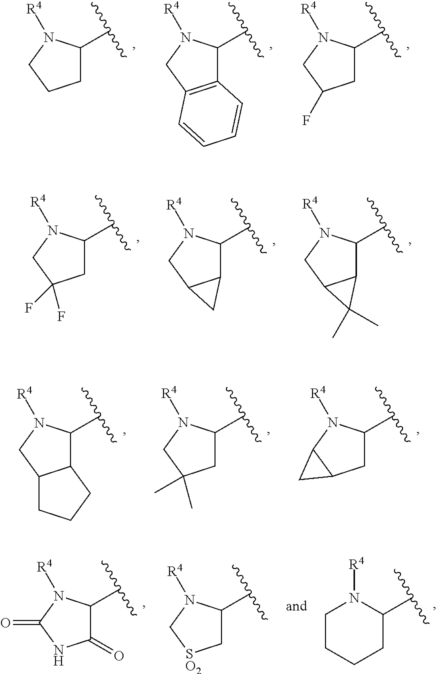

or at least one of A and C is —CH(R^{a})—N(R¹¹)(R¹³), wherein R^{a} is H, alkyl, haloalkyl, cycloalkyl or aryl.

In another embodiment, for the compounds of formula (Ib), A and C are each:

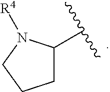

In still another embodiment, for the compounds of formula (Ib), at least one of A and C is selected from:

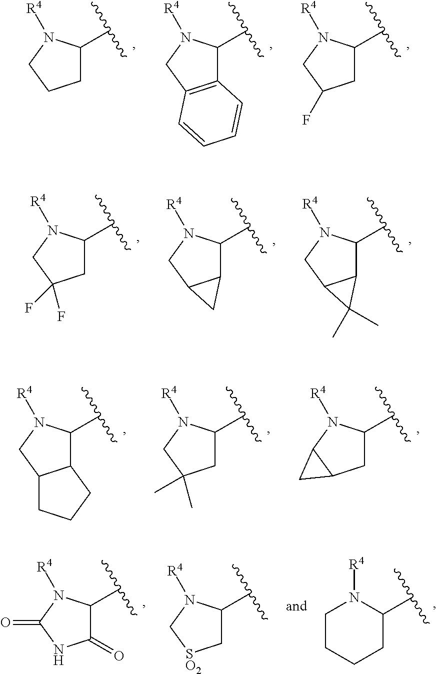

or at least one of A and C is —CH(R^{a})—N(R¹¹)(R¹³), wherein R^{a} is H, alkyl, haloalkyl, cycloalkyl or aryl; and each occurrence of R⁴ is independently selected from:

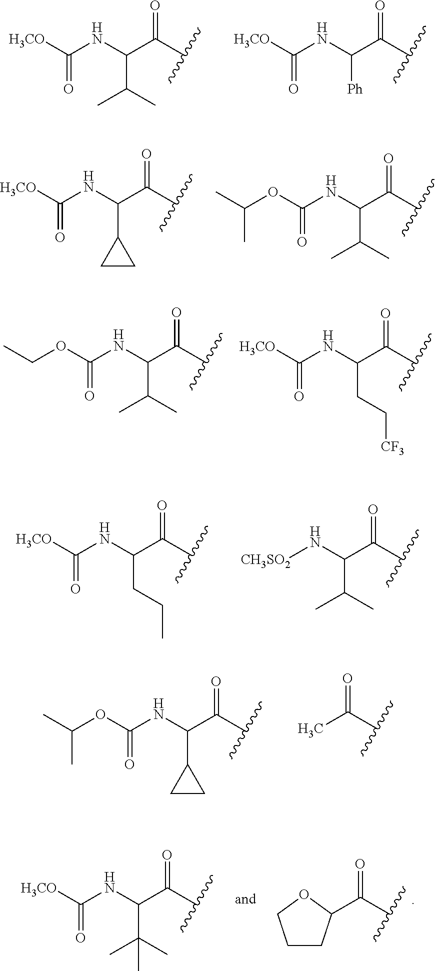

In another embodiment, for the compounds of formula (Ib), at least one of A and C is selected from:

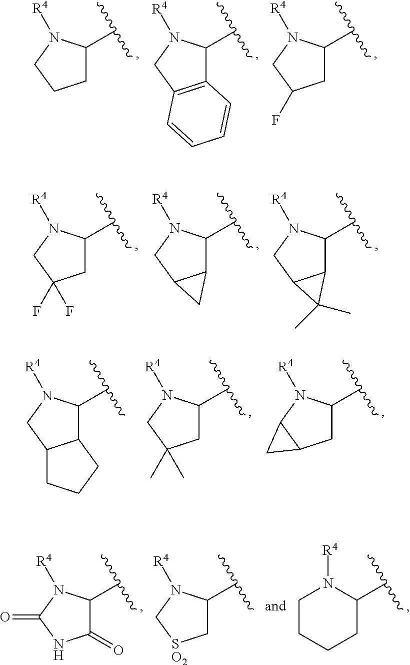

or at least one of A and C is —CH(R^{a})—N(R¹¹)(R¹³), wherein R^{a} is H, alkyl, haloalkyl, cycloalkyl or aryl; and each occurrence of R⁴ is independently:

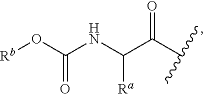

wherein R^{a} is H, alkyl, haloalkyl, cycloalkyl or aryl, and R^{b} is alkyl, haloalkyl or cycloalkyl.

In yet another embodiment, for the compounds of formula (Ib), at least one of A and C is selected from:

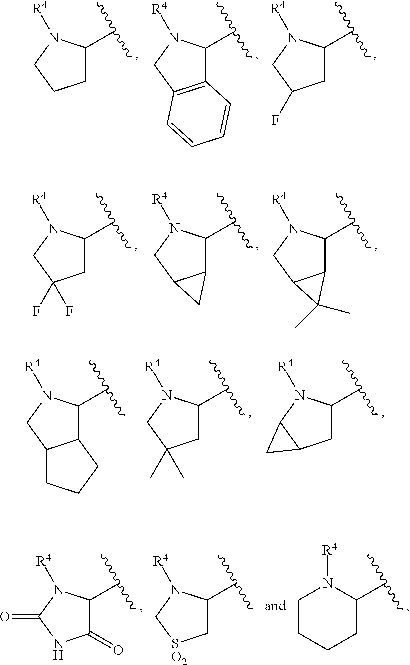

or at least one of A and C is —CH(R^{a})—N(R¹¹)(R¹³), wherein R^{a} is H, alkyl, haloalkyl, cycloalkyl or aryl; and each occurrence of R⁴ is independently:

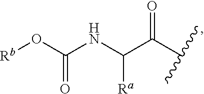

wherein R^{a} is H, methyl, ethyl, propyl, isopropyl, —CH(CH₃)(OCH₃), t-butyl, cyclopropyl, —CH₂CH₂CF₃ or phenyl, and R^{b} is methyl, ethyl, propyl, isopropyl, t-butyl, cyclopropyl, or —CH₂CH₂CF₃.

In a further embodiment, for the compounds of formula (Ib), at least one of A and C is selected from:

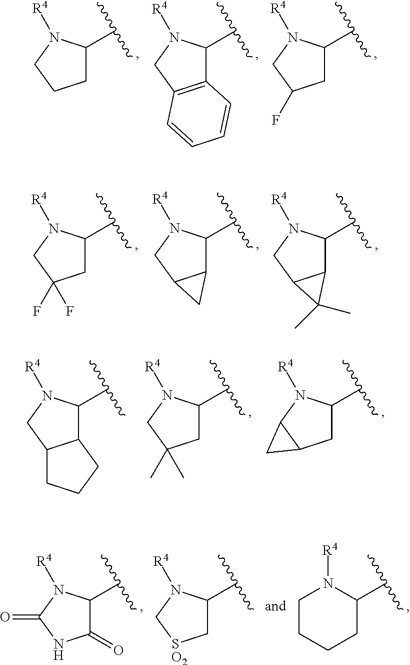

or at least one of A and C is —CH(R^{a})—N(R¹¹)(R¹³), wherein R^{a} is H, alkyl, haloalkyl, cycloalkyl or aryl; and each occurrence of R⁴ is:

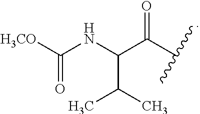

In another embodiment, for the compounds of formula (Ib), at least one of A and C is selected from:

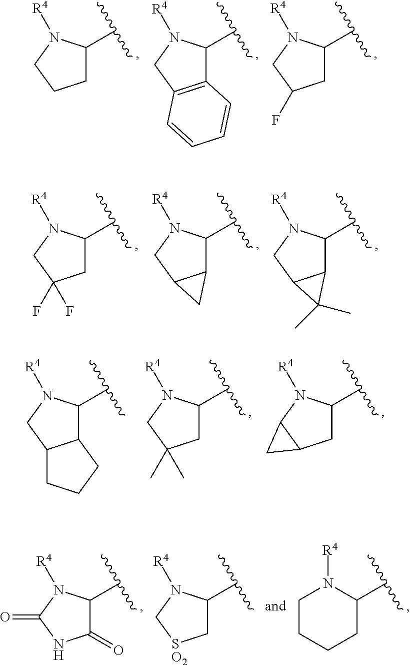

or at least one of A and C is —CH(R^{a})—N(R¹¹)(R¹³), wherein R^{a} is H, alkyl, haloalkyl, cycloalkyl or aryl; each occurrence of R⁴ is independently:

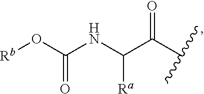

wherein R^{a} is H, alkyl, haloalkyl, cycloalkyl or aryl, and R^{b} is alkyl, haloalkyl or cycloalkyl; and B is a bond,

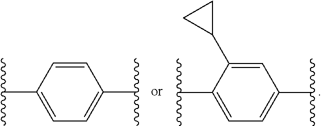

In another embodiment, for the compounds of formula (Ib), at least one of A and C is selected from:

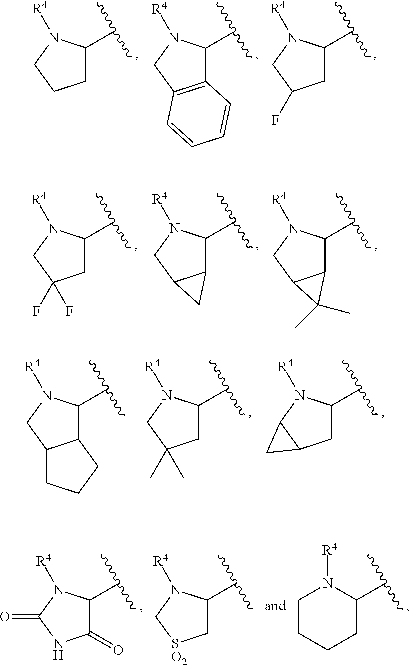

or at least one of A and C is —CH(R^{a})—N(R¹¹)(R¹³), wherein R^{a} is H, alkyl, haloalkyl, cycloalkyl or aryl; each occurrence of R⁴ is independently:

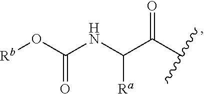

wherein R^{a} is H, alkyl, haloalkyl, cycloalkyl or aryl, and R^{b} is alkyl, haloalkyl or cycloalkyl; and B is a bond.

In still another embodiment, for the compounds of formula (Ib), A and C are each:

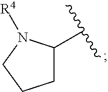

each occurrence of R⁴ is

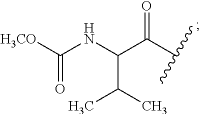

and B is a bond.

Non-limiting examples of the Compounds of Formula (I) include compounds 1-69 as set forth below:

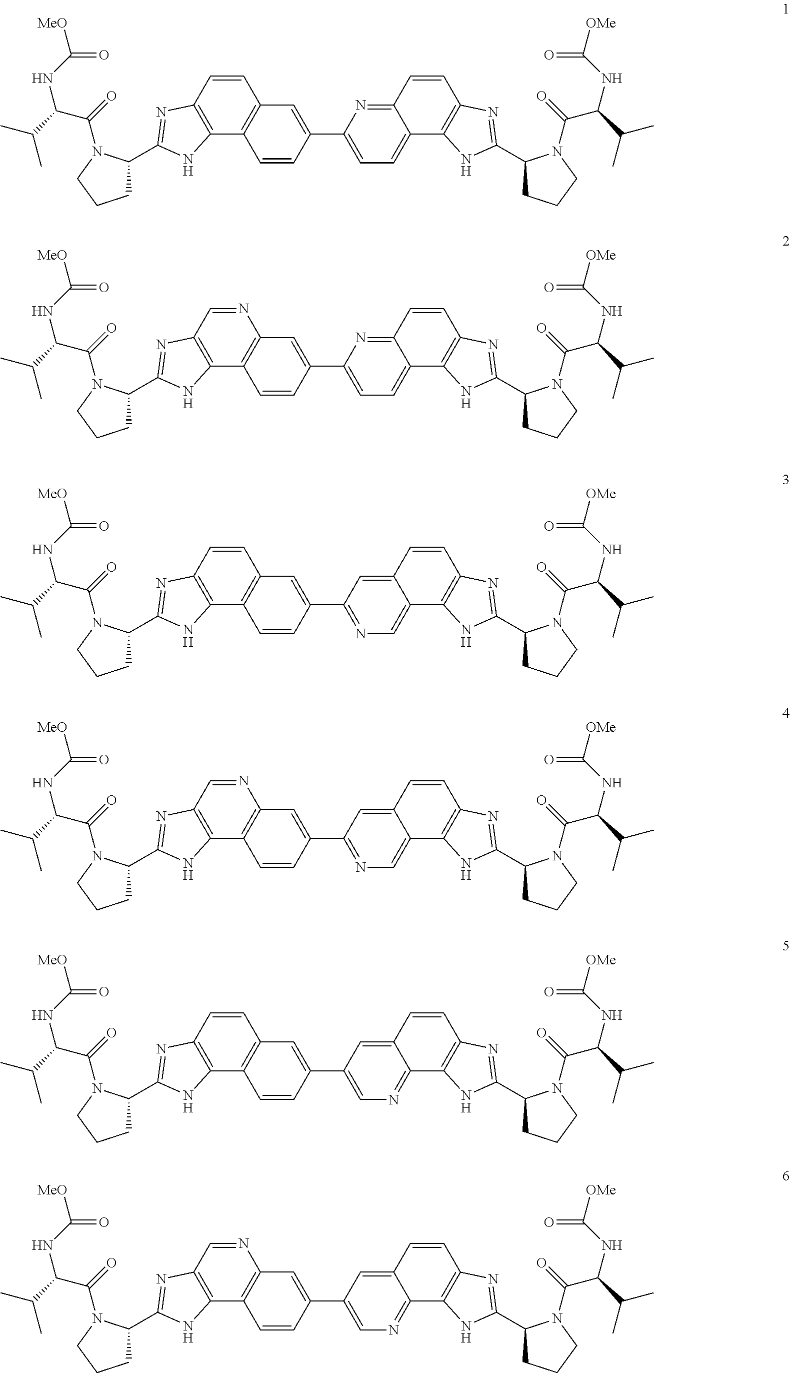

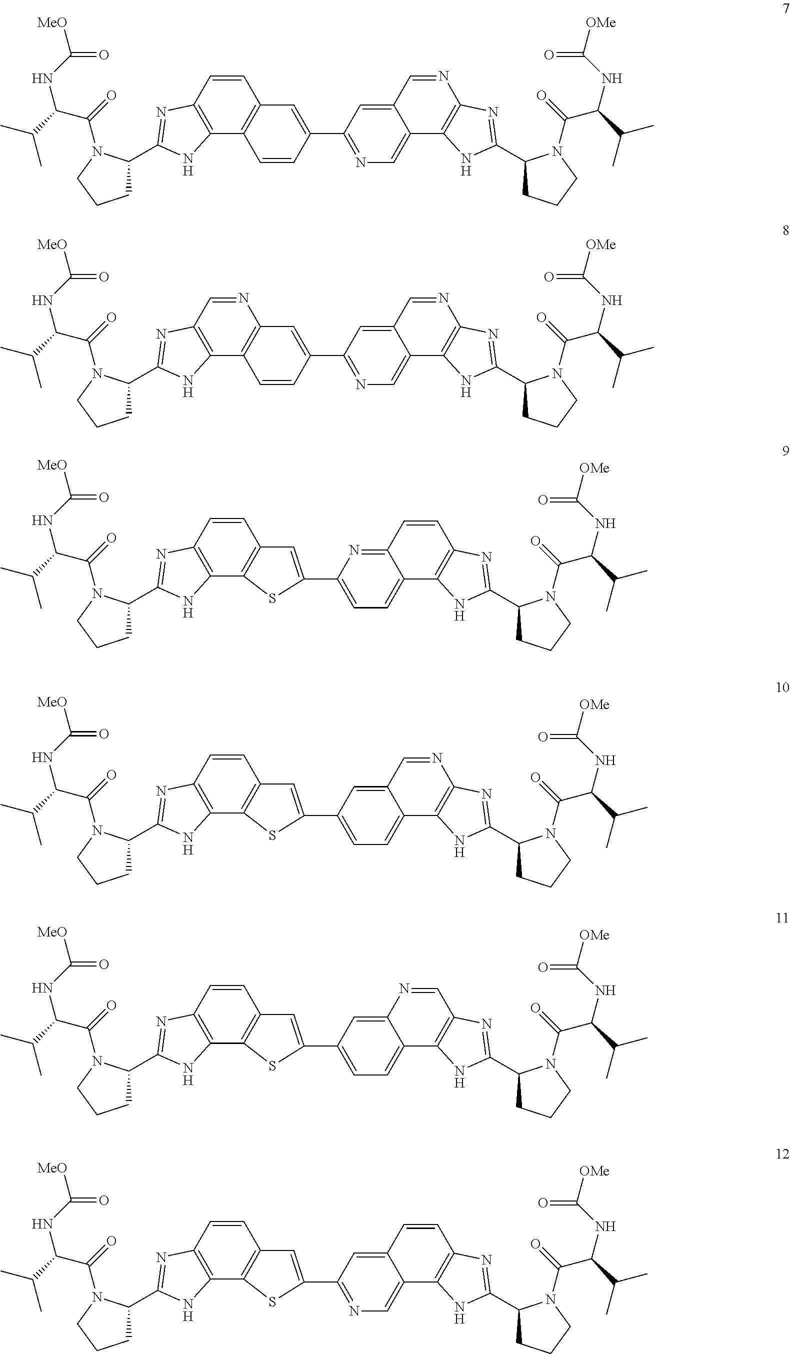

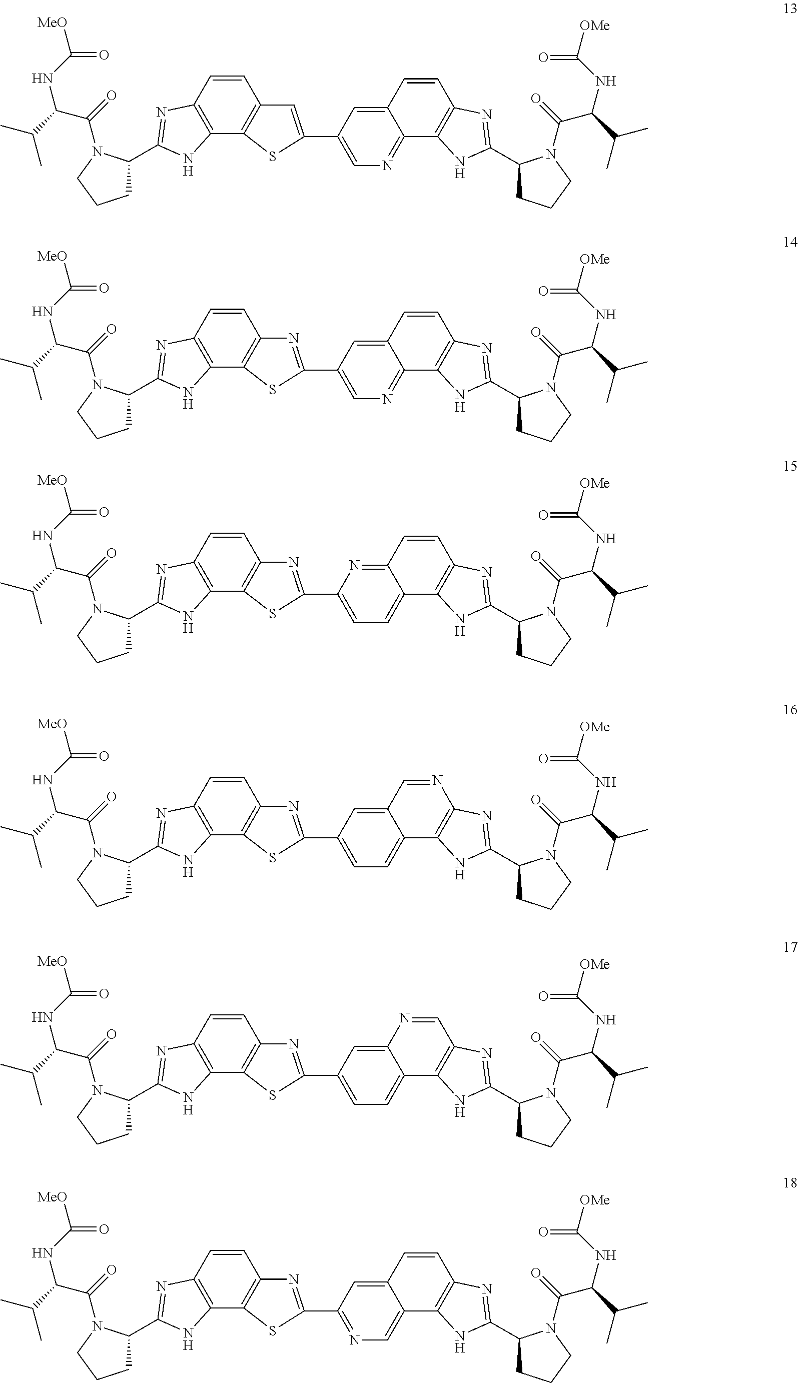

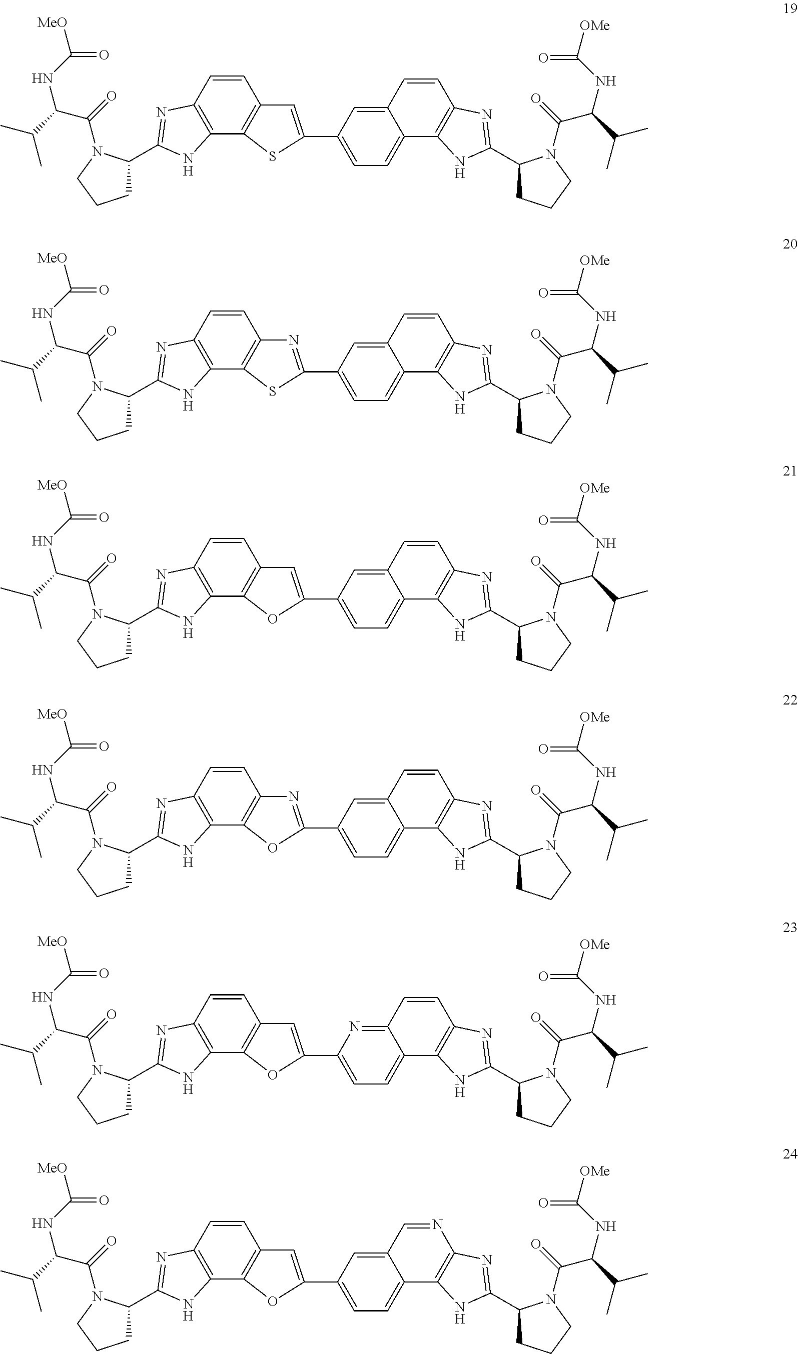

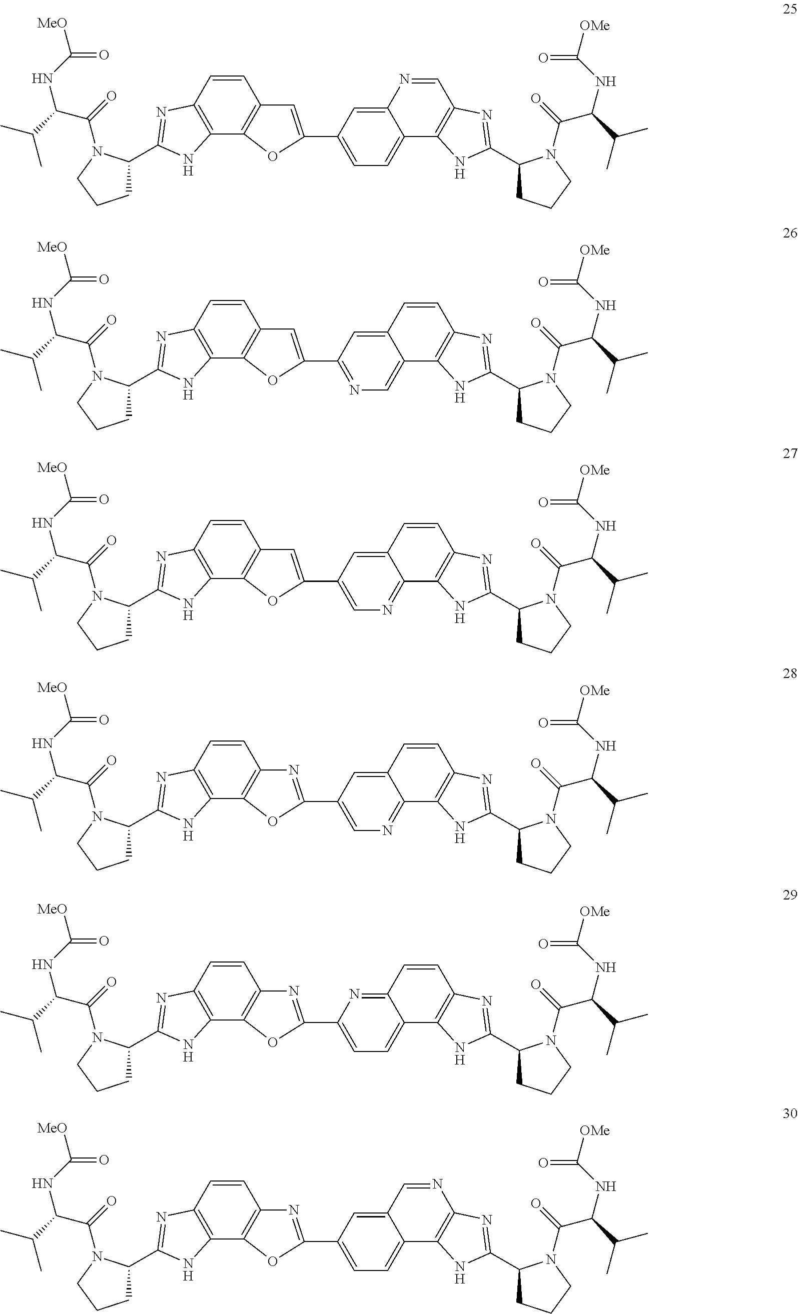

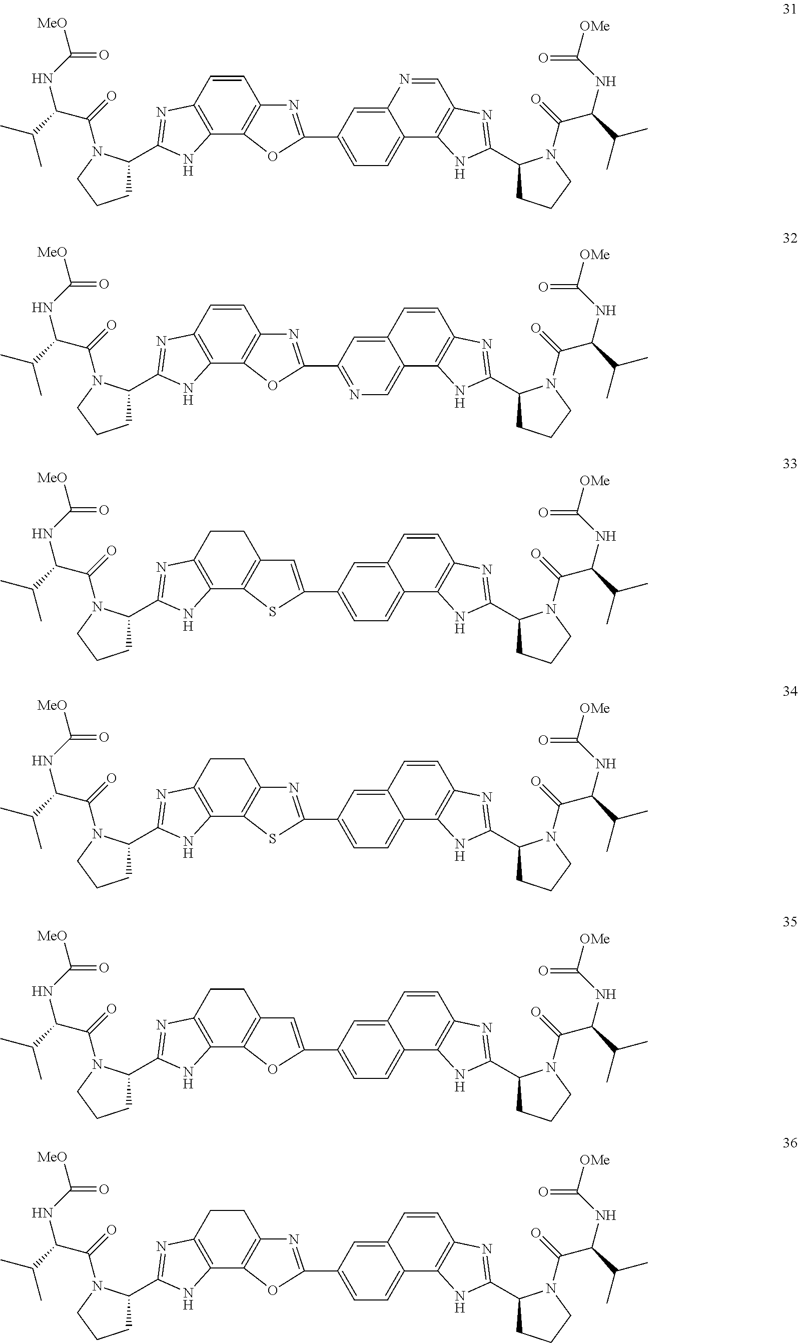

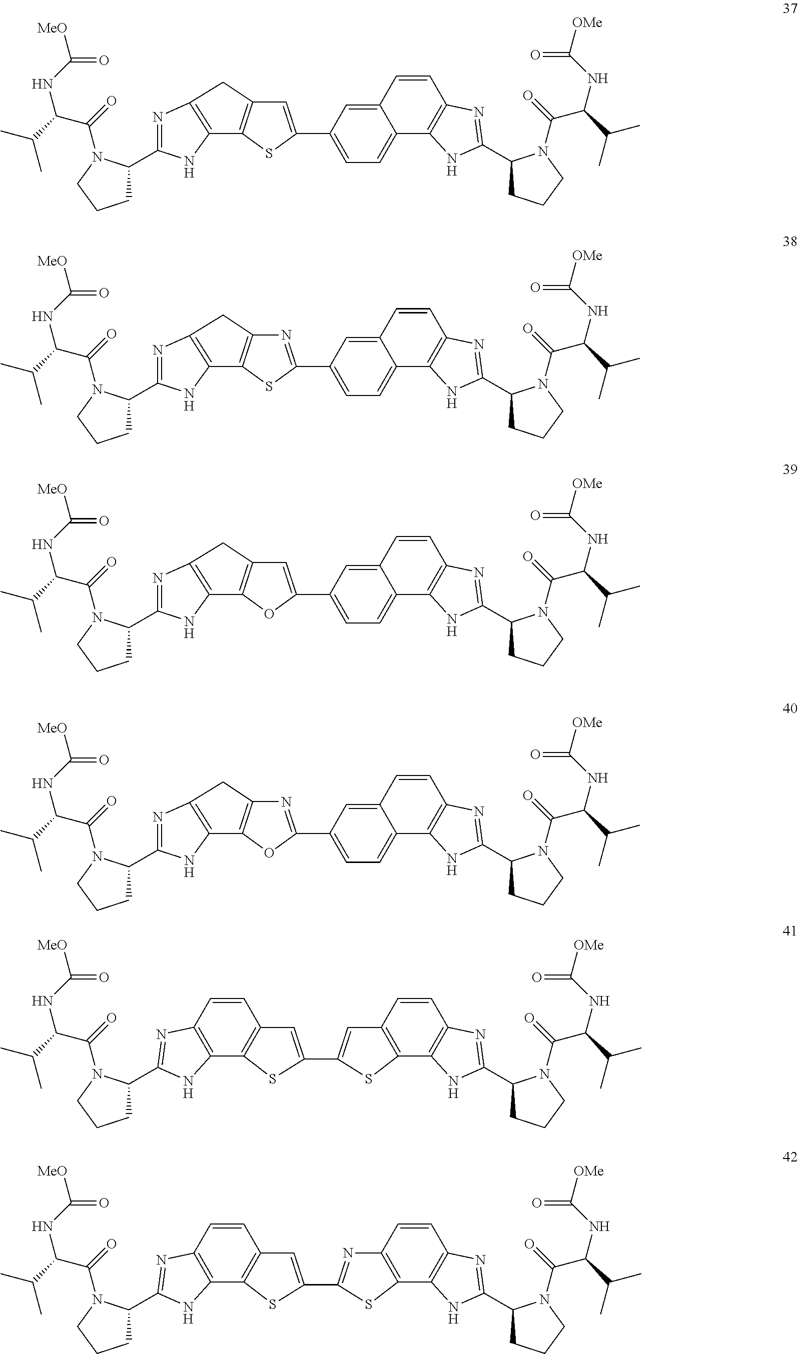

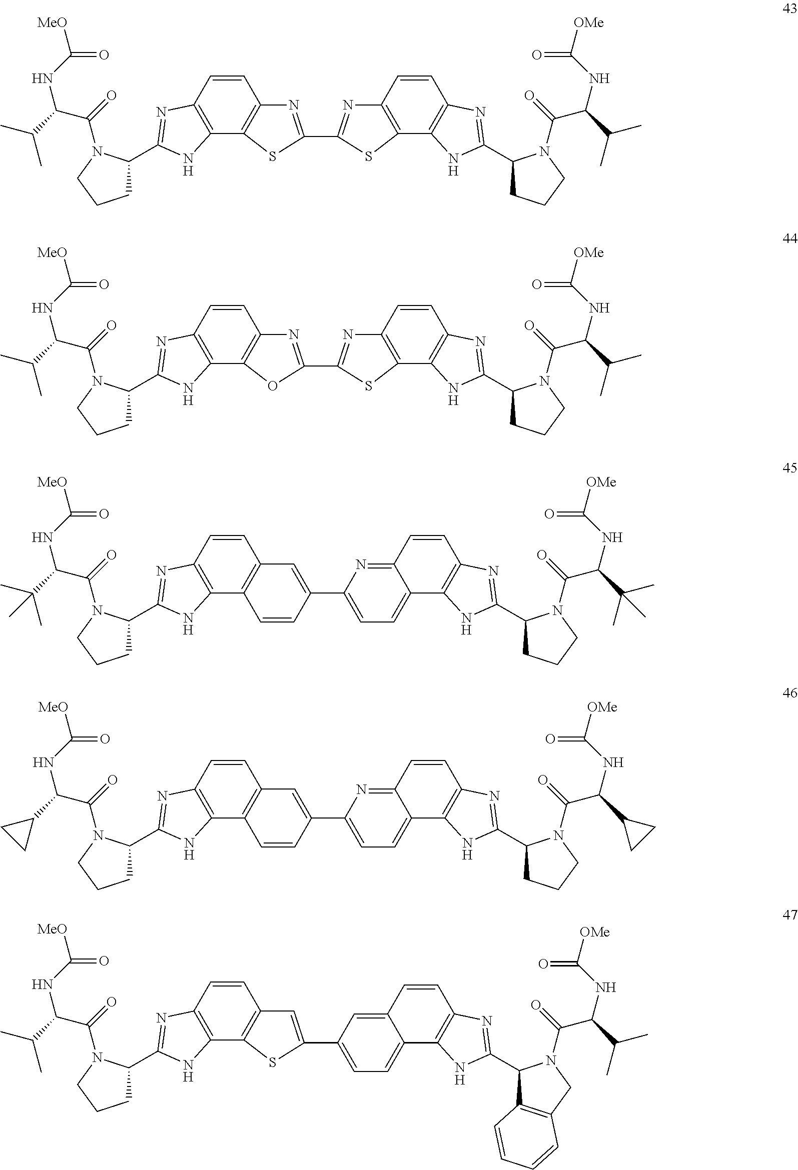

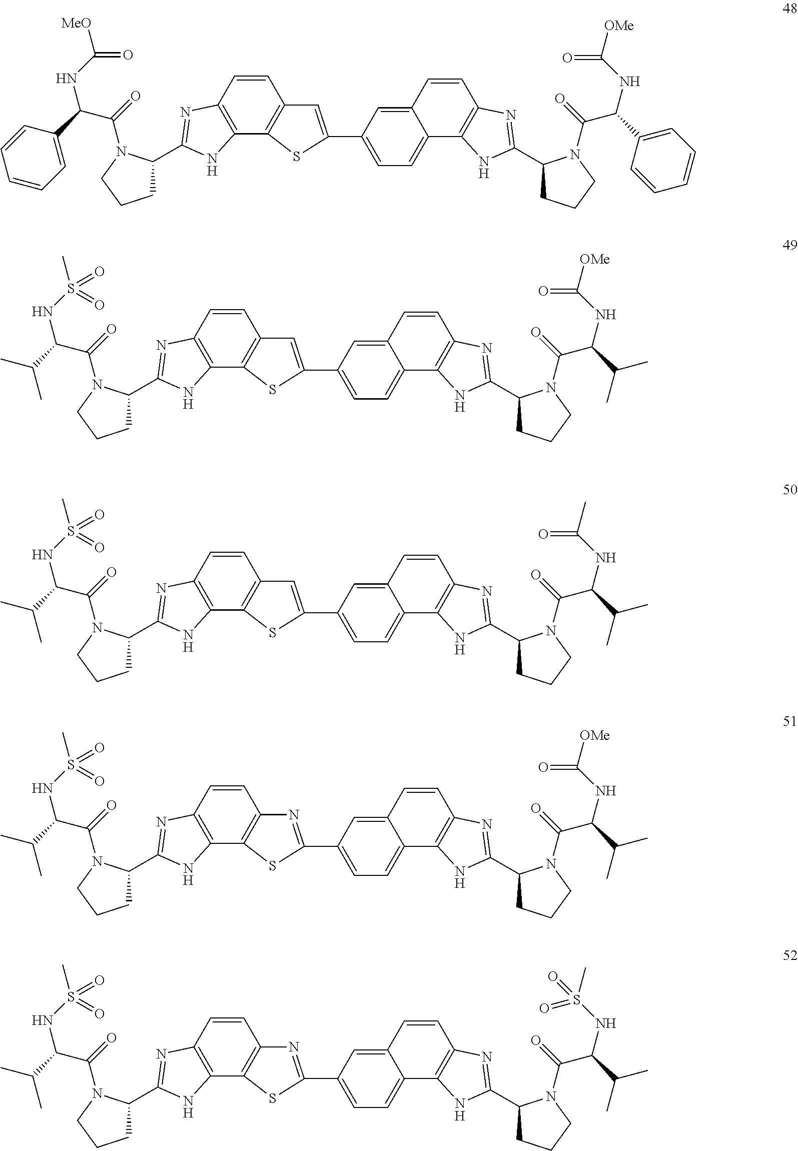

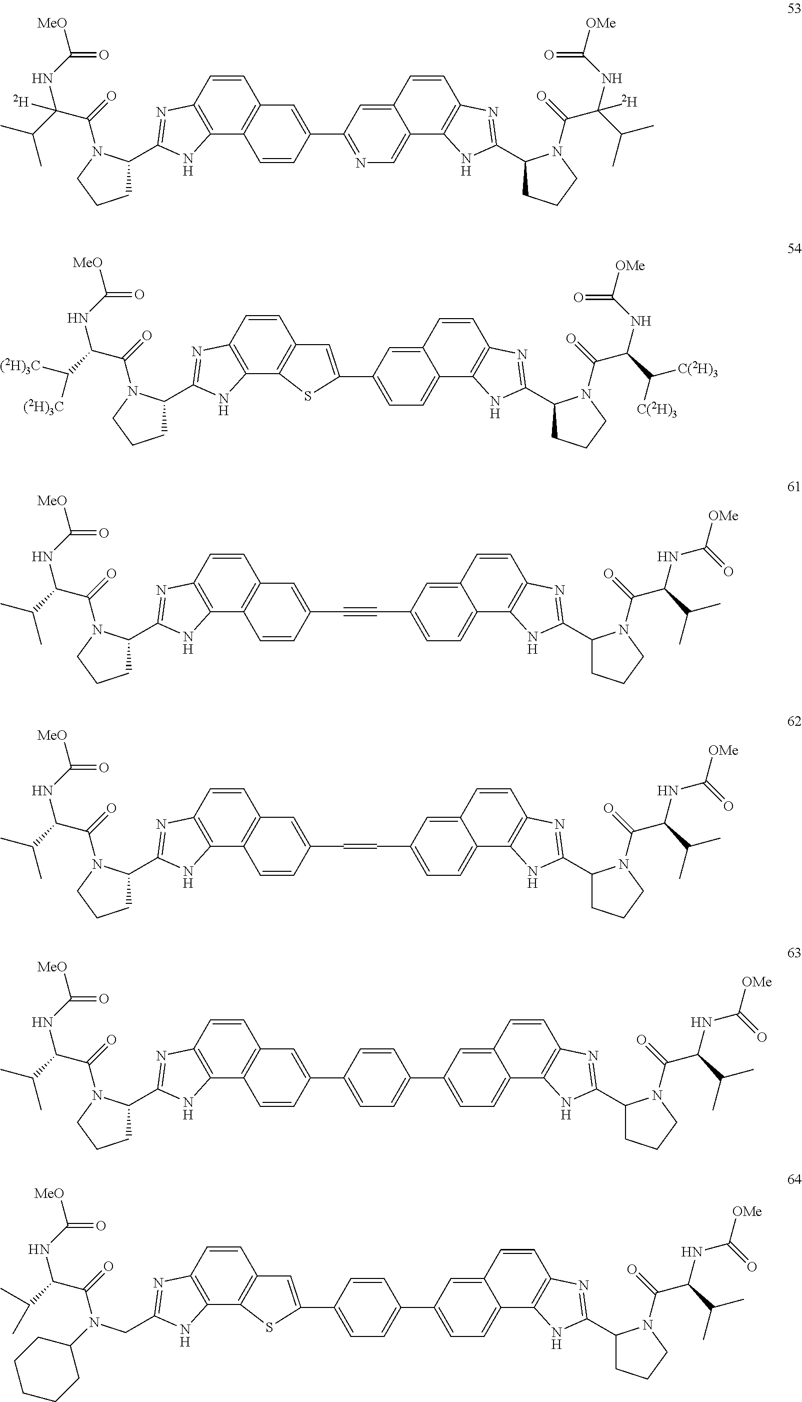

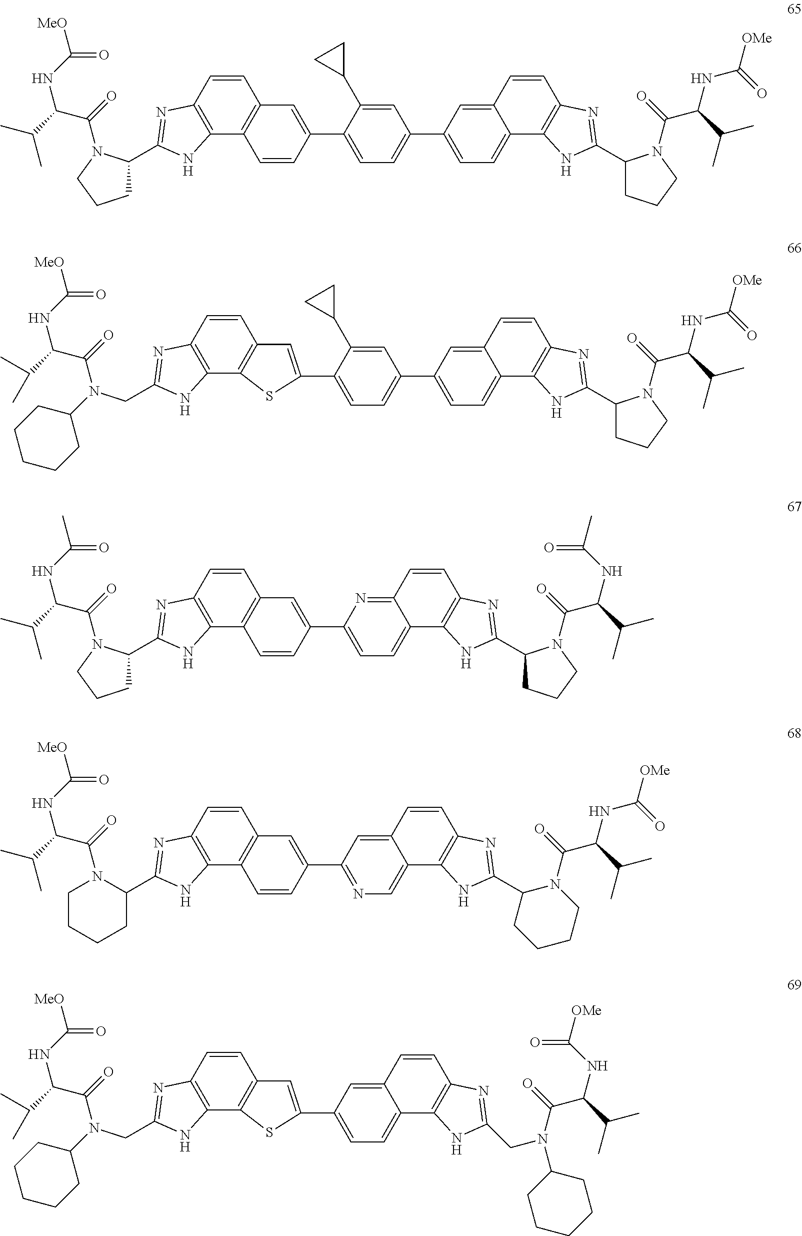

and pharmaceutically acceptable salts, solvates, prodrugs, esters and stereoisomers thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized below in Schemes 1-5. Alternative synthetic pathways and analogous structures will be apparent to those skilled in the art of organic synthesis. All stereoisomers and tautomeric forms of the compounds are contemplated.

Some commercially available starting materials and intermediates used for the synthesis of the Compounds of Formula (I) are available which contain intact fused tricyclic tricyclic ring systems. These starting materials and intermediates are available from commercial suppliers such as Sigma-Aldrich (St. Louis, Mo.) and Acros Organics Co. (Fair Lawn, N.J.). Such starting materials and intermediates compounds are used as received. When such fused tricyclic moieties are not commercially available, they can be prepared using methods well-known to those skilled in the art of organic synthesis. Such synthetic methods include, but are not limited to, those described in Kricka et al., J. Chem. Soc. Perkin Trans I, 859-863 (1973); Kricka et al., Chem. Rew., 74, 101-123, (1974); Kurfuerst et al., Coll. Czech. Chem. Comm., 54, 1705-1715, (1989); Saroja et al., J. Org. Chem. 69, 987-990, (2004); Fanta et al., Synth. 9-21, (1974), U.S. Patent Publication No. US2005038037; and International Publication No. WO2004039859.

Scheme 1 shows a method useful for making the naphtyl imidazole compounds of formula A6 and A7, which are useful intermediates for making the Compounds of Formula (I).

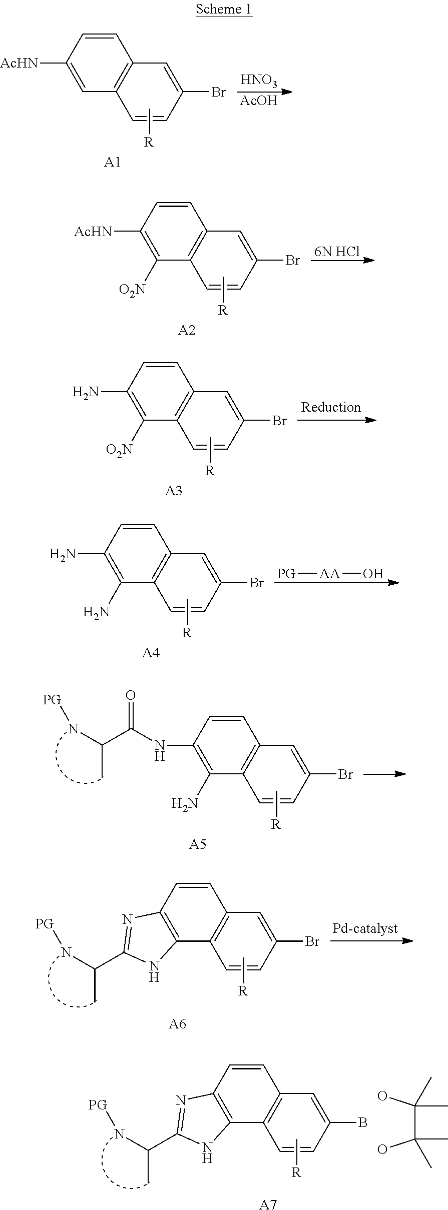

wherein PG-AA-OH refers to an amino acid that has its N-terminus protected by a protecting group (PG), such as Boc.

Nitration of bromonaphthal acetamide A1 provides nitro analog A2 (J. Am. Chem. Soc, 73:4297 (1997)). The removal of acetyl group under acidic conditions followed by reduction of the nitro group should afford diaminonaphthalene A4. Coupling of the aniline to an amino acid gives an amide, which upon heating in acetic acid will cyclize to provide tricyclic bormonaphthalimidazole A6. The bromide could be converted to a boronate A7 with a palladium catalyst.

Scheme 2 shows a method useful for making the quinolineimidazole compounds of formula B6, which are useful intermediates for making the Compounds of Formula (I).

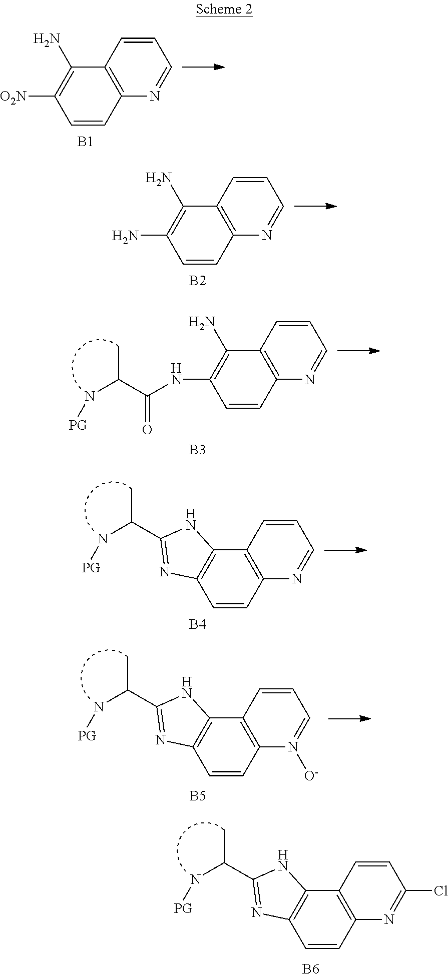

Commercially available aminonitroquinoline B1 can be reduced to diaininoquinoline B2, which is then coupled to an amino acid to provide an amide B3. It can then be cyclized to quinolineimidazole B4 under acidic conditions. N-oxide B5 can then be obtained with m-chloroperbenzoic acid. Upon treatment with phosphorous oxychloride, B5 should give the desired chloroquinoline B6, which can used in Suzuki coupling reactions.

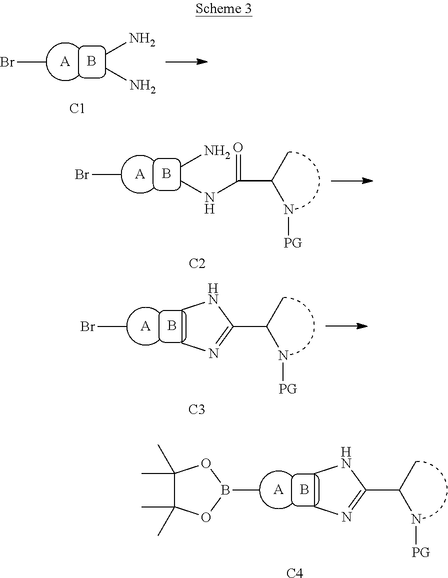

Similar to A4 and B2, an aromatic diamine C1 could be converted to a bicyclic imidazole C3 through a two step coupling-cyclization procedure (Scheme 3). The corresponding boronate C4 can then be obtained from bromide C3. Both C3 and C4 can be used in a Suzuki coupling to provide the precursors for the final target molecules.

Scheme 4 shows methods useful for making the Compounds of Formula (I) via a Suzuki Coupling process.

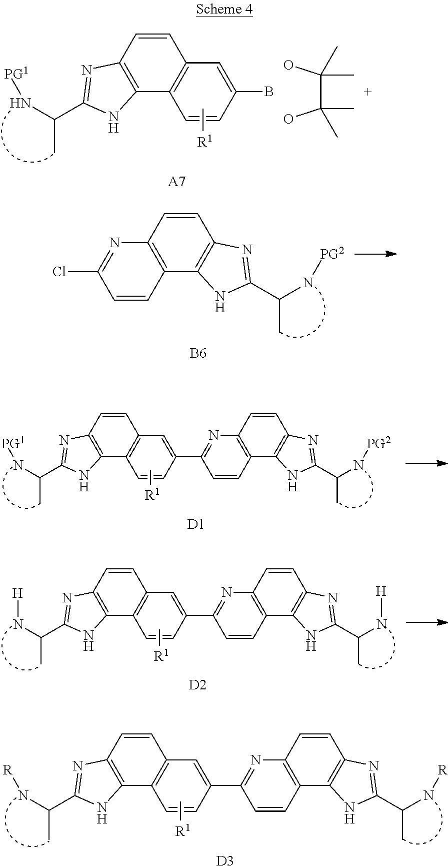

With the advanced coupling partners on hand, the Suzuki coupling between an aryl halide (e.g., A6 and B6) and an aryl boronate (e.g., A7) should provide biaryl products such as D1 (Scheme 4) using, for example the methods disclosed in Angew Chem. Int. Ed. Engl., 40, 4544 (2001). After removal of the protecting groups at nitrogen at both ends, an appropriate cap can be introduced to the resulted bis-amine using reactions including, but not limited to acylation (with an acyl chloride or amino acid coupling reagent such as HATU or HOBt/EDCI), sulfonylation (with a sulfonyl chloride) or alkylation (with alkyl halide or reductive amination) to provide the desired compounds of type D3.

Scheme 5 shows methods useful for making the Compounds of Formula (I) via a Suzuki coupling process.

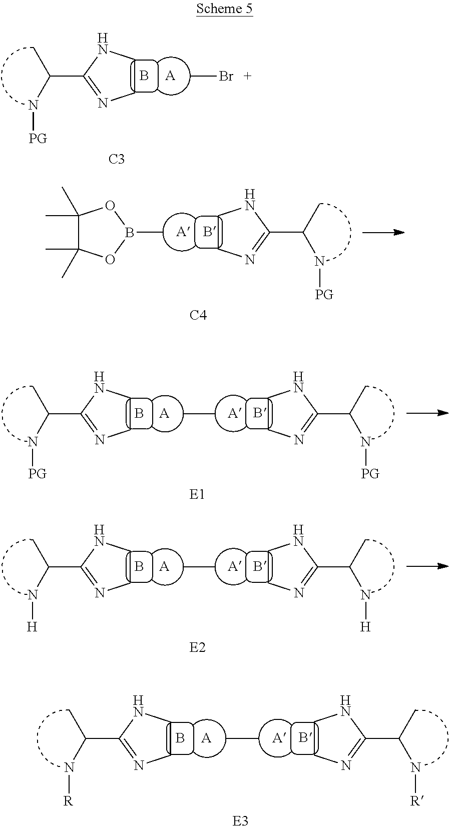

A Suzuki coupling between protected fused tricyclic boronate C4 (or boronic acid, not shown) and the fused tricyclic aryl halide such as bromide C3) using the methods similar to, but not limited to those reported in Angew Chem. Int. Ed. Engl., 40, 4544 (2001) should give compound of type E1, wherein at least one of the ring A or A′ is a 5- or 6-membered heterocycle. The product E1 is processed to the final targets E3 after removal the protecting groups and introducing the capping groups on the two amines using the method similar to the preparation of D3.

In some of bicyclic and fused tricyclic compounds contemplated in Scheme 1-5, the amino acids (such as, but not limited to proline, 4,4-difluoroproline, (S)-2-piperidine carboxylic acid, valine, alanine, norvaline, etc.) are incorporated as part of structures. Methods have been described in the general literature as well as in Banchard US 2009/0068140 (Published Mar. 9, 2009) for the preparation of such amino acid-derived intermediates.

One skilled in the art of organic synthesis will recognize that the synthesis of fused tricyclic cores in Formula (I) may require protection of certain functional groups (i.e., derivatization for the purpose of chemical compatibility with a particular reaction condition). Suitable protecting groups for the various functional groups of these compounds and methods for their installation and removal can be found in Greene et al., Protective Groups in Organic Synthesis, Wiley-Interscience, New York, (1999).

One skilled in the art of organic synthesis will also recognize that one route for the synthesis of fused bi-aryl tricyclic cores in Formula (I) may be more desirable depending on the choice of appendage substituents. Additionally, one skilled in the art will recognize that in some cases the order of reactions may differ from that presented herein to avoid functional group incompatibilities and can amend the synthetic route accordingly.

One skilled in the art of organic synthesis will recognize that the synthesis of certain fused tricyclic cores in Formula (I) require the construction of an amide bond. Methods useful for making such amide bonds, include but are not limited to, the use of a reactive carboxy derivative (e.g., an acid halide, or ester at elevated temperatures) or the use of an acid with a coupling reagent (e.g., HOBt, EDCI, DCC, HATU, PyBrop) with an amine.

The preparation of ring systems contemplated in this invention have been described in the literature and in compendia such as “Comprehensive Heterocyclic Chemistry” editions I, II and III, published by Elsevier and edited by A. R. Katritzky & R J K Taylor. Manipulation of the required substitution patterns have also been described in the available chemical literature as summarized in compendia such as “Comprehensive Organic Chemistry” published by Elsevier and edited by D H R. Barton and W. D. Ollis; “Comprehensive Organic Functional Group Transformations” edited by edited by A. R. Katritzky & R J K Taylor and “Comprehensive Organic Transformation” published by Wily-CVH and edited by R. C. Larock.

The starting materials used and the intermediates prepared using the methods set forth in the Schemes above may be isolated and purified if desired using conventional techniques, including but not limited to filtration, distillation, crystallization, chromatography and alike. Such materials can be characterized using conventional means, including physical constants and spectral data.

### EXAMPLES

### General Methods

Solvents, reagents, and intermediates that are commercially available were used as received. Reagents and intermediates that are not commercially available were prepared in the manner as described below. ¹H NMR spectra were obtained on a Bruker Avance 500 (500 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7mm ID; gradient flow: 0 min—10% CH₃CN, 5 min—95% CH₃CN, 5-7 min—95% CH₃CN, 7 min—stop. The observed parent ions are given. Flash column chromatography was performed using pre-packed normal phase silica from Biotage, Inc. or bulk silica from Fisher Scientific. Unless otherwise indicated, column chromatography was performed using a gradient elution of hexanes/ethyl acetate, from 100% hexanes to 100% ethyl acetate.

### Example 1

### Preparation of Intermediate Compound 1G

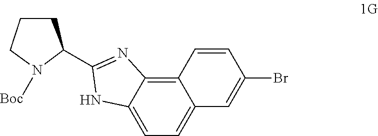

### Step A—Preparation of Compound 1A

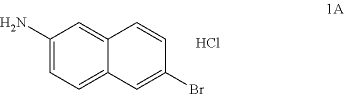

A mixture of 6-bromo-2-naphthoic acid (80.3 g, 319 mmol), diphenylphosphorylazide (71 mL, 352 mmol) and triethylamine (50 mL, 358 mmol) in tert-butanol (400 mL) was heated to reflux for 15 hours then cooled to room temperature. The reaction mixture was poured over saturated NaHCO₃ (600 mL) and stirred vigorously for 30 minutes. The solids were filtered, washed with water (200 mL) and dried under vacuum at 65° C. The resulting white solid was suspended in MeOH (500 mL), then HCl_{(g)} was bubbled into the mixture at −78° C. until saturated. The reaction mixture was stirred at room temperature for 15 hours, then the resulting solids were collected by filtration, and washed with ice-cold MeOH (100 mL) to provide Compound 1A as an off-white solid (74.8 g, 91%). ¹H NMR (DMSO-d₆) δ 10.5-10.0 (br s, 3H), 8.23 (s, 1H), 7.99 (d, J=9.0 Hz, 1H), 7.92 (d, J=9.0 Hz, 1H), 7.84 (s, 1H), 7.68-7.65 (m, 1H), 7.56-7.51 (m, 1H). LRMS: (M+2H)⁺=223.

Step B—Preparation of compound 1B

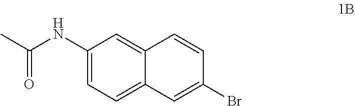

To the solution of Compound 1A (74.8 g, 289 mmol) and triethylamine (120 mL, 860 mmol) in CH₂Cl₂ (500 mL) at 0° C. was added acetic anhydride (27.5 mL, 292 mmol). The reaction mixture was warmed to room temperature and allowed to stir at this temperature for 1.5 hours. The mixture was filtered and the filtrate concentrated in vacuo. The resulting residue was triturated with hexanes (500 mL) and the solids were filtered, washed with hexanes (100 mL) and dried in vacuo at 55° C. to provide Compound 1B as an off-white solid (60.6 g, 79%). ¹H NMR (DMSO-d₆) δ 10.1 (s, 1H), 8.30 (s, 1H), 8.09 (s, 1H), 7.85-7.76 (m, 2H), 7.62-7.53 (m, 2H), 2.10 (s, 3H). LRMS: (M+H)⁺=265.

### Step C—Preparation of Compound 1C

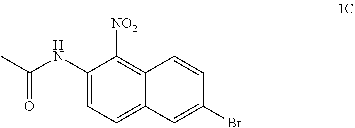

To a solution of compound 1B (60.6 g, 229 mmol) and acetic anhydride (120 mL) in acetic acid (500 mL) at 0° C. was added a solution of fuming nitric acid (36 mL) in AcOH (84 mL) dropwise over 2 hours. The resulting mixture was warmed to room temperature and stirred vigorously for 4.5 hours. The reaction mixture was filtered and the collected solids were washed with water (100 mL). The washed solids were recrystallized from EtOH (1.4 L) to provide Compound 1C as an off-white solid (58.5 g, 83%). ¹H NMR (DMSO-d₆) δ 8.95 (br s, 1H), 8.46 (d, J=9.0 Hz, 1H), 8.00 (s, 1H), 7.92-7.87 (m, 2H), 7.72-7.67 (m, 1H), 218 (s, 3H).

### Step D—Preparation of Compound 1D

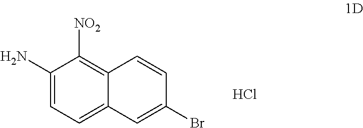

To a solution of Compound 1C (58.5 g, 189 mmol) in MeOH (150 mL) was added 6 N HCl (150 mL) and the mixture was heated to 75° C. and allowed to stir at this temperature for 6 hours, then cooled to room temperature. The resulting solids were filtered, rinsed with water (100 mL) and dried in vacuo at 55° C. to provide Compound 1D as a yellow solid (47.9 g, 95%). ¹H NMR (DMSO-d₆) δ 8.45(d, J=9.6 Hz, 1H), 8.09-8.00 (m, 3H), 7.84 (d, J=9.6 Hz, 1H), 7.73-7.67 (m, 1H), 7.21 (d, J=9.6 Hz, 1H), 3.33 (br s, 1H).

### Step E—Preparation of Compound 1E

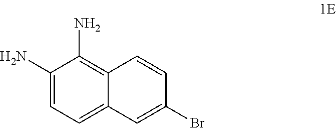

To a solution of Compound 1D (47.9 g, 179 mmol) and ammonium chloride (14.4 g, 269 mmol) in water (100 mL) and THF (250 mL) was added iron powder (50 g, 895 mmol). The resulting mixture was heated to 60° C. and allowed to stir vigorously at this temperature for 3 hours, then cooled to room temperature. The reaction mixture was filtered over Celite® and rinsed with MeOH until the Celite® was colorless. The filtrate was concentrated in vacuo and purified immediately on a silica gel plug (18 cm L×14 cm W) eluting with 1% MeOH/CH₂Cl₂ (7 L) to provide Compound 1E as a crude brown solid (40.5 g, 95%). ¹H NMR (DMSO-d₆) δ 7.85-7.79 (m, 2H), 7.32-7:29 (m, 1H), 7.03-6.96 (m, 2H), 4.86 (br s, 4H). LRMS: (M+H)⁺=238.

### Step F—Preparation of Compound 1F

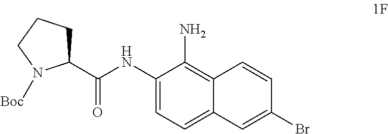

To a solution of compound 1E (40.5 g, 171 mmol), N-Boc-proline (45.0 g, 209 mmol) and N,N-diisopropylethylamine (90 mL, 517 mmol) in anhydrous DMF (1 L) at 0° C. was added HATU (78 g, 205 mmol). The resulting mixture was warmed to room temperature then stirred at this temperature for 9 hours. Water (1.5 L) was added to the reaction mixture and the resulting aqueous solution was extracted with MTBE (3×1.5 L). The combined organics were washed with brine (3×1 L), dried over Na₂SO₄, filtered and concentrated in vacuo. The resulting residue was dissolved in MeOH (75 mL) and water (1.5 L) was added. The resulting heterogeneous mixture was stirred vigorously for 2 hours and filtered. The filter cake was washed with water (1 L) and dried in vacuo at 55° C. to provide Compound 1F as an off-white solid (66.5 g, 90%). ¹H NMR (DMSO-d₆) δ 9.45-9.42 (m, 1H), 8.12-8.09 (m, 1H), 8.00 (s, 1H), 7.52-7.47 (m, 1H), 7.36-7.33 (m, 1H), 7.19-7.08 (m, 1H), 5.58 (s, 1H), 5.45 (s, 1H), 4.35-4.21- (m, 1H), 3.45-3.31 (m, 2H), 2.33-2.13 (m, 1H), 2.0-1.75 (m, 3H), 1.46-1.38 (m, 9H).

### Step G—Preparation of Compound 1G

A solution of compound 1F (66.5 g, 153 mmol) and AcOH (500 mL) was heated to 60° C. and allowed to stir at this temperature for 1 hour. After cooling to room temperature, water (1 L) was added and the resulting solution was adjusted to pH 8 using solid sodium carbonate. The resulting mixture was extracted with CH₂Cl₂ (2×1 L), dried over Na₂SO₄, filtered and concentrated in vacuo to provide Compound 1G as a crude brown solid (63.7 g, quant.), which was used without further purification. ¹H NMR (DMSO-d₆) δ 13.0-12.5 (n, 1H), 8.34 (d, J=9.0 Hz, 1H), 8.25-8.23 (m, 1H), 7.78-7.60 (m, 3H), 5.11-4.93 (m, 1H), 3.70-3.56 (m, 1H), 3.51-3.39 (m, 1H), 2.45-2.24 (m, 1H), 2.13-1.85 (m, 3H), 1.49-0.95 (m, 9H). LRMS: (M+H)⁺=416.

### Example 2

### Preparation of Intermediate Compound 2A

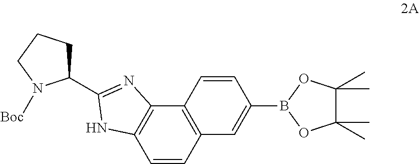

To a solution of compound 1G (21 g, 50.4 mmol), bis(pinacolato)diboron (14.1 g, 55.5 mmol), and KOAc (7.5 g, 76.4 mmol) in 1,4-dioxane (20 mL) was added a premixed solution of Pd(dba)₂ (1.16 g, 2.01 mmol) and tricyclohexylphosphine (1.14 g, 4.06 mmol) in 1,4-dioxane (10 mL). The resulting mixture was heated to 100° C. and allowed to stir at this temperature for 4 hours then cooled to room temperature. The reaction mixture was filtered through Celite®, the pad was washed with CH₂Cl₂ (100 mL) and the filtrate concentrated in vacuo. The residue obtained was purified using flash chromatography on an ISCO 330 g Redi-Sep column using a gradient of 0-70% EtOAc/hexanes as eluent to provide Compound 2A as a yellow solid (19 g, 82%). ¹H NMR (DMSO-d₆) δ 13.0-12.5 (m, 1H), 8.40-8.36 (m, 2H), 7.84-7.63 (m, 3H), 5.13-4.93 (m, 1H), 3.73-3.57 (m, 1H), 3.51-3.41 (m, 1H), 2.44-2.25 (m, 1H), 2.18-1.95 (m, 3H), 1.40-1.02 (m, 2H). LRMS: (M+H)⁺=464.

### Example 3

### Preparation of Intermediate Compound 3E

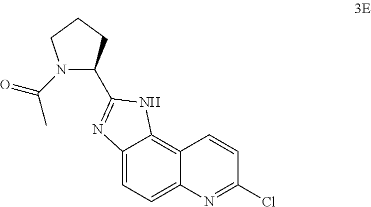

### Step A: Preparation of Compound 3A

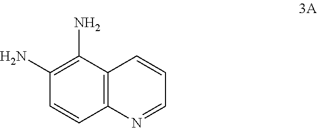

To a solution of 50% palladium on carbon (10% wet, 250 mg) in absolute ethanol (100 mL) under nitrogen was added 5-amino-6-nitroquinoline (5.00 g, 26.4 mmol). With stirring, the solution was placed under vacuum for 30 seconds and then was opened to a hydrogen gas balloon. After stirring for 2 hours under a hydrogen atmosphere, the reaction mixture was evacuoted under vacuum and placed under nitrogen, then sonicated for 10 minutes. Methanol was then added to the reaction mixture and the resulting solution was placed under hydrogen atmosphere and stirred for an additional 2 hours. The reaction mixture was then filtered through a Celite pad and the pad washed with methanol (2×200 mL). The filtrate was concentrated in vacuo and the resulting residue was dissolved in CH₂Cl₂ (75 mL) and the solution was purified via chromatography using an ISCO 330-g Redi-Sep column using 0-10% methanol/CH₂Cl₂ as eluent to provide Compound 3A as a yellow solid (3.76 g, 89%).

### Step B: Preparation of Compound 3B

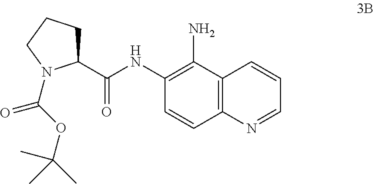

To a solution of 5,6-diaminoquinoline (1.00 g, 6.28 mmol), HATU (2.63 g, 6.91 mmol), N,N-diisopropylethylamine (3.28 mL, 18.8 mmol) and anhydrous DMF (20 mL) was added Boc-Pro-OH (1.49 g, 6.91 mmol). The reaction was placed under nitrogen atmosphere and allowed to stir at room temperature for 17 hours. The reaction mixture was then partitioned between EtOAc (100 mL) and saturated aqueous NaCl solution (100 mL) and the aqueous layer was extracted with EtOAc (4×100 mL). The combined organic extracts were washed with brine (4×100 mL), dried over Na₂SO₄, filtered and concentrated in vacuo. The residue obtained was dissolved in CH₂Cl₂ (10 mL) and purified via chromatography on an ISCO 80-g Redi-Sep column using 0-5% methanol/CH₂Cl₂ as eluent to provide Compound 3B as an orange oil (0.713 g, 32%). ESI-LRMS: (M+H—C₄H₉O₂)⁺=257.

### Step C—Preparation of Compound 3C

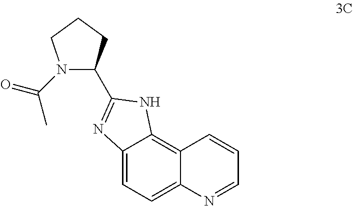

A solution of compound 3B (3.00 g, 8.41 mmol) CH₃COOH (70 mL) was put under nitrogen atmosphere, then heated to reflux and allowed to stir at this temperature for 18 hours. The reaction was cooled to room temperature, then concentrated in vacuo to provide a brown oil, which was diluted with CH₂Cl₂ and neutralized by adding saturated NaHCO₃ solution (125 mL). The resulting biphasic mixture was stirred for 1 hour, then was diluted with water and extracted with CH₂Cl₂ (2×200 mL). The combined organics were dried over MgSO₄, filtered and concentrated in vacuo to provide Compound 3C as an orange foam (2.04 g, 86%). ¹H NMR (CDCl₃) δ 11.61 (br s, 0.32H), 11.04 (br s, 0.68H), 8.93-8.85 (m, 1.68 H), 8.38-8.30 (m, 0.32H), 8.08-7.70 (m, 2H), 7.53-7.40 (m, 1H), 5.51-5.43 (m, 1H), 3.64-3.51 (m, 2H), 3.34-3.13. (m, 1H), 2.51-2.11 (m, 6H). LCMS: (M+H)⁺=281.

### Step D—Preparation of Compound 3D

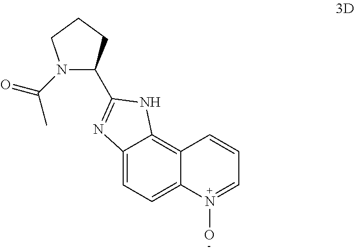

A solution of compound 3C (2.03 g, 7.24 mmol) in CH₂Cl₂ (75 mL) was placed under nitrogen atmosphere and cooled to 0° C. To the cooled solution was added 3-chloroperoxybenzoic acid (1.50 g, 8.69 mmol) and the resulting reaction was allowed to warm to room temperature while stirring for 18 hours. The reaction mixture was then cooled to 0° C. and quenched by adding 10% Na₂SO₃ solution (25 mL). The solvent from the reaction mixture was removed in vacuo and the remaining aqueous solution was loaded directly onto an ISCO 80 g Redi-Sep column and purified using flash chromatography using 0-10% CH₃OH/CH₂Cl₂ as the eluent to provide a bright yellow foam. The bright yellow foam was then purified again using a second flash chromatography purification using an ISCO 80 g Redi-Sep column and 0-10% CH₃OH/CH₂Cl₂ as the eluent to provide Compound 3D as a light yellow foam (1.85 g, 86%). ¹H NMR (CDCl₃) δ 11.69 (br s, 0.17H), 11.12 (br s, 0.83H), 8.59-8.38 (m, 2.83H), 8.04 7.96 (d, J=9.5 Hz, 0.17H), 7.88-7.81 (d, J=8.2 Hz, 0.17H), 7.75-7.67 (d, J=9.4 Hz, 0.83H), 7.36-7.23 (m, 1H), 5.43-5.34 (m, 1H), 3:56-3.48 (m, 2H), 3.24-3.06 (m, 1H), 2.43-2.06 (m, 6H).

### Step E—Preparation of Compound 3E

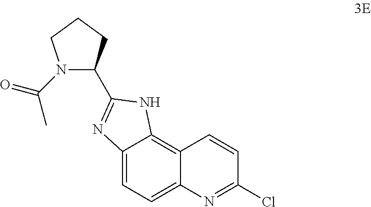

A solution of compound 3D (1.84 g, 6.20 mmol) in CH₂Cl₂ (20 mL) was placed under nitrogen atmosphere and cooled to 0° C. To the cooled solution was added triethylamine (1.04 mL, 7.45 mmol), the resulting reaction was stirred for 10 minutes at 0° C., then a solution of phosphoryl chloride (1.14 g, 7.45 mmol) in CH₂Cl₂ (10 mL) was added dropwise over 10 minutes. The reaction was stirred for an additional 2 hours at 0° C., then quenched by the dropwise addition of water (3.0 mL). The resulting reaction mixture was neutralized to pH 7 using 2N NaOH (˜15 mL), then loaded directly onto a 120 g Redi-Sep column and purified using flash chromatography using 0-10% CH₃OH/CH₂Cl₂as the eluent to provide a yellow solid product, consisting of two isomers. The two isomers were further separated using semi-preparative HPLC (Luna C18, CH₃CN/water with 0.05% TFA) and the isomerically pure fractions were combined with saturated NaHCO₃ solution (10 mL) and the organic portion was saved. The remaining aqueous portion was extracted with EtOAc (3×100 mL) and the combined organics were dried over Na₂SO₄, filtered and concentrated in vacuo. The resulting residue was dissolved in a mixture of CH₃CN and water and the solution was freeze-dried overnight to provide Compound 3E as an off-white solid (463 mg, 23%). ¹H NMR (CDCl₃) δ 11.10 (br s, 1H), 8.87 (br s, 1H), 7.89-7.68 (m, 2H), 7.53-7.42 (d, J=8.6 Hz, 1H), 5.52-5.40 (d, J=8.0 Hz, 1H), 3.69-3.53 (m, 2H), 3.26 (br s, 1H), 2.52-2.11 (m, 6H).

### Example 4

### Preparation of Compound 1

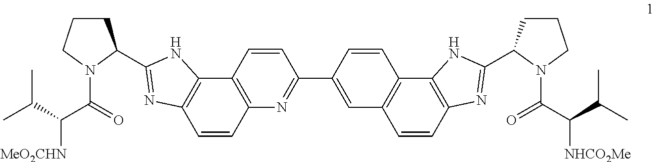

### Step A—Preparation of Compound 4A

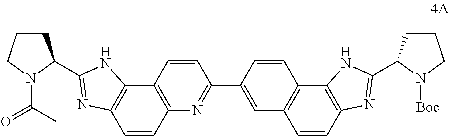

A mixture of Compound 3E (100 mg, 0.317 mmol), Compound 2A (150 mg, 0.323 mmol), Pd(PPh₃)₄ (37 mg, 0.032 mmol) and Na₂CO₃ (68 mg, 0.635 mmol) in 2:1 DME:H₂O (6 mL) was heated to 100° C. and allowed to stir at this temperature for 4 hours. The reaction mixture was cooled to room temperature, then diluted with saturated NH₄Cl (50 mL) and extracted with CH₂Cl₂ (2×50 mL). The combined organics were concentrated in vacuo and the resulting residue was purified using flash chromatography on an ISCO 12 g Redi-Sep column using 0-8% CH₃OH/CH₂Cl₂ as the eluent to provide Compound 4A as a yellow foam (161 mg, 82%). ¹H NMR (DMSO-d₆) δ 13.29-12.52 (m, 2 NH), 8.97-8.83 (m, 2H), 8.63-8.49 (m, 2H), 8.49-8.33 (m, 1H), 8.04-7.68 (m, 4H), 5.43-5.21 (m, 1H), 5.14-4.94 (m, 1H), 3.90-3.42 (m, 4H), 2.43-1.80 (m, 11H), 1.53-0.95 (m, 9H). MS (ESI): (M+H)⁺=616.

### Step B—Preparation of Compound 4B

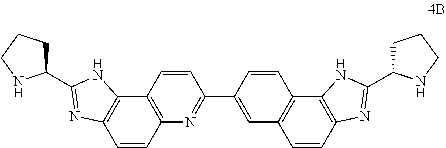

Compound 4A (159 mg, 0.258 mmol) was dissolved in 6N HCl (8 mL) and the resulting solution was heated to 90° C. and allowed to stir at this temperature for 4 hours. The reaction mixture was then cooled to room temperature, concentrated in vacuo, and the resulting yellow oily residue was dissolved in a mixture of water (15 mL) and CH₃CN (10 mL). The resulting solution was freeze-dried overnight to provide Compound 4B as a brownish yellow solid (216 mg, quant.). ¹H NMR (DMSO-d₆) δ 10.17 (br s, 2NH), 9.38 (br s, 2NH), 9.06-8.93 (m, 2H), 8.68-8.57 (m, 2H), 8.54-8.45 (m, 1H), 8.15-7.79 (m, 4H), 5.16-4.98 (m, 2H), 2.43-2.00 (m, 12H). MS (ESI): (M+H)⁺=474.

### Step C—Preparation of Compound 1

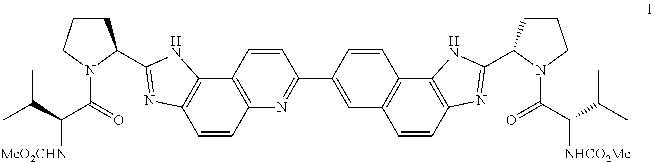

A solution of compound 4B (157 mg, 0.253 mmol) in DMF (5 mL) was placed under nitrogen atmosphere and cooled to 0° C. To the cooled solution was added N,N-diisopropylethylamine (0.39 mL, 2.28 mmol) and the resulting mixture was allowed to stir at 0° C. for 5 minutes. (S)-2-(methoxycarbonylamino)-3-methylbutanoic acid (107 mg, 0.608 mmol) and HATU (232 mg, 0.608 mmol) were then added to the reaction mixture and the resulting reaction was allowed to stir at 0° C. for 1 hour. The reaction mixture was then poured into a mixture of cold water (25 mL), saturated NaHCO₃ solution (25 mL) and EtOAc (50 mL). The resulting suspension was further diluted with brine (50 mL) and EtOAc (25 mL). The layers were partitioned and the aqueous portion extracted with EtOAc (75 mL). The combined organic extracts were concentrated in vacuo and the resulting solution of product in remaining DMF was diluted with 100 mL water, heated to 40° C. and allowed to stir overnight while cooling to room temperature on its own. The resulting suspension was then filtered and the collected brown solid was purified using flash chromatography on an ISCO 40 g Redi-Sep column using a gradient from 100% CH₂Cl₂ to 1% NH₄OH/9% CH₃OH/90% CH₂Cl₂ as the eluent. The resulting yellow oil was dissolved in a mixture of CH₃CN and water and the solution was freeze-dried overnight to provide Compound 1 as a yellow solid (99 mg, 51%). ¹H NMR (DMSO-d₆) δ 13.3-12.3 (m, 2NH), 8.96-8.74 (m, 2H), 8.64-8.31 (m, 3H), 8.03-7.63 (m, 4H), 7.53-7.17 (m, 2NH), 5.41-5.19 (m, 2H), 4.25-4.04 (m, 2H), 4.03-3.66 (m, 4H), 3.66-3.48 (m, 6H), 2.43-1.77 (m, 10H), 1.09-0.72 (m, 12H). MS (ESI): (M+H)⁺=788.8.

### Example 5

### Cell-Based HCV Replicon Assay

To measure cell-based anti-HCV activity of selected compounds of the present invention, replicon cells were seeded at 5000 cells/well in 96-well collagen I-coated Nunc plates in the presence of the test compound. Various concentrations of test compound, typically in 10 serial 2-fold dilutions, were added to the assay mixture, with the starting concentration ranging from 250 μM to 1 μM. The final concentration of DMSO was 0.5%, fetal bovine serum was 5%, in the assay media. Cells were harvested on day 3 by the addition of 1× cell lysis buffer (Ambion cat #8721). The replicon RNA level was measured using real time PCR (Taqman assay). The amplicon was located in 5B. The PCR primers were: 5B.2F, ATGGACAGGCGCCCTGA; 5B.2R, TTGATGGGCAGCTTGGTTTC; the probe sequence was FAM-labeled CACGCCATGCGCTGCGG. GAPDH RNA was used as endogenous control and was amplified in the same reaction as NS5B (multiplex PCR) using primers and VIC-labeled probe recommended by the manufacturer (PE Applied Biosystem). The real-time RT-PCR reactions were run on ABI PRISM 7900HT Sequence Detection System using the following program: 48° C. and allowed to stir at this temperature for 30 min, 95° C. and allowed to stir at this temperature for 10 min, 40 cycles of 95° C. and allowed to stir at this temperature for 15 sec, 60° C. and allowed to stir at this temperature for 1 minute. The ΔCT values (CT_{5B}-CT_{GAPDH}) were plotted against the concentration of test compound and fitted to the sigmoid dose-response model using XLfit4 (MDL). EC₅₀ was defined as the concentration of inhibitor necessary to achieve ΔCT=1 over the projected baseline; EC₉₀ the concentration necessary to achieve ΔCT=3.2 over the baseline. Alternatively, to quantitate the absolute amount of replicon RNA, a standard curve was established by including serially diluted T7 transcripts of replicon RNA in the Taqman assay. All Taqman reagents were from PE Applied Biosystems. Such an assay procedure was described in detail in e.g. Malcolm et al., Antimicrobial Agents and Chemotherapy 50: 1013-1020 (2006).

Using this method, Compound 1 demonstrated an EC₅₀ value of less than 1 nM.

### Uses of the Fused Tricyclic Compounds

The Fused Tricyclic Compounds are useful in human and veterinary medicine for treating or preventing a viral infection or a virus-related disorder in a patient. In accordance with the invention, the Fused Tricyclic Compounds can be administered to a patient in need of treatment or prevention of a viral infection or a virus-related disorder.

Accordingly, in one embodiment, the invention provides methods for treating a viral infection in a patient comprising administering to the patient an effective amount of at least one Fused Tricyclic Compound or a pharmaceutically acceptable salt, solvate, ester or prodrug thereof. In another embodiment, the invention provides methods for treating a virus-related disorder in a patient comprising administering to the patient an effective amount of at least one Fused Tricyclic Compound or a pharmaceutically acceptable salt, solvate, ester or prodrug thereof.

### Treatment or Prevention of a Viral Infection

The Fused Tricyclic Compounds can be useful for treating or preventing a viral infection. In one embodiment, the Fused Tricyclic Compounds can be inhibitors of viral replication. In a specific embodiment, the Fused Tricyclic Compounds can be inhibitors of HCV replication. Accordingly, the Fused Tricyclic Compounds are useful for treating viral infections, such as HCV.

Examples of viral infections that can be treated or prevented using the present methods, include but are not limited to, hepatitis A infection, hepatitis B infection and hepatitis C infection.

In one embodiment, the viral infection is hepatitis C infection.

In one embodiment, the hepatitis C infection is acute hepatitis C. In another embodiment, the hepatitis C infection is chronic hepatitis C.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al, J Gen Virol, 74(Pt11):2391-2399 (1993) is widely used and classifies isolates into six major genotypes, 1 through 6, with two or more related subtypes, e.g., 1a and 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al., J Gen Virol, 78(Pt1):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### Treatment or Prevention of a Virus-Related Disorder

The Fused Tricyclic Compounds can be useful for treating or preventing a virus-related disorder. Accordingly, the Fused Tricyclic Compounds are useful for treating disorders related to the activity of a virus, such as liver inflammation or cirrhosis. Virus-related disorders include, but are not limited to, RNA-dependent polymerase-related disorders and disorders related to HCV infection.

### Treatment or Prevention of a RNA-Dependent Polymerase-Related Disorder

The Fused Tricyclic Compounds can be useful for treating or preventing a RNA dependent polymerase (RdRp) related disorder in a patient. Such disorders include viral infections wherein the infective virus contains a RdRp enzyme.

Accordingly, in one embodiment, the present invention provides a method for treating a RNA dependent polymerase-related disorder in a patient, comprising administering to the patient an effective amount of at least one Fused Tricyclic Compound or a pharmaceutically acceptable salt, solvate, ester or prodrug thereof.

### Treatment or Prevention of a Disorder Related to HCV infection

The Fused Tricyclic Compounds can be useful for treating or preventing a disorder related to a HCV infection. Examples of such disorders include, but are not limited to, cirrhosis, portal hypertension, ascites, bone pain, varices, jaundice, hepatic encephalopathy, thyroiditis, porphyria cutanea tarda, cryoglobulinemia, glomerulonephritis, sicca syndrome, thrombocytopenia, lichen planus and diabetes mellitus.

Accordingly, in one embodiment, the invention provides methods for treating a HCV-related disorder in a patient, wherein the method comprises administering to the patient a therapeutically effective amount of at least one Fused Tricyclic Compound, or a pharmaceutically acceptable salt, solvate, ester or prodrug thereof.

### Combination Therapy

In another embodiment, the present methods for treating or preventing a viral infection or a virus-related disorder can further comprise the administration of one or more additional therapeutic agents which are not Substituted Fused Tricyclic Compounds.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one Substituted Fused Tricyclic Compound, or a pharmaceutically acceptable salt, solvate, ester or prodrug thereof, and (ii) at least one additional therapeutic agent that is other than a Substituted Fused Tricyclic Compound, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Substituted Fused Tricyclic Compound and an additional therapeutic agent may be present in fixed amounts (dosage amounts) in a single dosage unit (e.g., a capsule, a tablet and the like). A commercial example of such single dosage unit containing fixed amounts of two different active compounds is VYTORIN® (available from Merck Schering-Plough Pharmaceuticals, Kenilworth, N.J.).

In one embodiment, the at least one Substituted Fused Tricyclic Compound is administered during a time when the additional therapeutic agent(s) exert their prophylactic or therapeutic effect, or vice versa.

In another embodiment, the at least one Substituted Fused Tricyclic Compound and the additional therapeutic agent(s) are administered in doses commonly employed when such agents are used as monotherapy for treating a viral infection.

In another embodiment, the at least one Substituted Fused Tricyclic Compound and the additional therapeutic agent(s) are administered in doses lower than the doses commonly employed when such agents are used as monotherapy for treating a viral infection.

In still another embodiment, the at least one Substituted Fused Tricyclic Compound and the additional therapeutic agent(s) act synergistically and are administered in doses lower than the doses commonly employed when such agents are used as monotherapy for treating a viral infection.

In one embodiment, the at least one Substituted Fused Tricyclic Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The at least one Substituted Fused Tricyclic Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Substituted Fused Tricyclic Compound and the additional therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In one embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor.

In another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In another embodiment, the additional therapeutic agent is an HCV entry inhibitor.

In another embodiment, the additional therapeutic agent is an HCV assembly inhibitor.

In one embodiment, the additional therapeutic agents comprise a protease inhibitor and a polymerase inhibitor.

In still another embodiment, the additional therapeutic agents comprise a protease inhibitor and an immunomodulatory agent.

In yet another embodiment, the additional therapeutic agents comprise a polymerase inhibitor and an immunomodulatory agent.

In another embodiment, the additional therapeutic agents comprise a protease inhibitor and a nucleoside.

In another embodiment, the additional therapeutic agents comprise an immunomodulatory agent and a nucleoside.

In one embodiment, the additional therapeutic agents comprise a protease inhibitor and a NS5A inhibitor.

In another embodiment, the additional therapeutic agents comprise a nucleoside and a NS5A inhibitor.

In another embodiment, the additional therapeutic agents comprise a protease inhibitor, an immunomodulatory agent and a nucleoside.

In still another embodiment, the additional therapeutic agents comprise a protease inhibitor, a nucleoside and a NS5A inhibitor.

In a further embodiment, the additional therapeutic agents comprise a protease inhibitor, a polymerase inhibitor and an immunomodulatory agent.

In another embodiment, the additional therapeutic agent is ribavirin.

HCV polymerase inhibitors useful in the present compositions and methods include, but are not limited to, VP-19744 (Wyeth/ViroPharma), PSI-7851 (Pharmasset), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716 (ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125.

Interferons useful in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™ from Schering-Plough Corporation), interferon alpha-2b-XL (e.g., as sold under the trade name PEG-Intron™), interferon alpha-2c (Berofor Alpha™, Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), IFN-α-2b-XL (Flamel Technologies), and consensus interferon as defined by determination of a consensus sequence of naturally occurring interferon alphas (Infergen™, Amgen, Thousand Oaks, Calif.).

Antibody therapy agents useful in the present compositions and methods include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like).

Examples of viral protease inhibitors useful in the present compositions and methods include, but are not limited to, an HCV protease inhibitor.

HCV protease inhibitors useful in the present compositions and methods include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and International Publication Nos. WO 03/006490, WO 03/087092, WO 04/092161 and WO 08/124148.

Additional HCV protease inhibitors useful in the present compositions and methods include, but are not limited to, SCH503034 (Boceprevir, Schering-Plough), SCH900518 (Schering-Plough), VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), MK-7009 (Merck), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), PHX1766 (Phenomix), amprenavir, atazanavir, fosemprenavir, indinavir, lopinavir, ritonavir, nelfinavir, saquinavir, tipranavir, Kaletra (a combination of ritonavir and lopinavir) and TMC114.

Additional examples of HCV protease inhibitors useful in the present compositions and methods include, but are not limited to, those disclosed in Landro et al., Biochemistry, 36(31):9340-9348 (1997); Ingallinella et al., Biochemistry, 37(25):8906-8914 (1998); Llinàs-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); BioWorld Today, 9(217):4 (Nov. 10, 1998); U.S. Patent Publication Nos. US2005/0249702 and US 2007/0274951; and International Publication Nos. WO 98/14181, WO 98/17679, WO 98/17679, WO 98/22496 and WO 99/07734 and WO 05/087731.

Further examples of HCV protease inhibitors useful in the present compositions and methods include, but are not limited to, the following compounds:

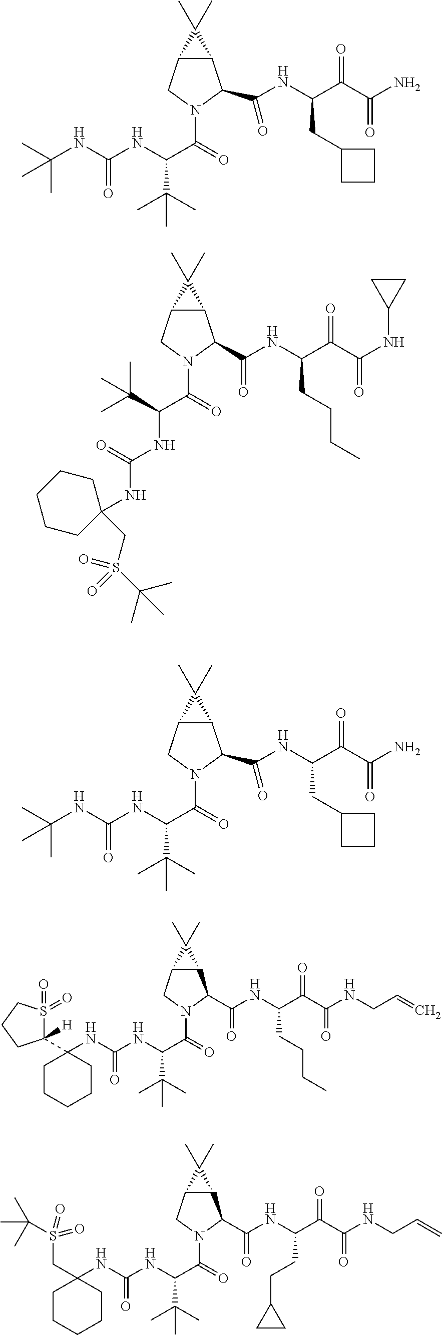

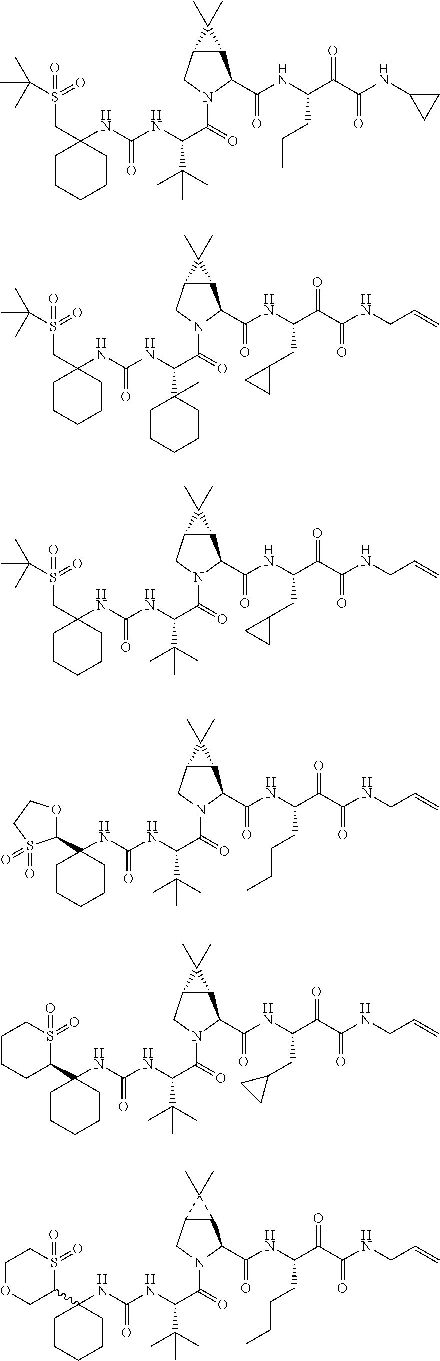

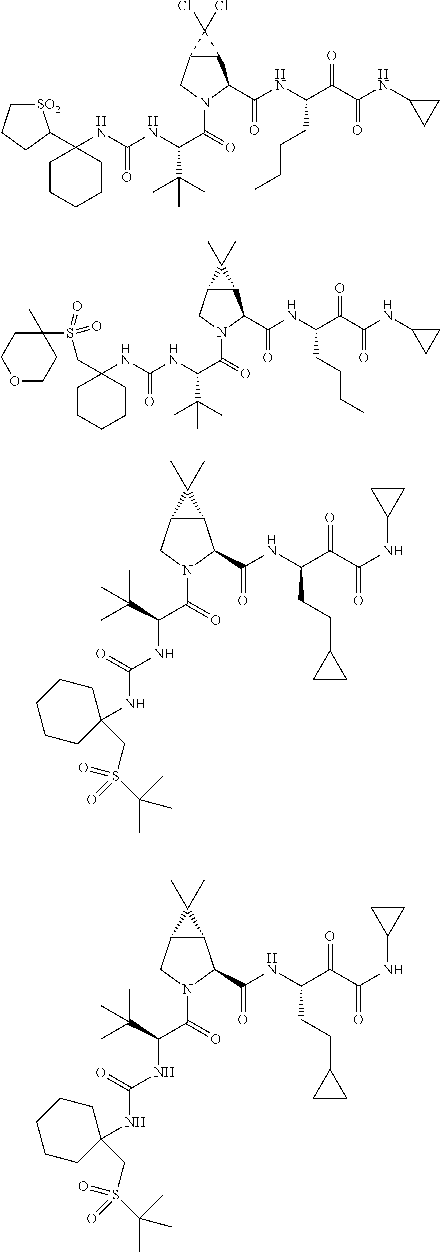

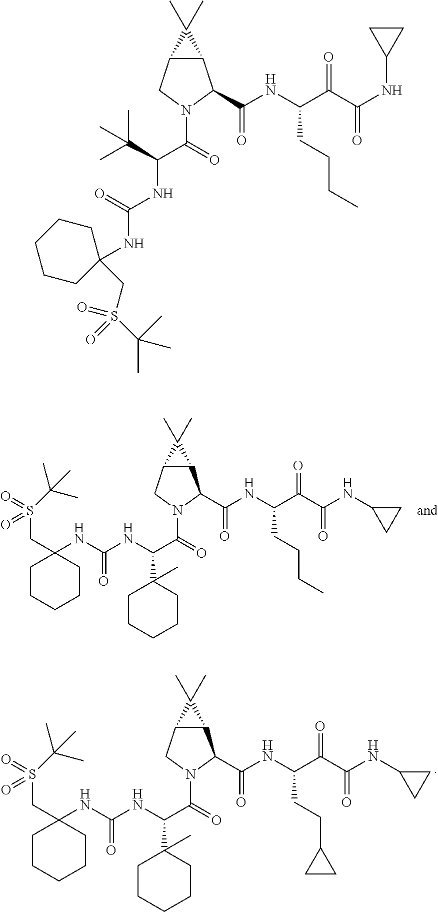

Viral replication inhibitors useful in the present compositions and methods include, but are not limited to, HCV replicase inhibitors, IRES inhibitors, NS4A inhibitors, NS3 helicase inhibitors, NS5A inhibitors, ribavirin, AZD-2836 (Astra Zeneca), BMS-790052 (Bristol-Myers Squibb), viramidine, A-831 (Arrow Therapeutics); an antisense agent or a therapeutic vaccine.

In one embodiment, viral replication inhibitors useful in the present compositions and methods include, but are not limited to, HCV replicase inhibitors, IRES inhibitors, NS4A inhibitors, NS3 helicase inhibitors and NS5A inhibitors.

HCV NS4A inhibitors useful in the useful in the present compositions and methods include, but are not limited to, those disclosed in U.S. Pat. Nos. 7,476,686 and 7,273,885; U.S. Patent Publication No. US20090022688; and International Publication Nos. WO 2006/019831 and WO 2006/019832. Additional HCV NS4A inhibitors useful in the useful in the present compositions and methods include, but are not limited to, AZD2836 (Astra Zeneca) and ACH-806 (Achillon Pharmaceuticals, New Haven, Conn.).

HCV replicase inhibitors useful in,the useful in the present compositions and methods include, but are not limited to, those disclosed in U.S. Patent Publication No. US20090081636.

Therapeutic vaccines useful in the present compositions and methods include, but are not limited to, IC41 (Intercell Novartis), CSL123 (Chiron/CSL), GI 5005 (Globeimmune), TG-4040 (Transgene), GNI-103 (GENimmune), Hepavaxx C (ViRex Medical); ChronVac-C (Inovio/Tripep), PeviPROTM (Pevion Biotect), HCV/MF59 (Chiron/Novartis) and Civacir (NABI).

Examples of further additional therapeutic agents useful in the present compositions and methods include, but are not limited to, TT033 (Benitec/Tacere Bio/Pfizer), Sirna-034 (Sirna Therapeutics), GNI-104 (GENimmune), GI-5005 (GlobeImmune), IDX-102 (Idenix), Levovirin™ (ICN Pharmaceuticals, Costa Mesa, Calif.); Humax (Genmab), ITX-2155 (Ithrex/Novartis), PRO 206 (Progenies), HepaCide-I (NanoVirocides), MX3235 (Migenix), SCY-635 (Scynexis); KPE02003002 (Kemin Pharma), Lenocta (VioQuest Pharmaceuticals), IET—Interferon Enhancing Therapy (Transition Therapeutics), Zadaxin (SciClone Pharma), VP 50406™ (Viropharma, Incorporated, Exton, Pa.); Taribavirin (Valeant Pharmaceuticals); Nitazoxanide (Romark); Debio 025 (Debiopharm); GS-9450 (Gilead); PF-4878691 (Pfizer); ANA773 (Anadys); SCV-07 (SciClone Pharmaceuticals); NIM-881 (Novartis); ISIS ¹⁴⁸⁰³™ (ISIS Pharmaceuticals, Carlsbad, Calif.); Heptazyme™ (Ribozyme Pharmaceuticals, Boulder, Colo.); Thymosin™ (SciClone Pharmaceuticals, San Mateo, Calif.); Maxamine™ (Maxim Pharmaceuticals, San Diego, Calif.); NKB-122 (JenKen Bioscience Inc., North Carolina); Alinia (Romark Laboratories), INFORM-1 (a combination of R7128 and ITMN-191); and mycophenolate mofetil (Hoffman-LaRoche, Nutley, N.J.).

The doses and dosage regimen of the other agents used in the combination therapies of the present invention for the treatment or prevention of a viral infection or virus-related disorder can be determined by the attending clinician, taking into consideration the approved doses and dosage regimen in the package insert; the age, sex and general health of the patient; and the type and severity of the viral infection or related disease or disorder. When administered in combination, the Substituted Fused Tricyclic Compound(s) and the other agent(s) can be administered simultaneously (i.e., in the same composition or in separate compositions one right after the other) or sequentially. This is particularly useful when the components of the combination are given on different dosing schedules, e.g., one component is administered once daily and another every six hours, or when the preferred pharmaceutical compositions are different, e.g., one is a tablet and one is a capsule. A kit comprising the separate dosage forms is therefore advantageous.

Generally, a total daily dosage of the at least one Substituted Fused Tricyclic Compound(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In one embodiment, when the additional therapeutic agent is INTRON-A interferon alpha 2b (commercially available from Schering-Plough Corp.), this agent is administered by subcutaneous injection at 3 MIU (12 mcg)/0.5 mL/TIW for 24 weeks or 48 weeks for first time treatment.

In another embodiment, when the additional therapeutic agent is PEG-INTRON interferon alpha 2b pegylated (commercially available from Schering-Plough Corp.), this agent is administered by subcutaneous injection at 1.5 mcg/kg/week, within a range of 40 to 150 mcg/week, for at least 24 weeks.

In another embodiment, when the additional therapeutic agent is ROFERON A interferon alpha 2a (commercially available from Hoffmann-La Roche), this agent is administered by subcutaneous or intramuscular injection at 3 MIU (11.1 mcg/mL)/TIW for at least 48 to 52 weeks, or alternatively 6 MIU/TIW for 12 weeks followed by 3 MIU/TIW for 36 weeks.

In still another embodiment, when the additional therapeutic agent is PEGASUS interferon alpha 2a pegylated (commercially available from Hoffmann-La Roche), this agent is administered by subcutaneous injection at 180 mcg/1 mL or 180 mcg/0.5 mL, once a week for at least 24 weeks.

In yet another embodiment, when the additional therapeutic agent is INFERGEN interferon alphacon-1 (commercially available from Amgen), this agent is administered by subcutaneous injection at 9 mcg/TIW is 24 weeks for first time treatment and up to 15 mcg/TIW for 24 weeks for non-responsive or relapse treatment.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

In one embodiment, one or more compounds of the present invention are administered with one or more additional therapeutic agents selected from an HCV protease inhibitor, an HCV replication inhibitor, a nucleoside, an interferon, a pegylated interferon and ribavirin. The combination therapies can include any combination of these additional therapeutic agents.

In another embodiment, one or more compounds of the present invention are administered with one additional therapeutic agent selected from an HCV protease inhibitor, an HCV replication inhibitor, a nucleoside, an interferon, a pegylated interferon and ribavirin.

In another embodiment, one or more compounds of the present invention are administered with two additional therapeutic agents selected from an HCV protease inhibitor, an HCV replication-inhibitor, a nucleoside, an interferon, a pegylated interferon and ribavirin.

In a specific embodiment, one or more compounds of the present invention are administered with an HCV protease inhibitor and ribavirin. In another specific embodiment, one or more compounds of the present invention are administered with a pegylated interferon and ribavirin.

In another embodiment, one or more compounds of the present invention are administered with three additional therapeutic agents selected from an HCV protease inhibitor, an HCV replication inhibitor, a nucleoside, an interferon, a pegylated interferon and ribavirin.

In one embodiment, one or more compounds of the present invention are administered with one or more additional therapeutic agents selected from an HCV polymerase inhibitor, a viral protease inhibitor, an interferon, and a viral replication inhibitor. In another embodiment, one or more compounds of the present invention are administered with one or more additional therapeutic agents selected from an HCV polymerase inhibitor, a viral protease inhibitor, an interferon, and a viral replication inhibitor. In another embodiment, one or more compounds of the present invention are administered with one or more additional therapeutic agents selected from an HCV polymerase inhibitor, a viral protease inhibitor, an interferon, and ribavirin.

In one embodiment, one or more compounds of the present invention are administered with one additional therapeutic agent selected from an HCV polymerase inhibitor, a viral protease inhibitor, an interferon, and a viral replication inhibitor. In another embodiment, one or more compounds of the present invention are administered with ribavirin.

In one embodiment, one or more compounds of the present invention are administered with two additional therapeutic agents selected from an HCV polymerase inhibitor, a viral protease inhibitor, an interferon, and a viral replication inhibitor.

In another embodiment, one or more compounds of the present invention are administered with ribavirin, interferon and another therapeutic agent.

In another embodiment, one or more compounds of the present invention are administered with ribavirin, interferon and another therapeutic agent, wherein the additional therapeutic agent is selected from an HCV polymerase inhibitor, a viral protease inhibitor, and a viral replication inhibitor.

In still another embodiment, one or more compounds of the present invention are administered with ribavirin, interferon and a viral protease inhibitor.

In another embodiment, one or more compounds of the present invention are administered with ribavirin, interferon and an HCV protease inhibitor.

In another embodiment, one or more compounds of the present invention are administered with ribavirin, interferon and boceprevir or telaprevir.

In a further embodiment, one or more compounds of the present invention are administered with ribavirin, interferon and an HCV polymerase inhibitor.

### Compositions and Administration

Due to their activity, the Fused Tricyclic Compounds are useful in veterinary and human medicine. As described above, the Fused Tricyclic Compounds are useful for treating or preventing a viral infection or a virus-related disorder in a patient in need thereof.

When administered to a patient, the Fused Tricyclic Compounds can be administered as a component of a composition that comprises a pharmaceutically acceptable carrier or vehicle. The present invention provides pharmaceutical compositions comprising an effective amount of at least one Fused Tricyclic Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage fowls suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly, before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

The Fused Tricyclic Compounds of the present invention may also be deliverable transdermally. The transdermal compositions can take the form of creams, lotions, aerosols and/or emulsions and can be included in a transdermal patch of the matrix or reservoir type as are conventional in the art for this purpose.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Fused Tricyclic Compounds are administered orally.

In another embodiment, the one or more Fused Tricyclic Compounds are administered intravenously.

In another embodiment, the one or more Fused Tricyclic Compounds are administered topically.

In still another embodiment, the one or more Fused Tricyclic Compounds are administered sublingually.

In one embodiment, a pharmaceutical preparation comprising at least one Fused Tricyclic Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Fused Tricyclic Compound(s) by weight or volume. In various embodiments, the present compositions can contain, in one embodiment, from about 1% to about 70% or from about 5% to about 60% of the Fused Tricyclic Compound(s) by weight or volume.

The quantity of Fused Tricyclic Compound in a unit dose of preparation may be varied or adjusted from about I mg to about 2500 mg. In various embodiment, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Fused Tricyclic Compounds will be regulated according to the judgment of the attending clinician considering such factors as age, condition and size of the patient as well as severity of the symptoms being treated. Generally, a total daily dosage of the Fused Tricyclic Compounds range from about 0.1 to about 2000 mg per day, although variations, will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Fused Tricyclic Compound or a pharmaceutically acceptable salt, solvate, ester or prodrug thereof; (ii) one or more additional therapeutic agents that are not a Fused Tricyclic Compound; and (iii) a pharmaceutically acceptable carrier, wherein the amounts in the composition are together effective to treat a viral infection or a virus-related disorder.

### Kits

In one aspect, the present invention provides a kit comprising a therapeutically effective amount of at least one Fused Tricyclic Compound, or a pharmaceutically acceptable salt, solvate, ester or prodrug of said compound and a pharmaceutically acceptable carrier, vehicle or diluent.

In another aspect the present invention provides a kit comprising an amount of at least one Fused Tricyclic Compound, or a pharmaceutically acceptable salt, solvate, ester or prodrug of said compound and an amount of at least one additional therapeutic agent listed above, wherein the amounts of the two or more active ingredients result in a desired therapeutic effect. In one embodiment, the one or more Fused Tricyclic Compounds and the one or more additional therapeutic agents are provided in the same container. In one embodiment, the one or more Fused Tricyclic Compounds and the one or more additional therapeutic agents are provided in separate containers.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula: 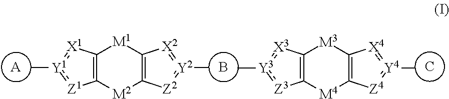
or a pharmaceutically acceptable salt thereof, wherein each dotted line represents an optional and additional bond, such that only one optional and additional bond can be attached to each of Y¹, Y², Y³ and Y⁴, and wherein:
A is -alkylene-N(R¹¹)(R¹³) or heterocycloalkyl, wherein said heterocycloalkyl group can be optionally and independently substituted with from one to three R⁴ groups, and wherein said heterocycloalkyl group can be optionally fused to a cycloalkyl group or a benzene group;
B is a bond, C₁-C₃ alkylene, —C(R⁵)═C(R⁵)—, —C≡C—, phenylene, monocyclic cycloalkylene, monocyclic cycloalkenylene, monocyclic heterocycloalkylene, monocyclic heterocycloalkenylene or monocyclic heteroarylene, wherein said phenylene group or said monocyclic cycloalkylene group can be optionally and independently substituted with one or more R¹⁴ groups, and wherein said phenylene group or said monocyclic cycloalkylene group can be optionally and independently substituted on one or more ring nitrogen atoms with R⁶ and on one or more ring carbon atoms with R¹⁴;
C is -alkylene-N(R¹¹)(R¹³) or heterocycloalkyl, wherein said heterocycloalkyl group can be optionally and independently substituted with from one to three R⁴ groups, and wherein said heterocycloalkyl group can be optionally fused to a cycloalkyl group or a benzene group;
M¹ is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—;
M² is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—, such that at least one of M¹ and M² is other than a bond, and such that the central ring of formula (I) that contains M¹ and M² has from 5 to 10 total ring atoms, and wherein two vicinal R⁷ groups of M¹ or M² together with the carbon atoms to which they are attached, can optionally join to form a 3- to 7-membered cycloalkyl group, a 3- to 7-membered heterocycloalkyl group, or a 5- to 6-membered heteroaryl group;
M³ is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—;
M⁴ is a bond, —[C(R⁷)₂]_{q}—, —[C(R⁷)₂]ₘ—C(R⁷)═C(R⁷)—[C(R⁷)₂]ₘ—, —C(R⁷)═N—, —N═C(R⁷)—, —[C(R⁷)₂]ₘ—O—[C(R⁷)₂]ₘ, —O—[C(R⁷)₂]_{q}—O—, —[C(R⁷)₂]ₘ—N(R⁶)—[C(R⁷)₂]ₘ—, —S—, —[C(R⁷)₂]ₘ—S(O)ₘ—[C(R⁷)₂]ₘ—, —[C(R⁷)₂]ₘ—OC(O)N(R⁶)—[C(R⁷)₂]ₘ—, —[C(R⁷)₂]ₘ—N(R⁶)C(O)N(R⁶)—[C(R⁷)₂]ₘ—, —[C(R⁷)₂]ₘ—S(O)₂N(R⁶)—[C(R⁷)₂]ₘ— or —[C(R⁷)₂]ₘ—N(R⁶)S(O)₂N(R⁶)—[C(R⁷)₂]ₘ—, such that at least one of M³ and M⁴ is other than a bond, and such that the central ring of formula (I) that contains M³ and M⁴ has from 5 to 10 total ring atoms, and wherein two vicinal R⁷ groups of M³ or M⁴ together with the carbon atoms to which they are attached, can optionally join to form a 3- to 7-membered cycloalkyl group, a 3- to 7-membered heterocycloalkyl group, or a 5- to 6-membered heteroaryl group;
X¹ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X¹ is not present, and X¹ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to X¹ is present, such that X¹ and Z¹ cannot each be a bond, and such that when X¹ is —C(R⁵)— or —N—, then Z¹ is other than a bond;
X² is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X² is not present, and X² is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to X² is present, such that X² and Z² cannot each be a bond, and such that when X² is —C(R⁵)— or —N—, then Z² is other than a bond;
X³ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X³ is not present, and X³ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to X³ is present, such that X³ and Z³ cannot each be a bond, and such that when X³ is —C(R⁵)— or —N—, then Z³ is other than a bond;
X⁴ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to X⁴ is not present, and X⁴ is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to X⁴ is present, such that X⁴ and Z⁴ cannot each be a bond, and such that when X⁴ is —C(R⁵)— or —N—, then Z⁴ is other than a bond;
Y¹ is —C— when an optional and additional bond to Y¹ is present, and Y¹ is —CH— when an optional and additional bond to Y¹ is absent;
Y² is —C— when an optional and additional bond to Y² is present, and Y² is —CH— when an optional and additional bond to Y² is absent;
Y³ is —C— when an optional and additional bond to Y³ is present, and Y³ is —CH— when an optional and additional bond to Y³ is absent;
Y⁴ is —C— when an optional and additional bond to Y⁴ is present, and Y⁴ is —CH— when an optional and additional bond to Y⁴ is absent;
Z¹ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z¹ is not present, and Z¹ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to Z¹ is present, such that such that the ring in formula (I) containing X¹, Y¹ and Z¹ has 5 or 6 total ring atoms;
Z² is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z² is not present, and Z² is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to Z² is present, such that the ring in formula (I) containing X², Y² and Z² has 5 or 6 total ring atoms;
Z³ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z³ is not present, and Z³ is —C(R⁵)(CH(R⁵))ₘ—, —N—, —N—CH(R⁵)CH(R⁵)—, —C(R⁵)NHCH(R⁵)—, —C(R⁵)CH(R⁵)NH—, —C(R⁵)O—, —C(R⁵)N(R⁶)—, —N—N(R⁶)—, —C(R⁵)S— or —C(R⁵)S(O)₂— when the optional and additional bond to Z³ is present, such that such that the ring in formula (I) containing X³, Y³ and Z³ has 5 or 6 total ring atoms;
Z⁴ is a bond, —C(R⁵)═C(R⁵)—, —N═C(R⁵)—, —C(R⁵)═NC—, —C(R⁵)═N—, —O—, —N(R⁶)—, —S— or —S(O)₂— when the optional and additional bond to Z⁴ is not present, and Z⁴ is —(CH(R⁵))ₘC(R⁵)—, —N—, —CH(R⁵)CH(R⁵)N—, —CH(R⁵)NHC(R⁵)—, —NHCH(R⁵)C(R⁵)—, —O—C(R⁵)—, —N(R⁶)C(R⁵)—, —N(R⁶)—N—, —S—C(R⁵)— or —S(O)₂C(R⁵)— when the optional and additional bond to Z⁴ is present, such that the ring in formula (I) containing X⁴, Y⁴ and Z⁴ has 5 or 6 total ring atoms;
each occurrence of R¹ is independently alkyl, haloalkyl, aryl, cycloalkyl, heterocycloalkyl or heteroaryl, wherein said aryl group, said cycloalkyl group, said heterocycloalkyl group or said heteroaryl group can be optionally and independently substituted with R²;
each occurrence of R² is independently alkyl, halo, haloalkyl, aryl, heterocycloalkyl, heteroaryl, —CN, —OR³, —N(R³)₂, —C(O)R¹², —C(O)OR³, —C(O)N(R³)₂, —NHC(O)R¹², —NHC(O)NHR³, —NHC(O)OR³, —OC(O)R¹², —SR³ or —S(O)₂R¹²;
each occurrence of R³ is independently H, alkyl, haloalkyl, aryl, cycloalkyl, heterocycloalkyl or heteroaryl;
each occurrence of R⁴ is independently halo, —C(O)—[C(R⁵)₂]_{q}N(R⁶)₂, —C(O)—[CH(R⁵)]_{q}N(R⁶)C(O)—R¹, —C(O)—[CH(R⁵)]_{q}N(R⁶)C(O)O—R¹, —C(O)—[CH(R⁵)]_{q}C(O)O—R¹, —C(O)[CH(R⁵)]_{q}N(R⁶)SO₂—R¹ or -alkylene-N(R⁶)—[CH(R⁵)]_{q}—N(R⁶)—C(O)O—R¹;
each occurrence of R⁵ is independently H, —C₁-C₆ alkyl, 3 to 7-membered cycloalkyl, aryl or heteroaryl;
each occurrence of R⁶ is independently H, —C₁-C₆ alkyl, 3 to 7-membered cycloalkyl, 4 to 7-membered heterocycloalkyl, aryl, or heteroaryl, wherein said 3 to 7-membered cycloalkyl group, said 4 to 7-membered heterocycloalkyl group, said aryl group or said heteroaryl group can be optionally and independently substituted with up to two R⁸ groups, and wherein two R⁶ groups that are attached to a common nitrogen atom, together with the nitrogen atom to which they are attached, can optionally join to form a 4 to 7-membered heterocycloalkyl group;
each occurrence of R⁷ is independently H, —C₁-C₆ alkyl, 3 to 7-membered cycloalkyl, 3 to 7-membered heterocycloalkyl, aryl or heteroaryl, wherein said 3 to 7-membered cycloalkyl group, said 3 to 7-membered heterocycloalkyl group, said aryl group or said heteroaryl group can be optionally and independently substituted with up to 3 substituents, which can be the same or different, and are selected from C₁-C₆ alkyl, halo, —C₁-C₆ haloalkyl, —C₁-C₆ hydroxyalkyl, —OH, —C(O)NH—(C₁-C₆ alkyl), —C(O)N(C₁-C₆ alkyl)₂, —O—(C₁-C₆ alkyl), —NH₂, —NH(C₁-C₆ alkyl), —N(C₁-C₆ alkyl)₂ and —NHC(O)—(C₁-C₆ alkyl), and wherein two geminal R⁷ groups, together with the common carbon atom to which they are attached, can optionally join to form —C(O)—, —C(S)—, —C(═NR⁹)—, —C(═NOR⁹)—, a 3 to 7-membered cycloalkyl group or a 3 to 7-membered heterocycloalkyl group, such that no two adjacent —C(R⁷)₂— groups can join to form a —C(O)—C(O)—, —C(S)—C(S)—, —C(O)—C(S)— or —C(S)—C(O)— group;
each occurrence of R⁸ is independently H or —C₁-C₆ alkyl;
each occurrence of R⁹ is independently H, —C₁-C₆ alkyl, 3 to 6-membered cycloalkyl or 4 to 6-membered heterocycloalkyl;
R¹¹ is 3 to 7-membered cycloalkyl, 3 to 7-membered heterocycloalkyl, aryl or heteroaryl, wherein said 3 to 7-membered cycloalkyl group, said 3 to 7-membered heterocycloalkyl group, said aryl group or said heteroaryl group can be optionally and independently substituted with up to three R² groups;
each occurrence of R¹² is independently alkyl, haloalkyl, aryl, cycloalkyl, heterocycloalkyl or heteroaryl;
each occurrence of R¹³ is independently —C(O)—[C(R⁷)₂]_{q}N(R⁶)₂, —C(O)—[C(R⁷)₂]_{q}N(R⁶)C(O)—R¹, —C(O)—[C(R⁷)₂]_{q}N(R⁶)C(O)O—R¹, —C(O)—[C(R⁷)₂]_{q}C(O)O—R¹, —C(O)[C(R⁷)₂]_{q}N(R⁶)SO₂—R¹ or -alkylene-N(R⁶)—[C(R⁷)₂]_{q}N(R⁶)—C(O)O—R¹;
each occurrence of R¹⁴ is H, —C₁-C₆ alkyl, 3 to 7-membered cycloalkyl, 3 to 7-membered heterocycloalkyl, aryl, heteroaryl, halo, haloalkyl, —CN, —OR³, —N(R³)₂, —C(O)R¹², —C(O)OR³, —C(O)N(R³)₂, —NHC(O)R¹², —NHC(O)NHR³, —NHC(O)OR³, —OC(O)R¹², —SR³ or —S(O)₂R¹²;
each occurrence of m is independently an integer ranging from 0 to 2; and
each occurrence of q is independently an integer ranging from 1 to 4,
such that at least one of the rings containing (i) X², Y² and Z² or (ii) X³, Y³ and Z³ is other than a benzene ring.

2. The compound of claim 1, wherein:
B is a bond, phenylene, monocyclic cycloalkylene, monocyclic cycloalkenylene, monocyclic heterocycloalkylene, monocyclic heterocycloalkenylene or monocyclic heteroarylene, wherein said phenylene group or said monocyclic cycloalkylene group can be optionally and independently substituted with one or more R¹⁴ groups, and wherein said phenylene group or said monocyclic cycloalkylene group can be optionally and independently substituted on one or more ring nitrogen atoms with R⁶ and on one or more ring carbon atoms with R¹⁴;
M¹ is a bond, —C(R⁷)₂—, —[C(R⁷)₂]₂—, —C(R⁷═C(R⁷)—, —C(R⁷)═N—, —N═C(R⁷)—, —O—, —C(R⁷)₂—O—, —N(R⁶)—, —C(R⁷)₂—N(R⁶)—, —S—, —S(O)₂—, —C(R⁷)₂—S(O)₂—, —OC(O)N(R⁶)—, —N(R⁶)C(O)N(R⁶)—, —S(O)₂N(R⁶)— or —N(R⁶)S(O)₂N(R⁶)—;
M² is a bond, —C(R⁷)₂—, —[C(R⁷)₂]₂—, —C(R⁷═C(R⁷)—, —C(R⁷)═N—, —N═C(R⁷)—, —O—, —C(R⁷)₂—O—, —N(R⁶)—, —C(R⁷)₂—N(R⁶)—, —S—, —S(O)₂—, —C(R⁷)₂—S(O)₂—, —OC(O)N(R⁶)—, —N(R⁶)C(O)N(R⁶)—, —S(O)₂N(R⁶)— or —N(R⁶)S(O)₂N(R⁶)—, such that at least one of M¹ and M² is other than a bond, and such that the central ring of formula (I) that contains M¹ and M² has from 5 to 10 total ring atoms, and wherein two vicinal R⁷ groups of M¹ or M² together with the carbon atoms to which they are attached, can optionally join to form a 3- to 7-membered cycloalkyl group, a 3- to 7-membered heterocycloalkyl group, or a 5- to 6-membered heteroaryl group;
M³ is a bond, —C(R⁷)₂—, —[C(R⁷)₂]₂—, —C(R⁷═C(R⁷)—, —C(R⁷)═N—, —N═C(R⁷)—, —O—, —C(R⁷)₂—O—, —N(R⁶)—, —C(R⁷)₂—N(R⁶)—, —S—, —S(O)₂—, —C(R⁷)₂—S(O)₂—, —OC(O)N(R⁶)—, —N(R⁶)C(O)N(R⁶)—, —S(O)₂N(R⁶)— or —N(R⁶)S(O)₂N(R⁶)—;
M⁴ is a bond, —C(R⁷)₂—, —[C(R⁷)₂]₂—, —C(R⁷═C(R⁷)—, —C(R⁷)═N—, —N═C(R⁷)—, —O—, —C(R⁷)₂—O—, —N(R⁶)—, —C(R⁷)₂—N(R⁶)—, —S—, —S(O)₂—, —C(R⁷)₂—S(O)₂—, —OC(O)N(R⁶)—, —N(R⁶)C(O)N(R⁶)—, —S(O)₂N(R⁶)— or —N(R⁶)S(O)₂N(R⁶)—, such that at least one of M³ and M⁴ is other than a bond, and such that the central ring of formula (I) that contains M³ and M⁴ has from 5 to 10 total ring atoms, and wherein two vicinal R⁷ groups of M³ or M⁴ together with the carbon atoms to which they are attached, can optionally join to form a 3- to 7-membered cycloalkyl group, a 3- to 7-membered heterocycloalkyl group, or a 5- to 6-membered heteroaryl group;
Y¹ is —C—, and an optional and additional bond to Y¹ is present;
Y² is —C—, and an optional and additional bond to Y² is present;
Y³ is —C—, and an optional and additional bond to Y³ is present; and
Y⁴ is —C—, and an optional and additional bond to Y⁴ is present.

3. The compound of claim 1 having the formula: 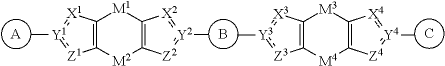
wherein the group: 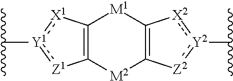
has the structure: 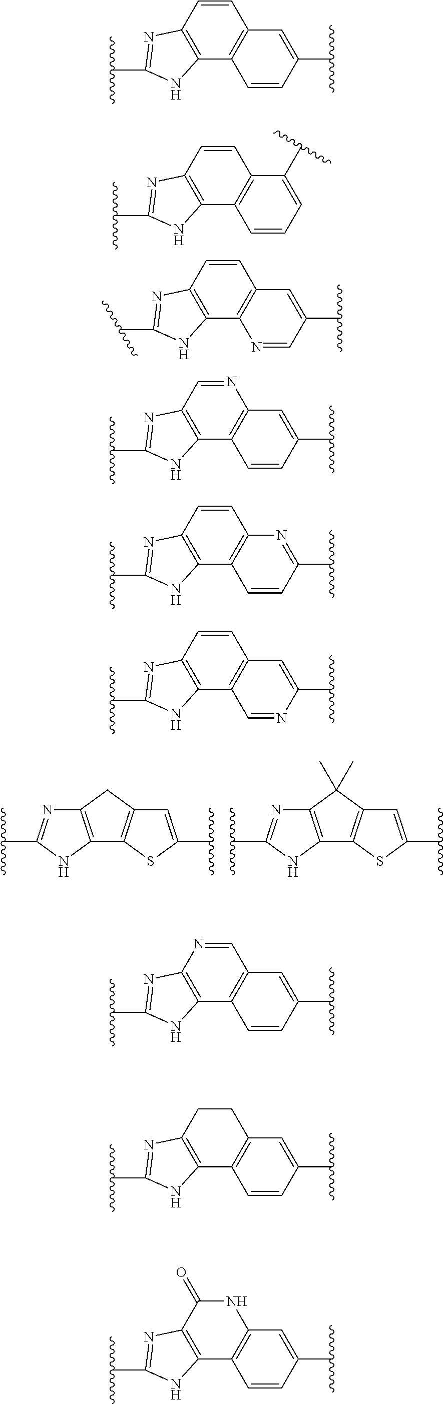 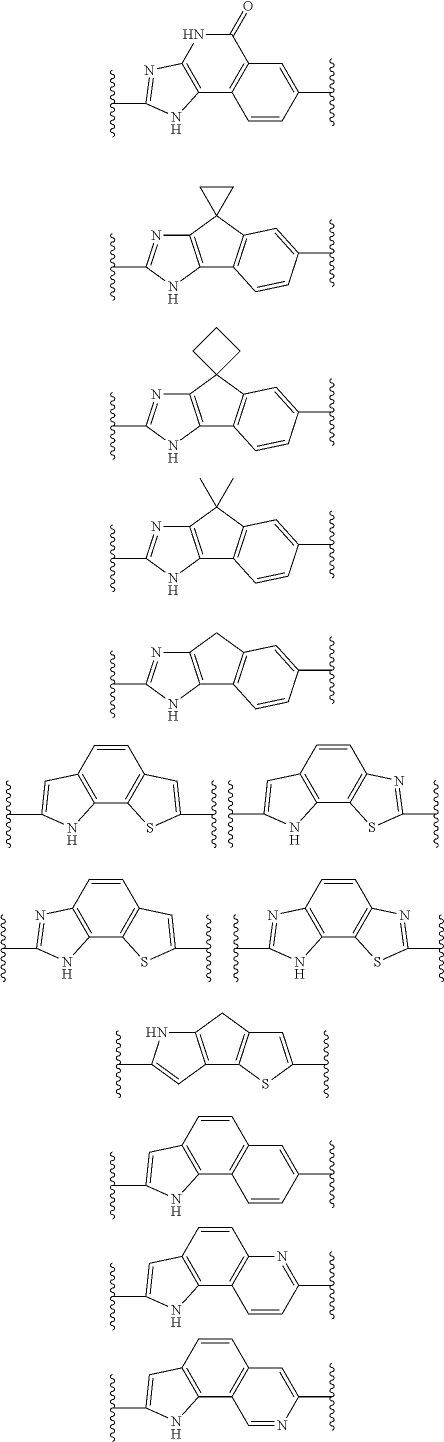 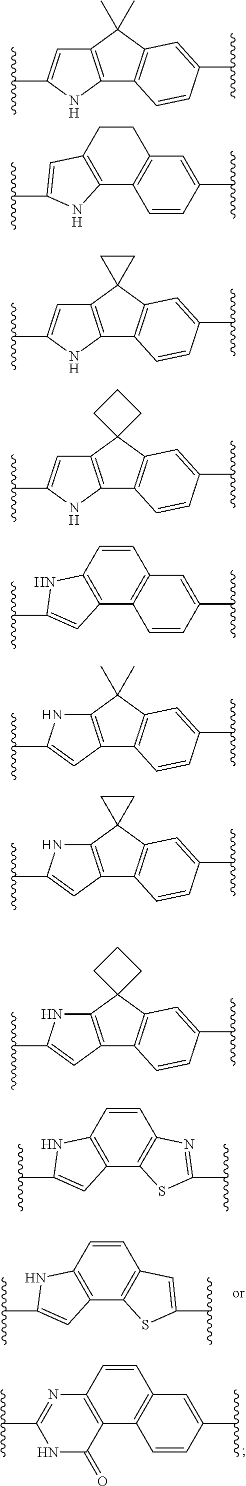
the group: 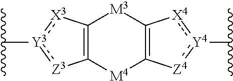
has the structure: 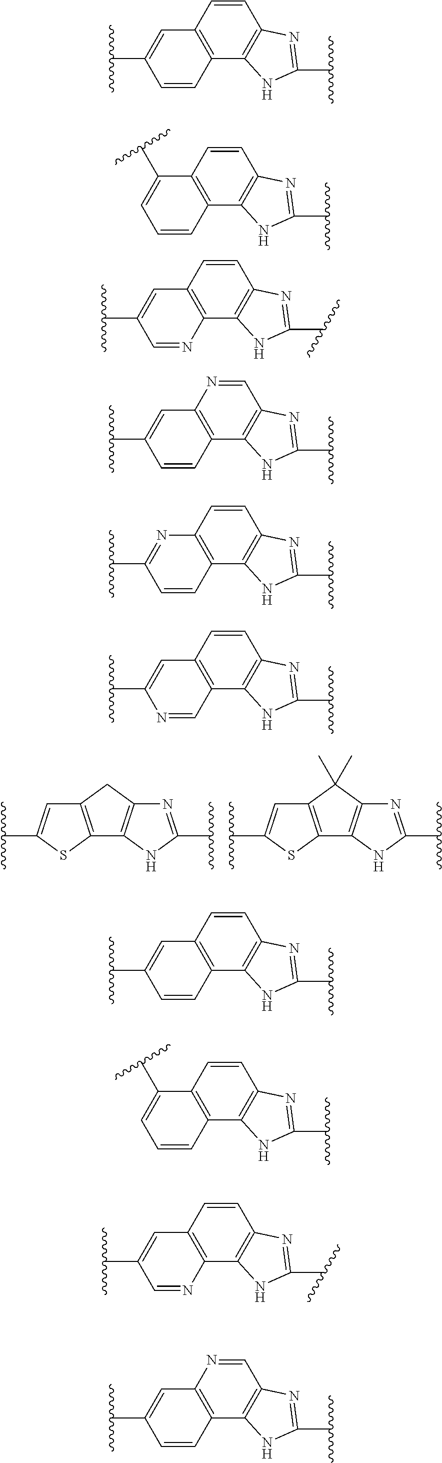 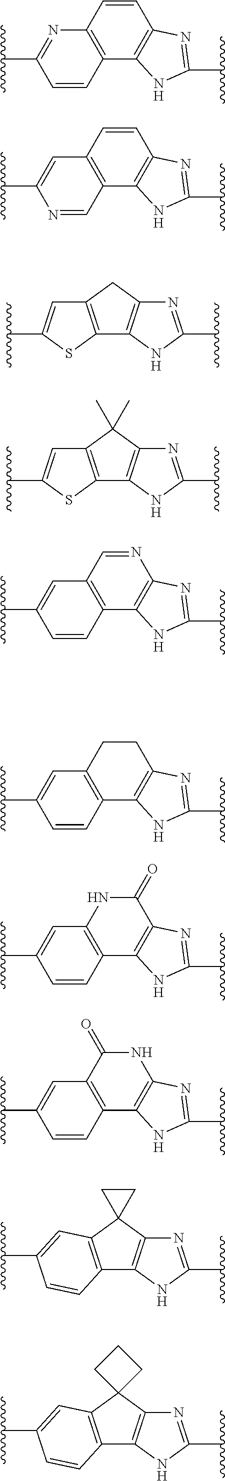 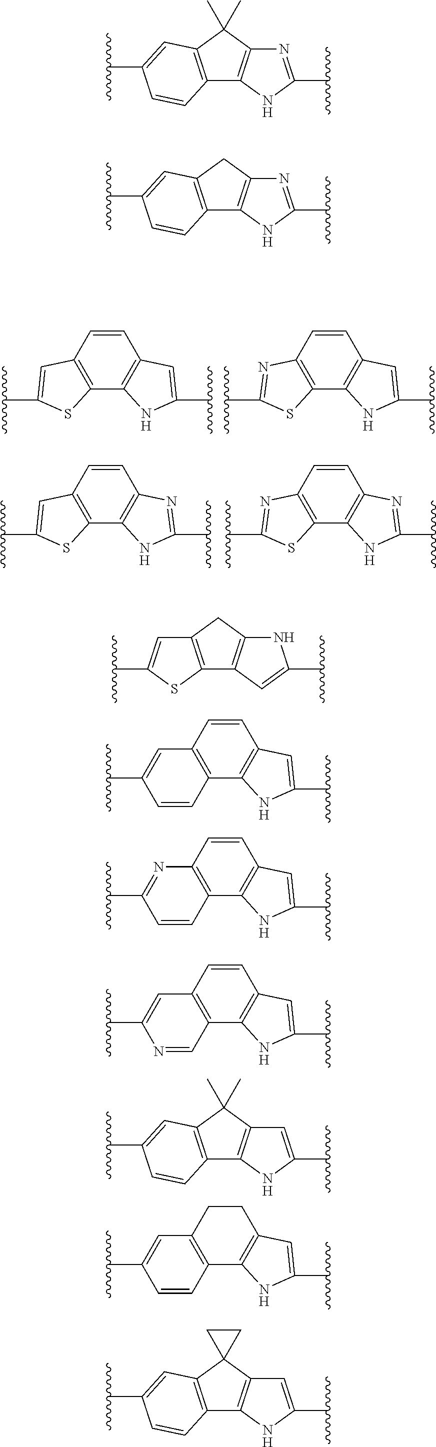 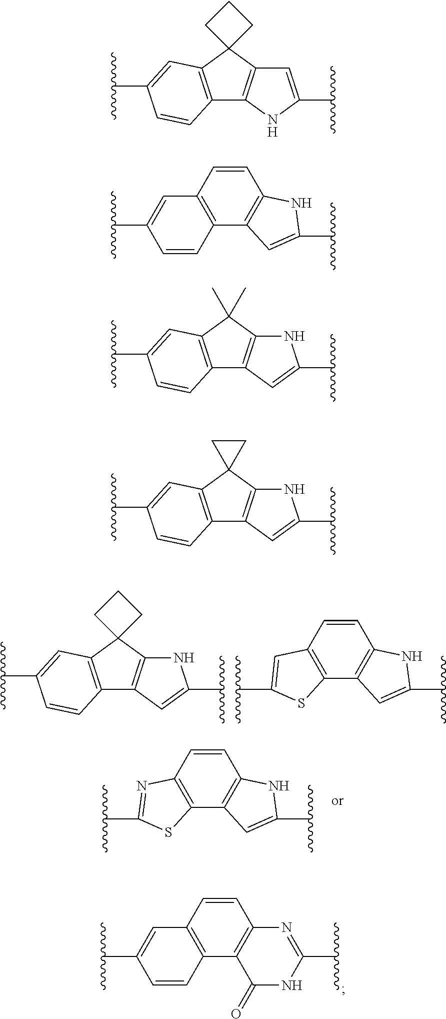
A is selected from: 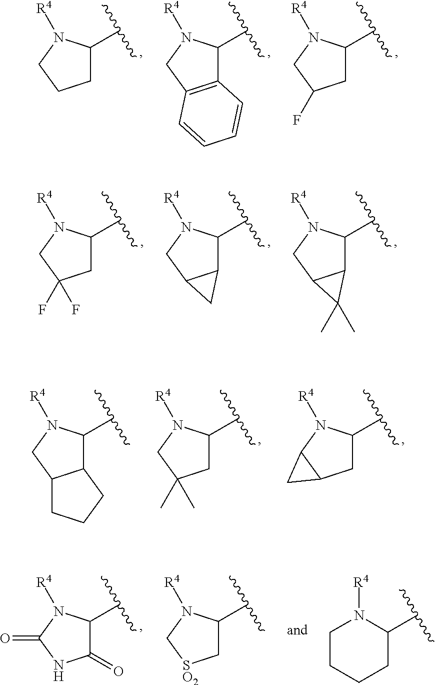
or A is —CH(R^{a})—N(R¹¹)(R¹³), wherein R^{a} is H, alkyl, haloalkyl, cycloalkyl or aryl;
B is a bond or phenylene, wherein a phenylene can be optionally and independently substituted with one or more R¹⁴ groups; and
C is selected from: 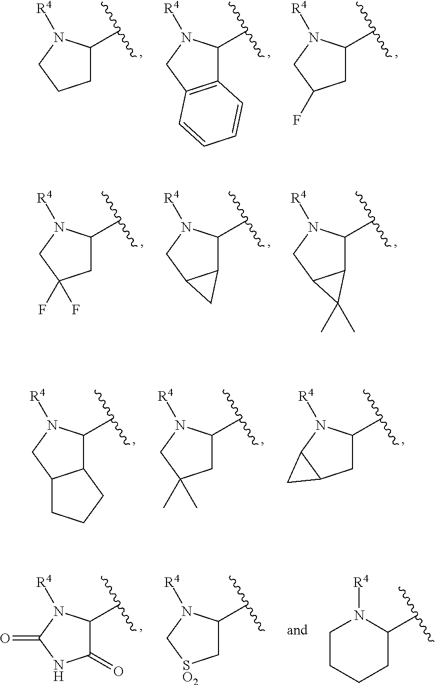
or C is —CH(R^{a})—N(R¹¹)(R¹³), wherein R^{a} is H, alkyl, haloalkyl, cycloalkyl or aryl.

4. The compound of claim 3, wherein the group: 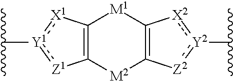
has the structure: 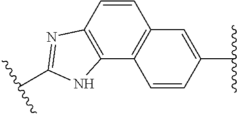
or the group 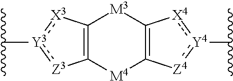
has the structure:

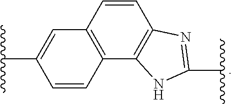

5. The compound of claim 3, wherein A and C are each:

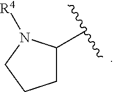

6. The compound of claim 5, wherein each occurrence of R⁴ is independently selected from:

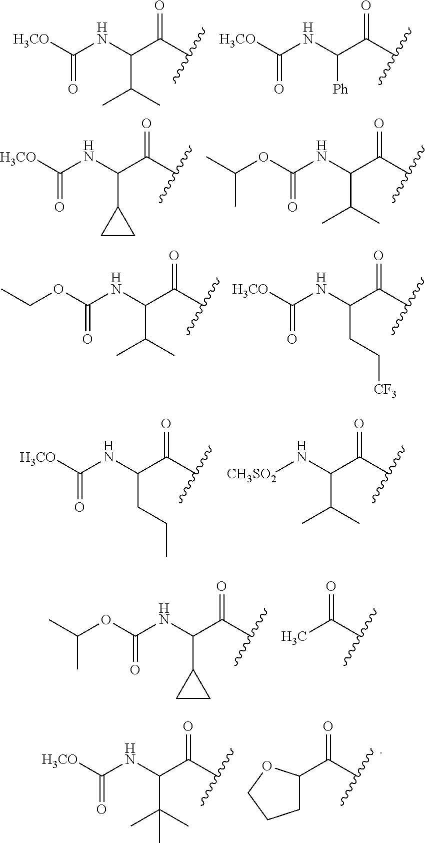

7. The compound of claim 5, wherein each occurrence of R⁴ is independently: 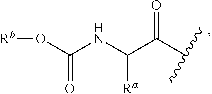
wherein R^{a} is H, alkyl, haloalkyl, cycloalkyl or aryl, and R^{b} is alkyl, haloalkyl or cycloalkyl.

8. The compound of claim 7, wherein each occurrence of R⁴ is:

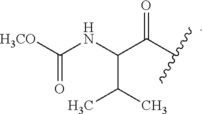

9. The compound of claim 8, wherein B is a bond.

10. A compound having the structure: 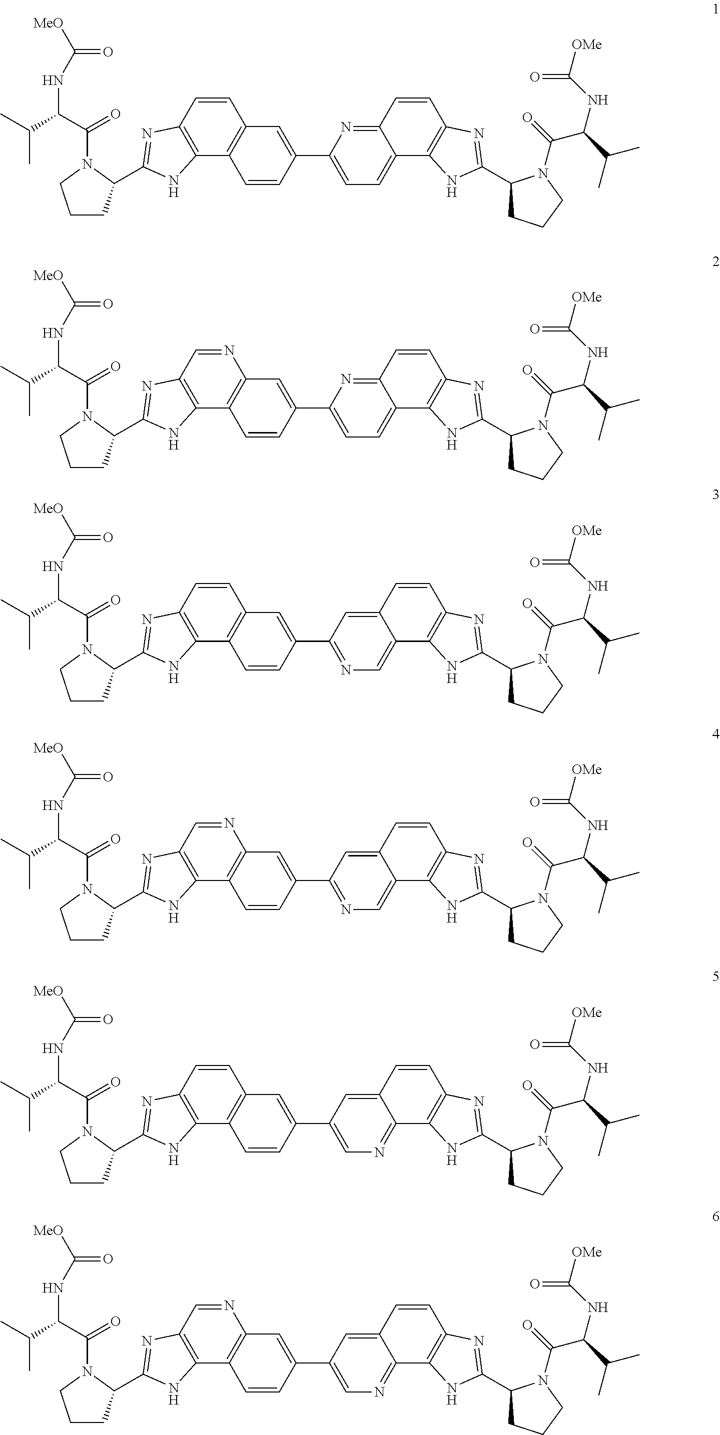 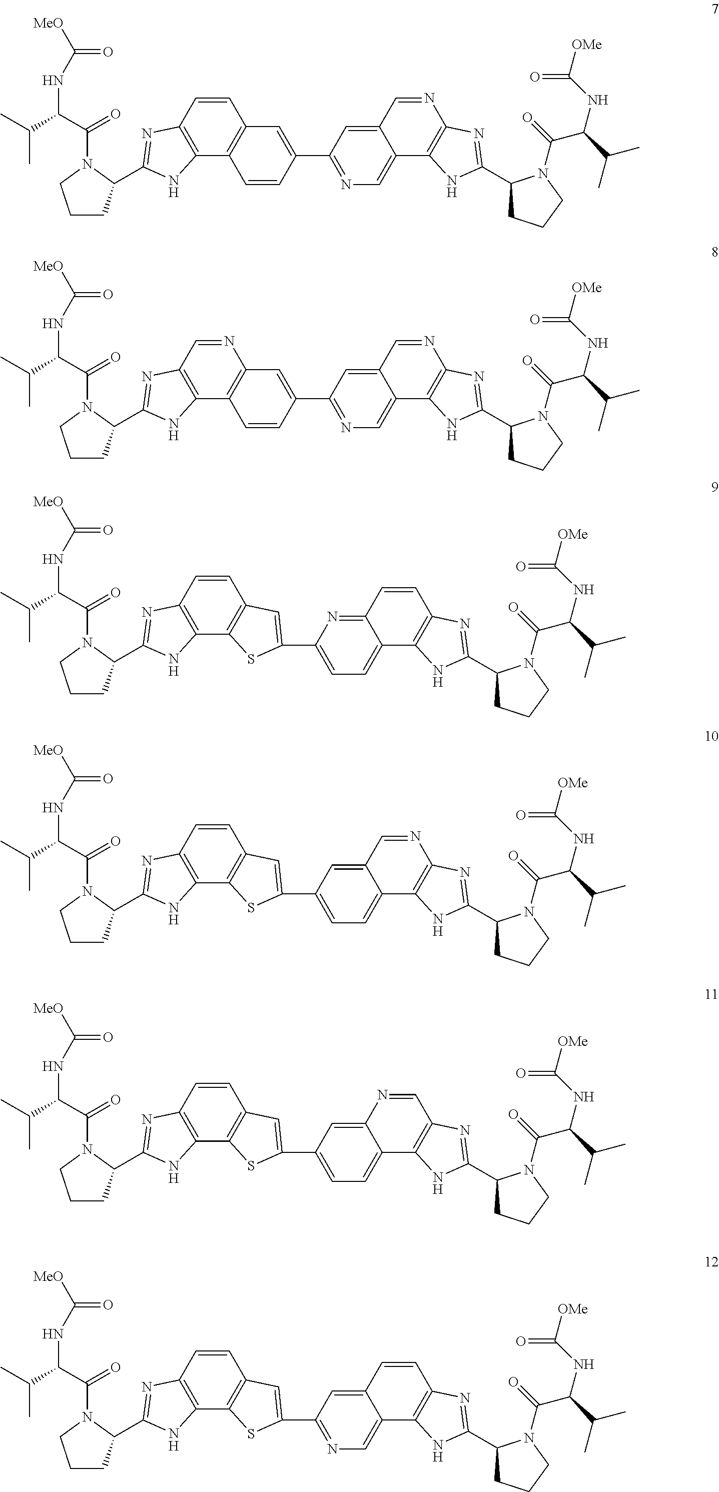 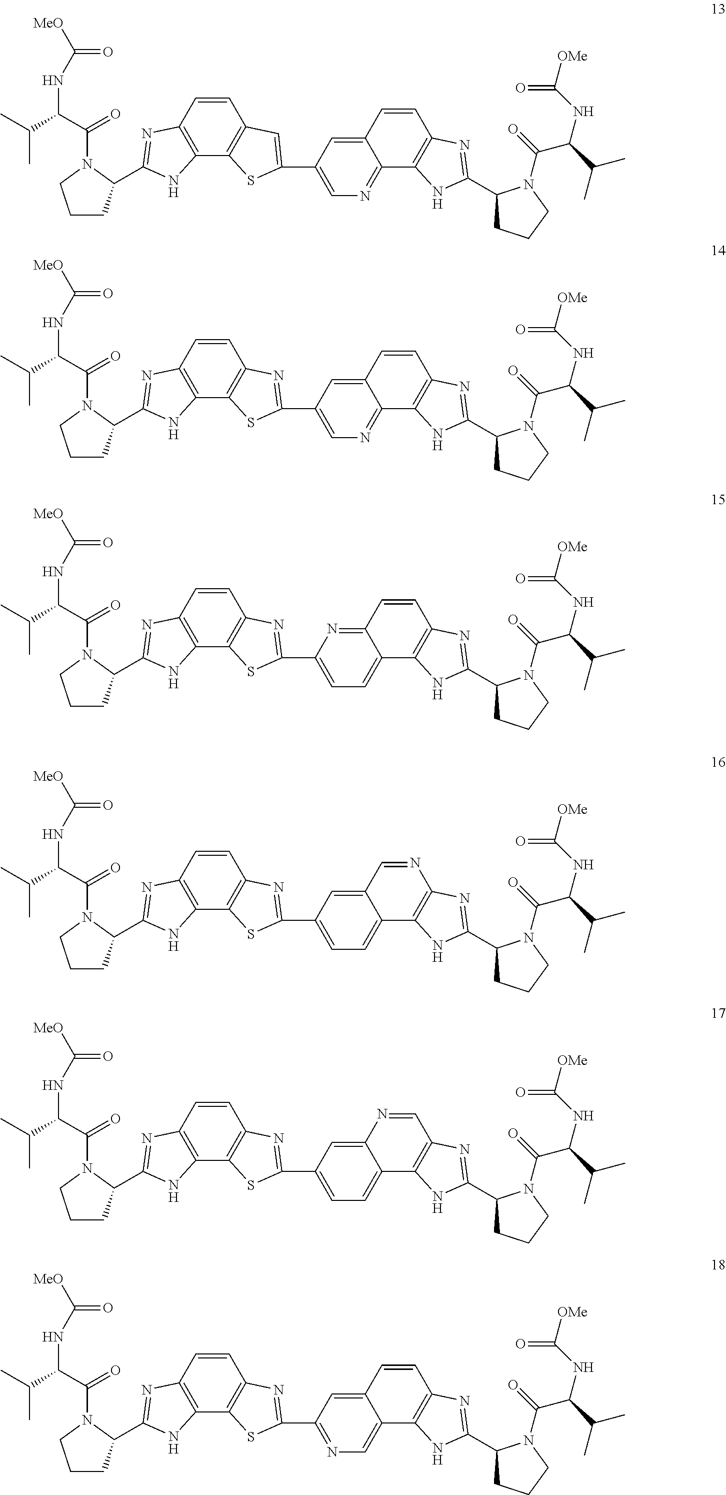 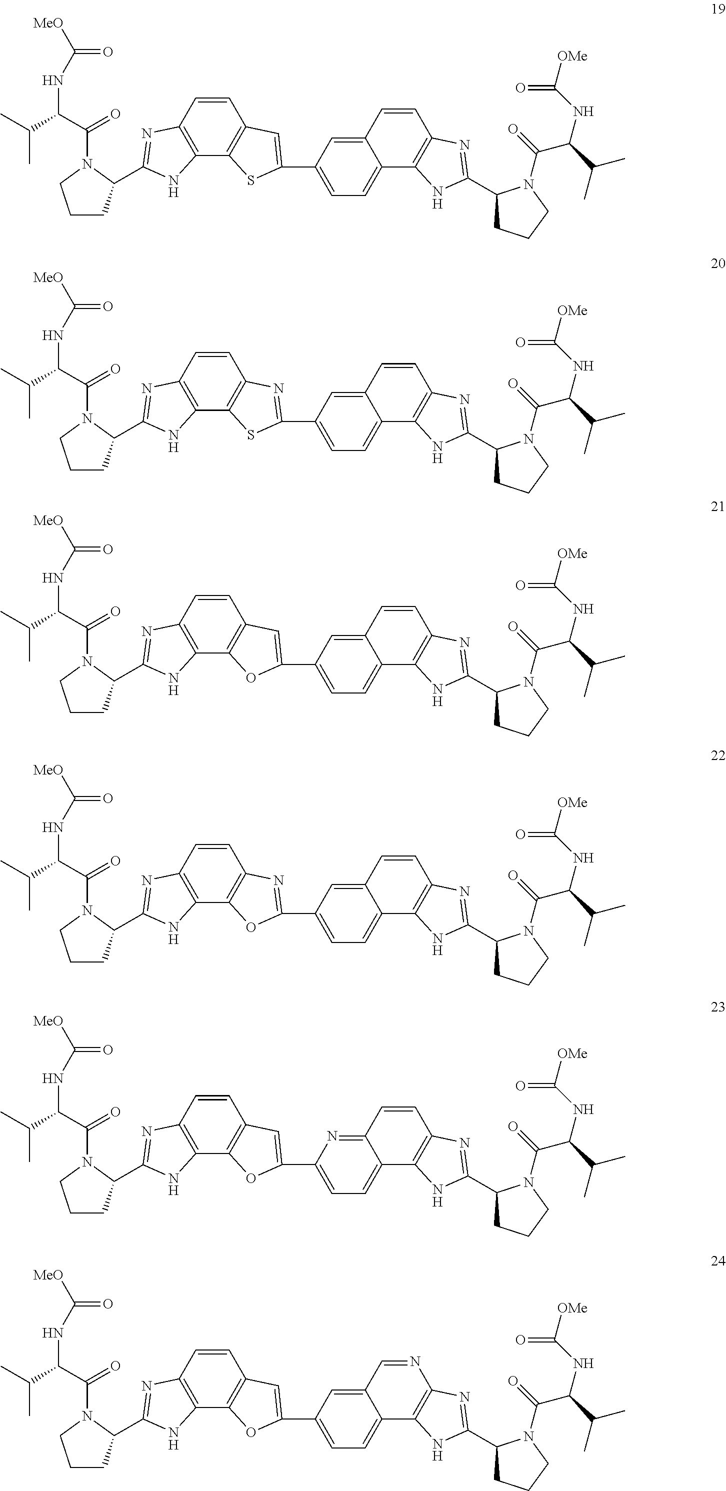 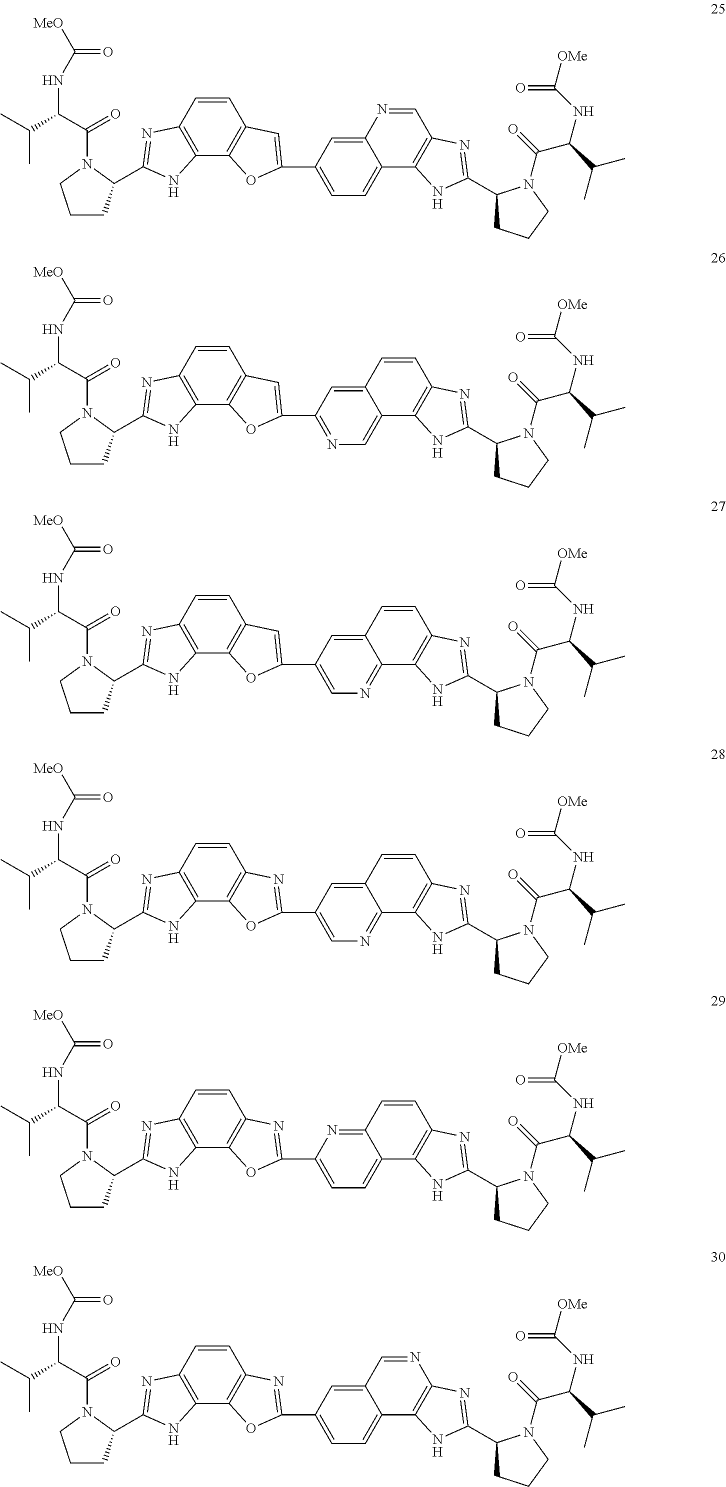 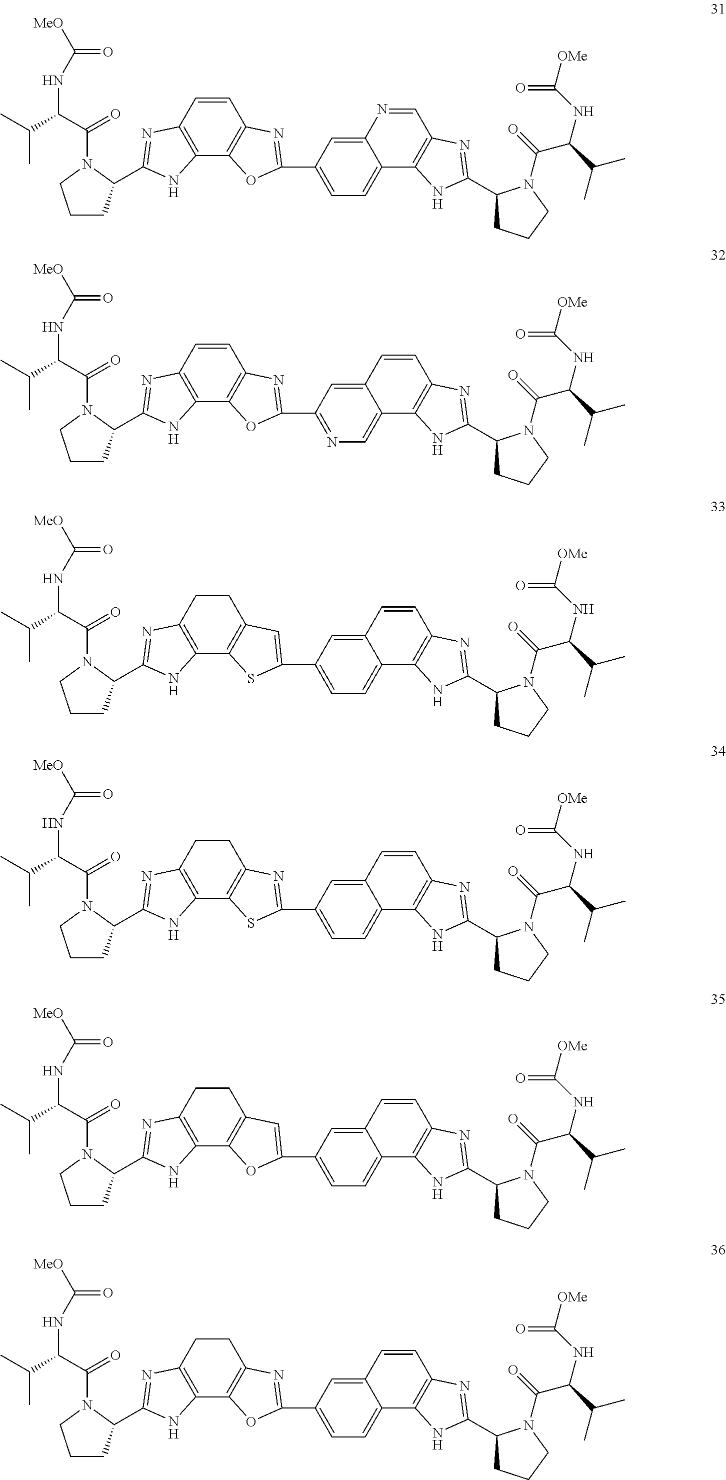 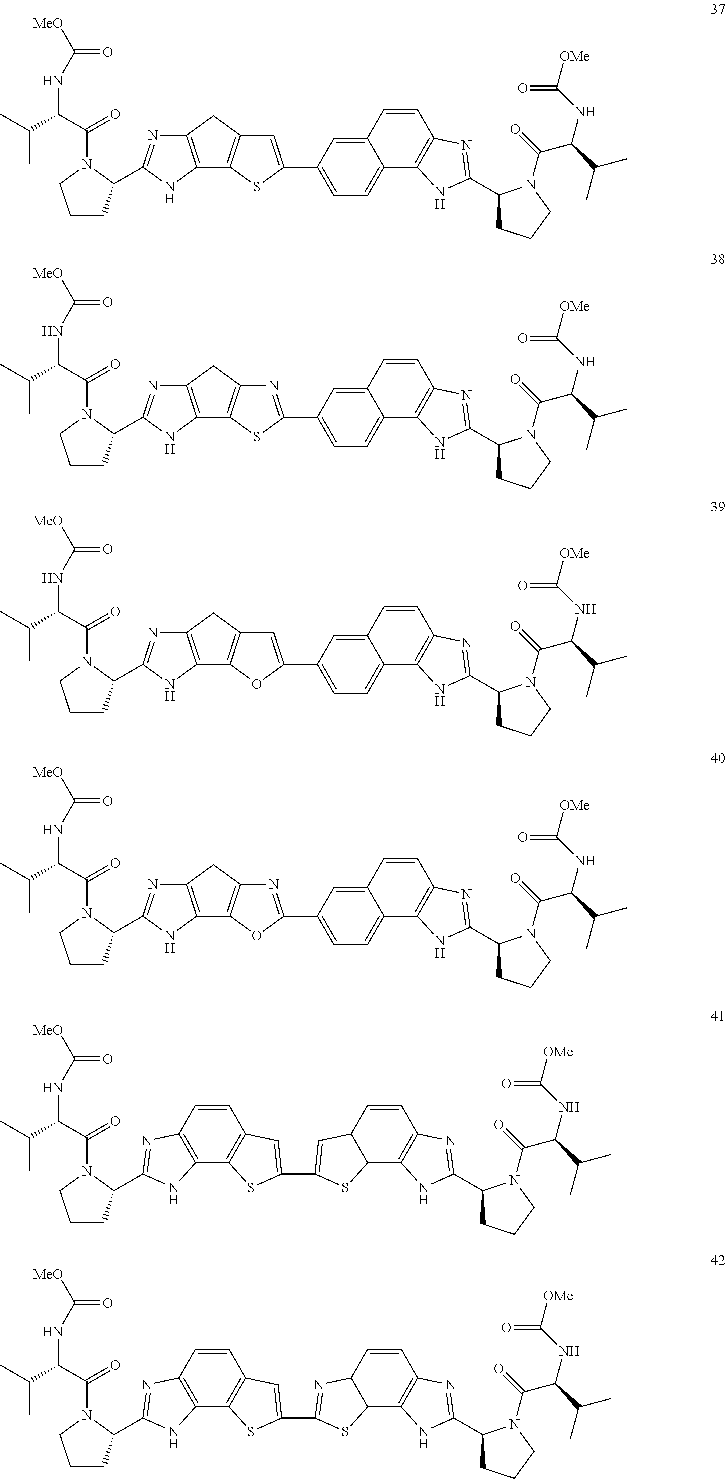 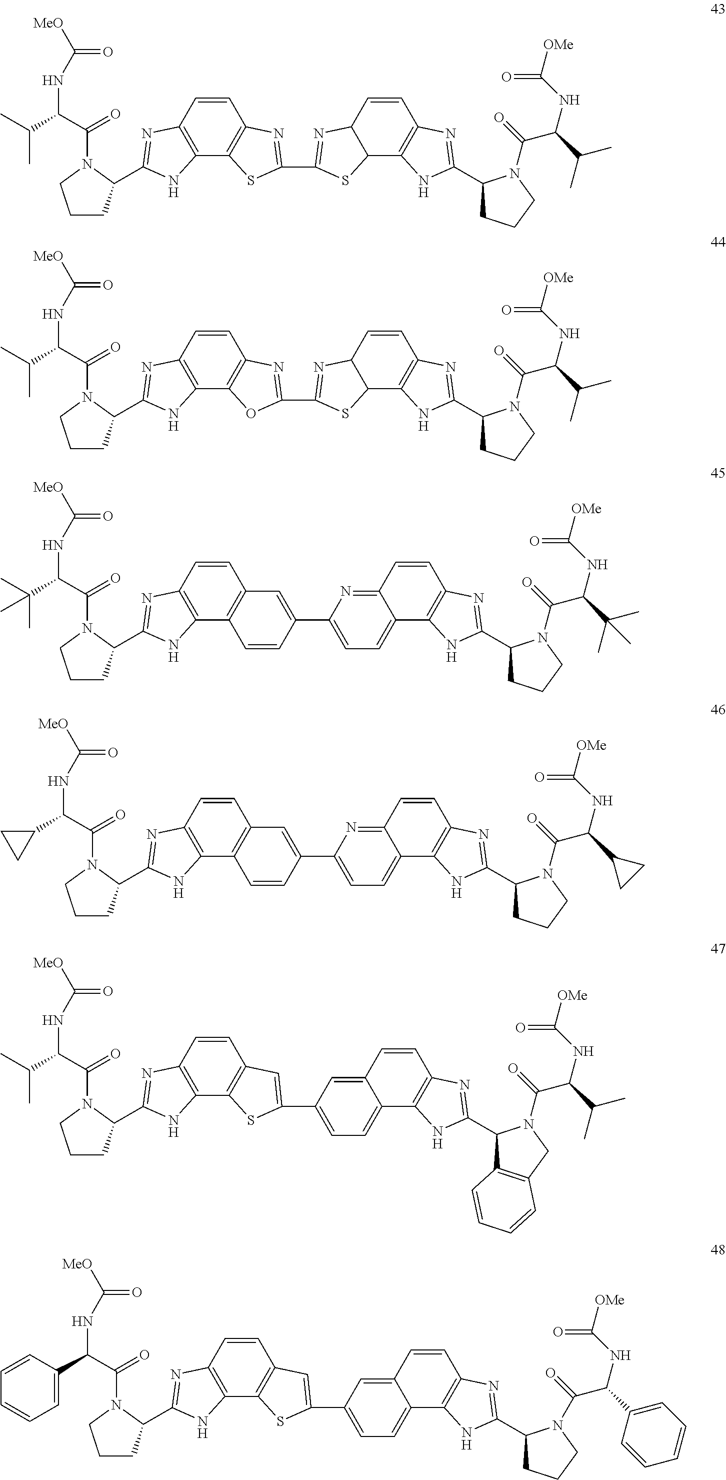 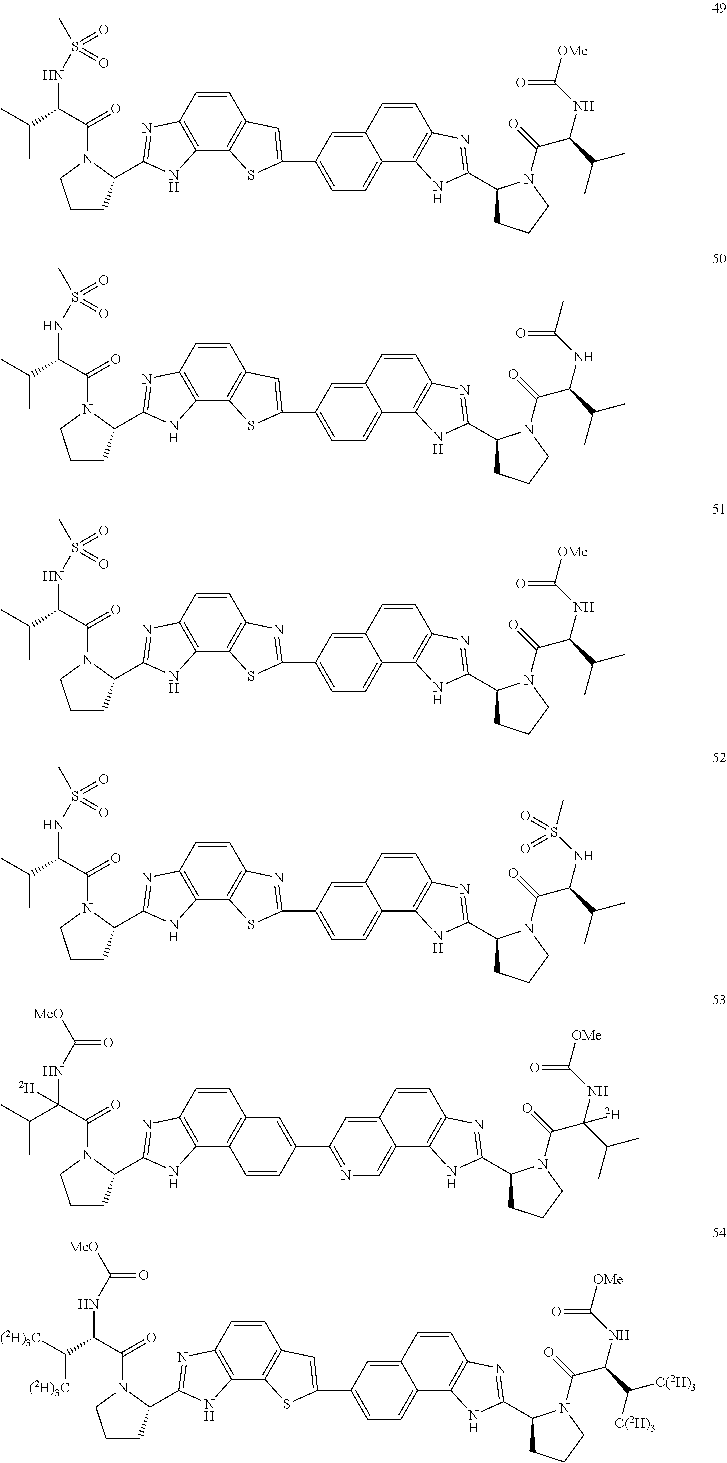 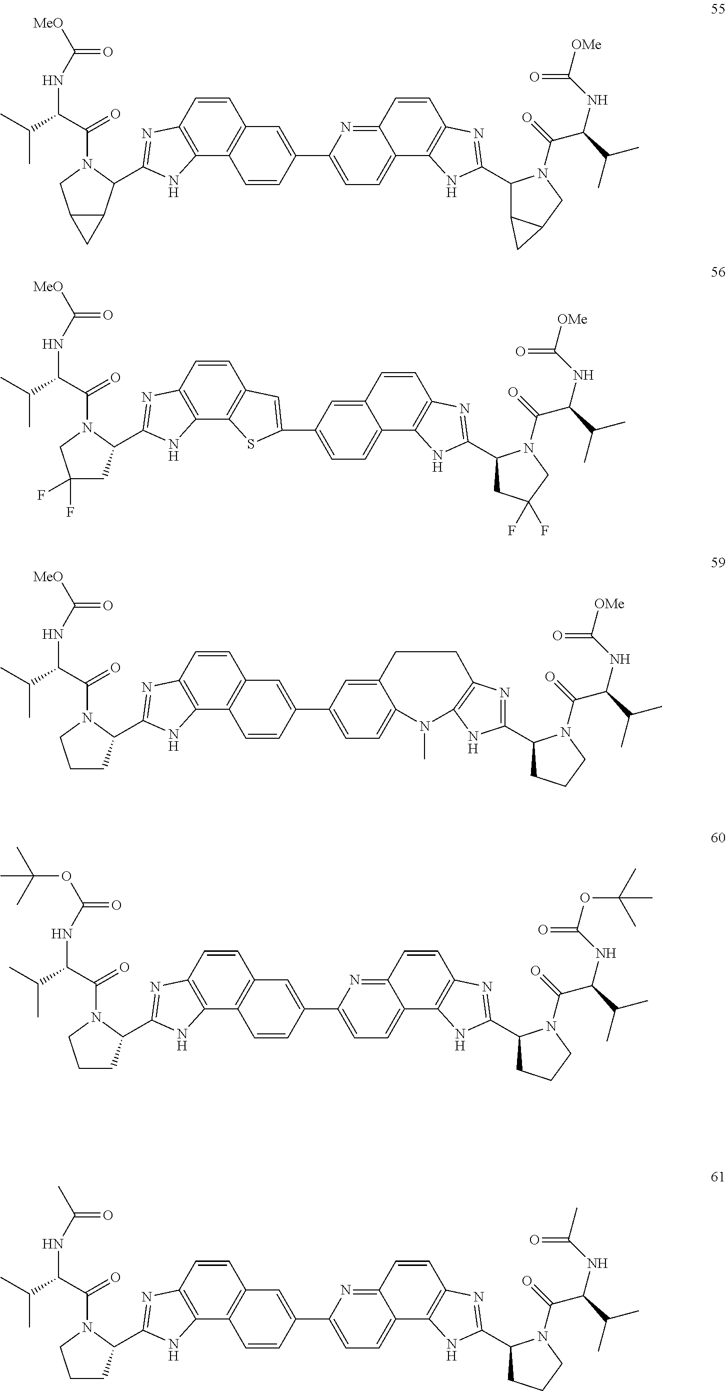 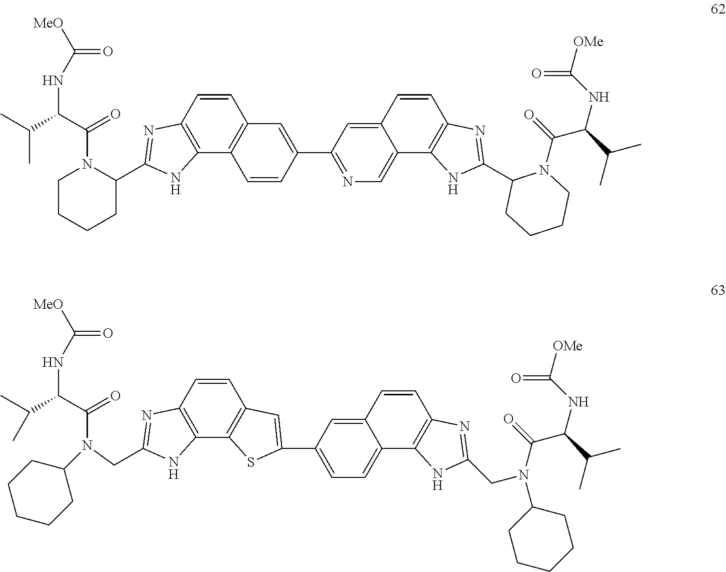
or a pharmaceutically acceptable salt thereof.

11. A pharmaceutical composition comprising at least one compound of claim 1 or a pharmaceutically acceptable salt thereof, and at least one pharmaceutically acceptable carrier.

12. The pharmaceutical composition of claim 11, further comprising at least one additional therapeutic agent, wherein the at least one additional therapeutic agent is not a compound of claim 1, wherein the at least one additional therapeutic agent is selected from: an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

13. A method for treating a viral infection in a patient, the method comprising administering to the patient an effective amount of at least one compound of claim 1 or a pharmaceutically acceptable salt thereof.

14. The method of claim 13, further comprising administering to the patient at least one additional therapeutic agent, wherein the at least one additional therapeutic agent is not a compound of claim 1, wherein the at least one additional therapeutic agent is selected from: an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.
